SEQUENCE LISTING (97 sequences)

SEQ ID NO: 1 - protein, 8 aa, synthetic construct
DYKDDDDK

SEQ ID NO: 2 - protein, 6 aa, synthetic construct
HHHHHH

SEQ ID NO: 3 - protein, 13 aa, synthetic construct
GAPVPYPDPLEPR

SEQ ID NO: 4 - DNA, 35 bp, synthetic construct
  source 1..35: mol_type = other DNA
cttagtgctagccaggtgcagctcgtggagacagg

SEQ ID NO: 5 - DNA, 59 bp, synthetic construct
  source 1..59: mol_type = other DNA
ggtgcgggatcctgaagagacggtgacctgaatgccctgaccccatgactcagggtgac

SEQ ID NO: 6 - DNA, 34 bp, synthetic construct
  source 1..34: mol_type = other DNA
gcctatgctagccaggtgcagctggcggagtcgg

SEQ ID NO: 7 - DNA, 35 bp, synthetic construct
  source 1..35: mol_type = other DNA
gaacacggatcctgaggagacggtcacctgggtcc

SEQ ID NO: 8 - DNA, 60 bp, synthetic construct
  source 1..60: mol_type = other DNA
gtggaggcggtagcggaggcggagggtcggctagccaactgcaactagtagaaagtgggg

SEQ ID NO: 9 - DNA, 59 bp, synthetic construct
  source 1..59: mol_type = other DNA
aagtcctcttcagaaataagcttttgttcggatcctgaggatactgttacttgcgtccc

SEQ ID NO: 10 - DNA, 35 bp, synthetic construct
  source 1..35: mol_type = other DNA
gcatcagctagccaggtgcagctcgtggagtcagg

SEQ ID NO: 11 - DNA, 35 bp, synthetic construct
  source 1..35: mol_type = other DNA
ggtgagggatcctgaggagacggtgacctgggtcc

SEQ ID NO: 12 - DNA, 21 bp, synthetic construct
  source 1..21: mol_type = other DNA
gttccagactacgctctgcag

SEQ ID NO: 13 - DNA, 23 bp, synthetic construct
  source 1..23: mol_type = other DNA
gattttgttacatctacactgtt

SEQ ID NO: 14 - DNA, 37 bp, synthetic construct
  source 1..37: mol_type = other DNA
ggagggtcggctagctaggtgcagctcgtggagacag

SEQ ID NO: 15 - DNA, 37 bp, synthetic construct
  source 1..37: mol_type = other DNA
ctgtctccacgagctgcacctagctagccgaccctcc

SEQ ID NO: 16 - DNA, 37 bp, synthetic construct
  source 1..37: mol_type = other DNA
ccatgggctggtagcgccagactccaggaaattcgcg

SEQ ID NO: 17 - DNA, 37 bp, synthetic construct
  source 1..37: mol_type = other DNA
cgcgaatttcctggagtctggcgctaccagcccatgg

SEQ ID NO: 18 - DNA, 45 bp, synthetic construct
  source 1..45: mol_type = other DNA
gagttggtcgcggtgatgaatctttagggcgtcataagctatggg

SEQ ID NO: 19 - DNA, 45 bp, synthetic construct
  source 1..45: mol_type = other DNA
cccatagcttatgacgccctaaagattcatcaccgcgaccaactc

SEQ ID NO: 20 - DNA, 43 bp, synthetic construct
  source 1..43: mol_type = other DNA
gtaatggtatgagactatagacccggggcagtgttcgtcaccc

SEQ ID NO: 21 - DNA, 43 bp, synthetic construct
  source 1..43: mol_type = other DNA
gggtgacgaacactgccccgggtctatagtctcataccattac

SEQ ID NO: 22 - DNA, 48 bp, synthetic construct
  source 1..48: mol_type = other DNA
ctatatacccggggcagtgtttagcaccctgagtcatggggtcagggc

SEQ ID NO: 23 - DNA, 48 bp, synthetic construct
  source 1..48: mol_type = other DNA
gccctgaccccatgactcagggtgctaaacactgccccgggtatatag

SEQ ID NO: 24 - DNA, 38 bp, synthetic construct
  source 1..38: mol_type = other DNA
ggtatgtcataaggtggtagcgccaggctccagggaag

SEQ ID NO: 25 - DNA, 44 bp, synthetic construct
  source 1..44: mol_type = other DNA
gcctggcggtaccaccttatgacctacctattaaacgtgaatcc

SEQ ID NO: 26 - DNA, 50 bp, synthetic construct
  source 1..50: mol_type = other DNA
cacgtttaataggtatgtcatatagtggtaccgccaggctccagggaagg

SEQ ID NO: 27 - DNA, 50 bp, synthetic construct
  source 1..50: mol_type = other DNA
ccttccctggagcctggcggtaccactatatgacatacctattaaacgtg

SEQ ID NO: 28 - skipped

SEQ ID NO: 29 - DNA, 38 bp, synthetic construct
  source 1..38: mol_type = other DNA
cttccctggagcctggcgctaccaccttatgacatacc

SEQ ID NO: 30 - DNA, 48 bp, synthetic construct
  source 1..48: mol_type = other DNA
ccgtctattactgcagcgccctcaattaggaagatatggaatattggg

SEQ ID NO: 31 - DNA, 48 bp, synthetic construct
  source 1..48: mol_type = other DNA
cccaatattccatatcttcctaattgagggcgctgcagtaatagacgg

SEQ ID NO: 32 - DNA, 46 bp, synthetic construct
  source 1..46: mol_type = other DNA
gcagcgccctcaatttagaagattaggaatattggggccaggggac

SEQ ID NO: 33 - DNA, 46 bp, synthetic construct
  source 1..46: mol_type = other DNA
gtcccctggccccaatattcctaatcttctaaattgagggcgctgc

SEQ ID NO: 34 - DNA, 42 bp, synthetic construct
  source 1..42: mol_type = other DNA
ggcggagggtcggctagctagctgcaactagtagaaagtggg

SEQ ID NO: 35 - DNA, 42 bp, synthetic construct
  source 1..42: mol_type = other DNA
cccactttctactagttgcagctagctagccgaccctccgcc

SEQ ID NO: 36 - DNA, 53 bp, synthetic construct
  source 1..53: mol_type = other DNA
gccgcatctgggaacatctagtccatttattacatgggctggtatagacaagc

SEQ ID NO: 37 - DNA, 53 bp, synthetic construct
  source 1..53: mol_type = other DNA
gcttgtctataccagcccatgtaataaatggactagatgttcccagatgcggc

SEQ ID NO: 38 - DNA, 46 bp, synthetic construct
  source 1..46: mol_type = other DNA
gggaacatcttctccatttagtacatgggctggtatagacaagccc

SEQ ID NO: 39 - DNA, 46 bp, synthetic construct
  source 1..46: mol_type = other DNA
gggcttgtctataccagcccatgtactaaatggagaagatgttccc

SEQ ID NO: 40 - DNA, 47 bp, synthetic construct
  source 1..47: mol_type = other DNA
gctggtatagacaagcccccggaaaatagagagaaatggtagctatc

SEQ ID NO: 41 - DNA, 47 bp, synthetic construct
  source 1..47: mol_type = other DNA
gatagctaccatttctctctattttccgggggcttgtctataccagc

SEQ ID NO: 42 - DNA, 47 bp, synthetic construct
  source 1..47: mol_type = other DNA
gtactattgtaacgcaggaaaactttagagaaccaccgggtgggggc

SEQ ID NO: 43 - DNA, 47 bp, synthetic construct
  source 1..47: mol_type = other DNA
gcccccacccggtggttctctaaagttttcctgcgttacaatagtac

SEQ ID NO: 44 - DNA, 31 bp, synthetic construct
  source 1..31: mol_type = other DNA
gggctagacgattagtggggtcaggggacgc

SEQ ID NO: 45 - DNA, 31 bp, synthetic construct
  source 1..31: mol_type = other DNA
gcgtcccctgaccccactaatcgtctagccc

SEQ ID NO: 46 - DNA, 37 bp, synthetic construct
  source 1..37: mol_type = other DNA
ggagggtcggctagctaggtgcagctcgtggagtcag

SEQ ID NO: 47 - DNA, 37 bp, synthetic construct
  source 1..37: mol_type = other DNA
ctgactccacgagctgcacctagctagccgaccctcc

SEQ ID NO: 48 - DNA, 52 bp, synthetic construct
  source 1..52: mol_type = other DNA
gaaatggtcgcagatatttagtctggcggtagtacagactatgcaggctccg

SEQ ID NO: 49 - DNA, 52 bp, synthetic construct
  source 1..52: mol_type = other DNA
cggagcctgcatagtctgtactaccgccagactaaatatctgcgaccatttc

SEQ ID NO: 50 - DNA, 50 bp, synthetic construct
  source 1..50: mol_type = other DNA
gcagatattttttctggcggttagacagactatgcaggctccgtgaaggg

SEQ ID NO: 51 - DNA, 50 bp, synthetic construct
  source 1..50: mol_type = other DNA
cccttcacggagcctgcatagtctgtctaaccgccagaaaaaatatctgc

SEQ ID NO: 52 - DNA, 40 bp, synthetic construct
  source 1..40: mol_type = other DNA
cagactatgcaggctccgtgtagggccgattcaccatctc

SEQ ID NO: 53 - DNA, 40 bp, synthetic construct
  source 1..40: mol_type = other DNA
gagatggtgaatcggccctacacggagcctgcatagtctg

SEQ ID NO: 54 - DNA, 37 bp, synthetic construct
  source 1..37: mol_type = other DNA
ggagcggtgactactagggccaggggacccaggtcac

SEQ ID NO: 55 - DNA, 37 bp, synthetic construct
  source 1..37: mol_type = other DNA
gtgacctgggtcccctggccctagtagtcaccgctcc

SEQ ID NO: 56 - DNA, 54 bp, synthetic construct
  source 1..54: mol_type = other DNA
ggaaccctggtcaccgtctcctcgggatccgaacaaaagcttatttctgaagaa

SEQ ID NO: 57 - DNA, 58 bp, synthetic construct
  source 1..58: mol_type = other DNA
gttacatctacactgttgttatcagatctcgagctattaatggtgatggtggtgatgg

SEQ ID NO: 58 - DNA, 40 bp, synthetic construct
  source 1..40: mol_type = other DNA
caagagagaagctcggccggctagccaggtgcagctggcg

SEQ ID NO: 59 - DNA, 46 bp, synthetic construct
  source 1..46: mol_type = other DNA
tcagaaataagcttttgttcggatcctgaggagacggtcacctggg

SEQ ID NO: 60 - DNA, 20 bp, synthetic construct
  source 1..20: mol_type = other DNA
gttttgattgtcttgttggc

SEQ ID NO: 61 - DNA, 22 bp, synthetic construct
  source 1..22: mol_type = other DNA
catgggaaaacatgttgtttac

SEQ ID NO: 62 - DNA, 366 bp, synthetic construct
  source 1..366: mol_type = other DNA
caactgcaactagtagaaagtgggggcgggcttgtacaacccggtggaagcctacgtctatcatgtgccgcatctgggaacatcttctccatttattacatgggctggtatagacaagcccccggaaaacagagagaaatggtagctatcatcaatagtaacggtatcactaactatggggatttcgttaaaggcagatttaccattagtagagacaacgctgagaacagtgcttatctacaaatgaataaccttacgccggaggataccgcagtgtactattgtaacgcaggaaaacttaggagaaccaccgggtgggggctagacgattattggggtcaggggacgcaagtaacagtatcctca

SEQ ID NO: 63 - DNA, 125 bp, synthetic construct
  source 1..125: mol_type = other DNA
ggatccgaacaaaagcttatttctgaagaagacttgggtggtggagggtccggaggactgcccgaaacgggcggccatcaccaccatcaccattaatagctcgagatctgataacaacagtgtag

SEQ ID NO: 64 - protein, 122 aa, synthetic construct
QVQLVETGGGLVQAGGSLRLSCTASGADFSFYAMGWYRQTPGNSRELVAVMNLNGVISYGDSARGRFDISRDGTKNIVFLQMNSLKPEDTGVYYCNGMRLYTRGSVRHPESWGQGIQVTVSS

SEQ ID NO: 65 - protein, 117 aa, synthetic construct
QVQLAESGGGLVQPGGSLRLSCAASGFTFNRYVIRWYRQAPGKERELVAGISRSGDSGRYVDSVKGRFTISRDNDKNMAYLQMSSLKPDDTAVYYCSALNLEDMEYWGQGTQVTVSS

SEQ ID NO: 66 - protein, 122 aa, synthetic construct
QLQLVESGGGLVQPGGSLRLSCAASGNIFSIYYMGWYRQAPGKQREMVAIINSNGITNYGDFVKGRFTISRDNAENSAYLQMNNLTPEDTAVYYCNAGKLRRTTGWGLDDYWGQGTQVTVSS

SEQ ID NO: 67 - protein, 114 aa, synthetic construct
QVQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSS

SEQ ID NO: 68 - protein, 115 aa, synthetic construct
SGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSS

SEQ ID NO: 69 - protein, 111 aa, synthetic construct
SGGGSVQPGGSLRLSCAAIGSVFTMYTTAWYRQTPGNLRELVASITDEHRTNYAASAEGRFTISRDNAKHTVDLQMTNLKPEDTAVYYCKLEHDLGYYDYWGQGTQVTVSS

SEQ ID NO: 70 - protein, 119 aa, synthetic construct
SGGGLVQPGGSLRLSCATSGFTLEYYAIGWFRQAPGKGREGVACMNSSGGGTNYADSVKGRFTISRDNAKKMVYLQMNSLKSEDTAVYYCVVDDFRCGSRWAAYLRSSWGQGTQVTVSS

SEQ ID NO: 71 - protein, 118 aa, synthetic construct
SGGGLVQPGGSLRLSCVVSGSDFNTYIMGWYRQVPGKPRELVADITTEGKTNYGGSVKGRFTISRDNAKNTVYLQMFGLKPEDAGNYVCNADWKMGAWTAGDYGIDYWGKGTLVTVSS

SEQ ID NO: 72 - protein, 114 aa, synthetic construct
SGGGLVQPGGSLRLSCAASGFTLGSRYMSWVRQAPGEGFEWVSSIEPSGTAWDGDSAKGRFTTSRDDAKNTLYLQMSNLQPEDTGVYYCATGYRTDTRIPGGSWGQGTQVTVSS

SEQ ID NO: 73 - protein, 108 aa, synthetic construct
SGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSS

SEQ ID NO: 74 - protein, 118 aa, synthetic construct
SGGGLVQPGGSLRLSCAASGFTLDDYAIGWFRQVPGKEDEGVSCMSRSGDTYYPHSVKGRFTISVDNAKNTMYLQMNNLKPEDTAVYYCAIDFPPVRPMCIQAAPKKRSRGTQVTVSA

SEQ ID NO: 75 - protein, 116 aa, synthetic construct
TGGGLVQAGGSLRLSCTASGADFSFYAMGWYRQTPGNSRELVAVMNLNGVISYGDSARGRFDISRDGTKNIVFLQMNSLKPEDTGVYYCNGMRLYTRGSVRHPESWGQGIQVTVSS

SEQ ID NO: 76 - protein, 124 aa, synthetic construct
TGGGLVQAGDSLTLSCAATGRTLDYYALGWFRQVPGNKREFVAAINWLGGSTYYADSVRGRFTLSRDNSKSTLYLNMNNLIPDDTAVYYCAADFSIAYSGTYPPAYAEYDYDYWGQGTQVTVSS

SEQ ID NO: 77 - protein, 115 aa, synthetic construct
SGGLVQPGGSLKLSCAHSGSPLSIWVMGWYRQAPGKQRELVALINLNGITSYGDSVKGRFTISRDYAENTAYLQMNSLKFEDTAVYYCNAEPLGPRGKKSGKEYWGTGTQVTVSL

SEQ ID NO: 78 - protein, 105 aa, synthetic construct
TGGALVQPGQSLTLSCTTSENVFGIYGMAWLRQAPGRQRELVASITSRGTAHYHDSVKGRFTISRESGKTTAYLQTTSVNPEDTAIYYCNSGPYWGQGTQVTVSS

SEQ ID NO: 79 - protein, 111 aa, synthetic construct
SGGGLVQPGGSLRLSCAASGFTFNRYVIRWYRQAPGKERELVAGISRSGDSGRYVDSVKGRFTISRDNDKNMAYLQMSSLKPDDTAVYYCSALNLEDMEYWGQGTQVTVSS

SEQ ID NO: 80 - protein, 116 aa, synthetic construct
SGGGLVQPGGSLRLSCAASGNIFSIYYMGWYRQAPGKQREMVAIINSNGITNYGDFVKGRFTISRDNAENSAYLQMNNLTPEDTAVYYCNAGKLRRTTGWGLDDYWGQGTQVTVSS

SEQ ID NO: 81 - protein, 119 aa, synthetic construct
SGGGTVQPGGTLRLSCAASGFTLDEYAIGWFRQAPGKEREGVSCISSSASISYADSVKGRFTISRDNAKNTVYLTMNSLKPEDTGVYYCARAFLACGPVAGWGTEYDYWGQGTQVTVSS

SEQ ID NO: 82 - protein, 115 aa, synthetic construct
TGGGLVQPGGSLRLSCTASTTISDFYSMGWFRQTPGNQRELVAIVRRGGDTKSGDSVKGRFTISRDNTRSTVYLQMDNLKPEDTAVYYCYANLQKSSDELGPYYWGQGTQVTVSS

SEQ ID NO: 83 - protein, 119 aa, synthetic construct
SGGGLVQSGGSLRLSCAASLLTLEYYAIGWFRQAPGKEREGVSCTGSSGGSTVYIDSVKGRFTVVRDNAKNMVYLQMDNLQPEDTAVYYCAADDLRCGRGWSSYFRGSWGQETQVTVSS

SEQ ID NO: 84 - protein, 111 aa, synthetic construct
TGGGLVQPGGSLRLACVASESVFEMYTVAWYRQAPGKQRELVAGITDEGRTNYADFVKGRFTISRDNSKKTVHLQMDNLNPEDTAVYYCKLEHDLGYYDYWGQGTQVTVSS

SEQ ID NO: 85 - protein, 121 aa, synthetic construct
SGGGLVQPGGSLRLSCAASGLTLDYYAIGWFRQAPGKEREGVSCISSGSSMSIHADSVKGRFTISRDNAKNTVYLQMNSLKPEDTAVYYCAADDFTCGSRWSDWAHTFGFWGQGTQVTVSS

SEQ ID NO: 86 - protein, 116 aa, synthetic construct
SGGLVQPGGSLTLSCVVSGGIFSTYIMGWYRQVPGRQREMVATISNHTTDYADFVQGRFTISRDIAKKAVYLQMHSLKPDDTGRYVCNADWMVGAWTAGDYGVDYWGKGILVTVSS

SEQ ID NO: 87 - protein, 491 aa, synthetic construct
MGHHHHHHHHGAGENLYFQGAGGPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLSVRGIITSKGAGENLYFQGAGWSHPQFEKGAGWSHPQFEK

SEQ ID NO: 88 - DNA, 1473 bp, synthetic construct
  source 1..1473: mol_type = other DNA
atgggtcatcatcatcatcatcatcatcacggtgcgggcgagaatctgtacttccagggcgccggcggtccgtttgtgaacaagcagttcaactacaaagacccggtgaacggcgtggatatcgcttacatcaagatcccgaacgccggtcagatgcagccggtgaaggctttcaaaatccacaacaaaatctgggttatcccggagcgcgacaccttcaccaacccggaggaaggcgatctgaacccgccgccggaggccaagcaggtgccggtgagctactacgatagcacctacctgagcaccgacaacgagaaagataactacctgaagggtgtgaccaaactgttcgaacgtatctacagcaccgacctgggccgcatgctgctgaccagcatcgtgcgtggtatcccgttctggggcggtagcaccatcgacaccgagctgaaagtgatcgataccaactgcatcaacgtgatccagccggacggcagctaccgtagcgaggagctgaacctggttatcatcggtccgagcgccgatatcatccagttcgagtgcaagagcttcggccacgaagtgctgaacctgacccgcaacggctacggtagcacccagtacatccgtttcagcccggacttcaccttcggtttcgaggaaagcctggaagtggataccaacccgctgctgggtgctggcaagtttgctaccgacccagctgtgaccctggctcacgaactgattcacgctggtcaccgtctgtatggtatcgctatcaacccgaaccgcgtgttcaaggtgaacaccaacgcctactacgagatgagcggcctggaagtgagcttcgaggaactgcgcaccttcggcggtcacgacgcgaaattcatcgatagcctgcaggagaacgaattccgtctgtactactacaacaagttcaaagatatcgccagcaccctgaacaaggcgaaaagcatcgtgggcaccaccgcgagcctgcagtacatgaagaacgtgttcaaggagaaatacctgctgagcgaagacaccagcggtaaattcagcgtggacaagctgaaattcgataagctgtacaaaatgctgaccgagatctacaccgaagacaacttcgtgaagttcttcaaagtgctgaaccgcaagacctacctgaacttcgataaggccgtgttcaagatcaacatcgtgccgaaagtgaactacaccatctacgacggcttcaacctgcgtaacaccaacctggccgcgaacttcaacggtcagaacaccgagatcaacaacatgaacttcaccaagctgaaaaacttcaccggcctgttcgaattctacaagctgctgagcgtgcgcggtatcatcaccagcaaaggtgcgggcgagaacctgtacttccagggtgctggctggagccacccgcagttcgaaaagggtgctggttggagccatccgcagtttgagaaa

SEQ ID NO: 89 - DNA, 366 bp, synthetic construct
  source 1..366: mol_type = other DNA
caggtgcagctcgtggagacagggggaggcttggttcaggctggggggtctctgagactctcctgtacagcctctggagccgacttcagtttctatgccatgggctggtaccgccagactccaggaaattcgcgcgagttggtcgcggtgatgaatcttaatggcgtcataagctatggggactccgctcggggccgattcgacatctccagggacggcaccaagaacatagtgtttctgcaaatgaacagcctgaaacctgaagacacgggcgtttattactgtaatggtatgagactatatacccggggcagtgttcgtcaccctgagtcatggggtcaggggatccaggtcaccgtctcttca

SEQ ID NO: 90 - DNA, 351 bp, synthetic construct
  source 1..351: mol_type = other DNA
caggtgcagctggcggagtcgggaggaggcctggtgcagccgggggggtctctgagactctcctgtgcagcctccggattcacgtttaataggtatgtcataaggtggtaccgccaggctccagggaaggagcgcgaattggtcgcaggtatttcgcggtctggtgattctggaaggtatgtcgactccgtgaagggccgattcaccatctccagagacaatgacaagaacatggcctatctacaaatgagctccctaaaacctgacgacacggccgtctattactgcagcgccctcaatttagaagatatggaatattggggccaggggacccaggtgaccgtctcctca

SEQ ID NO: 91 - DNA, 296 bp, synthetic construct
  source 1..296: mol_type = other DNA
tggaggcttggtgcaggttggggggtctctgagactctcctgtgtagtttctggaagcgacatcagtggcattgcgatgggctggtaccgccaggctccagggaagcggcgcgaaatggtcgcagatattttttctggcggtagtacagactatgcaggctccgtgaagggccgattcaccatctccagagacaacgccaagaagacgagctatctgcaaatgaacaacgtgaaacctgaggacaccggagtctactactgtaggctgtacgggagcggtgactactggggccagg

SEQ ID NO: 92 - protein, 25 aa, synthetic construct
EARPASQVQLAESGGGLVQPGGSLR

SEQ ID NO: 93 - protein, 12 aa, synthetic construct
LSCAASGFTFNR

SEQ ID NO: 94 - protein, 4 aa, synthetic construct
YVIR

SEQ ID NO: 95 - protein, 23 aa, synthetic construct
NMAYLQMSSLKPDDTAVYYCSAL

SEQ ID NO: 96 - protein, 23 aa, synthetic construct
NLEDMEYWGQGTQVTVSSGSEQK

SEQ ID NO: 97 - protein, 26 aa, synthetic construct
LISEEDLGGGGSGGLPETGGHHHHHH

(19) United States
(12) Patent Application Publication
(10) Pub. No.: US 2025/0066459 A1
(43) Pub. Date: Feb. 27, 2025

(54) LINKABLE SINGLE DOMAIN ANTIBODIES THAT COVALENTLY BIND TO A TARGET ANTIGEN AND METHODS OF USE THEREOF

(71) Applicant: Trustees of Tufts College, Medford, MA (US)

(72) Inventors: James Van Deventer, Medford, MA (US); Charles Shoemaker, Medford, MA (US); Rafael de Jesus Alcala-Torano, Medford, MA (US)

(73) Assignee: Trustees of Tufts College, Medford, MA (US)

(21) Appl. No.: 18/943,537

(22) Filed: Nov. 11, 2024

Related U.S. Application Data
(63) Continuation of application No. PCT/US2023/066990, filed on May 15, 2023.
(60) Provisional application No. 63/342,341, filed on May 16, 2022.

(51) Int. Cl. C07K 16/12; C12N 15/10

(52) U.S. Cl. CPC C07K 16/1282; C12N 15/1037; C07K 2317/569; C07K 2317/76; C07K 2317/92

(57) ABSTRACT

Provided are single-domain antibodies (sdAbs), also called heavy-chain-only variable domains (VHHs) or camelids, that contain reactive, non-canonical amino acids (ncAAs) and crosslink to target antigen protein. The target antigen may be a toxin or a neurotoxin produced by a pathogenic organism or microorganism that causes or is associated with a disease or pathology. Compositions and methods using the reactive ncAA-containing sdAbs to treat subjects afflicted with a pathology caused by a toxin- or neurotoxin-producing pathogen, such as Botulinum, are provided. In some cases, the compositions and methods comprising the sdAbs treat or prevent intoxication and inhibit and/or neutralize toxin or neurotoxin activity.

### CROSS REFERENCE TO RELATED APPLICATIONS

The present application is a continuation under 35 U.S.C. § 111(a) of PCT International Patent Application No. PCT/US2023/066990, filed May 15, 2023, designating the United States and published in English, which claims priority to and the benefit of U.S. Provisional Application No. 63/342,341, filed May 16, 2022, the entire contents of each of which are incorporated by reference herein.

### STATEMENT REGARDING FEDERALLY SPONSORED RESEARCH

This invention was made with government support under Grant No. AI093504 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on May 6, 2023, is named “167774-013001_PCT_SL.xml” and is 92,278 bytes in size.

### BACKGROUND

Botulinum neurotoxins (BoNTs) are extremely potent toxins to humans and cause flaccid paralysis. All BoNTs have a catalytic light chain (LC), which is a Zn²⁺-endopeptidase that specifically cleaves neuronal SNARE proteins and is mainly responsible for BoNT's neurotoxic effects, while the heavy chain (HC) mediates toxin attachment to neurons and delivers the LC into the cytosol. More effective botulism antidotes and reagents are needed to treat toxicity caused by BoNT infection, particularly those that target light chain A (LC/A) of Botulinum neurotoxin. Also needed in the art are techniques for generating stable and target-specific reagents that possess high potency and specificity to inhibit the toxicity of BoNT LC (LC/A).

### SUMMARY

Provided by the aspects and embodiments described herein are single-domain antibodies (sdAbs), which comprise heavy-chain-only variable domains (VHHs), substituted with reactive noncanonical amino acids (ncAAs) that are capable of forming covalent bonds with nearby groups on a target antigen protein based on their reactivity. The terms “sdAb” and “VHH” are used interchangeably herein. In an embodiment, the ncAA incorporated in the sdAb is 4-azidophenylalanine (AzF), which forms covalent bonds with nearby groups on a target antigen protein following photoillumination, e.g., UV radiation. In an embodiment, the ncAA incorporated in the sdAb is O-(2-bromoethyl)tyrosine (OBeY), which is spontaneously reactive and forms covalent bonds with nearby groups on a target antigen protein. As described herein, numerous ncAA substitutions were well-tolerated, and examples of both UV-crosslinkable and spontaneously crosslinkable sdAb clones that exhibited target specificity and further benefits were conferred by the ncAAs. By way of example, a high-throughput protein discovery platform involving the use of yeast display, in combination with noncanonical amino acids (ncAAs), were used to identify irreversible variants (also called mutants herein) of sdAbs targeting Botulinum neurotoxin light chain A (LC/A). The platform was used to evaluate the properties of these sdAbs that were substituted with reactive noncanonical amino acids (ncAAs). The yeast display platform as described herein in combination with genetic code expansion in the discovery of chemically enhanced antibodies, such as sdAbs, provide advantageous techniques that can be used to detect and identify covalent protein-based binding agents with improved pharmacological properties against antigen targets of interest.

In an aspect, a single domain antibody (sdAb) comprising at least one reactive, non-canonical amino acid (ncAA) that is capable of forming a covalent interaction between the ncAA of the sdAb and a target antigen bound by the sdAb is provided. In an embodiment, the sdAb specifically binds to Botulinum neurotoxin (BoNT).

In another aspect, a single domain antibody (sdAb) comprising at least one reactive, non-canonical amino acid (ncAA) that is capable of forming a covalent interaction between the ncAA of the sdAb and a Botulinum neurotoxin target antigen bound by the sdAb is provided.

In an embodiment of the above-delineated aspects and the embodiments thereof, the sdAb comprises at least one detectable tag. In an embodiment, the at least one detectable tag comprises a c-myc tag and/or a hemagglutinin tag. In an embodiment, the ncAA is photoreactive or spontaneously reactive. In an embodiment, the ncAA comprises 4-azidophenylalanine (AzF) or O-(2-bromoethyl)tyrosine (ObeY), respectively. In an embodiment, the BoNT is Botulinum neurotoxin light chain A (LC/A). In an embodiment, the at least one ncAA installed in the sdAb is AzF which is incorporated in the sdAb amino acid sequence at one or more amino acid positions photo-crosslinkable to the target antigen LC/A selected from Q1, Y32, Y37, R35, F29, F52, N54, S56, Y101, L101, Q44, K64, R107, M104, R101, W104, or Y111. In embodiments, ncAAs are incorporated at the following amino acid positions, which are designated in bold font in the sdAb sequence:
(i) Q1, Y37, N54, Y101, R107 in sdAb JPU-A5, which comprises the amino acid sequence

(SEQ ID NO: 64)
QVQLVETGGGLVQAGGSLRLSCTASGADFSFYAMGWYRQTPGNSRELVA
VMNLNGVISYGDSARGRFDISRDGTKNIVFLQMNSLKPEDTGVYYCNGM
RLYTRGSVRHPESWGQGIQVTVSS;

(ii) Y32, R35, Y37, L101, M104 in sdAb JPU-C1, which comprises the amino acid sequence

(SEQ ID NO: 65)
QVQLAESGGGLVQPGGSLRLSCAASGFTFNRYVIRWYRQAPGKERELVA
GISRSGDSGRYVDSVKGRFTISRDNDKNMAYLQMSSLKPDDTAVYYCSA
LNLEDMEYWGQGTQVTVSS;

(iii) Q1, F29, Y32, Q44, R101, Y111 in sdAb JPU-C10, which comprises the amino acid sequence

(SEQ ID NO: 66)
QLQLVESGGGLVQPGGSLRLSCAASGNIFSIYYMGWYRQAPGKQREMVA
IINSNGITNYGDFVKGRFTISRDNAENSAYLQMNNLTPEDTAVYYCNAG
KLRRTTGWGLDDYWGQGTQVTVSS;

and
(iv) Q1, F52, S56, K64, W104 sdAb JDQ-H7, which comprises the amino acid sequence

(SEQ ID NO: 67)
QVQLVESGGGLVQVGGSLRLSCVVSGSDISGIAMGWYRQAPGKRREMVA
DIFSGGSTDYAGSVKGRFTISRDNAKKTSYLQMNNVKPEDTGVYYCRLY
GSGDYWGQGTQVTVSS.

In some cases, the sdAb JDQ-H7 is alternatively called ciA-H7 herein.

In an embodiment, the at least one ncAA of the sdAb is ObeY which is incorporated in the sdAb amino acid sequence at one or more amino acid positions spontaneously crosslinkable to the target antigen LC/A selected from R31, R53, D56, S57, R59, N100, E102, D103, and Y106. In an embodiment, the sdAb is JPU-C1. In an embodiment, the sdAb is in dimeric or multimeric form. In embodiments, the covalent interaction between the ncAA of the sdAb and the Botulinum neurotoxin target antigen comprises nucleophilically or electrophilically crosslinking the ncAA and an amino acid residue of the target antigen.

In another aspect, an isolated polynucleotide encoding the sdAb of any of the above-delineated aspects and embodiments thereof is provided.

In another aspect, an isolated polynucleotide encoding a dimeric or multimeric form of the sdAb of any of the above-delineated aspects and embodiments thereof is provided.

In another aspect, a vector comprising the isolated polynucleotide of the above-delineated aspects is provided. In an embodiment, the vector is an expression vector. In embodiments, the expression vector is a viral or a non-viral expression vector.

In another aspect, a cell comprising the vector of any one of the above-delineated aspects and embodiments thereof is provided.

In another aspect, a composition comprising the sdAb of any one of the above-delineated aspects and embodiments thereof; the isolated polynucleotide of any one of the above-delineated aspects and embodiments thereof; the vector of any one of the above-delineated aspects and embodiments thereof; or the cell of any one of the above-delineated aspects and embodiments thereof is provided. In an embodiment, the composition further includes a pharmaceutically acceptable carrier, diluent, or excipient.

In another aspect, a method of treating or preventing intoxication by a Botulinum toxin is provided, in which the method involves administering to a subject in need thereof an effective amount of the sdAb of any one of the above-delineated aspects and embodiments thereof; the isolated polynucleotide of any one of the above-delineated aspects and embodiments thereof; the vector of any one of the above-delineated aspects and embodiments thereof; or the cell of any one of the above-delineated aspects and embodiments thereof, or a pharmaceutically acceptable composition thereof.

In an aspect, a method of neutralizing toxicity of a Botulinum toxin is provided, in which the method involves administering to a subject in need thereof an effective amount of the sdAb of any one of the above-delineated aspects and embodiments thereof; the isolated polynucleotide of any one of the above-delineated aspects and embodiments thereof; the vector of any one of the above-delineated aspects and embodiments thereof; or the cell of any one of the above-delineated aspects and embodiments thereof; or a pharmaceutically acceptable composition thereof. In embodiments of the above methods, the subject has, is at risk of having, or is susceptible to infection or intoxication by Botulinum toxin. In embodiments of the method, the subject is a mammal, and, in particular, a human patient.

In an aspect, a method of reducing, ameliorating, abating, abrogating, or eradicating intoxication of a cell by a Botulinum neurotoxin is provided, in which the method involves contacting the cell with an effective amount of the sdAb of any one of the above-delineated aspects and embodiments thereof; the isolated polynucleotide of any one of the above-delineated aspects and embodiments thereof; the vector of any one of the above-delineated aspects and embodiments thereof; or the cell of any one of the above-delineated aspects and embodiments thereof; or a pharmaceutically acceptable composition thereof, thereby reducing, ameliorating, abating, abrogating, or eradicating intoxication of the cell by the Botulinum neurotoxin. In an embodiment of the method, the Botulinum toxin is LC/A protease. In embodiments of the method, the cell is in vitro, ex vivo, or in vivo. In an embodiment of the method, the sdAb polypeptide or a polynucleotide encoding the sdAb polypeptide is introduced into or delivered into the cell by a method used in the art. (See, e.g., P. McNutt et al., 2021, Science Translational Medicine, Vol. 13, No. 575; DOI: 10.1126/scitranslmed.abd7789).

In another aspect, a kit comprising the sdAb of any one of the above-delineated aspects and embodiments thereof; the isolated polynucleotide of any one of the above-delineated aspects and embodiments thereof; the vector of any one of the above-delineated aspects and embodiments thereof; or the cell of any one of the above-delineated aspects and embodiments thereof; or a pharmaceutically acceptable composition thereof, for treating or protecting against disease or intoxication and/or the symptoms thereof caused by Botulinum neurotoxin protease; and optionally comprising instructions for use.

In another aspect, a method of selecting single domain antibody (sdAb) which covalently crosslinks with a target antigen is provided, in which the method involves expressing on yeast cells a detectable sdAb comprising at least one reactive non-canonical amino acid (ncAA) capable of forming a covalent interaction with a target antigen bound by the sdAb; contacting the sdAb with the target antigen; activating the ncAA in the sdAb to crosslink the sdAb and the target antigen, thereby forming a covalent complex comprising the sdAb and the target antigen; denaturing the complex to remove unbound target antigen; and detecting and selecting the sdAb.

In another aspect, a method of detecting a target antigen is provided, in which the method comprises: expressing on yeast cells a detectable sdAb comprising at least one reactive non-canonical amino acid (ncAA) capable of crosslinking to a target antigen; contacting a sample with the yeast cells; activating the ncAA in the sdAb to crosslink the sdAb with the target antigen if present in the sample, thereby forming a covalent complex comprising the sdAb and the target antigen; denaturing the complex to remove unbound target antigen; and detecting the target antigen crosslinked to the sdAb, if present in the sample. In embodiments, the sample is a biological sample, a cell, or a cell extract.

In an embodiment of the above-delineated selecting and detecting methods, the target antigen is Botulinum neurotoxin. In an embodiment, the Botulinum neurotoxin is light chain A (LC/A) protein. In an embodiment, the detectable sdAb comprises a detectable tag, e.g., a cmyc tag, and the like, and wherein the detecting comprises flow cytometry. In embodiments, the reactive ncAA is photoreactive or spontaneously reactive. In an embodiment, the photoreactive ncAA crosslinks with a suitable amino acid residue on the target antigen upon exposure to ultraviolet illumination. In an embodiment, the spontaneously reactive ncAA crosslinks with a suitable amino acid residue on the target antigen upon exposure to a temperature increase, e.g., without limitation, room temperature, 35° C. or 37° C., or a lower temperature, e.g., at least about 15° C., 20° C., 25° C., 30° C., or 35° C., and values therebetween. In embodiments, the photoreactive ncAA is Azp and the spontaneously reactive ncAA is ObeY.

In another aspect, the use of yeast display expression system for expressing and selecting reactive, non-canonical amino acid (ncAA)-containing single-domain antibodies (sdAbs) that bind and crosslink to a target antigen is provided. In an embodiment, the sdAbs bind and crosslink to Botulinum neurotoxin LC/A protease (BoNT LC/A).

### Definitions

Unless defined otherwise, all technical and scientific terms used herein have the meaning commonly understood by a person skilled in the art to which the described aspects and embodiments belongs. The following references provide one of skill with a general definition of many of the terms used in the aspects and embodiments described herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). The following terms have the meanings ascribed to them below, unless specified otherwise.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in the specification and claim(s) herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions and products of the present disclosure can be used to achieve methods of the present disclosure.

Unless specifically stated or obvious from context, as used herein, the term “about” or “approximately” means within an acceptable error range for the particular value as determined by one of ordinary skill in the art, which will depend, in part, on how the value is measured or determined, i.e., the limitations of the measurement system. For example, “about” can mean within 1 standard deviation or more than 1 standard deviation, e.g., 2 standard deviations of the mean, as typically practiced in the art. Alternatively, and without intending to be limiting, “about” can mean a range of up to 20%, up to 10%, up to 5%, up to 2%, or up to 1% of a given value. Alternatively, and particularly for biological systems or processes, the term can mean within an order of magnitude, e.g., within 5-fold, within 3-fold, within 2.5-fold, or within 2-fold of a value. Where particular values are described in the application and claims, unless otherwise stated, the term “about” means within an acceptable error range for the particular value.

Reference herein to “some embodiments,” “an embodiment,” “one embodiment” or “other embodiments” means that a particular feature, structure, or characteristic described in connection with the embodiments is included in at least some embodiments, but not necessarily all embodiments, of the disclosure.

By “agent” is meant any small molecule chemical compound, antibody, nucleic acid molecule, or polypeptide (e.g., antibody or single-domain antibody), or fragments thereof.

By “ameliorate” is meant decrease, reduce, diminish, suppress, attenuate, arrest, or stabilize the development or progression of a disease or pathology.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. In some embodiments, the alteration in structure is one or more amino acid changes.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include a non-canonical amino acid.

By “antibody” is meant any immunoglobulin polypeptide, or fragment thereof, having immunogen or antigen binding ability. Antibody structure is well known in the art. Briefly, the variable (V) regions or domains of antibody heavy (H) and light (L) chains contain Complementarity-Determining Regions (CDRs), which bind to specific antigens or immunogens (e.g., protein antigens or immunogens). CDRs are situated within framework (FR) sequences of the V regions of the heavy (V_{H}) and light chains (V_{L}) of an antibody. CDRs are the most variable parts of antibodies and are critical components in the diversity of antigen specificities of antibodies produced by B lymphocytes. In general, three CDRs (CDR1, CDR2 and CDR3) are arranged consecutively in a V domain of an antibody. Because a VHH, such as a camelid VHH, is essentially a single chain antibody polypeptide, it contains three CDRs that bind to an antigen or target protein such as a toxin or neurotoxin (e.g., LC/A BoNT) in the context of four framework (FR) regions, as follows: FR1-CDR1-FR2-CDR2-FR3-CDR3-FR4. Thus, as would be appreciated by the skilled practitioner in the art, in a VHH polypeptide sequence, FRI comprises the amino acids positioned to the left of CDR1; FR2 comprises the amino acids positioned between CDR1 and CDR2; FR3 comprises the amino acids positioned between CDR2 and CDR3; and FR4 comprises the amino acids positioned to the right of CDR3. Because most of the sequence variability associated with immunoglobulins and antigen binding is found in the CDRs, these regions are sometimes referred to as hypervariable regions. Typically, CDR1, CDR2 and CDR3 of VHHs contribute to and/or do not interfere with antigen binding. The CDRs and FRs of a number of BoNT LC/A VHHs are shown herein (FIG. 8 and Table 1).

A “chimeric antibody” refers to an antibody in which the constant region of an antibody of one species (e.g., rodent, mouse or rat) is replaced with that from a human to achieve a more human-like antibody. Chimeric antibodies may be recombinantly generated by combining the variable light and heavy chain regions obtained from antibody producing cells of one species with the constant light and heavy chain regions from another. In general, chimeric antibodies utilize rodent (or other species, such as rabbit or camelid) variable regions and human constant regions in order to produce an antibody with predominantly human constant domains. The production of chimeric antibodies is well known in the art, and may be achieved by standard means, for example, as described in U.S. Pat. No. 5,624,659, incorporated fully herein by reference.

By “binding to” a molecule is meant having a physicochemical affinity for that molecule or a region of the molecule, e.g., an epitope. Binding may be measured by any of the methods practiced in the art, e.g., using an antibody binding assay or an in vitro translation binding assay.

“Detect” refers to identifying or determining the presence, absence or amount of an analyte to be detected.

By “detectable label” is meant a compound, substance, or composition that, when linked to a molecule of interest, renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include, without limitation, Botulinum serotype A or A1, Botulinum neurotoxin infection or intoxication, intoxication by Botulinum neurotoxin LC/A or A1, botulism, or one or more symptoms of these diseases. The production of toxins by the pathogenic and infectious Botulinum microorganisms results in intoxication of the subject (or patient), which is an abnormal state that is a poisoning of the subject (and the subject's cells, tissues and organs) by the presence and activity of the produced toxins.

By “effective amount” is meant the amount of a required to ameliorate, or optimally eliminate, the symptoms of a disease relative to an untreated patient. The effective amount of active compound(s) used to practice the present aspects and embodiments for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

An “epitope tag” refers to a peptide or amino acid sequence (epitope) that is fused, linked, or coupled to a protein, such as a recombinant protein produced by recombinant techniques, and that can be specifically bound by an antibody, e.g., an anti-tag monoclonal antibody or binding molecule that is directed to or generated against the tag peptide or amino acid sequence. Epitope tags are typically short peptide sequences (e.g., from about 5-30 amino acids, or sometimes up to 40 amino acids, that are selected because high-affinity antibodies can be reliably produced in many different species. Such anti-epitope tag antibodies are optimally not cross-reactive with other human peptides or polypeptides and typically do not generate an antibody response, e.g., an anti-tag antibody response, when administered or delivered to a subject. An epitope tag sequence that is fused to a protein provides for the detection and/or purification of the protein using an antibody, e.g., a monoclonal antibody or sdAb, that specifically binds to the epitope tag. In an embodiment, the protein to which an epitope tag is fused, linked, or coupled is an antibody or VHH protein, e.g., a recombinantly produced antibody or VHH protein. In an embodiment, the VHH is an anti-LC/A BoNT VHH antibody that binds BoNT LC/A. In an embodiment, the protein, or a dimeric or multimeric form thereof, may include one or more epitope tags. In an embodiment, an epitope tag is coupled to the amino (NH) terminus of the protein, e.g., a VHH antibody as described herein. In an embodiment, an epitope tag is coupled to the carboxy (COOH) terminus of the protein, e.g., a VHH antibody as described herein. In an embodiment, an epitope tag is coupled to the NH and the COOH termini of the protein, e.g., a VHH antibody as described herein. In an embodiment, a dimeric or multimeric form of the protein includes one or more, e.g., two, three or four, epitope tags linked to one or more of the VHHs comprising the dimeric or multimeric form of the protein. Such epitope tags may be coupled to the VHH components at locations within the dimer or multimer molecule, or at the NH and/or COOH termini of the molecule. In some embodiments, two or more epitope tags may be coupled to a VHH protein in tandem within or at the termini of the VHH protein or dimeric or multimeric form thereof. Examples of epitope tags include, without limitation, FLAG tags (peptide sequence DYKDDDDK (SEQ ID NO: 1) recognized by an anti-FLAG antibody), polyHistidine (His) tags (5-10 histidine residues (HHHHHH (SEQ ID NO: 2)) bound by a nickel or cobalt chelate), E-tag, a peptide comprising amino acid sequence GAPVPYPDPLEPR (SEQ ID NO: 3) recognized by an antibody; myc-tag, or c-myc tag, and the epitope tag sequences described herein, which are bound by anti-epitope tag antibodies, forming complexes which may facilitate clearance of the protein containing the tags from the body or system. (See, also, B. Brizzard and R. Chubet, 2001, Curr Protoc Neurosci., Chapter 5, Unit 5.8; DOI: 10.1002/0471142301.ns0508s00; R. Hernan et al., 2000, Biotechniques, 28(4):789-793; C. E. Fritze et al., 2000, Meths Enzymol., 327:3-16; doi: 10.1016/s0076-6879(00)27263-7; A. Einhauer et al., 2001, J Biochem Biophys Methods, 49(1-3):455-65, doi: 10.1016/s0165-022x(01)00213-5)).

A “framework (FR) region” or “FR region” includes amino acid residues that are adjacent to the CDRs in V_{H}, and V_{L} regions, and in VHHs. For example, FR region residues may be present in VHHs as described herein, human antibodies, rodent-derived antibodies (e.g., murine and rat antibodies), humanized antibodies, primatized antibodies, chimeric antibodies, antibody fragments (e.g., Fab fragments), VHHs, single-chain antibody fragments (e.g., scFv fragments), antibody domains, and bispecific antibodies, among others. Also by way of example, in a VHH polypeptide sequence, FRI comprises the amino acids positioned to the left of CDR1; FR2 comprises the amino acids positioned between CDR1 and CDR2; FR3 comprises the amino acids positioned between CDR2 and CDR3; and FR4 comprises the amino acids positioned to the right of CDR3.

By “fragment” is meant a portion of a polypeptide or nucleic acid molecule. This portion contains, preferably, at least 10%, 20%, 30%, 40%, 50%, 60%, 70%, 80%, 90%, or 95% of the entire length of the reference nucleic acid molecule or polypeptide, including percent values between those enumerated. A fragment may contain 10, 20, 30, 40, 50, 60, 70, 80, 90, or 100, 200, 300, 400, 500, 600, 700, 800, 900, or 1000 nucleotides or amino acids. In an embodiment, a fragment or portion possesses or retains activity or function of the polypeptide from which it is derived. In embodiments, a fragment is derived from a BoNT LC/A toxin.

The term “humanized” antibodies refers to forms of non-human (e.g., murine) antibodies, camelid-derived single domain antibody (sdAb) binding molecules, which are comprised of the heavy chain variable (V_{H}) region of heavy-chain-only antibodies (Abs) or VHHs. Humanized antibodies include chimeric immunoglobulins, immunoglobulin chains or fragments thereof (such as Fv, Fab, Fab′, F(ab′)2 or other target-binding subdomains of antibodies) which contain minimal sequences derived from non-human immunoglobulin. In general, a humanized antibody or VHH may comprise substantially all of at least one variable domain (or two variable domains in the case of non-VHH antibodies), in which all or substantially all of the CDR regions correspond to those of a non-human immunoglobulin. All or substantially all of the FR regions of a humanized antibody may also be derived from a human immunoglobulin sequence. In the case of non-VHH antibodies, a humanized antibody can also comprise at least a portion of an immunoglobulin constant region (Fc), which may be that of a human immunoglobulin consensus sequence. Techniques and protocols for humanizing antibodies (as well as VHHs) are known and practiced in the art, as described, for examples, in Riechmann et al., Nature, 332:323-7, 1988; Kasmiri et al., Methods, 36(1):25-34, 2005; U.S. Pat. Nos. 5,530,101; 5,585,089; 5,693,761; 5,693,762; and 6,180,370 to Queen et al; EP239400; WO 1991/09967; U.S. Pat. No. 5,225,539; EP592106; and EP519596, the contents of which are incorporated herein by reference. Humanized antibodies or VHHs are molecularly engineered to contain even more human-like immunoglobulin domains and incorporate only the CDRs of the VHH or animal-derived monoclonal antibody by carefully examining the sequence of the hyper-variable loops of the V regions of the monoclonal antibody or VHH, and fitting them to the structure of the human antibody chains. This process is routinely and commonly carried out by one having skill in the art. See, e.g., U.S. Pat. No. 6,187,287, the contents of which are incorporated by reference herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the aspects and embodiments disclosed and described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

A “non-canonical amino acid (ncAA)” refers to an unnatural, non-proteinogenic amino acid that is either found naturally in organisms, synthetically made, or biosynthetically made using metabolic engineering and/or synthetic biology to assemble the ncAAs in cells or in a cell-free system. An ncAA is an amino acid analog that acts as a surrogate for a naturally occurring amino acid. In one embodiment, a noncanonical amino acid is an isostructural analog of a canonical amino acid. The terms “noncanonical amino acid”, “unnatural amino acid”, “nonnatural amino acid”, “nonstandard amino acid”, or “nonproteinogenic amino acid” are interchangeable. NcAAs are amino acids that are not located in the genetic code of naturally occurring organisms and can be incorporated into proteins at defined positions of the amino acid sequence of the protein, i.e., site-specific incorporation of ncAAs into proteins. They can be used to investigate the structure and dynamics of proteins, to study their interactions and functions, to expand the chemical space of peptides and proteins, to control their activity in living cells, and to introduce novel functions in proteins that are not achievable in nature. NcAAs and forms thereof, e.g., photoreactive or photoactivatable, can be synthesized and are commercially available (e.g., Bio-techne Corp./Tocris, Minneapolis, MN; R&D Systems) and are utilized by those skilled in the art (A. M. Saleh, et al, 2019, J. Biol. Engineering, 13(43), doi.org/10.1186/si3036-019-0166-3). Non-limiting examples of noncanonical amino acids include O-methyl-L-tyrosine (OmeY); p-acetyl-L-phenylalanine (AcF); p-azido-L-phenylalanine (AzF); p-propargyloxy-L-phenylalanine (OPG); 4-azidomethyl-L-phenylalanine (AzMF); 4-borono-L-phenylalanine (BPhe); 3,4-dihydroxy-L-phenylalanine (DOPA); O-(2-Bromoethyl)-tyrosine; 4-iodo-L-phenylalanine (IPhe); L-α-aminocaprylic acid (AC); NE-azido-L-lysine (AzK); 3-Amino-L-tyrosine (ATyr); 4-Amino-L-phenylalanine (APhe); dimethyl-L-lysine (DMK); Boc-L-lysine (BocK); (S)-2-amino-6-((2-azidoethoxy)carbonylamino)hexanoic acid (LysN3); and 2-Amino-6-(prop-2-ynoxycarbonylamino)hexanoic acid (LysAlk), H-L-photo-lysine, O-(2-Bromoethyl)-L-tyrosine, O-Sulfo-L-tyrosine (SY), 2-amino-3-[4-(carboxymethyl) phenyl]propanoic acid (CMF), L-p-hydroxy-phenyllactic acid (Ester), L-2-Amino-4-phosphonobutyric acid (PSA), O-phospho-L-serine (OPS), and Acetyl-L-lysine (AcK). Nonlimiting examples of structures of ncAAs include the following:
“O-methyl-L-tyrosine (OmeY)” having the structure

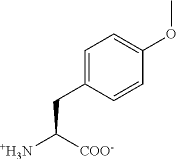

or an analog thereof,
“p-acetyl-L-phenylalanine (AcF)” having the structure

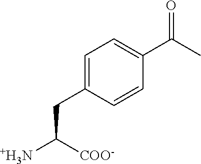

or an analog thereof;
“p-azido-L-phenylalanine (AzF)” having the structure

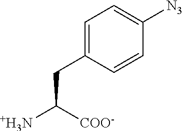

or an analog thereof,
“p-propargyloxy-L-phenylalanine (OPG)” having the structure

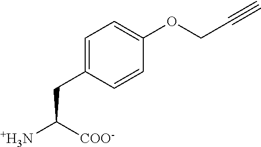

or an analog thereof;
“4-azidomethyl-L-phenylalanine (AzMF)” having the structure

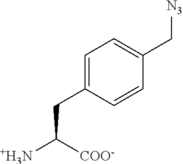

or an analog thereof;
“4-borono-L-phenylalanine (BPhe)” having the structure

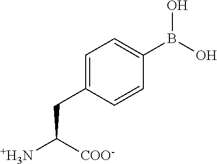

or an analog thereof;
“3,4-dihydroxy-L-phenylalanine (DOPA)” having the structure

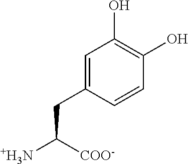

or an analog thereof;
“4-iodo-L-phenylalanine (IPhe)” having the structure

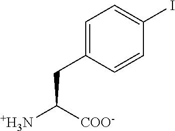

or an analog thereof;
“L-α-aminocaprylic acid (AC)” having the structure

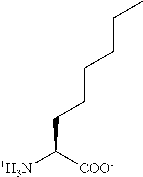

or an analog thereof,
“Nε-azido-L-lysine (AzK)” having the structure

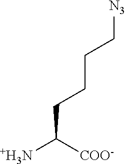

or an analog thereof;
“3-Amino-L-tyrosine (ATyr)” having the structure

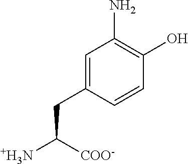

or an analog thereof;
“4-Amino-L-phenylalanine (APhe)” having the structure

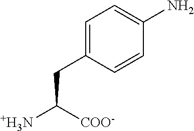

or an analog thereof;
“dimethyl-L-lysine (DMK)” is meant a noncanonical amino acid with the structure

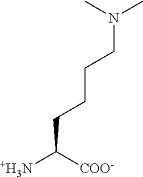

or an analog thereof;
“Boc-L-lysine (BocK)” having the structure

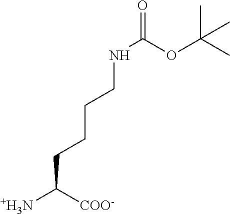

or an analog thereof;
“(S)-2-amino-6-((2-azidoethoxy)carbonylamino)hexanoic acid (LysN3)” having the structure

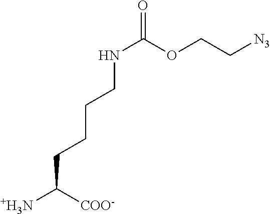

or an analog thereof;
“2-Amino-6-(prop-2-ynoxycarbonylamino)hexanoic acid (LysAlk)” having the structure

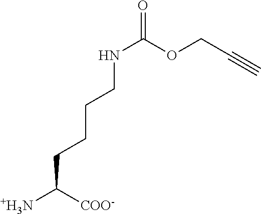

or an analog thereof;
“O-Sulfo-L-tyrosine (SY)” having the structure

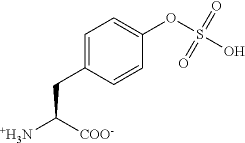

or an analog thereof;
“2-amino-3-[4-(carboxymethyl) phenyl]propanoic acid (CMF)” having the structure

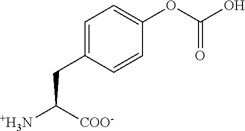

or an analog thereof;
“L-p-hydroxy-phenyllactic acid (Ester)” having the structure

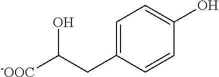

or an analog thereof;
“L-2-Amino-4-phosphonobutyric acid (PSA)” having the structure

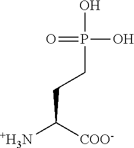

or an analog thereof;
“O-phospho-L-serine (OPS)” having the structure

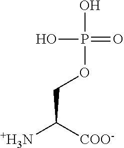

or an analog thereof;
“Acetyl-L-lysine (AcK)” having the structure

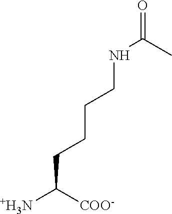

or an analog thereof;
“H-L-photo-lysine” having the structure

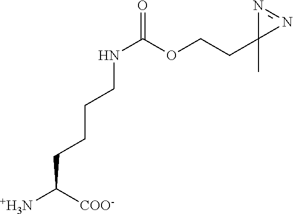

or an analog thereof; or
“O-(2-Bromoethyl)-L-tyrosine” (OBeY) having the structure

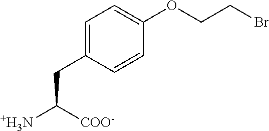

or an analog thereof.

In embodiments, the ncAA is AzF or ObeY. Analogs of the above structures have the functionality of the original structure.

The reactivity of genetically encoded ncAAs incorporated in sdAbs as described herein refers to the ability of the ncAA with electrophilic moieties to engage in crosslinking reactions with other amino acid residues, e.g., nucleophilic amino acid residues such as Cys, Asp, Glu, Ser, Thr, His, and Tyr, in binder proteins, e.g., a target antigen protein. In an embodiment, the target protein antigen is neurotoxin protease (e.g., LC/A neurotoxin) produced by Botulinum microorganisms.

As used herein, the terms “polynucleotide,” “DNA molecule” or “nucleic acid molecule” include both sense and anti-sense strands, cDNA, genomic DNA, recombinant DNA, RNA, mRNA, and wholly or partially synthesized nucleic acid molecules. A nucleotide “variant” is a sequence that differs from the recited nucleotide sequence in having one or more nucleotide deletions, substitutions or additions. Such modifications are readily introduced using standard mutagenesis techniques, such as oligonucleotide-directed site-specific mutagenesis as described, for example, in Adelman et al., 1983, DNA 2:183. Nucleotide variants are naturally-occurring allelic variants, or non-naturally occurring variants. Variant nucleotide sequences in various embodiments exhibit at least about 70%, 75%, 80%, 85%, 90%, 95%, 98%, 99%, or 100% sequence homology or sequence identity to the recited sequence. Such variant nucleotide sequences hybridize to the recited nucleotide sequence under stringent hybridization conditions. In one embodiment, “stringent conditions” refers to prewashing in a solution of 6×SSC, 0.2% SDS; hybridizing at 65° Celsius, 6×SSC, 0.2% SDS overnight; followed by two washes of 30 minutes each in 1×SSC, 0.1% SDS at 65° C., and two washes of 30 minutes each in 0.2×SSC, 0.1% SDS at 65° C.

By “isolated polynucleotide” is meant a nucleic acid (e.g., DNA, cDNA, RNA, mRNA) that is free of the genes, which, in the naturally-occurring genome of the organism from which the nucleic acid molecule of the aspects and embodiments described herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, e.g., mRNA, as well as a recombinant DNA that is part of a hybrid gene encoding additional polypeptide sequence.

The terms “protein”, “peptide” and “polypeptide” are used herein to describe any chain of amino acid residues, regardless of length or post-translational modification (for example, glycosylation or phosphorylation). Thus, these terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length proteins, which may be, but need not be, naturally occurring, as well as recombinantly or synthetically produced polypeptides that correspond to a full-length protein, or to particular domains or portions of a protein, which may be, but need not be, naturally occurring. The term also encompasses mature proteins which have an added amino-terminal methionine to facilitate expression in prokaryotic cells. The binding molecules of the aspects and embodiments described herein are encoded by polynucleotides and can be chemically synthesized or synthesized by recombinant DNA methods. In various embodiments, conservative amino acid substitutions may be made to a polypeptide to provide functionally equivalent variants, or homologs of the polypeptide. In some aspects and embodiments, sequence alterations that result in conservative amino acid substitutions are embraced. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By an “isolated polypeptide” is meant a polypeptide of the aspects and embodiments described herein that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the aspects and embodiments described herein. An isolated polypeptide of the aspects and embodiments described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, deriving, isolating, or otherwise acquiring the agent.

By “operably linked” is meant the connection between regulatory elements and one or more polynucleotides (genes) or a coding region. That is, gene expression is typically placed under the control of certain regulatory elements, including constitutive or inducible promoters, tissue-specific regulatory elements, and enhancers. A polynucleotide (gene or genes) or coding region is said to be “operably linked to” or “operatively linked to” or “operably associated with” the regulatory elements, meaning that the polynucleotide (gene or genes) or coding region is controlled or influenced by the regulatory elements. The one or more polynucleotides may be separated by spacers or linkers.

By “pathogen” is meant any harmful microorganism, bacterium, virus, fungus, or protozoan capable of interfering with the normal function of a cell. Pathogens as referred to herein produce toxins, e.g., protein toxins, that intoxicate the cells, tissues and organs of a host or recipient organism and cause disease and pathology, often severe, unless they are bound by, neutralized and eliminated from the organism to the extent possible, such as by action of the VHH binding molecules (antibodies) described herein. As described herein, the bacterial Botulinum pathogen produces neurotoxin proteins that intoxicate a subject after infection. The catalytic light chain (LC) of the toxin is a Zn²⁺-endopeptidase that specifically cleaves neuronal SNARE proteins and is mainly responsible for BoNT's neurotoxic effects, while the heavy chain (HC) mediates the attachment of toxin to neurons and delivers the LC into the cytosol. In particular, the VHH antibodies described herein are directed against and bind the BoNT LC/A protease, such as LC/A1 protease. In embodiments, the anti-LC/A VHHs described herein inhibit, block, or reduce neurotoxicity and toxic effects in vitro or in vivo. In an embodiment, the anti-LC/A VHHs described herein neutralize neurotoxicity and toxic effects caused by Botulinum toxin, such as LC/A, in vitro or in vivo.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative or lowering alteration of at least 5%, 10%, 15%, 10%, 25%, 50%, 75%, or 100%.

By “reference” is meant a standard or control condition typically used as a comparator in an assay, test, experiment, or trial, as would be understood by one having skill in the pertinent art. In various nonlimiting embodiments, a reference or control is a different or nonpathogenic protein or cell, such as a non-toxin (or different toxin) protein or a normal cell, a wild-type (unmutated or unaltered) protein, or a healthy subject or individual.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound, molecule, antibody, or VHH that recognizes and binds a protein, peptide, or polypeptide (e.g., an amino acid sequence of the protein, peptide, or polypeptide), but which does not substantially recognize and bind other molecules in a sample, for example, a biological sample, which may contain the protein, peptide, or polypeptide that is specifically bound.

“Nucleic acid” (also called polynucleotide herein) refers to deoxyribonucleotides or ribonucleotides and polymers thereof in either single- or double-stranded form. The term encompasses nucleic acids (polynucleotides) containing known nucleotide analogs or modified backbone residues or linkages, which are synthetic, naturally occurring, and non-naturally occurring, which have similar binding properties as a reference nucleic acid, and which are metabolized in a manner similar to the reference nucleic acid. Examples of such analogs include, without limitation, phosphorothioates, phosphoramidates, methyl phosphonates, chiral methyl phosphonates, 2-O-methyl ribonucleotides, and peptide-nucleic acids (PNAs). Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., 1991, Nucleic Acid Res, 19:081; Ohtsuka et al., 1985, J Biol. Chem., 260:2600-2608; Rossolini et al., 1994, Mol. Cell Probes, 8:91-98). The term nucleic acid can be used interchangeably with gene, cDNA, mRNA, oligonucleotide, and polynucleotide.

Nucleic acid molecules or polynucleotides useful in the aspects and embodiments described herein include any nucleic acid molecule or polynucleotide that encodes a polypeptide, e.g., a heteromultimeric binding molecule, of the described aspects and embodiments, or a component or portion thereof. Nucleic acid molecules useful in the methods described herein include any polynucleotide or nucleic acid molecule that encodes a polypeptide e.g., heteromultimeric binding molecule, as described in the aspects and embodiments herein, or a component or portion thereof that has substantial identity to the binding molecule. Such nucleic acid molecules need not be 100% identical with the nucleic acid sequence of the binding molecule, but will typically exhibit substantial identity. Polynucleotides having “substantial identity” to a binding molecule sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, 1987, Methods Enzymol. 152:399; Kimmel, A. R., 1987, Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In an embodiment, hybridization will occur at 37° C. in 500 mM NaCl, 50 mM trisodium citrate, 1% SDS, 35% formamide, and 100 μg/ml denatured salmon sperm DNA (ssDNA). In an embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In an embodiment, wash steps will occur at 42° C. in 15 mM NaCl, 1.5 mM trisodium citrate, and 0.1% SDS. In an embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “homologous” in their various grammatical forms in the context of polynucleotides means that a polynucleotide comprises a sequence that has a desired identity, for example, at least 60% identity, at least 70% sequence identity, at least 80%, at least 85% identity, at least 90% identity; and at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like.

Substantial identity of amino acid sequences, for example, the anti-LC/A polypeptides (BoNT LC/A-binding VHH polypeptides) refers to sequence identity between or among amino acid sequences of at least 60%, at least 65%, at least 70%, at least 75%, at least 80%, at least 85%, at least 88%, at least 90%, at least 95%, at least 98%, at least 99% or greater sequence identity. In embodiments, 100% identity between or among the amino acid sequences, e.g., the CDR1-3 sequences of the LC/A—binding VHHs as described herein is not required for binding of these polypeptides to LC/A and/or neutralization of toxin activity or inhibition of toxin activity. In a particular embodiment, variations between or among VHH amino acid sequences encompass one or more conservative amino acid substitutions in the sequence. In an embodiment, one or more conservative amino acid substitutions in an anti-LC/A binding VHH amino acid sequence may be in one or more CDR sequences, one or more FR sequences, or a combination thereof.

As will be appreciated by the skilled practitioner in the art, some amino acids in a VHH antibody can be modified without significantly altering antigen binding of the VHH antibody. For example, such amino acid sequence modification occurs frequently during in vivo affinity maturation of VHH antibodies, and the best mutations, e.g., for specific and/or high affinity binding to antigen, are positively selected for in the animal during the molecular production of antibodies. It is possible to isolate different VHH intermediates in the affinity maturation process that possess acceptable and specific antigen binding properties and that have significant variations in their CDR sequences.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as, without limitation, a human, a non-human primate, or a bovine, equine, canine, ovine, or feline mammal. Other mammals include rabbits, goats, llamas, mice, rats, guinea pigs, camels and gerbils. In particular, a “subject” as used herein refers to a human subject, such as a human patient or individual. In some cases, the terms subject, patient and individual are used interchangeably herein.

A single-domain antibody (sdAb), also referred to herein as a “VHH binding molecule” or “VHH antibody,” or simply “VHH,” is, in general, a single-domain immunoglobulin molecule (antibody) isolated from camelid animals or alpacas, e.g., as described in Mass, D. R., 2007, J Immunol. Methods, 324(1-2):13-15). A VHH (or VHH antibody) corresponds to the heavy chain of a camelid antibody having a single variable domain (or single variable region), e.g., a camelid-derived single variable H (V_{H}) domain antibody. A VHH has a molecular weight (MW) of about 15 kDa. VHH technology is based on fully functional antibodies from camelids that lack light chains. These heavy-chain antibody molecules contain a single variable domain (VHH) and, typically, two constant domains (CH₂ and CH₃). A cloned (recombinantly produced) and isolated VHH domain is a stable polypeptide harboring the antigen-binding capacity of the original heavy-chain antibody. See, e.g., U.S. Pat. Nos. 5,840,526 and 6,015,695, each of which is incorporated by reference herein in its entirety. VHHs, called NANOBODIES™, may be produced commercially (Ablynx Inc., Ghent, Belgium).

VHHs are efficiently expressed in E. coli, coupled to detection markers, such as a fluorescent marker, or conjugated with enzymes. The small size of VHHs permits their binding to epitopes (antigenic determinants in antigen proteins), e.g., “hidden epitopes” that are not accessible to whole antibodies of much larger size. As a therapeutic, a VHH is capable of efficient penetration and rapid clearance. Its single domain nature allows a VHH to be expressed in a cell without a requirement for supramolecular assembly, as is needed for whole antibodies which are typically tetrameric (two heavy chains and two light chains, having a MW of about 150 kDa). VHHs are also exhibit stability over time and have a longer half-life versus non-VHH antibody molecules, which comprise disulfide bonds that are susceptible to chemical reduction or enzymatic cleavage. Similar to immunoglobulins, VHHs may be modified post-translationally, e.g., to add chemical linkers, detectable moieties, such as fluorescent dyes, enzymes, substrates, chemiluminescent moieties, etc., or specific binding moieties, such as streptavidin, avidin, or biotin, etc., for use in the compositions and methods described herein.

An anti-LC/A BoNT VHH polypeptide that specifically binds to and neutralizes the activity of LC/A BoNT, may also be referred to as a “VHH-based neutralizing agent (VNA)” a “VNA polypeptide or protein,” a “VNA binding molecule,” or a single-domain variable heavy-chain (VHH), sdAb, antibody.

As used herein, the terms “treat,” treating,” “treatment,” and the like refer to reducing, diminishing, abating, alleviating, improving, or ameliorating a disorder and/or symptoms associated therewith. It will be appreciated that, although not precluded, treating a disorder or condition does not require that the disorder, condition or symptoms associated therewith be completely eliminated.

The term “multimeric binding molecule” refers in general to a multi-component protein or polypeptide containing two (e.g., dimeric) or more, same or different, sdAb (VHH) binding molecules, which are coupled or linked, e.g., via spacer sequences, to each other and/or other components of the molecule. Multimeric binding molecules may be dimeric, in that the binding molecule contains two VHH polypeptides that bind to LC/A BoNT. A dimeric binding molecule may include two anti-LC/A VHH polypeptides that are the same or different. A dimeric binding molecule may include one anti-LC/A VHH polypeptide and one VHH polypeptide directed against another BoNT. The different anti-LC/A VHH polypeptides in a multimeric binding molecule may bind to different regions, portions, or epitopes (e.g., non-overlapping epitopes) of LC/A. In some embodiments, a heteromultimeric binding molecule contains two, three, four or more different anti-BoNT LC/A VHH polypeptides, each of which specifically binds to an LC/A BoNT, e.g., at different or non-overlapping epitopes. In embodiments, dimer and multimeric binding molecules comprising two or more anti-LC/A-VHHs bind to and neutralize the activity of the LC/A neurotoxin.

As used herein, the terms “prevent,” “preventing,” “prevention,” “prophylactic treatment,” “protection” and the like refer to reducing the probability of developing a disorder or condition in a subject, who does not have, but who is at risk of, is susceptible to, or disposed to (e.g., genetically disposed to), developing a disease, disorder, pathology, or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of the first and last values.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Although various features of the present disclosure can be described in the context of a single embodiment, the features can also be provided in separate embodiments, or in any suitable combination or combination of embodiments. The section headings used herein are for organizational purposes only and are not intended to be limiting to the subject matter described.

The features of the present disclosure are set forth with particularity in the appended claims. The features and advantages of the present disclosure will be better understood and obtained by reference to the detailed description infra, which sets forth illustrative embodiments, in which the principles of the disclosure are utilized, and in view of the accompanying drawings as described herein.

### BRIEF DESCRIPTION OF THE DRAWINGS

FIGS. 1A-1D provide chemical structures, a table, an illustration and crystal structures. FIG. 1A: Reactive ncAAs used in this work for photo-crosslinking (AzF) and spontaneous crosslinking (OBeY). FIG. 1B: Crystal structures of LC/A (gray surface, active site highlighted in cyan with catalytic Zn²⁺ shown as a sphere) bound to each wild type sdAb (shown as colored cartoons and transparent surfaces). The mutated residues are indicated and highlighted as sticks. FIG. 1C: Residue positions used for ncAA incorporation at each of the four sdAbs used in the studies and examples herein. By way of example, ncAAs were incorporated at the following amino acid positions in sdAb JPU-A5: Q1, Y37, N54, Y101, R107; ncAAs were incorporated at the following amino acid positions in sdAb JPU-C1: Y32, R35, Y37, L101, M104; ncAAs were incorporated at the following amino acid positions in sdAb JPU-C10: Q1, F29, Y32, Q44, R101, Y111; and ncAAs were incorporated at the following amino acid positions in sdAb JDQ-H7 (also termed ciA-H7): Q1, F52, S56, K64, W104. FIG. 1D: Representation of the strategy to evaluate ncAA-mediated covalent interactions between LC/A (shown in gray) and displayed sdAbs.

FIGS. 2A-2C present flow cytometry dot plots and graphs. FIG. 2A: Representative flow cytometry dot plots of displaying yeast cells after induction in the absence of ncAA (−ncAA) or presence of 1 mM AzF or OBeY after incubation with 200 nM LC/A. Cells were labeled against Strep-tag and cMyc tag for detection of LC/A (vertical axis) and full-length displayed antibody (horizontal axis), respectively. FIG. 2B: Median fluorescence intensity (MFI) of the LC/A detection of full-length sdAb populations as determined by flow cytometry analysis. The ncAA position is indicated in each case. This experiment was performed once. Error bars represent the robust coefficient of variation of the populations. FIG. 2C: Titration data for selected JPU-A5 (left) and JPU-C1 (right) proteins displayed on the yeast surface. One-site binding fit curves are shown as solid lines and their 95% confidence intervals drawn as shadowed areas. The estimated K_{d} values are indicated in each plot along with the 95% confidence interval for these estimates. The titrations were carried out in technical triplicates. Error bars represent the standard deviation of the sample. See, FIG. 13 for titration dot plots.

FIGS. 3A-3D present a schematic illustration and graphs related to the identification of sdAbs on the yeast surface. FIG. 3A: Scheme depicting the experiments used to characterize photo-crosslinking on the yeast surface. Yeast cells displaying the sdAbs were incubated with LC/A and subjected to 365 nm UV irradiation (1) which can result in successful crosslinking for some AzF clones (top), but no covalent bond was formed without irradiation, WT, or unsuccessful AzF mutants (bottom). Denaturation (2) removes noncovalently bound LC/A, leaving covalent adducts on the cell surface for detection via flow cytometry. FIG. 3B: Median Fluorescence Intensity (MFI) of LC/A detection in full-length sdAb populations measured via flow cytometry experiments after irradiation and denaturation of yeast displaying the indicated sdAbs, where the residue numbers represent the position where AzF was incorporated. This experiment was performed once. See, FIG. 15 for all flow cytometry plots. FIG. 3C: Representative flow cytometry dot plots of WT and selected photoreactive sdAbs. The binding control plots show the behavior of binding behaviors of displayed sdAbs in the dark, without irradiation or denaturation. The no irradiation and 3 min irradiation panels show the populations of cells after the denaturing step. See, FIG. 17 for all plots. FIG. 3D: Time course showing the MFI corresponding to LC/A detection via flow cytometry after increasing irradiation times of each of the indicated nanobodies and denaturation. These experiments were carried out in technical triplicates. The error bars represent the standard deviations of the sample.

FIGS. 4A-4D present schematic illustrations, bar graphs, and plots related to the identification of spontaneous crosslinking of sdAbs and target antigen on the yeast surface. FIG. 4A: Scheme representing the evaluation of spontaneous crosslinking on the yeast surface. Cells displaying the corresponding sdAbs were incubated with LC/A at 37° C. (1). Certain OBeY-containing clones can form a covalent bond with residues of the nearby LC/A (top), whereas constructs with non-reactive amino acids or lacking the requirements for reactivity maintain a non-covalent interaction (bottom). After denaturation (2), irreversibly bound LC/A can be detected via flow cytometry, while reversible interactions are disrupted, and no LC/A is detected. FIG. 4B: MFI of LC/A detection in full-length sdAb populations measured via flow cytometry experiments after incubation with LC/A at 37° C. for 24 h followed by denaturation. Indicated residues correspond to the position where OBeY was incorporated. See, FIG. 18 for all flow cytometry dot plots. FIG. 4C: Representative flow cytometry dot plots of WT and OBeY mutants capable of spontaneous crosslinking to LC/A. The corresponding OPG controls are included. The flow cytometry analysis was carried out after the denaturation step following different incubation times with LC/A. See, FIG. 19 and FIG. 20 for flow cytometry dot plots. FIG. 4D: Time course experiment showing the MFI corresponding to LC/A detection via flow cytometry after different incubation times followed by denaturation. These experiments were performed in technical triplicates. The error bars represent the standard deviations of the samples.

FIGS. 5A-5D present western blots and a plot of results associated with detection of the sdAbs that bind and crosslink to BoNT LC/A antigen. FIG. 5A: SDS-PAGE and western blots of JPU-C1 WT and L101AzF after incubation with LC/A and following 280 nm UV irradiation for 0 or 2 min in solution. A band corresponding to the crosslinked adduct is detected only after irradiation of the L101AzF protein. The western blot results are consistent with the formation of an LC/A-sdAb adduct (FIGS. 22A-22C). FIG. 5B: SDS-PAGE and western blots of soluble forms of JPU-C1 WT, Y32OBeY, and M104OBeY after incubation with LC/A at 37 C. The adduct band was observed only in the OBeY-containing camelids, and the western blots further corroborate the identity of the adduct (FIGS. 23A-23C). FIG. 5C: Time course of the reaction between JPU-C1 L101AzF 1 μM and LC/A 200 nM at different irradiation times as visualized by Coomassie stained SDS-PAGE (FIGS. 24A and 24B). LC/A lanes marked with +* indicate inactive LC/A, i.e., in the absence of Zn²⁺. Western blotting was carried out by probing with streptactin-DY488 for LC/A detection and mouse a-cMyc HRP conjugate. FIG. 5D: Band intensity of the adduct in the experiment of FIG. 5C normalized to that of the LC/A control without incubation or irradiation. This experiment was carried out in technical triplicate. Error bars represent the standard deviations of the samples. LC/A lanes marked with +* indicate inactive LC/A, i.e. in the absence of Zn2+. Western blotting was carried out by probing with Strep-Tactin DY488 1:500 for LC/A detection and mouse a-cMyc HRP conjugate 1:2,500 for sdAb detection.

FIGS. 6A and 6B present a graph and legend related to the evaluation of inhibitory properties of soluble ncAA-sdAbs. FIG. 6A: dose-response curves and FIG. 6B: estimated IC₅₀ values for each sdAb when incubated with 250 μM LC/A followed by addition of 100 nM of fluorescent reporter. Fractional activities were calculated by normalizing the initial rate to that of uninhibited enzyme and fitted to a dose-response model. 95% confidence intervals are shown in parentheses. The indicated (*) p-value indicates statistical significance at the 95% level when comparing the IC₅₀ value to that of WT.

FIGS. 7A-7C present Western blots probed for the sdAb C-terminal cMyc tag after incubation in HepG2 lysate with varying concentrations and JPU-C1 L101AzF (FIG. 7A), JPU-C1 Y32OBeY (FIG. 7B), or JPU-C1 M104OBeY (FIG. 7C), in the presence or absence of LC/A 200 nM. The samples in FIG. 7A were analyzed after a 1 h incubation at room temperature and with or without 365 nm light irradiation for 30 s. Samples in panels FIG. 7B and FIG. 7C were incubated for 24 h at 37° C. The membranes were probed with mouse α-cMyc-HRP conjugate.

FIG. 8 shows an alignment of the amino acid sequences of various sdAbs having binding specificity for Botulinum toxin LC/A. SEQ ID NOs: 68-86 designate the amino acid sequences of the sdAbs presented FIG. 8, as well as in Table 1. Several of the sdAbs shown were used in the generation of sdAbs molecularly engineered to contain ncAAs. The sequences showing alignment of conserved framework regions (FRI-FR4); CDRs are indicated. The terminal six amino acids of FRI are not shown as they are altered during PCR to amplify the sdAb coding DNA during sdAb-display phage library construction.

FIGS. 9A-9D show illustrations, scatter plots, bar graphs and equations for quantifying relative readthrough efficiency (RRE) and maximum misincorporation frequency (MMF). FIG. 9A: Scheme showing the dual fluorescent reporter (top) and suppression machinery (bottom) used to verify incorporation of OBeY by AcFRS. The fluorescent reporters BYG and BXG consist of tethered BFP and GFP proteins wherein the linker contains a TAC or TAG codon, respectively. FIG. 9B: Representative dot plots showing the N-terminal (vertical axis) vs C-terminal (horizontal axis) detection of the fluorescent reporters in the absence (−ncAA) or presence of 1 mM OBeY in the induction medium. FIG. 9C: Formulae used to quantify the relative readthrough efficiency (RRE) and maximum misincorporation frequency (MMF) of AcFRS for OBeY incorporation. FIG. 9D: RRE (left) and MMF (right) values calculated for OBeY incorporation with AcFRS. Error bars correspond to the propagated standard deviation of the sample. The experiment was done in biological triplicates.

FIG. 10 provides flow cytometry dot plots of JPU-A5 (left) and JPU-C1 (right) displayed mutants when induced in the absence (−ncAA) or presence of 1 mM AzF or OBeY, as indicated, and incubated with 200 nM LC/A. Residue numbers indicate the position substituted with the ncAA-encoding TAG codon. The horizontal axis represents detection of the c-Myc epitope at the C-terminus of the sdAb, while the vertical axis corresponds to the detection of the LC/A bound to the yeast surface, as detected via its C-terminal Strep-tag. This screening experiment was performed once.

FIG. 11 provides flow cytometry dot plots of JPU-C10 (left), ciA-H7 (top right), also termed JDQ-H7, displayed mutants, and FAPB2.3.6 non-binder control protein (bottom right) when induced in the absence (−ncAA) or presence of 1 mM AzF or OBeY, as indicated, and incubated with 200 nM LC/A. Residue numbers indicate the position substituted with the ncAA-encoding TAG codon. The horizontal axis represents detection of the cMyc epitope at the C-terminus of the sdAb, while the vertical axis corresponds to the detection of the LC/A bound to the yeast surface, as detected via its C-terminal Strep-tag. This screening experiment was performed once.

FIGS. 12A and 12B depict bar graphs showing corrected median fluorescence intensity (MFI) of LC/A (FIG. 12A) and displayed full-length sdAb (FIG. 12B) for the investigated sdAbs (camelids) when induced in the absence (−ncAA, gray bars) or presence of 1 mM AzF (middle bar in WT and leftmost bar in the sets of assessed sdAbs (camelids) shown on the x-axis) or OBeY (rightmost bar in the WT and in the set of assessed sdAbs (camelids) shown on the x-axis) in cMyc-positive populations as determined by flow cytometry. The horizontal axis indicates the mutation site for ncAA incorporation; WT indicates the “wild type” sdAb, with no TAG codons in the encoding sequence. This screening experiment was performed once. Error bars indicate the propagated robust coefficient of variation of the population.

FIG. 13 provides representative dot plots corresponding to the titrations done on the yeast surface for the clones indicated. Briefly, 3,000 displaying cells were incubated with different concentrations of LC/A for 24 h and the cells labeled for flow cytometry. Experiments were done in technical triplicates. The gates of the analyzed populations are indicated and were chosen to minimize outliers. A detailed description of the analysis is provided in the Methods herein. FIG. 2C describes the binding curves.

FIGS. 14A and 14B provide flow cytometry dot plots and a bar graph. FIG. 14A: Representative dot plots of the flow cytometry analysis of LC/A binding on yeast cells displaying the WT constructs of the indicated sdAbs before (− denaturation) and after (+ denaturation) treatment with 8M urea/200 mM EDTA/25 mM Tris pH 8.0 to remove noncovalently bound LC/A. FIG. 14B: Average median fluorescence intensity (MFI) of the LC/A detection in cMyc positive populations after incubation with LC/A (binding, solid bars) and after incubation followed by the denaturing wash (binding+denaturation, light gray dotted bars; lower MFI values). Experiments were done in technical triplicate with incubation of cells for 24 h at 37° C., and denaturation at room temperature for 16 h. Error bars represent the standard deviation of the sample.

FIG. 15 provides flow cytometry dot plots corresponding to the photo-crosslinking screening experiment with the TAG clones induced in presence of AzF. The analysis was carried out after incubation with LC/A and irradiation for 2 min, followed by a denaturing wash and labeling. The screening was performed once.

FIGS. 16A-16D present the results of an analysis of apparent crosslinking as a function of Cα-Cα distances related to the mutated residues in the described sdAbs. FIG. 16A: Table showing the nearest LC/A residues to the mutated residues in each sdAb. The residues in this table show the shortest Cα-Cα distance between the residues based on crystallographic data. FIGS. 16B and 16C: Scatter plots showing the relationship between the normalized LC/A fluorescence detection levels during photo-crosslinking screening (FIG. 16B, refer to FIG. 3B) or spontaneous crosslinking (FIG. 16C, refer to FIG. 4B) and the measured Cα-Cα distance. FIG. 16D: Scatter plots showing the normalized LC/A fluorescence detection levels during spontaneous crosslinking against the Cα-Cα distances of each sdAb mutant to the nearest of each possible type of nucleophile in LC/A. The data labels indicate the corresponding LC/A residue for each distance. By way of example, labels in the upper leftmost box correspond to residues in the active site of LC/A. Distances were determined based on available crystallographic data. Distances to residue L2 in JPU-C10 are shown since Q1 is not resolved in the crystal structure.

FIG. 17 provides representative dot plots corresponding to the time course photo-crosslinking study on the yeast surface for the shown sdAb. Yeast cells were incubated with LC/A and irradiated for up to 5 min as shown. Samples were then treated with denaturant, washed, and labeled for flow cytometry. The binding control was not irradiated nor treated with denaturant. The no irradiation control was not irradiated and was treated with denaturant. Experiments were carried out in technical triplicates.

FIG. 18 provides flow cytometry dot plots corresponding to the spontaneous screening experiment with the TAG clones induced in presence of OBeY. The analysis was carried out after incubation with LC/A for 24 h at 37° C., followed by a denaturing wash and labeling. The screening was performed once.

FIG. 19 provides dot plots of binding controls in the time course analysis of spontaneous crosslinking with the indicated WT, OPG (controls), and OBeY clones. Cells were incubated with LC/A at 37° C. for the indicated amount of time, at which point they were washed and kept at 4° C. until the last time point was taken. Cells were then labeled for flow cytometry and analyzed. Experiments were carried out in technical triplicates.

FIG. 20 provides flow cytometry dot plots of the time course analysis of spontaneous crosslinking with cells displaying the indicated sdAbs. Cells were incubated with LC/A at 37° C. and at each time point an aliquot taken and washed. After time points were taken, they were washed under denaturing conditions, washed again, and labeled for flow cytometry. Experiments were performed in technical triplicates.

FIGS. 21A and 21B present a gel and a graph related to the characterization of soluble sdAbs. FIG. 21A: Typical Coomassie-stained SDS-PAGE for quantification of sdAbs relative to myoglobin standards. FIG. 21B: Example of calibration curve derived from the band intensities in the SDS-PAGE for the myoglobin standards.

FIGS. 22A-22C present full images of a Coomassie-stained SDS-PAGE gel (FIG. 22A); a cMyc-HRP-probed western blot (FIG. 22B); and a streptactin-DY488-probed western blot (FIG. 22C) of the photo-crosslinking analysis in solution with JPU-C1 WT or L101AzF shown in FIG. 5A. For FIG. 22B, the opacity on the left-hand portion of the image was reduced to show the superimposed molecular weight marker.

FIGS. 23A-23C present an SDS-PAGE gel and western blots. Full images of Coomassie-stained SDS-PAGE (FIG. 23A), streptactin-DY488-probed western blot (FIG. 23B) and cMyc-HRP-probed western blot (FIG. 23C) of the spontaneous crosslinking analysis in solution with JPU-C1 WT, Y32OBeY or M104OBeY shown in FIG. 5B.

FIGS. 24A and 24B present full images corresponding to the Coomassie-stained SDS-PAGE of irradiation-dependence photo-crosslinking in solution. FIG. 24A shows the full image of the gel presented in FIG. 5C, and FIG. 24B shows the controls with the individual proteins irradiated for different amounts of time.

FIGS. 25A-25C present SDS-PAGE gels and a plot of band intensity data.: FIGS. 25A and 25B show Coomassie-stained SDS-PAGE gels of time and pH-dependence incubation of LC/A 200 nM (FIG. 25A) and co-incubation of JPU-C1 M104OBeY 1 μM with LC/A 200 nM at 37° C. (FIG. 25B). FIG. 25C shows a plot of band intensity of the adduct band normalized to the LC/A band at time 0 h, pH 7.1. The experiment was performed one time.

FIGS. 26A-26D show kinetic traces monitoring LC/A (250 μM) activity in solution in the presence of the indicated amounts of each sdAb: (FIG. 26A): JPU-C1 WT, (FIG. 26B): JPU-C1 Y32OBeY, (FIG. 26C): JPU-C1 L101AzF, and (FIG. 26D): M104OBeY. The vertical axis shows the fluorescence ratio of the FRET substrate as described in the methods. The left-side panels correspond to the entire monitored timeframe. The blank sample corresponds to the fluorescence ratio of the reporter alone, while the LC/A 10 nM trace corresponds to fully cleaved reporter obtained by treating with 10 nM LC/A. The right-side panels show the initial traces for each sample along with the linear fits used to calculate the initial velocity in each case. Error bars represent the standard deviation of each sample, done in technical triplicate.

FIGS. 27A-27C provide Coomassie stained gels incubation of HepG2 lysate with varying concentrations of LC/A 200 nM as shown in FIGS. 7A-7C, related to the selectivity of varying concentrations of JPU-C1 L101AzF (FIG. 27A), JPU-C1 Y32OBeY (FIG. 27B), or JPU-C1 M104OBeY (FIG. 27C) when incubated in the presence of HepG2 lysate. The samples in FIG. 27A were analyzed after a 1 hour incubation at room temperature and with or without 365 nm light irradiation for 30 s. The samples in FIG. 27B and FIG. 27C were incubated for 24 hours at 37° C. The membranes were probed with mouse a-cMyc-HRP conjugate.

FIG. 28 presents bar graphs showing the median fluorescence intensity (MFI) of LC/A detection in full-length sdAb populations measured via flow cytometry experiments after irradiation with a handheld lamp for 6 hours, followed by denaturation of yeast displaying the indicated sdAbs. The residue numbers represent the position where AzF was incorporated. This experiment was performed once. FIG. 29 provides flow cytometry dot plots related to these graphic results.

FIG. 29 provides flow cytometry dot plots corresponding to the photocrosslinking screening experiment with the TAG clones induced in the presence of AzF. The analysis was carried out after incubation with LC/A and irradiation for 6 hours with a handheld lamp, followed by a denaturation and labeling, as described for FIG. 28. The screening was performed once.

FIGS. 30A and 30B provide graphs showing the median fluorescence intensity (MFI) corresponding to the LC/A detection of cMyc-positive populations in the photocrosslinking time-course experiment on the yeast surface with the indicated. FIG. 30A: clone JPU-A5 and FIG. 30B: clone JPU-C1. Binding controls represent the samples that were neither irradiated nor denatured. The “no irradiation” sample was not irradiated but was treated with denaturant, and the rest of the samples were irradiated for the indicated amount of time before denaturation. Error bars represent the standard deviation of the sample for each technical triplicate set.

FIGS. 31A-31D show a gel western blot, spectra tracings, and a table related to the characterization of soluble sdAbs. FIG. 31A shows a Coomassie-stained SDS-PAGE gel (left) and a streptavidin-AF488-probed western blot after copper catalyzed click chemistry of 1 μM WT and L101AzF sdAbs in presence (+) or absence (−) of an alkyne-containing biotin probe. The arrow indicates the band corresponding to biotinylated camelid, which confirms the presence of the azido group in AzF incorporated into the protein. FIGS. 31B and 31C show MALDI-TOF-MS spectra of the tryptic digest mixture of JPU-C1 (FIG. 31B) WT and Y32OBeY (FIG. 31C). The right panels show a detailed view of the 540-665 m/z region. The assigned mass of the OBeY-containing peptide is 656.5 (FIG. 31C). FIG. 31D presents a table containing the identified relevant sequences for identification of OBeY incorporation into the Y32OBeY sdAb. Residue positions are indicated above the sequences, with negative numbers representing the secretory N-terminal sequence. The XVIR peptide (X=OBeY) was identified and shows typical M, M+2 peaks with similar intensities as expected from the natural isotope abundance of bromine. The amino acid sequences of FIG. 31D are designated as follows: EARPASQVQLAESGGGLVQPGGSLR (SEQ ID NO: 92); LSCAASGFTFNR (SEQ ID NO: 93); YVIR (SEQ ID NO: 94); NMAYLQMSSLKPDDTAVYYCSAL (SEQ ID NO: 95); NLEDMEYWGQGTQVTVSSGSEQK (SEQ ID NO: 96); and LISEEDLGGGGSGGLPETGGHHHHHH (SEQ ID NO: 97).

FIGS. 32A-32C present full images corresponding to the western blots shown in FIGS. 7A-C showing the cMyc detection in selectivity experiments with varying concentrations of JPU-C1 AzF (FIG. 32A), JPU-C1 Y32OBeY (FIG. 32B), or JPU-C1 M104OBeY (FIG. 32C) when incubated in presence of HepG2 lysate. The samples in FIG. 32A were analyzed after a 1 hour incubation at room temperature and with or without 365 nm light irradiation for 30 seconds. Samples in FIGS. 32B and 32C were incubated for 24 hours at 37° C. Blot membranes were probed with mouse a-c-Myc-HRP conjugate at a 1:2500 dilution. The opacity of a rectangular portion of the left side of each blot was reduced to show the superimposed molecular weight ladder.

FIGS. 33A-33C present western blots showing the Strep-tag detection of LC/A in selectivity experiments with varying concentrations of JPU-C1 AzF (FIG. 33A), JPU-C1 Y32OBeY (FIG. 33B), or JPU-C1 M104OBeY (FIG. 33C) when incubated in presence of HepG2 lysate. The samples in FIG. 33A were analyzed after a 1 hour incubation at room temperature and with or without 365 nm light irradiation for 30 seconds. Samples in panels FIGS. 33B and 33C were incubated for 24 hours at 37° C. The right image in FIG. 33B shows an exposed version of the image on the left to better show the adduct band. Blot membranes were probed with Strep-Tactin DY488 1:500.

FIGS. 34A-34C present band intensity profiles for the western blot experiment shown in FIGS. 7A-C related to the evaluation of crosslinking specificity in the presence of 250 μg/mL HepG2 lysate. FIG. 34A corresponds to photocrosslinking with L101AzF (1 h incubation at room temperature, irradiation for 30 s), while FIGS. 34B and 34C correspond to incubation for 24 h at 37° C. with Y32OBeY or M104OBeY, respectively. The “No lysate control” sample was carried out in presence of 200 nM LC/A and 1 μM sdAb and irradiated for 30 s (FIG. 34A) or incubated for 24 h at 37° C. Both intensity plots in FIG. 34A include UV-irradiated “no lysate control” samples for reference. The light gray arrows indicate the position of the sdAb and the sdAb-LC/A adduct as determined from the “No lysate control.” The arrow above the right peaks shows a marker band present in the lysate that binds to the mouse a-cMyc-HRP probing antibody. The darker arrows in FIGS. 34A and 34C show possible crosslinked off-target entities, while the dark gray arrows in FIGS. 34B and 34C indicate the presumptive adduct between the sdAb and autolyzed LC/A. As can be observed in FIG. 34C, the position of the crosslinked “No lysate control” adduct in the intensity plot shown in appears at a higher molecular weight than in the other band intensity profiles. This is attributed to an apparent shift to a nonuniform (“smiling”) electrophoretic profile. This is also apparent in the position of the adduct in the +LC/A sample series. A line has been added to indicate the presumptive position of the adduct in this series of samples.

### DESCRIPTION OF THE EMBODIMENTS

While covalent drug discovery is reemerging as an important route to small molecule therapeutic leads, strategies for the discovery and engineering of protein-based irreversible binding agents remain limited. As described herein, the use of yeast display in combination with noncanonical amino acids (ncAAs) were used to identify irreversible variants of single-domain antibodies (sdAbs), also called VHHs and nanobodies, targeting botulinum neurotoxin light chain A (LC/A). Starting from a series of previously described, structurally characterized sdAbs, the properties of antibodies substituted with reactive ncAAs capable of forming covalent bonds with nearby groups after UV irradiation (when using 4-azido-L-phenylalanine) or spontaneously (when using O-(2-bromoethyl)-L-tyrosine) were evaluated. Systematic evaluations in yeast display format of more than 40 ncAA-substituted variants revealed numerous clones that retained binding function while gaining either UV-mediated or spontaneous crosslinking capabilities. Solution-based analyses indicated that ncAA-substituted clones exhibited site-dependent target specificity and crosslinking capabilities uniquely conferred by ncAAs. Interestingly, not all ncAA substitution sites resulted in crosslinking events, and data showed no apparent correlation between detected crosslinking levels and distances between sdAbs and LC/A residues. These findings highlight the power of yeast display in combination with genetic code expansion in the discovery and characterization of binding agents that covalently engage their targets. This platform streamlines the discovery and characterization of antibodies with therapeutically relevant properties that cannot be accessed in the conventional genetic code.

Described and featured herein are single domain antibodies (sdAbs), also called VHHs herein, containing a noncanonical amino acid (ncAA) as part of their sequence. In embodiments, the ncAA is 4-azidophenylalanine (AzF) or O-(2-bromoethyl)tyrosine (OBeY). These antibodies bind to a target antigen, e.g., one or more epitopes thereof, and are capable of forming a covalent bond to the target antigen, resulting in the formation of a covalent adduct between the antibody and the target antigen. In an aspect, the described sdAbs bind to Botulinum neurotoxin light chain A1 (LC/A) protease as the target antigen and form a covalent bond to the LC/A toxin protein, resulting in the formation of a covalent adduct between the sdAb and the neurotoxin light chain (target antigen). Antibodies, e.g., sdAbs, containing AzF form the covalent bond upon irradiation with UV light at 365 nm (photo-illumination), while those containing OBeY spontaneously react with the target after coincubation at 37° C.

The sdAbs described herein were discovered by rational mutagenesis of the parent sdAb sequences JPU-A5, JPU-C1, JPU-C10, and JDQ-H7, followed by screening on the yeast surface using flow cytometry analysis to identify successful formation of a covalent bond with the target after stringent denaturation conditions. AzF-substituted forms of each of the sdAbs exhibited UV-dependent crosslinking behavior on the yeast surface. Two sdAbs containing the ncAA, AzF, i.e., JPU-A5 N5AzF and JPU-C1 L101AzF, were further characterized on the yeast surface, and one was characterized in soluble form (JPU-C1 L101AzF). Three sdAbs containing the ncAA, OBeY, were identified on the yeast surface (JPU-A5 Y101OBeY, JPU-C1 Y32OBeY, and JPU-C1 M104OBeY), and both JPU-C1 mutants were characterized in solution. The ncAA-containing sdAbs were expressed in S. cerevisiae RJY100 using a modified secretory plasmid pRS314 and purified using Ni-NTA resin. The covalent adducts were confirmed by SDS-PAGE Coomassie stain and western blotting against the tags present in LCA (StrepTag) or sdAb (cMyc tag). The antibodies behaved as inhibitors of the LCA autolytic activity and preserved inhibition of the enzymatic cleavage of a fluorescent reporter containing the native SNAP25 cleavage site. Furthermore, the antibodies displayed low off-target effects when incubated in presence of a mammalian cell lysate.

sdAbs for the Treatment of Intoxication by Botulinum Neurotoxin

Botulinum neurotoxin enzymes, e.g., proteases, are challenging targets for current therapeutics, as they have long-lasting neurological effects due to their long intracellular lifetimes. Irreversible inhibition has been proposed as a solution to tackle this issue. Some small molecules can engage covalently with Botulinum neurotoxin enzyme, but they are likely to cause side effects due to off-target interactions. Antibodies have shown to selectively bind to their target in complex biological systems, offering an alternative to overcome this downside. The antibodies, e.g., sdAbs, described and featured herein are capable of engaging in covalent interactions, and thus advantageously result in irreversible inhibitors that specifically and selectively bind to their target.

The establishment of antibodies as new therapeutic agents has both promoted and benefited from modern biotechnology strategies that seek to improve antibody “drug-likeness” for pharmaceutical applications. Particularly, high-throughput display technologies have been used to engineer not only the high potency and selectivity of antibodies and alternative binding scaffolds,¹⁻⁸, but also other desirable physicochemical characteristics such as stability,⁹⁻¹¹ solubility,¹² and even environmental responsiveness.¹³⁻¹⁵.

Although many properties can now be introduced into antibodies, the limited range of chemical functionalities in the genetic code still constrains antibody properties. In particular, covalent target engagement is a property that is nearly impossible to access in antibodies. On the other hand, a growing number of small molecule drugs and drug leads possess chemical groups that facilitate the formation of covalent bonds with their respective biological targets. Covalent bond formation is useful in extended duration of action and sustained inhibition of target function and has even been shown to overcome acquired drug resistance.¹⁶⁻²¹ The systematic introduction of these functionalities into proteins provides opportunities for leveraging the exquisite specificities of antibodies while introducing antibodies reactivities beyond functionalities contained within the conventional genetic code.

Primary strategies for generating covalent protein adducts involve the use of either photocrosslinkable or spontaneously crosslinkable functional groups. Upon irradiation with light, photoreactive groups form reactive species that covalently engage with nearby residues and can convert a noncovalent interaction into a covalent one. Photo-crosslinking has been proven useful for in vitro investigations and protein profiling²²⁻³⁰, but its dependence on short-wavelength irradiation can limit its usage in therapeutics and other in vivo applications. Conversely, spontaneous crosslinking in the form of proximity-enhanced reactivity is an attractive strategy for therapeutics since it obviates the need for external stimuli to promote the reaction^{32, 31, 36, 35, 33, 37}.

Two main approaches have been exploited to expand the chemical landscape of proteins with groups that can participate in covalent binding. The first one relies in the installation of chemical warheads by targeting designed cysteine residues³⁸⁻³⁹, while the other is based on the incorporation of reactive noncanonical amino acids (ncAAs)^{32, 31, 35, 33, 40} via genetic code expansion, which can themselves react with the intended target. While each of these strategies has demonstrated the usefulness of engineering protein-based irreversible binding agents, each relies on rational design and solution-phase measurements to validate crosslinking events. Thus, the engineering of such reactivity has yet to benefit from high throughput platforms that can streamline discovery, engineering, and characterization processes prior to performing more detailed in-solution characterizations of promising leads.

As described herein, yeast display with flow cytometry, coupled with high resolution binding site structural information, were used to identify and characterize protease-inhibiting camelid single domain antibodies (sdAbs) that are capable of covalently binding to Botulinum neurotoxin, in particular, Botulinum neurotoxin light chain A (LC/A), via a reactive ncAA. LC/A is a zinc-dependent protease responsible for the paralytic effects of botulism from exposures to Botulinum neurotoxins (BoNTs), such as Botulinum toxin LC/A, which are attractive targets for therapy and treatment of intoxication, due to the long-lasting effects of the toxin in the neuronal cytosol. Furthermore, sdAbs can be delivered to intoxicated neurons to treat botulism in animals using an atoxic BoNT delivery vehicle.^{45, 46}

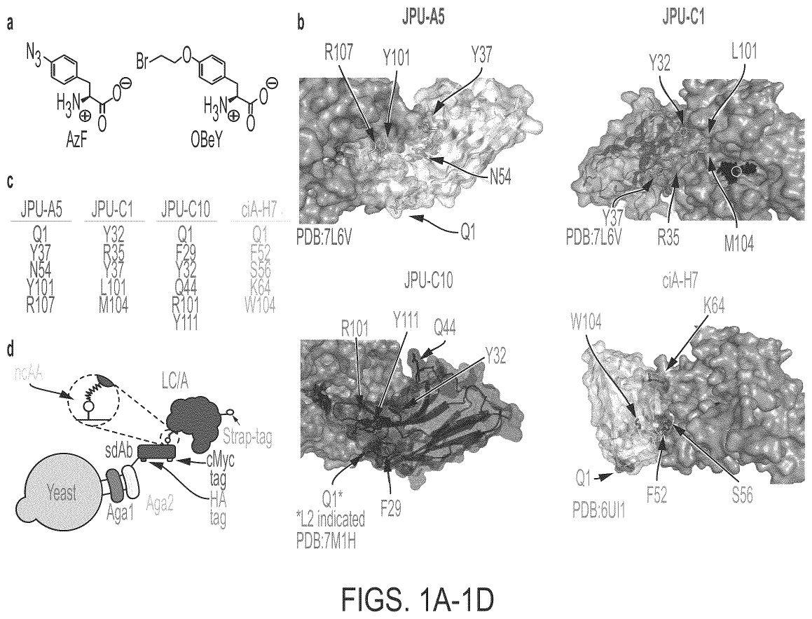

In embodiments, two ncAAs, 4-azidophenylalanine (AzF) and O-(2-bromoethyl)tyrosine (OBeY) (FIG. 1A), which are capable of light-mediated and spontaneous crosslinking, respectively, were employed. By corroborating the observations on the yeast surface with solution-phase measurements, this platform was able to discriminate between reversible and irreversible covalent binding agents and to characterize their time-dependent behavior. Experiments with the purified sdAbs also revealed that the reactive sdAbs retained their inhibitory properties against LC/A. In an embodiment, the ncAA position can impact the selectivity of the sdAbs in complex mixtures. The use of yeast display provides a robust platform for the discovery of chemically active sdAbs and provides new opportunities to further develop these tools for directed evolution and maturation of both the selectivity and reactivity of new and useful macromolecules.

The antibodies, e.g., sdAbs, described herein are the first examples of covalent antibodies against LCA. In addition, the use of a high-throughput-capable display platform to select for suitable sequences that allow for crosslinking between antibody and target is a newly provided feature. These artificial antibodies leverage the selectivity and potency of antibodies resulting in a decrease in off-target effects when compared to small molecule-based covalent inhibitors that are currently in development. Their crosslinking capabilities can advantageously result in longer inhibitory activity when compared to traditional alternatives based on reversible binding.

As described herein, two ncAAs, 4-azido-L-phenylalanine (AzF) and O-(2-bromoethyl)-L-tyrosine (OBeY) (FIG. 1A), were used to introduce crosslinking functionality into sdAbs via light-mediated and spontaneous crosslinking, respectively.^{24, 30-33} Assays in yeast display format revealed numerous ncAA-substituted sdAb variants that retained binding function and led to the identification of photocrosslinkable and spontaneously crosslinkable variants exhibiting time-dependent crosslinking behaviors. Corroboration of key observations on the yeast surface with solution-phase experiments indicated that this display platform can be used to discriminate between reversible and irreversible covalent binding agents and to characterize their time-dependent behavior. Further experiments with purified sdAbs revealed that the reactive sdAbs retain their inhibitory properties against LC/A, and that the ncAA substitution position can impact crosslinking selectivity in complex mixtures. These findings not only underscore the utility of yeast display for the discovery of chemically augmented sdAbs, but also open up new opportunities to further engineer the selectivity and reactivity of these novel macromolecules using tools with high throughput capabilities.

The antibodies described herein provide new therapies against Botulinum neurotoxin A1. The antibodies can also be used for detection purposes and in detection techniques against the Botulinum toxin A1 pathogenic agent.

### Therapeutic Methods

Therapeutic and prophylactic methods of treating disease, conditions, pathology and/or symptoms thereof associated with a pathogenic target protein or with infection by a pathogenic microorganism that produces a pathogenic target protein are provided. In an embodiment, the methods involve treating a disease caused by Botulinum neurotoxin (e.g., LC/A protease neurotoxin) produced following infection by a Botulinum microorganism with one or more ncAA-containing sdAbs as described herein. The methods comprise administering a therapeutically effective amount of one or more ncAA-containing sdAbs as described herein, or a pharmaceutical composition comprising the one or more ncAA-containing sdAbs to a subject (e.g., a mammal such as a human). The method includes the step of administering to a mammal, e.g., a human patient, a therapeutic amount of the one or more ncAA-containing sdAbs as described herein sufficient to treat the disease, illness, condition, disorder and/or symptom thereof, under conditions such that the disease or disorder is treated.

The therapeutic methods (which include prophylactic treatment) in general comprise the administration of a therapeutically effective amount of the one or more ncAA-containing sdAbs as described herein, to a subject or patient in need thereof. A subject or patient is meant to include an animal, particularly a mammal, and more particularly, a human. In an embodiment, such one or more ncAA-containing sdAbs used as treatment will be suitably administered to subjects or patients suffering from, having, susceptible to, or at risk for a disease, disorder, or symptom thereof caused by or associated with infection by Botulinum microorganisms and the ensuing producing of neurotoxin, e.g., LC/A, but the Botulinum microorganisms. Determination of patients who are “at risk” can be made by any objective or subjective determination obtained by the use of a diagnostic test or based upon the opinion of a patient or a health care provider (e.g., genetic test, enzyme or protein marker, family history, and the like). The ncAA-containing sdAbs as described herein may be also used in the treatment of any other disorders in which one or more target protein toxins may be implicated.

The methods herein include administering to the subject (including a subject identified as in need of such treatment) an effective amount of the ncAA-containing sdAbs as described herein, or a composition described herein to produce such effect. Identifying a subject in need of such treatment can be in the judgment of a subject himself or herself, or of a health care/medical professional and can be subjective (e.g., opinion) or objective (e.g., measurable or quantifiable by a test or diagnostic method).

### Methods of Use

In an aspect, a method of monitoring treatment progress is provided. The method includes determining a level of toxin or neurotoxin protein as an indicator of disease or infection in a subject suffering from or susceptible to infection by, or disease or illness associated with infection by toxin- or neurotoxin-producing pathogens, in which the subject has been administered a therapeutic amount of one or more of the ncAA-containing sdAbs as described herein sufficient to treat the disease, infection, or symptoms thereof. According to the method, the level of the toxin or neurotoxin protein(s) (which serves as a marker of infection or disease) is detected, measured, or quantified in a biological sample obtained from the subject relative to known levels of the same toxin protein(s) in healthy normal controls and/or in other afflicted patients to establish the subject's treatment progress, disease progress, or disease status. In embodiments, the levels of toxin or neurotoxin protein(s) in the subject's sample are measured or quantified at one or more later time points (following the previous measurements), relative to the levels previously detected or measured in the subject, and/or relative to the levels in normal/healthy subjects or in other afflicted patient controls, to monitor the course, progression, or non-progression of disease or the efficacy of the therapy. In certain embodiments, a pre-treatment level of toxin or neurotoxin protein(s) in the subject is determined prior to beginning treatment according to the method; this pre-treatment level of toxin or neurotoxin protein(s) can then be compared to the level of the toxin or neurotoxin protein(s) in the subject after the treatment commences to monitor or determine the efficacy of the treatment.

Polynucleotides Encoding ncAA-Containing sdAbs

The compositions and methods described herein in various embodiments include an isolated polynucleotide sequence or an isolated polynucleotide molecule that encodes the ncAA-containing sdAbs (VHHs) described herein. Accordingly, the isolated polynucleotide sequence or isolated polynucleotide molecule comprises or consists of a polynucleotide sequence that encodes an ncAA-containing sdAb polypeptide, or a functional portion thereof, as described herein. In an embodiment, a composition comprises a combination of the isolated polynucleotide sequences, isolated polynucleotide molecules, or sdAb polypeptides as described herein.

Also encompassed by the aspects and embodiments described herein are polynucleotide sequences, DNA or RNA, which are substantially complementary to the DNA sequences encoding the polypeptides described herein, and which specifically hybridize with these DNA sequences under conditions of stringency as known to those of skill in the art. As referred to herein, substantially complementary means that the nucleotide sequence of the polynucleotide need not reflect the exact sequence of the original encoding sequences, but must be sufficiently similar in sequence to permit hybridization with a nucleic acid sequence under high stringency conditions. For example, non-complementary bases can be interspersed in a nucleotide sequence, or the sequences can be longer or shorter than the polynucleotide sequence, provided that the sequence has a sufficient number of bases complementary to the sequence to allow hybridization thereto. Conditions for stringency are described, e.g., in Ausubel, F. M., et al., Current Protocols in Molecular Biology, (Current Protocol, 1994), and Brown, et al., Nature, 366:575 (1993); and further defined in conjunction with certain assays.

Vectors, plasmids or viruses containing one or more of the polynucleotide molecules encoding the ncAA-containing sdAb polypeptides are also provided. Suitable vectors for use in eukaryotic and prokaryotic cells are known in the art and are commercially available or readily prepared by the skilled practitioner in the art. Additional vectors can also be found, for example, in Ausubel, F. M., et al., Ibid. and in Sambrook et al., “Molecular Cloning: A Laboratory Manual,” 2nd ED. (1989), and other editions.

Any of a variety of expression vectors (prokaryotic or eukaryotic) known to and used by those of ordinary skill in the art may be employed to express recombinant polypeptides described herein. Expression can be achieved in any appropriate host cell that has been transformed or transfected with an expression vector containing a DNA molecule that encodes a recombinant polypeptide. Suitable host cells include prokaryotes, yeast and higher eukaryotic cells. By way of example, the host cells employed include, without limitation, E. coli, yeast, insect cells, or a mammalian cell line such as COS, CHO, or Human Embryonic Kidney (HEK) cells. The DNA sequences expressed in this manner can encode any of the sdAb polypeptides described herein, including variants thereof.

Uses of plasmids, vectors or viruses containing polynucleotides encoding the sdAb protein molecules described herein include generation of mRNA or protein in vitro or in vivo. In related embodiments, host cells transformed with the plasmids, vectors, or viruses are provided, as described above. Nucleic acid molecules can be inserted into a construct (such as a prokaryotic expression plasmid, a eukaryotic expression vector, or a viral vector construct, which can, optionally, replicate and/or integrate into a recombinant host cell by known methods. The host cell can be a eukaryote or prokaryote and can include, for example, yeast (such as Pichia pastoris or Saccharomyces cerevisiae), bacteria (such as E. coli, or Bacillus subtilis), animal cells or tissue (Chinese hamster ovary (CHO) cells or COS cells), insect Sf9 cells (such as baculoviruses infected SF9 cells), or mammalian cells (somatic or embryonic cells, HEK cells, CHO cells, HeLa cells, human 293 cells and monkey COS-7 cells). Suitable host cells also include a mammalian cell, a bacterial cell, a yeast cell, an insect cell, and a plant cell.

An sdAb-encoding polynucleotide molecule can be incorporated or inserted into the host cell by known methods. Examples of suitable methods for transfecting or transforming host cells include, without limitation, calcium phosphate precipitation, electroporation, microinjection, infection, lipofection and direct uptake. “Transformation” or “transfection” refers to the acquisition of new or altered genetic features by the incorporation of additional nucleic acids, e.g., DNA, into cellular DNA. “Expression” of the genetic information of a host cell is a term of art which refers to the directed transcription of DNA to generate RNA that is, in turn, translated into a polypeptide. Procedures for preparing recombinant host cells and incorporating nucleic acids are described in more detail in, e.g., Sambrook et al., “Molecular Cloning: A Laboratory Manual,” Second Edition (1989) and Ausubel, et al. “Current Protocols in Molecular Biology,” (1992), and later editions.

A transfected or transformed host cell is maintained under suitable conditions for expression and recovery of the sdAb polypeptides described herein. In certain embodiments, the cells are maintained in a suitable buffer and/or growth medium or nutrient source for growth of the cells and expression (and secretion) of the gene product(s) into the growth medium. The type of growth medium is not critical and is generally known to those skilled in the art, such as, for example, growth medium and nutrient sources that include sources of carbon, nitrogen and sulfur. Examples include Luria-Bertani (LB) broth, Superbroth, Dulbecco's Modified Eagles Media (DMEM), RPMI-1640, M199, Grace's insect medium, and yeast culture media, e.g., yeast peptone dextrose (YPD) or yeast extract peptone dextrose (YEPD), yeast culture minimal media, synthetic defined medium with casamino acids (SD-CAA or SD-SCAA), (SigmaAldrich). The growth medium can contain a buffering agent, as commonly used in the art. The pH of the buffered growth medium may be selected and is generally a pH that is tolerated by, or optimal for, growth of the host cell, which is maintained under a suitable temperature and atmosphere.

In another aspect, an RNA polynucleotide, in particular, mRNA, encodes the sdAb polypeptides described herein. mRNA encoding an sdAb as described herein may contain a 5′ cap structure, a 5′ UTR, an open reading frame, a 3′ UTR and polyA sequence followed by a C30 stretch and a histone stem loop sequence (Thess, A. et al., 2015, Mol Ther, 23(9):1456-1464; Thran, M. et al., 2017, EMBO Molecular Medicine, DOI: 10.15252/emmm.201707678). Sequences may be codon-optimized for human use. In an embodiment, the mRNA sequences do not include chemically modified bases. mRNAs encoding the sdAbs as described herein may be capped enzymatically or further polyadenylated for in vivo studies or use.

Expression of proteins, which normally have a shortened serum half-life, by encoding mRNA, particularly sequence optimized, unmodified mRNA, advantageously prolongs the bioavailability of these proteins for in vivo activity. (See, e.g., K. Kariko et al, 2012, Mol. Ther., 20:948-953; Thess, A. et al., 2015, Mol Ther, 23(9):1456-1464;). Accordingly, in embodiments, an sdAb as described herein, or a dimeric or multimeric polypeptide comprising more than one sdAb, with an estimated serum half-life of 1-2 days (with albumin-binding), are likely to benefit from being encoded by mRNA and have, for example, a half-life of sdAb serum titer that may be 1.5-fold higher at one to three days after treatment.

In some embodiments, an ncAA-containing sdAb protein (an ncAA-containing sdAb protein monomer), can be modified, for example, by attachment (e.g., directly or indirectly via a linker or spacer) to another ncAA-containing sdAb protein monomer. In some embodiments, ncAA-containing sdAb monomer is attached or genetically (recombinantly) fused to another ncAA-containing sdAb protein monomer. Accordingly, the polynucleotide (DNA) that encodes one ncAA-containing sdAb protein monomer is joined (in reading frame) with the DNA encoding a second ncAA-containing sdAb protein monomer, and so on. In certain embodiments, additional amino acids are encoded within the polynucleotide between the ncAA-containing sdAb protein monomers so as to produce an unstructured region (e.g., a flexible spacer) that separates the VHH binding protein monomers, e.g., to better promote independent folding of each ncAA-containing sdAb protein monomer into its active conformation or shape. Commercially available techniques for fusing proteins (or their encoding polynucleotides) may be employed to recombinantly join or couple the ncAA-containing sdAb protein monomers into dimeric or multimeric binding proteins containing two or more of the same or different ncAA-containing sdAb proteins as described herein.

Polynucleotide sequences encoding the ncAA-containing sdAbs as described herein can be recombinantly expressed, and the resulting encoded ncAA-containing sdAb can be produced at high levels and isolated and/or purified. In an embodiment, the recombinant ncAA-containing sdAbs are produced in soluble form.

### Pharmaceutical Compositions

Also provided herein are methods for treating or preventing disease, pathologies, and the symptoms thereof, caused by or associated with infection by a pathogen and the resultant production of a disease-causing antigen, such as a toxin or neurotoxin, by the pathogen within cells and/or in a subject. In an embodiment, methods for treating or preventing disease, pathologies, and the symptoms thereof, caused by or associated with infection by a Botulinum pathogen and the resultant production of neurotoxin, such as LC/A neurotoxin, by the Botulinum pathogen within cells and/or in a subject are provided. The methods include administering to a subject in need thereof an effective amount of the ncAA-containing sdAbs described herein. The ncAA-containing sdAbs are covalently crosslinked to the target antigen, e.g., Botulinum toxin LC/A, following the reactivity of the ncAA in the sdAb. In an embodiment, the ncAA-containing sdAbs are provided, included, or used in a pharmaceutical composition. In an embodiment, the ncAA-containing sdAbs neutralize the activity, e.g., toxicity, of the target antigen.

Typically, a carrier, excipient, diluent, or vehicle is included in a composition as described herein, such as a pharmaceutically acceptable carrier, diluent, excipient, or vehicle, which includes, for example, sterile water, aqueous saline solution, aqueous buffered saline solutions, aqueous sucrose, dextrose, or mannose solutions, aqueous glycerol solutions, ethanol, calcium carbonate, albumin, starch, cellulose, silica gel, polyethylene glycol (PEG), dried skim milk, rice flour, magnesium stearate, and the like, or combinations thereof. The terms “pharmaceutically acceptable carrier” and a “carrier” refer to any generally acceptable excipient or drug delivery vehicle or device that is relatively inert and non-toxic.

The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like. Such methods also include administering an adjuvant, such as an oil-in-water emulsion, a saponin, a cholesterol, a phospholipid, a CpG, a polysaccharide, variants thereof, and a combination thereof, with a composition as described herein. Optionally, a formulation for prophylactic administration may also contain one or more adjuvants for enhancing the effect of, or an immune response to, an antigen or immunogen, e.g., binding proteins as described herein. Suitable adjuvants include, without limitation, complete Freund's adjuvant, incomplete Freund's adjuvant, saponin, alum, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvants QS-21 and MF59.

The administration of the ncAA-containing sdAbs as described herein, or a pharmaceutical composition thereof, as a therapeutic for the treatment or prevention of disease, infection, or pathology, e.g., caused by infection by a Botulinum pathogen and the ensuing production of neurotoxin by the Botulinum pathogen, may be by any suitable means that results in a concentration of the therapeutic that, combined with other components, if desired, is effective in ameliorating, reducing, abating, abrogating, stabilizing, or eliminating disease, pathology, and/or the symptoms thereof in a subject. The therapeutic may be administered systemically, for example, formulated in a pharmaceutically-acceptable composition or buffer such as physiological saline.

Routes of administration include, for example and without limitation, subcutaneous, intravenous, intraperitoneal, intramuscular, intrathecal, intraperitoneal, or intradermal injections that provide continuous, sustained levels of the therapeutic in the subject. Other routes include, without limitation, gastrointestinal, esophageal, oral, rectal, intravaginal, etc. The amount of the therapeutic to be administered varies depending upon the manner of administration, the age and body weight of the subject, and with the clinical symptoms of the bacterial infection or associated disease, pathology, or symptoms. Generally, amounts will be in the range of those used for other agents used in the treatment of disease or pathology associated with infection by a Botulinum pathogen and the ensuing production of neurotoxin by the Botulinum pathogen, although in certain instances, lower amounts may be suitable because of the increased range of protection and treatment afforded by the provision of the described ncAA-containing sdAbs as a therapeutic. A composition is administered at a dosage that ameliorates, decreases, diminishes, abates, alleviates, or eliminates the effects of the bacterial (microorganism) infection or disease (e.g., neurotoxicity and/or the symptoms thereof) as determined by a method known to one skilled in the art.

In embodiments, a therapeutic or prophylactic treatment agent may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, intrathecal, or intraperitoneal) administration route. The pharmaceutical compositions may be formulated according to conventional pharmaceutical practice (See, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the gut or gastrointestinal system; (v) formulations that allow for convenient dosing, such that doses are administered, for example, once every one or two weeks, or other time periods; and (vi) formulations that target a disease using carriers or chemical derivatives to deliver the therapeutic agent or drug to a particular cell type. For some applications, controlled release formulations obviate the need for frequent dosing during the day to sustain a therapeutic level in plasma, serum, or blood. In an embodiment, one or more ncAA-containing sdAb, or a dimeric or multimeric protein thereof, may be formulated with one or more additional components for administration to a subject in need.

Any of a number of strategies can be pursued in order to obtain controlled release of a therapeutic agent in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

A pharmaceutical composition may be administered parenterally by injection, infusion, or implantation (subcutaneous, intravenous, intramuscular, intradermal, intraperitoneal, intrathecal, or the like) in dosage forms, formulations, or via suitable delivery devices or implants containing conventional, non-toxic pharmaceutically acceptable carriers and adjuvants. A pharmaceutical composition may also be provided by oral, buccal, topical (e.g., via powders, ointments, or drops), rectal, mucosal, sublingual, intracisternal, intravaginal, rectal, ocular, or intranasal administration. The formulation and preparation of such compositions are well known to those skilled in the art of pharmaceutical formulation. Formulations can be found in Remington: The Science and Practice of Pharmacy, noted supra.

Compositions for parenteral or oral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device or vehicle for implantation, or it may be presented as a dry powder to be reconstituted with water or another suitable vehicle before use. The composition may include suitable parenterally acceptable carriers and/or excipients. In some cases, an active therapeutic agent(s) may be incorporated into microspheres, microcapsules, nanoparticles, liposomes, or the like, e.g., for controlled release. Furthermore, the composition may include suspending, solubilizing, stabilizing, pH-adjusting agents, tonicity adjusting agents, and/or dispersing, agents.

In some embodiments, a pharmaceutical composition comprising an active therapeutic is formulated for systemic delivery, intravenous delivery, e.g., intravenous injection, subcutaneous delivery, or local delivery (e.g., diffusion). To prepare such a composition, the suitable therapeutic(s) are dissolved or suspended in a parenterally acceptable liquid vehicle, excipient, or solvent. Among acceptable vehicles and solvents that may be employed are, for example, water; water adjusted to a suitable pH by the addition of an appropriate amount of hydrochloric acid, sodium hydroxide or a suitable buffer; 1,3-butanediol; Ringer's solution; and isotonic sodium chloride solution and dextrose solution. An aqueous formulation may also contain one or more preservatives (e.g., methyl, ethyl or n-propyl p-hydroxybenzoate). In cases in which a therapeutic agent is only sparingly or slightly soluble in water, a dissolution enhancing or solubilizing agent can be added, or the solvent may include 10-60% w/w of propylene glycol or the like.

In some embodiments, compositions comprising the binding proteins are sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. In some embodiments, the binding proteins are combined, where desired, with other active substances, e.g., enzyme inhibitors, to reduce metabolic degradation.

An effective amount of pharmaceutically acceptable compositions can vary according to the choice or type of the ncAA-containing sdAbs as described herein, the particular composition formulated, the mode of administration and the age, weight and physical health or overall condition of the patient, for example. In an embodiment, an effective amount of ncAA-containing sdAbs is an amount which is capable of reducing one or more symptoms of the disease or pathology caused by the infectious agent/disease target. Dosages for a particular patient are determined by one of ordinary skill in the art using conventional considerations, (e.g. by means of an appropriate, conventional pharmacological protocol).

In certain embodiments, a composition includes one or more polynucleotide sequences that encode one or more of the ncAA-containing sdAbs as described herein. In an embodiment, a polynucleotide sequence encoding an ncAA-containing sdAb is in the form of a DNA molecule. In an embodiment, a polynucleotide sequence encoding an ncAA-containing sdAb is in the form of an RNA molecule. In some embodiments, the composition includes a plurality of nucleotide sequences each encoding an ncAA-containing sdAb, or a combination of ncAA-containing sdAbs described herein, such that the ncAA-containing sdAb is expressed and produced in situ. In such compositions, a polynucleotide sequence is administered using any of a variety of delivery systems known to those of ordinary skill in the art, including eukaryotic, bacterial and viral vector nucleic acid expression systems. Suitable nucleic acid expression systems contain appropriate nucleotide sequences operably linked for expression in a patient (such as suitable promoter and termination signals). Bacterial delivery systems involve administration of a bacterium (such as Bacillus-Calmette-Guerrin) that expresses the polypeptide on its cell surface. In an embodiment, the ncAA-containing sdAb-encoding nucleic acid can be introduced using a viral expression system (e.g., vaccinia or other pox virus, retrovirus, adenovirus, adeno-associated virus, or recombinant adeno-associated virus), which uses a non-pathogenic (defective), replication competent virus. Techniques for incorporating nucleic acid (DNA) into such expression systems are well known to those of ordinary skill in the art. The nucleic acid (DNA) can also be “naked,” as described, for example, in Ulmer et al., 1993, Science, 259:1745-1749 and as reviewed by Cohen, 1993, Science 259:1691-1692. The uptake of naked DNA can be increased by coating the DNA onto biodegradable beads, which are efficiently transported into recipient cells.

### Kits

Provided are kits for use in the treatment or prevention of an infection or disease caused by or associated with pathogenic organisms or microorganisms that produce pathogenic proteins, such as toxins or neurotoxins. In an embodiment, the pathogenic microorganism is Botulinum toxin and the neurotoxin is LC/A protease. In some embodiments, the kit includes an effective amount of one or more ncAA-containing sdAbs as described herein, in unit dosage form. In other embodiments, the kit includes a therapeutic or prophylactic composition containing an effective amount of one or more ncAA-containing sdAbs in unit dosage form. In some embodiments, the kit comprises a device (e.g., an automated or implantable device for subcutaneous delivery; an implantable drug-eluting device, or a nebulizer or metered-dose inhaler) for dispersal of the composition or a sterile container which contains a pharmaceutical composition. Non-limiting examples of containers include boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition as described is provided together with instructions for administering the pharmaceutical composition containing one or more ncAA-containing sdAbs to a subject having or at risk of contracting or developing an infection or disease or pathology, and/or the symptoms thereof, e.g., associated with infection by Botulinum microorganisms that produce neurotoxin, such as LC/A protease. The instructions generally include information about the use of the composition, e.g., for the treatment or prevention of an infection and intoxication by Botulinum bacteria and the toxin proteins that they produce. In other embodiments, the instructions include at least one of the following: description of the therapeutic/prophylactic agent; dosage schedule and administration for treatment or prevention of infection, disease or symptoms thereof caused by the pathogen(s), e.g., by Botulinum bacteria and the toxin proteins that they produce; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the aspects and embodiments as described herein employs, unless otherwise indicated, conventional techniques of molecular biology (including recombinant techniques), microbiology, cell biology, biochemistry and immunology, which are well within the purview of the skilled practitioner in the art. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polypeptides (proteins) and polynucleotides as described, and, as such, may be considered in making and practicing the described and disclosed aspects and embodiments. Particularly useful techniques for particular aspects and embodiments are discussed in the sections that follow.

### EXAMPLES

### Example 1: Mutant Design and Orthogonal Translation System

The crystallographic and biochemical characterization of a suite of alpaca-derived sdAbs capable of binding and inhibiting the Botulinum neurotoxin A (BoNT/A) with high affinity are reported by Lam. K.-h. et al., 2022, PLOS Pathogens, 18 (1), e1010169. DOI: 10.1371/joumal.ppat.1010169 (47) and Lam, K.-h. et al., 2020, Cell Reports, 30 (8), 2526-2539.e2526. DOI: doi.org/10.1016/j.celrep.2020.01.107 (48), as well as described in U.S. Provisional Application No. 63/275,563, filed on Nov. 4, 2021, which are incorporated herein by reference in their entireties). Notably, the findings show different LC/A epitopes accessible to the sdAbs for binding and inhibition. As described herein, the targeting of different regions of LC/A with these sdAbs would likely increase the chances of finding appropriate sites conducive to covalent bond formation.

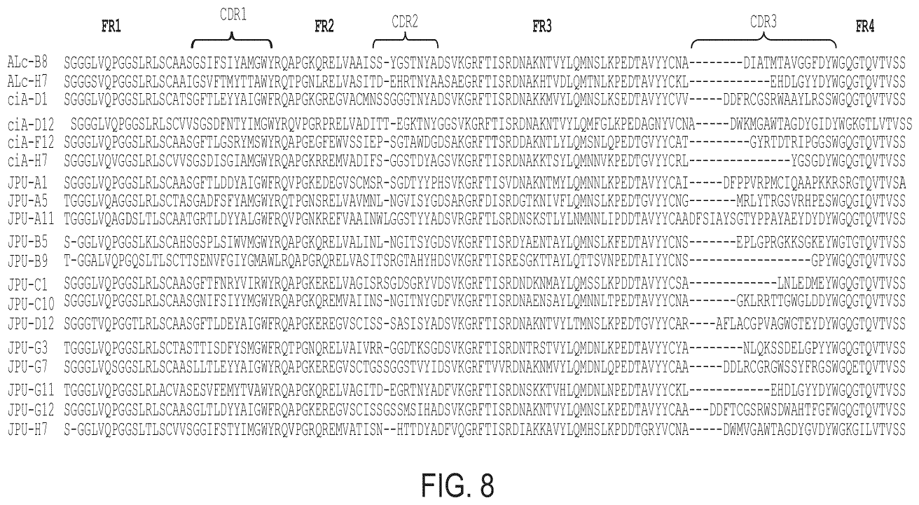

Based on the available crystal structures of high-affinity sdAb-LC/A complexes, (Ibid.), a collection of sdAbs was designed. FIG. 8 and Table 1 provide the amino acid sequences of sdAbs, including the parent anti-Botulinum LC/A sdAbs of the ncAA-containing sdAbs described herein, that can be used to generate the described sdAbs containing ncAAs. The parent sdAbs (VHHs) are described in U.S. Provisional Application No. 63/275,563, filed Nov. 4, 2021, the contents of which are incorporated by reference herein. Each of the designed sdAb contained ncAAs at positions likely to result in covalent interaction. Three of the chosen sdAbs, JPU-A5, JPU-C1, JPU-C10, were potent inhibitors of LC/A protease activity (FIGS. 1B; FIG. 8, and Table 1), (Lam. K.-h. et al., 2022, PLOS Pathogens, 18 (1), e1010169. DOI: 10.1371/joumal.ppat.1010169) and one LC/A-binding sdAb, ciA-H7, (also termed JDQ-H7), had no protease inhibition activity but prevented BoNT/A intoxication of neuronal cells (FIG. 1B), (Lam, K.-h. et al., 2020, Cell Reports, 30 (8), 2526-2539.e2526. DOI: doi.org/10.1016/j.celrep.2020.01.107).

Encouraged by developments in studying AzF-mediated photo-crosslinking events on the yeast surface, and with access to the AcFRS orthogonal suppression system to introduce this ncAA in response to the TAG codon,^{24, 49, 50} positions in sdAbs were chosen that were deemed likely to result in photo-crosslinking to LC/A when incorporating AzF. Guided by the structural information of the crystal structures of the sdAb-LC/A complexes and utilizing recent developments in studying AzF-mediated photocrosslinking events on the yeast surface, as well as access to the AcFRS orthogonal translation system (OTS) to introduce this ncAA²⁴ in response to the TAG codon in S. cerevisiae, five to six positions were chosen in each camelid sdAb proximal to the LC/A with aims at favoring a covalent interaction (FIGS. 1B, 1C). Also chosen were several mutants (JPU-A5 Q1; JPU-C10 Q1, Q44 and Y111; ciA-H7 Q1 (JDQ-H7 Q1)) that lie farther away from the sdAb-LC/A interface to investigate the properties of the clones in this work. In general, the mutations were localized to complementarity determining regions (CDRs), although several framework positions were also included, particularly when substituting aromatic residues in an effort to minimize structural disruptions, yet still present chemical functionality within proximity of the LC/A target.

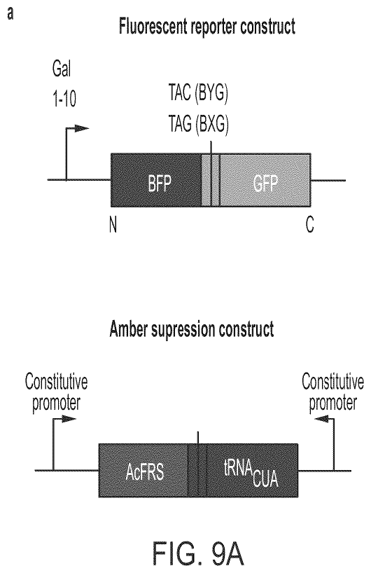

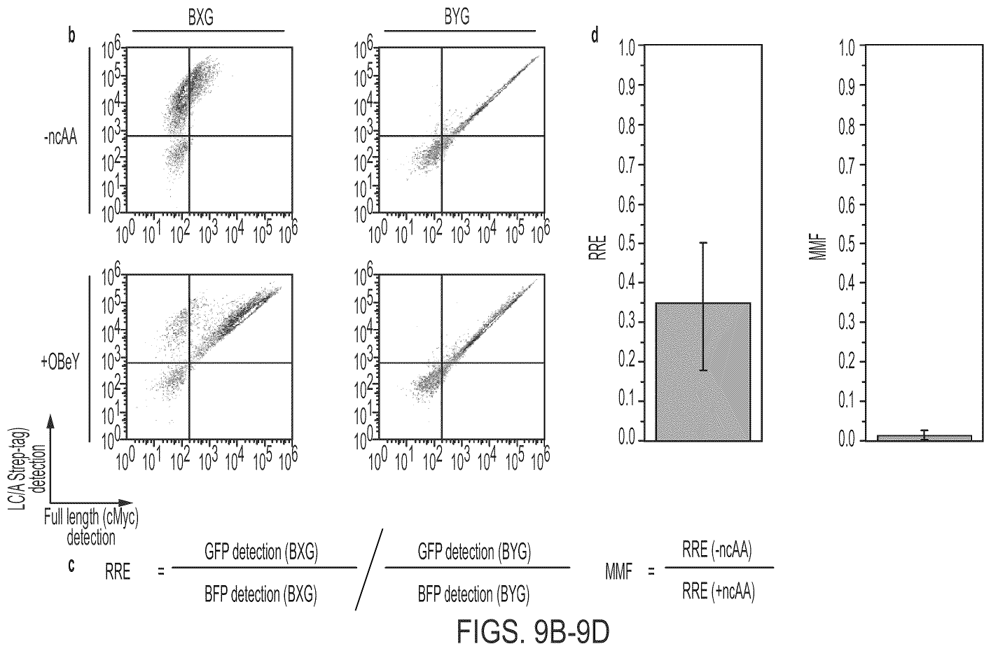

While performing studies to identify positions amenable for photo-crosslinked interactions, it was decided to probe the mutant collection in the context of spontaneous crosslinking. In view of the reactivity of electrophilic ncAAs,^{31, 32} O-(2-bromoethyl)tyrosine (OBeY) was utilized due to its structural similarity to other encodable amino acids in yeast and its commercial availability. Given the known polyspecificity of AcFRS,⁴⁹ the incorporation of OBeY was evaluated with this synthetase by using a dual fluorescent reporter.⁵¹ The results indicated that AcFRS supported OBeY incorporation in response to the TAG codon with low misincorporation events (FIGS. 9A-9D). Based on the crystallographic data, amino acid positions that could lead to spontaneous crosslinking were identified on the JPU-C1 sdAb and include the following: R31, R53, D56, S57, R59, N100, E102, D103, and Y106. With the designed mutants in hand and a suitable suppression machinery to incorporate the chosen ncAAs, the constructs were prepared in display format to evaluate their performance in binding to the target LC/A protease (FIG. 1D).

### Example 2: Display and Binding Validation

To streamline the investigations of ncAA-substituted camelids, all variants were prepared in yeast display format to evaluate the binding and crosslinking properties of all mutants. Each wild type (WT) gene was cloned into the pCTCON2 vector⁵² encoding for the sdAb antibody tethered to the C-terminus of the Aga2p protein. This particular display orientation was chosen due to the important protein contacts that occur between LC/A and the CDR3 loops of the sdAbs, which are located near the C-termini of the sdAbs. It is possible that displaying sdAbs as N-terminal fusions to Aga2p would position CDR3 loops close enough to Aga2p to interfere with LC/A binding. During secretion, Aga2p becomes covalently linked to the Aga1p protein and transferred to the yeast surface, resulting in display of the sdAb. The sdAb is flanked by HA and cMyc tags at the N terminus and C terminus, respectively, allowing for detection of the full-length sdAb by labeling for the cMyc tag with fluorescently labeled reagents (FIG. 1D). The mutant plasmids were constructed by introducing TAG codons at the selected positions using typical Gibson assembly procedures. S. cerevisiae RJY10053 was transformed with the corresponding pCTCON2 plasmid and pRS315_KanRMod_AcFRS,^{24, 49, 50} which encodes for the AcFRS tRNA aminoacyl synthetase and the corresponding orthogonal tRNA for incorporation of ncAAs in response to the TAG codon.

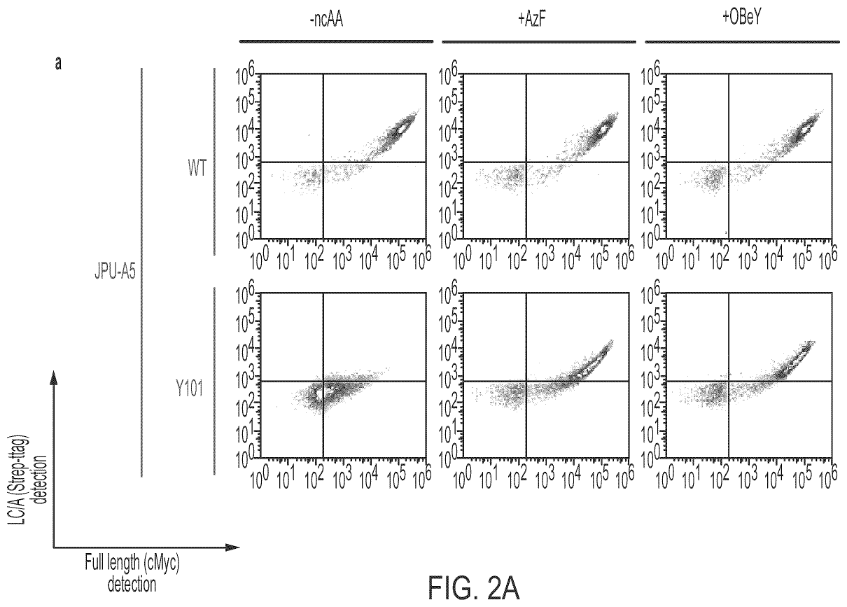

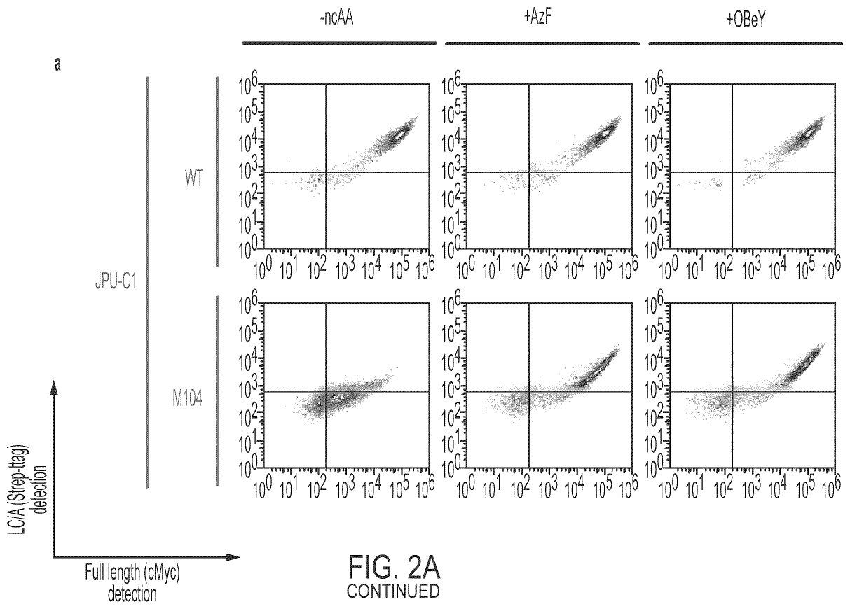

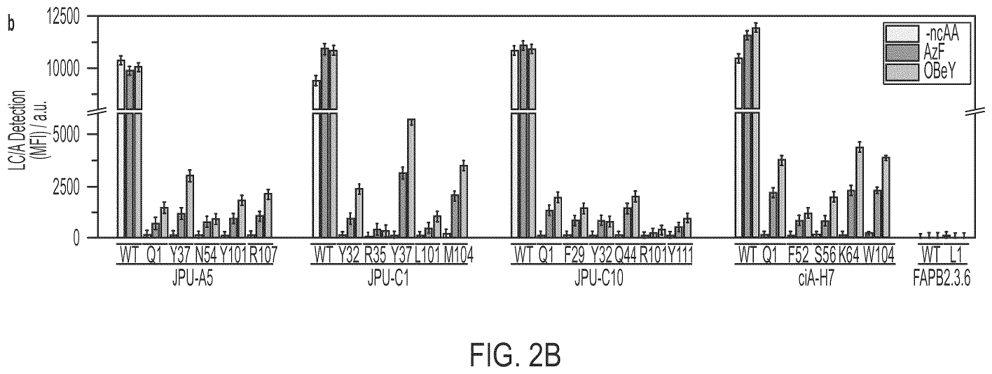

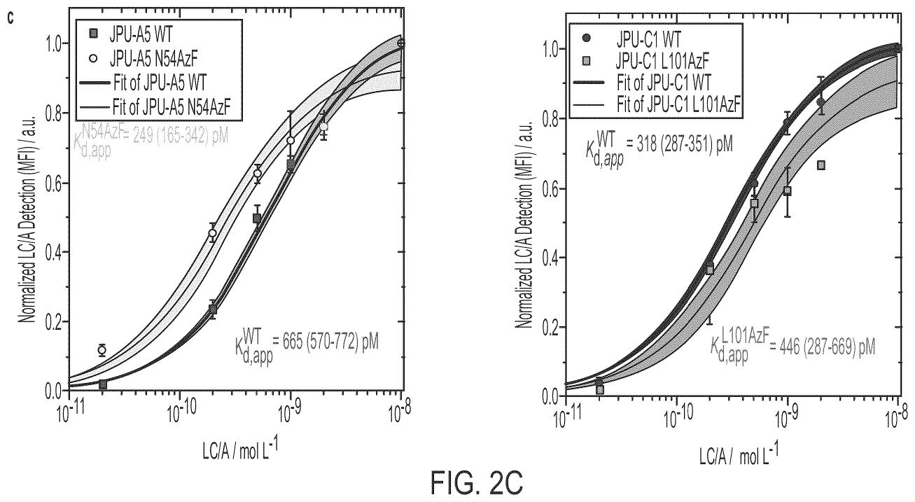

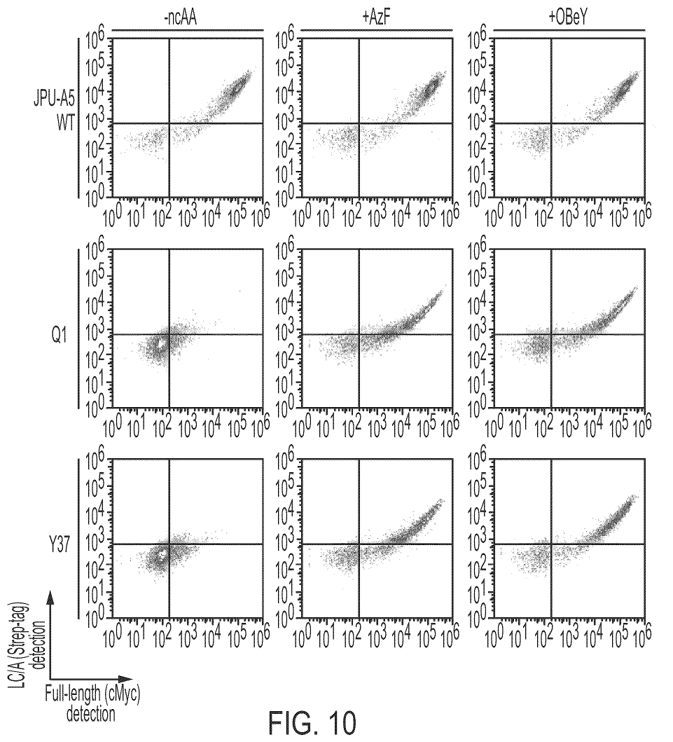

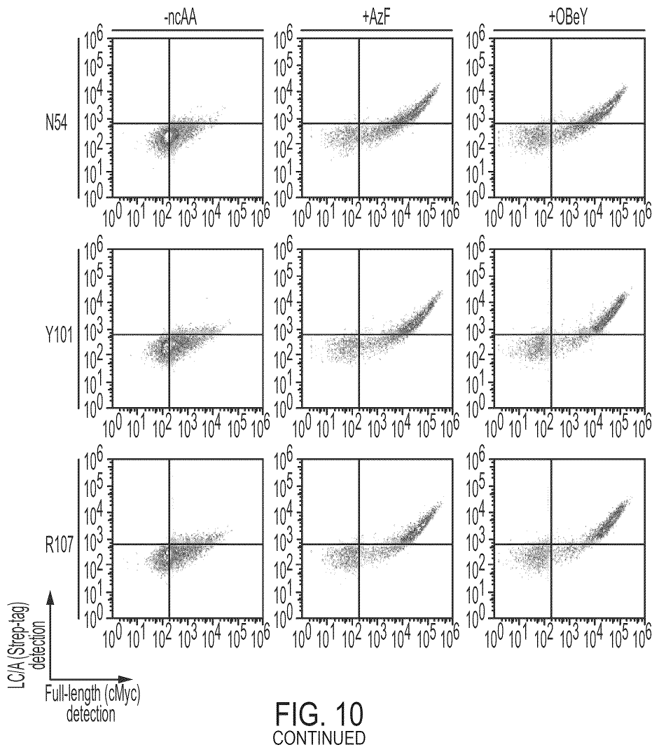

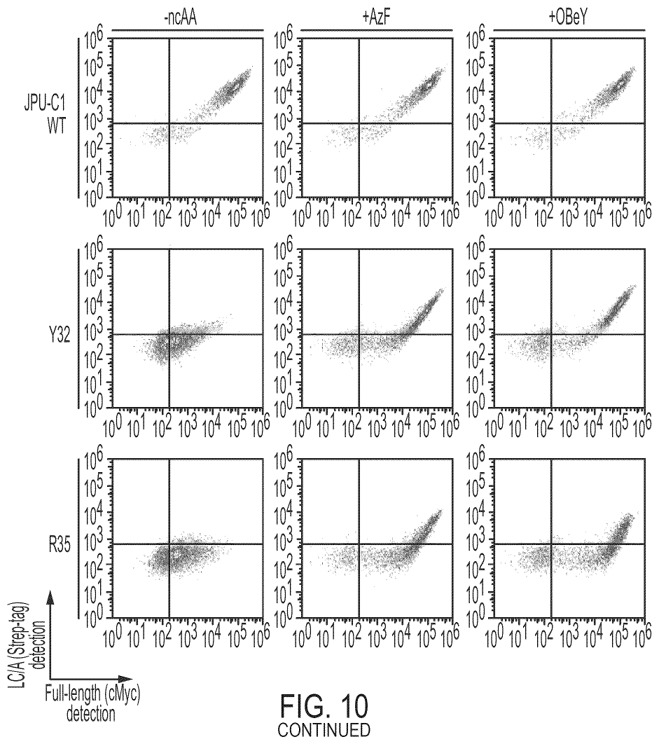

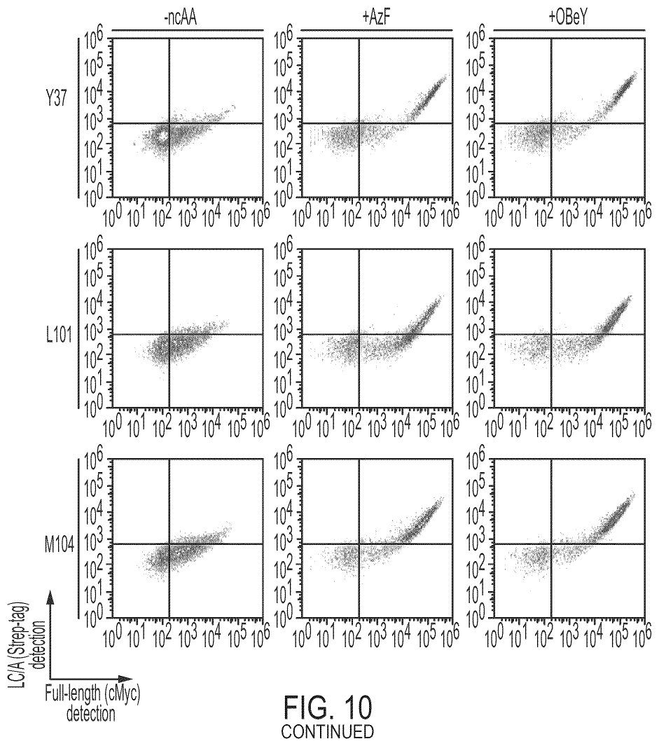

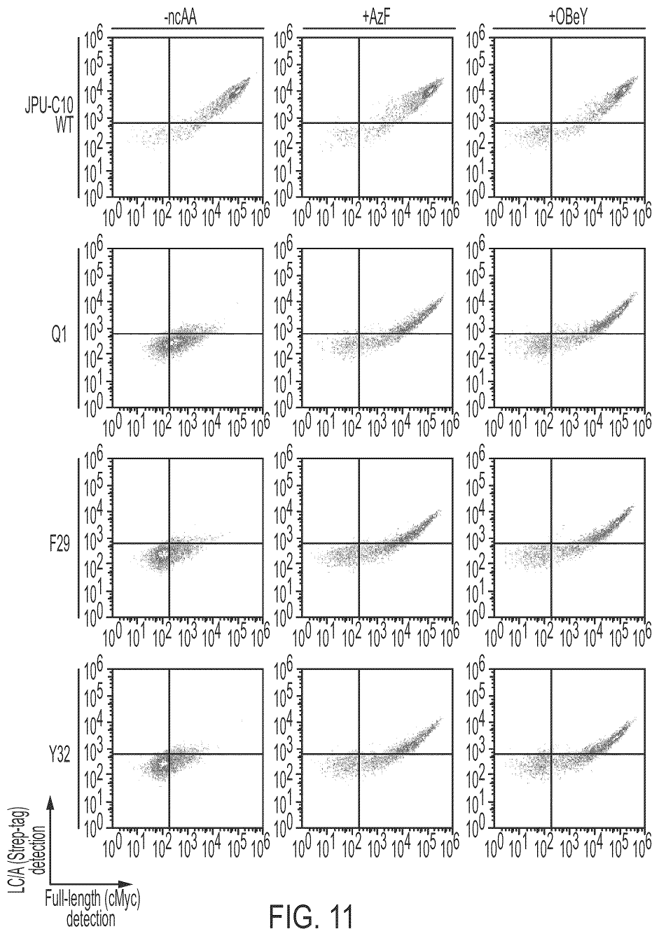

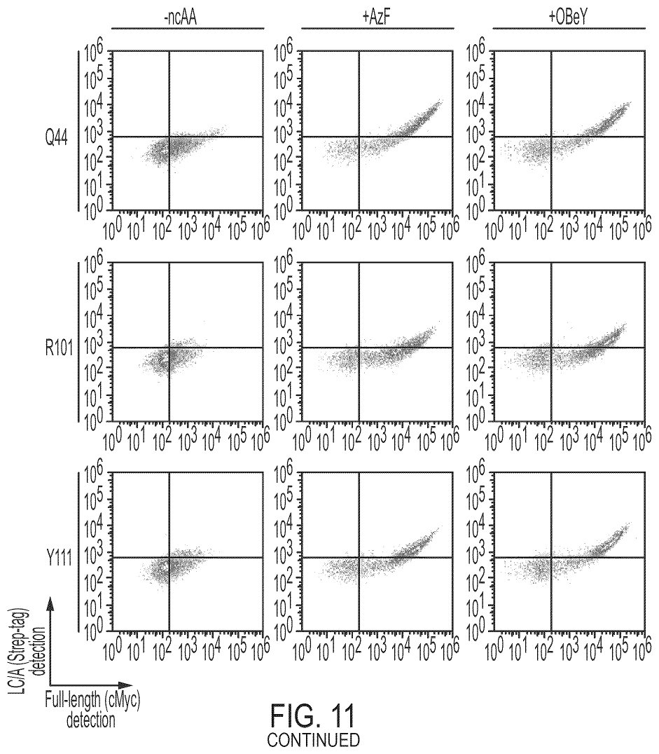

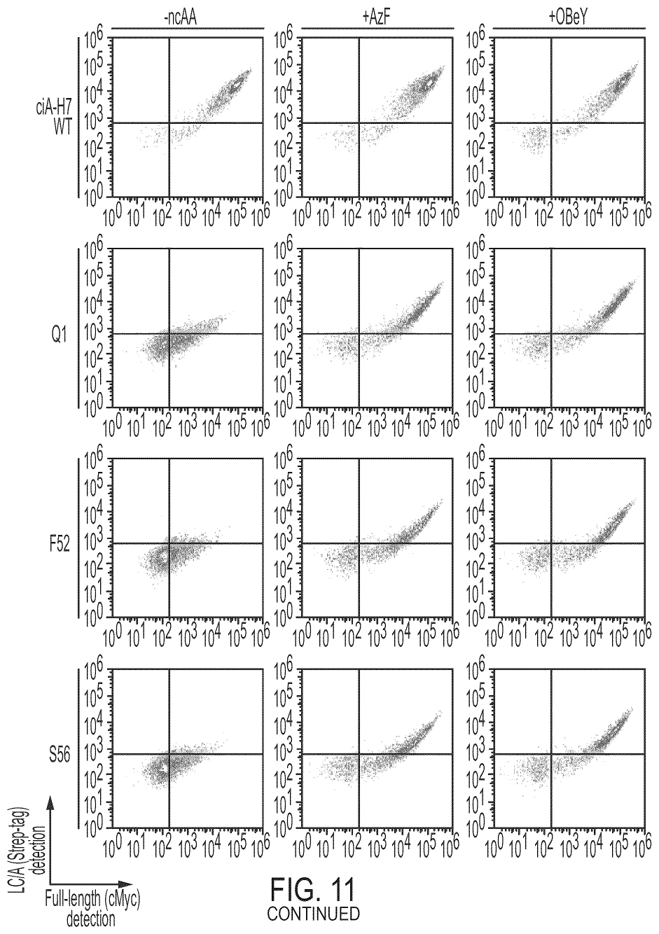

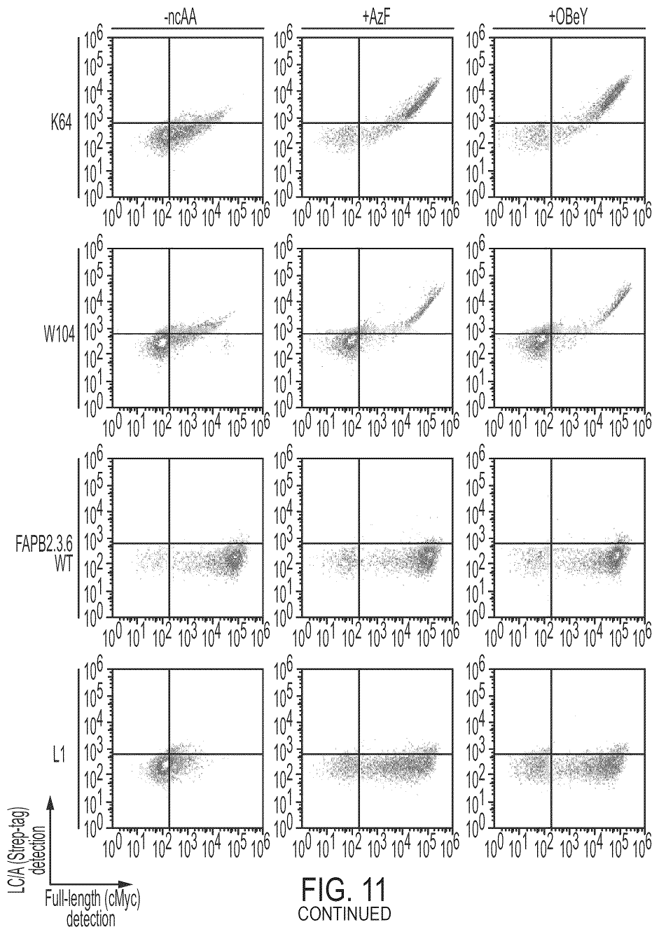

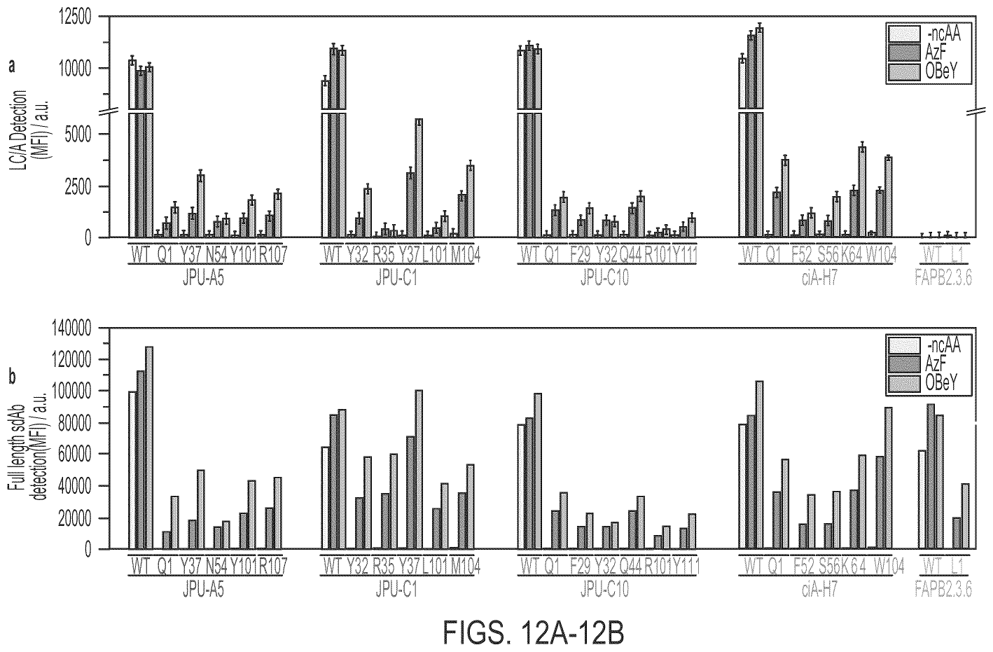

The display of the sdAbs and their ability to bind to LC/A after induction were evaluated in the absence or presence of 1 mM AzF or OBeY, respectively. After incubation of induced cells with LC/A and removal of excess protease, the cells were labeled against the C-terminal cMyc tag of the sdAb and the C-terminal Strep-tag of the LC/A. Flow cytometry analysis showed a dependence of cMyc signal on the presence of ncAA during induction for the TAG mutants, but not for the WT constructs, showcasing the expected behavior stemming from truncation by the lack of ncAA to enable translation (FIG. 2A, FIG. 2B, FIG. 10, FIG. 11, and FIGS. 12A and 12B). Binding to LC/A was detected for nearly all substitution sites incorporating AzF or OBeY, while no LC/A binding was observed in truncated proteins when inducing with no ncAA. Given that LC/A was not detectable in any of the non-binding protein controls (scFvs FAPB2.3.6 WT and L1TAG—where L1 represents position 1 of the light chain^{49, 51}), the results indicate that only the full-length sdAbs bound to LC/A, and that ncAA incorporation at these sites did not abolish the interaction between the proteins.

To further characterize the effect of the ncAAs on binding, two sdAbs were selected for quantitative examination of binding affinity via titration of LC/A to a fixed concentration of sdAb displayed on yeast cells⁵². The analysis was limited to AzF-containing clones, as spontaneous crosslinking from OBeY would not be expected to follow the standard behavior of reversible binding events and prevent determination of affinity constants. JPU-A5 N5AzF and JPU-C1 L101AzF were chosen, as they showed promising photoreactivity in the initial screening, as well as their corresponding WT counterparts for comparison. Titrations were carried out with a fixed concentration of induced cells with varying concentrations of LC/A, and excess LC/A was removed before fluorescent labeling for flow cytometry. The apparent dissociation constant (K_{d, app}) values were then estimated by fitting to a one-site binding model as shown in FIG. 2C. The fitted models resulted in similar values for the dissociation constants of the AzF mutants compared to their parent WT, supporting the earlier observations that ncAA incorporation does not significantly impair binding to the target.

These experiments demonstrate the binding capabilities of these sdAb variants by showing that ncAA incorporation therein does not result in a significant change in affinity toward the target antigen LC/A. In particular, in the case of the JPU-A5 N54AzF and JPU-C1 L101AzF mutants, these findings represent the foundation for the construction of ncAA-expanded libraries that can allow the selection of desirable covalent antibodies in future screening campaigns. Having demonstrated that the antibody collection retained binding toward LC/A, a platform to identify sdAbs candidates capable of crosslinking to LC/A either after UV exposure (in the case of AzF), or spontaneously (in the case of OBeY) was used.

### Example 3: Photo-crosslinking with AzF

AzF has been used extensively for its reactivity upon exposure to UV light and has been particularly useful to explore the molecular underpinnings of protein-protein interactions. Photo-crosslinking between scFvs incorporating AzF and their binding partners can be studied on the yeast surface.^{25-30, 54} (Islam, M., et al., 2021, ACS Chemical Biology, 16 (2), 344-359. DOI: 10.1021/acschembio.0c00865). Thus, the collection of AzF-containing sdAbs was evaluated to find positions that would result in covalent crosslinking events with LC/A.

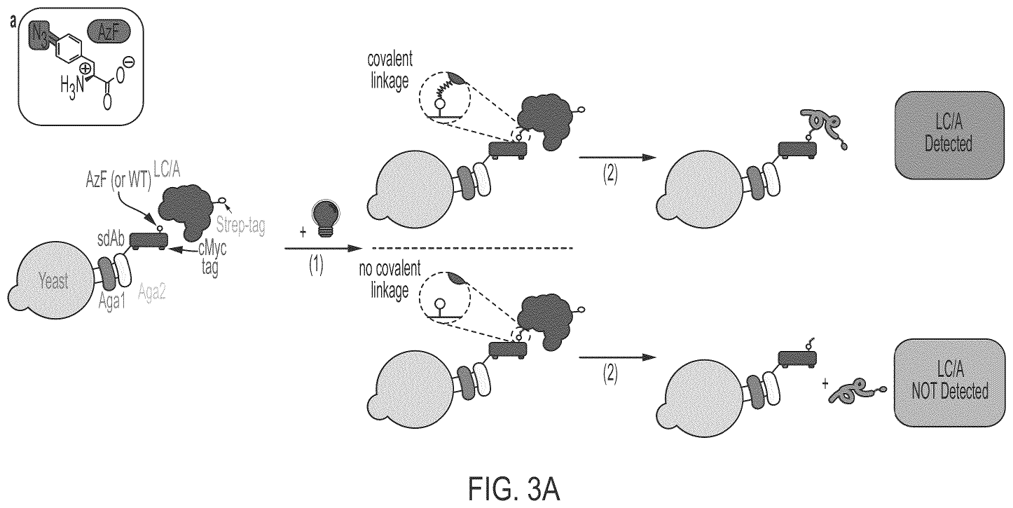

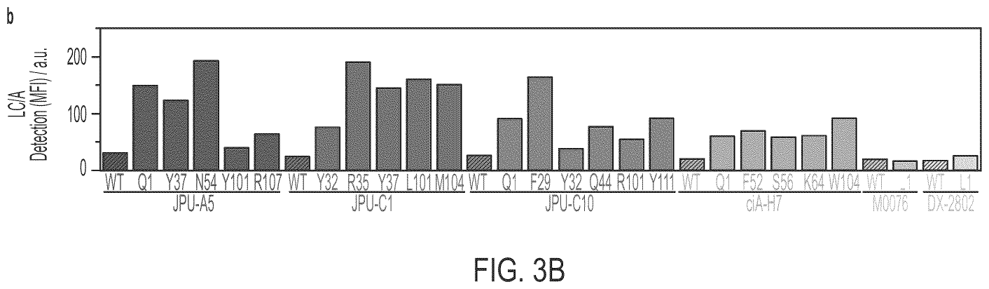

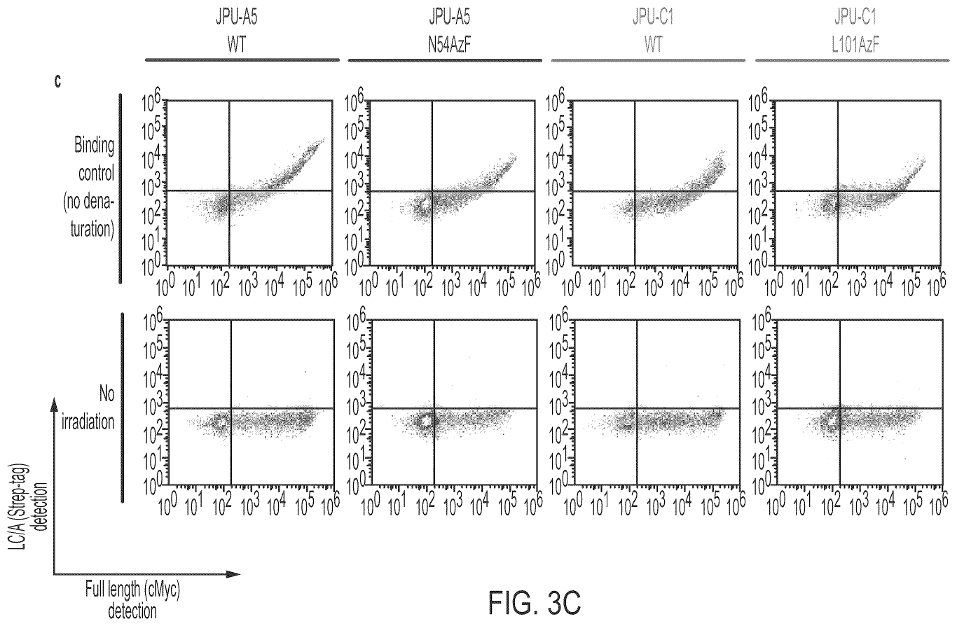

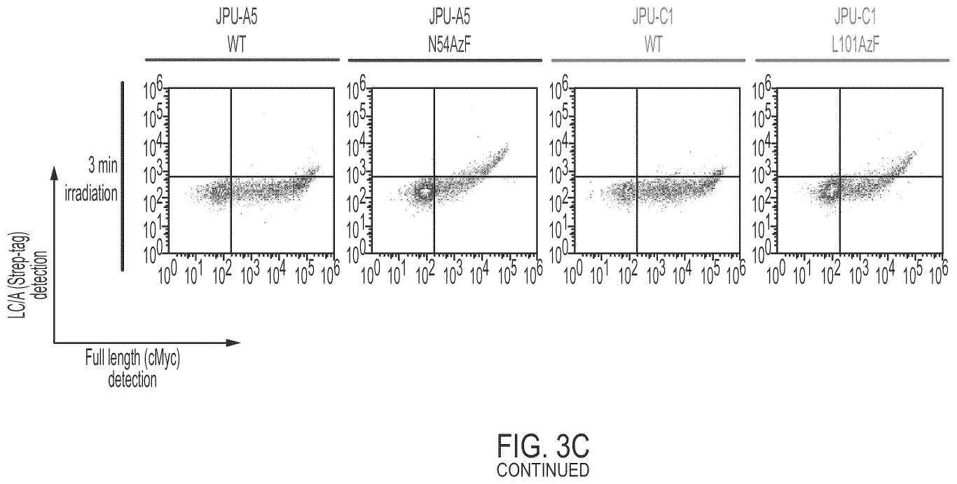

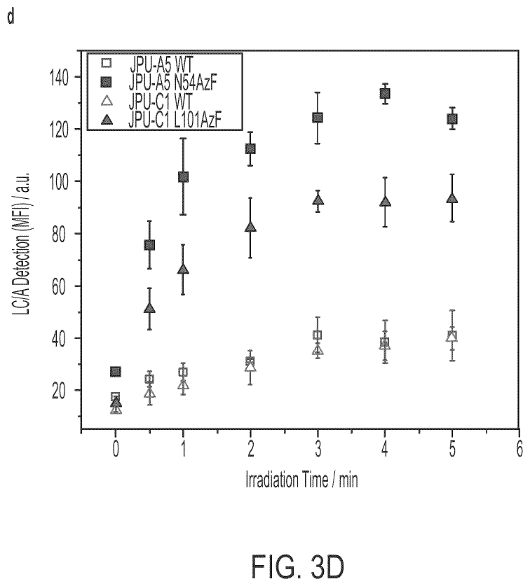

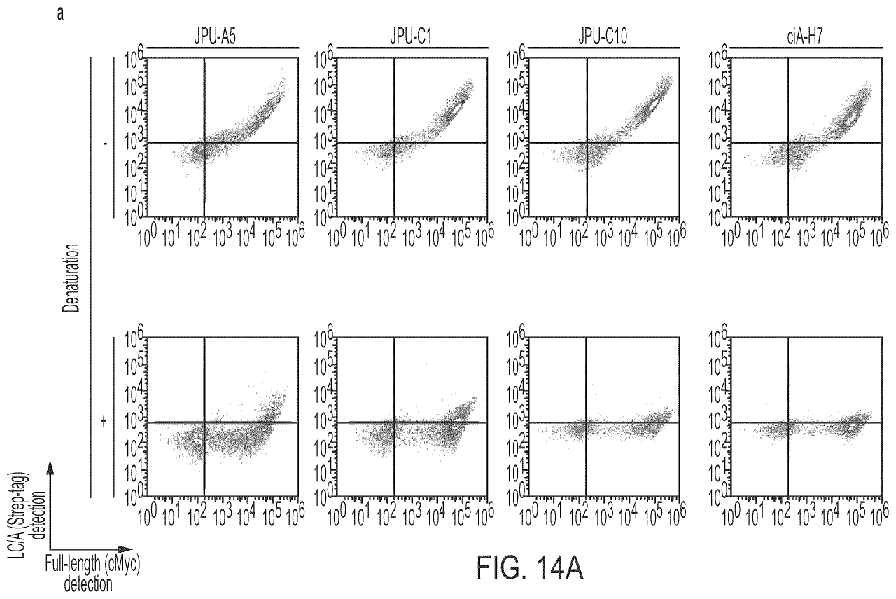

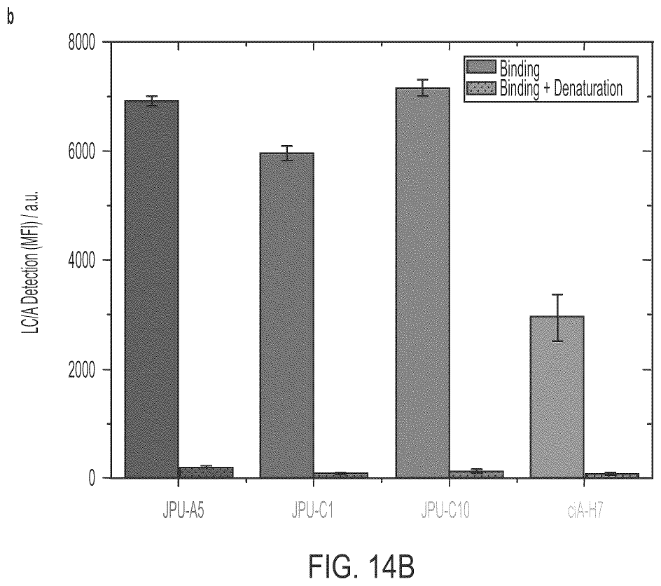

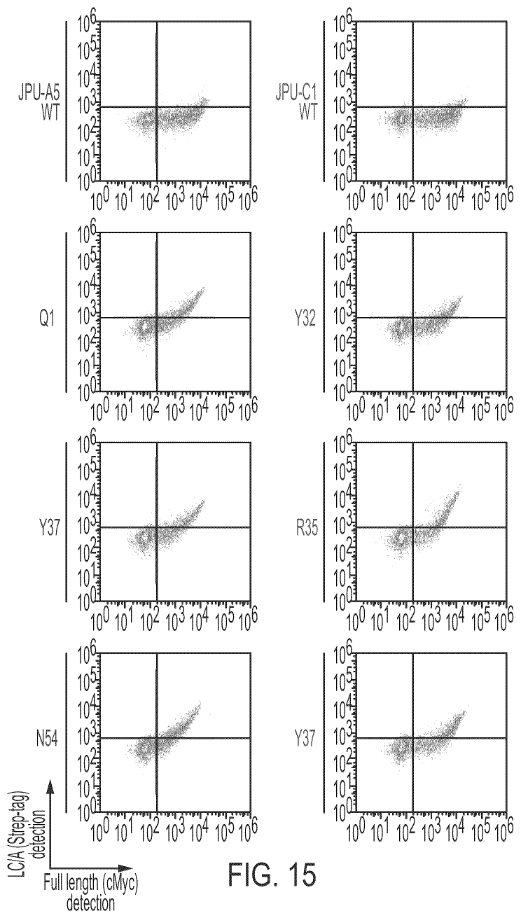

It was hypothesized that subjecting noncovalent LC/A-sdAb complexes on the yeast surface to stringent denaturation conditions would result in dissociation of the LC/A, while covalent complexes would remain detectable via flow cytometry. Yeast cells displaying AzF-substituted sdAbs were incubated with LC/A, followed by irradiation of the samples with 365 nm UV light. Thereafter, unbound excess LC/A was removed and the cells were subjected to denaturing conditions (urea 8M, EDTA 200 mM, Tris 25 mM, pH 8.0) before labeling and analyzing via flow cytometry (FIG. 3A). The fluorescence levels detected for LC/A after this process is shown in FIG. 3B. The WT constructs showed background levels of fluorescence after treating with denaturant, confirming the hypothesis (FIGS. 14A and 14B). Additionally, the control non-binding proteins, M0076 and DX2802 (scFvs, both WT and L1AzF—where L1 represents position 1 of the light chain),^{55, 56} also resulted in background levels of fluorescence, indicating that the presence of AzF on the yeast surface is not sufficient to retain LC/A after denaturation. Several AzF mutants showed LC/A binding levels above those detected for WT, indicating the presence of LC/A on the yeast surface even following denaturation, strongly suggesting crosslinking. While the screening results in seemingly different levels of photo-crosslinking across the variants, it is difficult to generalize these results given the possible different changes in affinity and avidity for each construct.

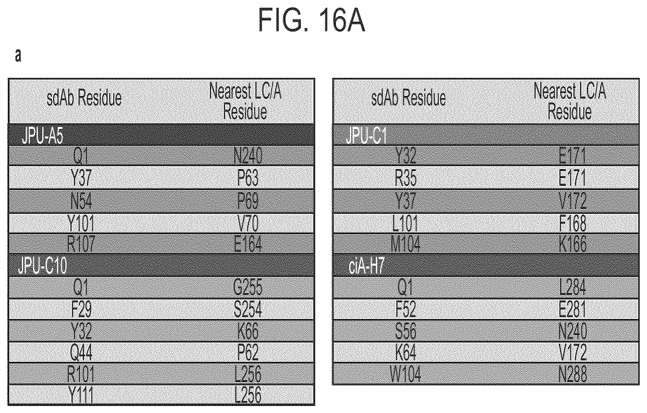

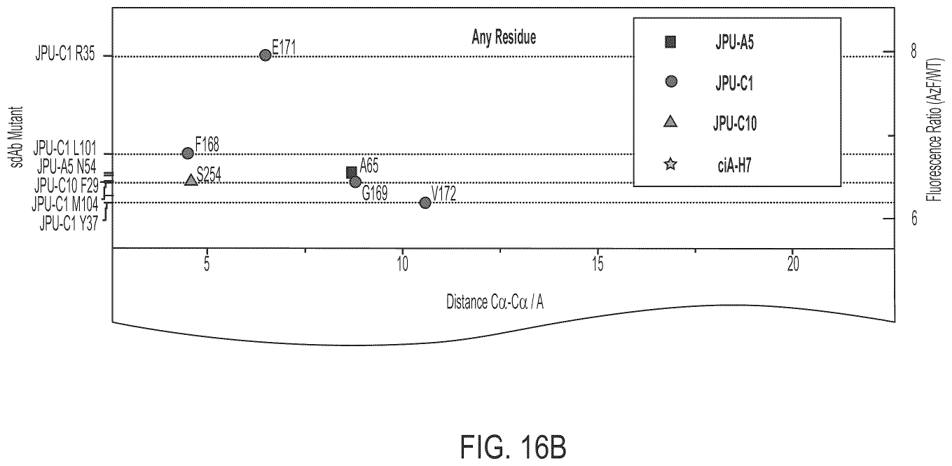

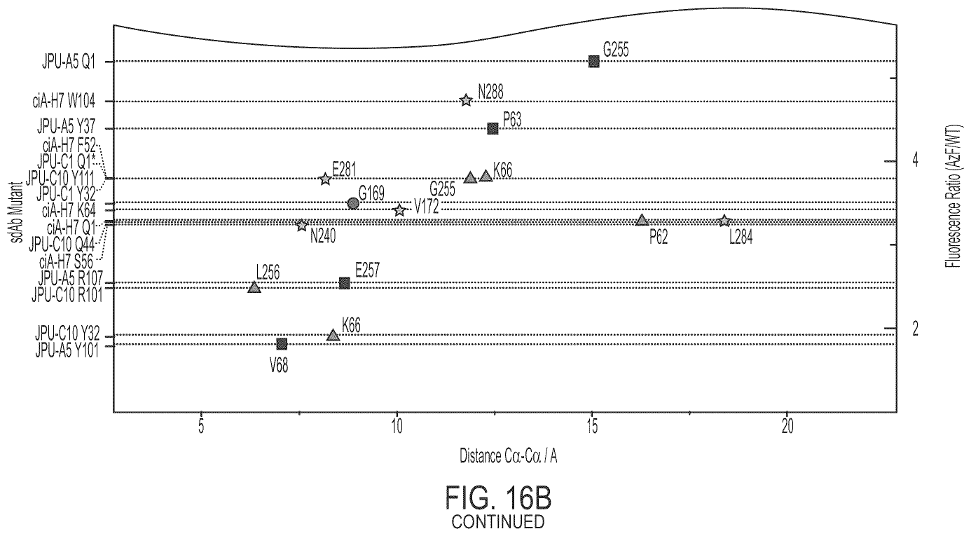

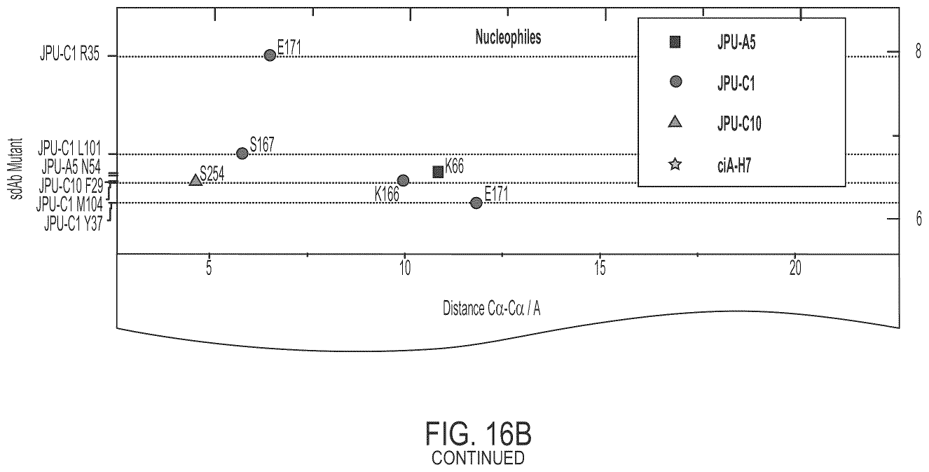

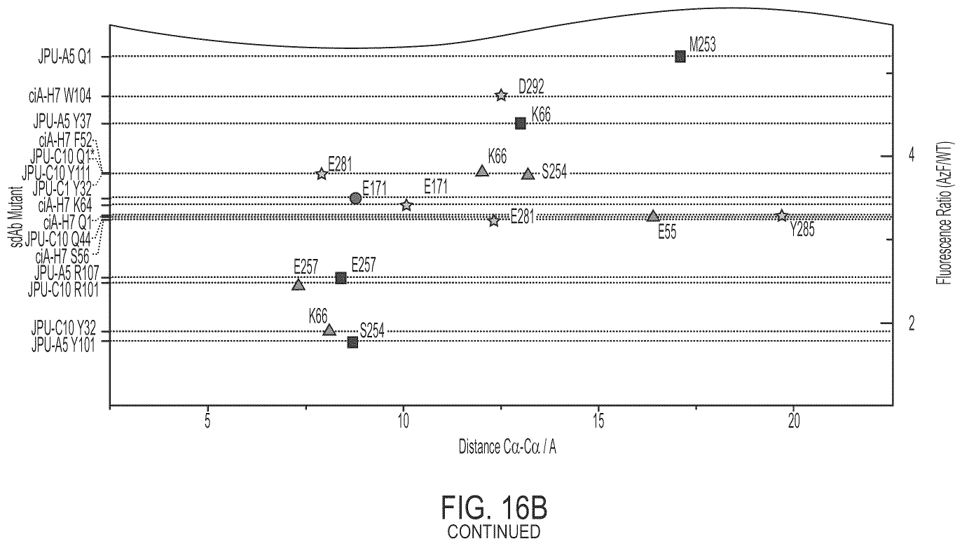

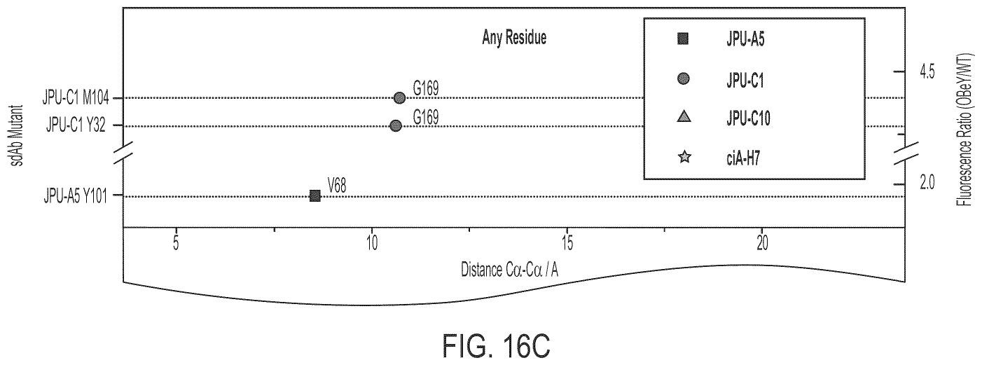

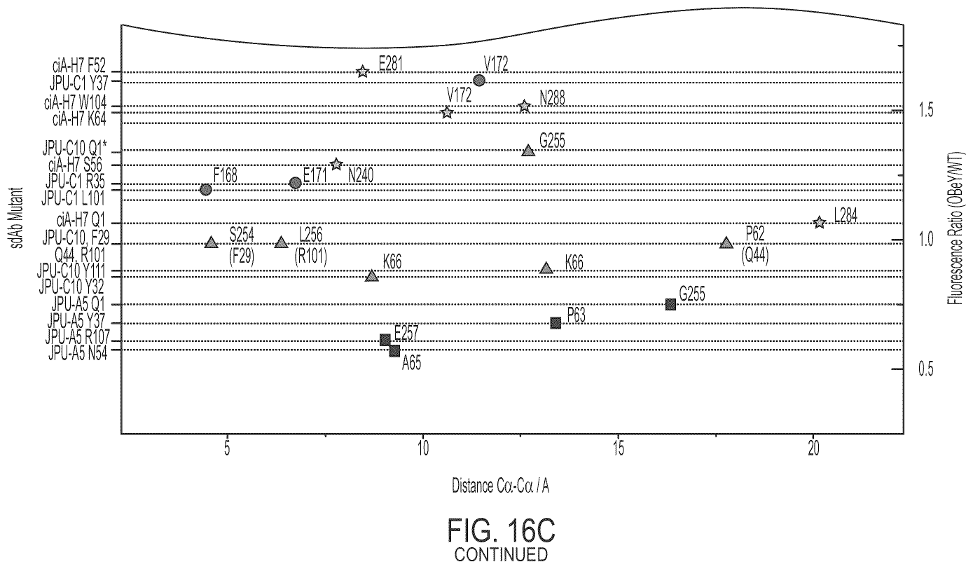

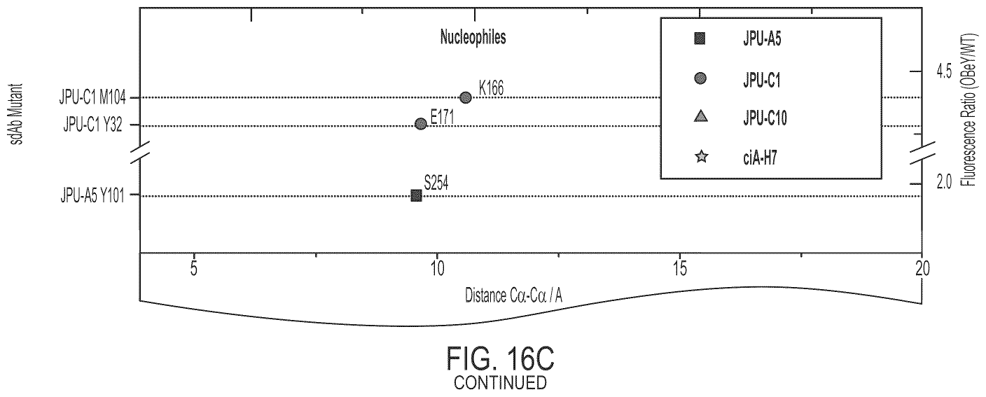

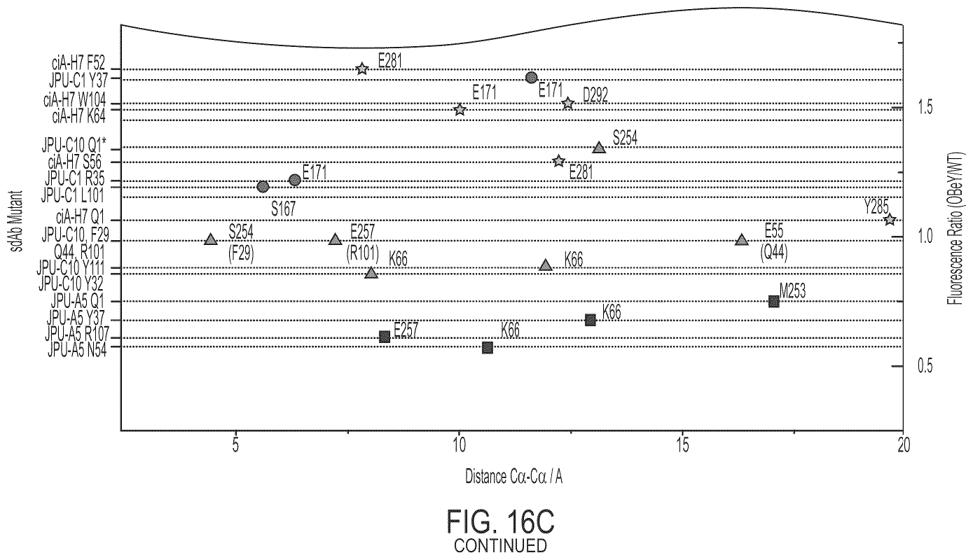

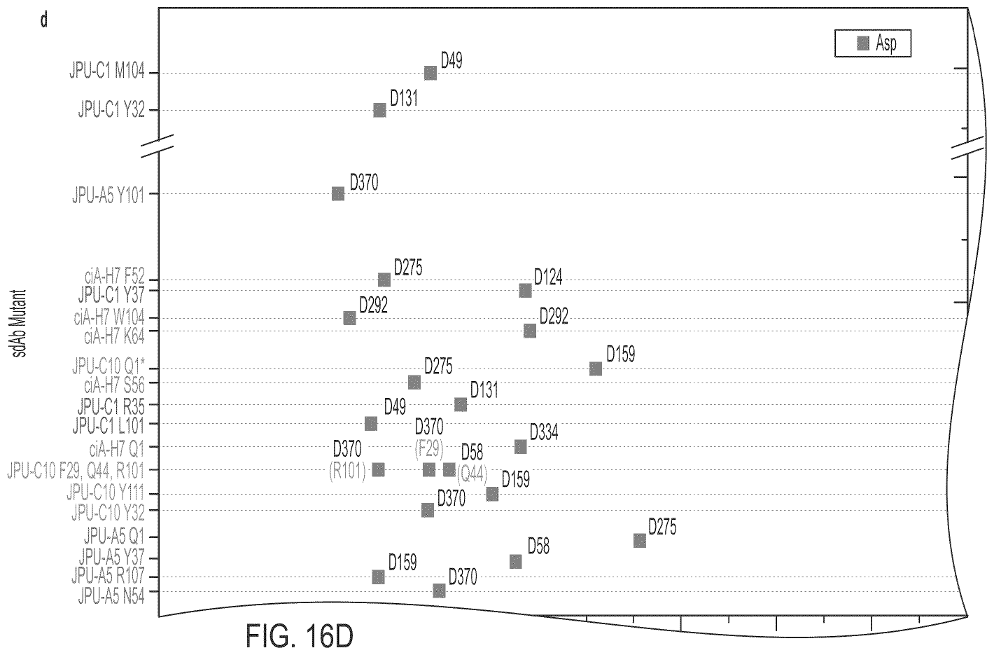

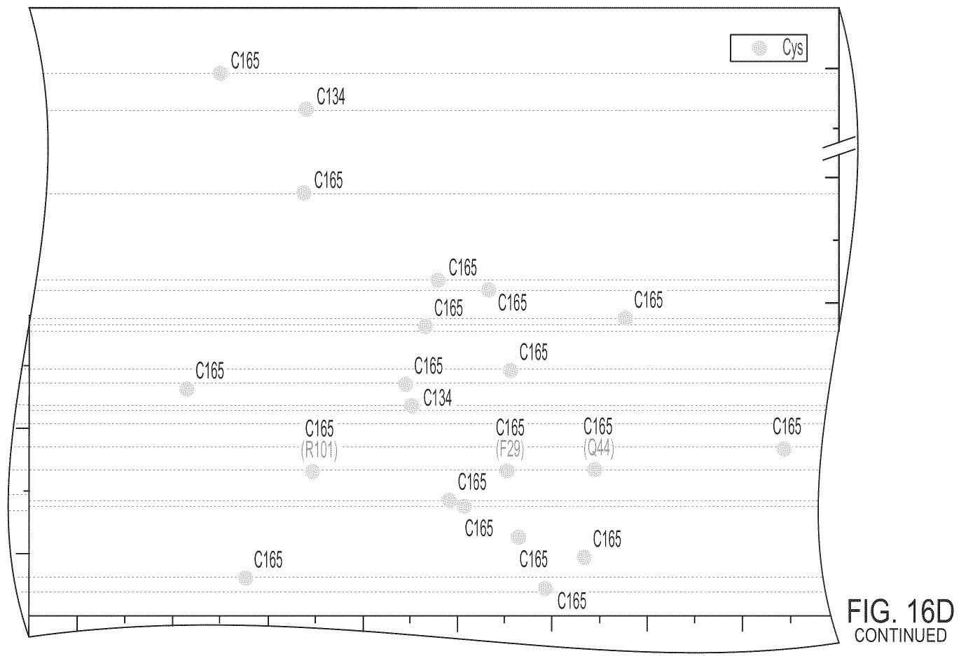

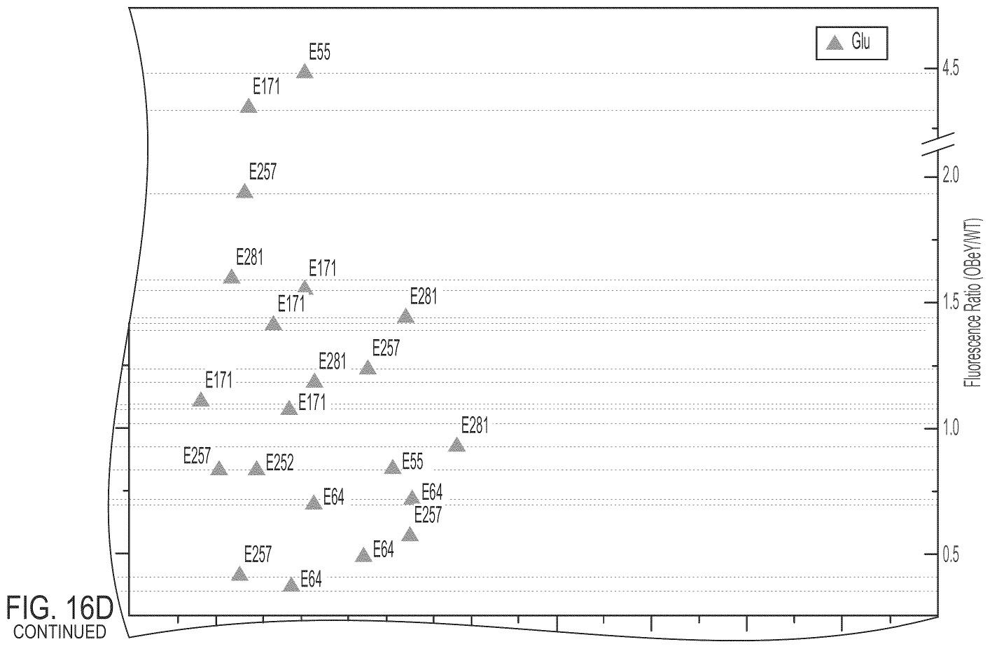

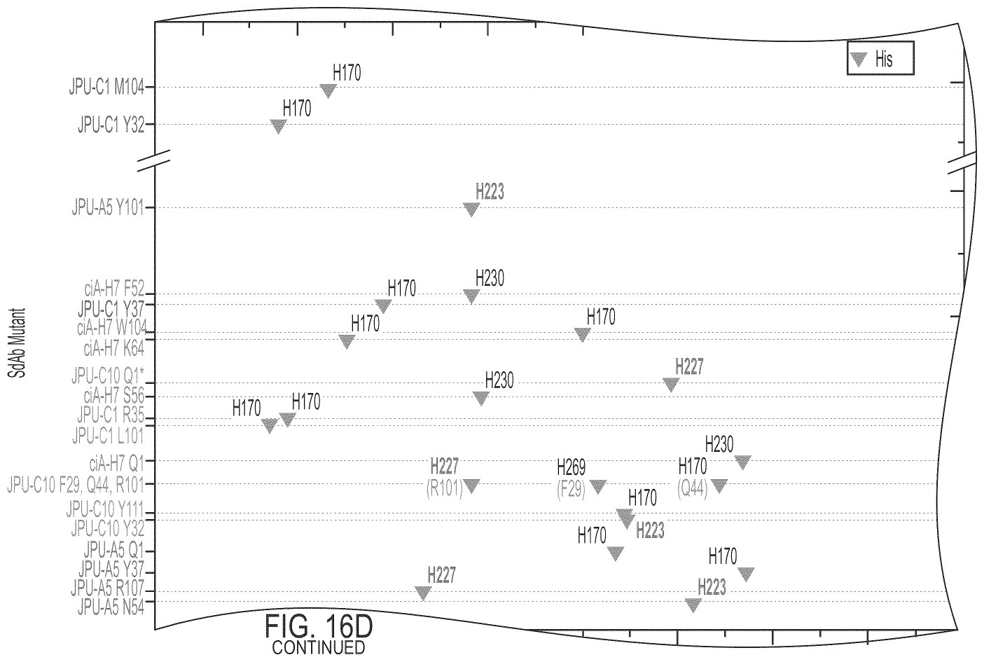

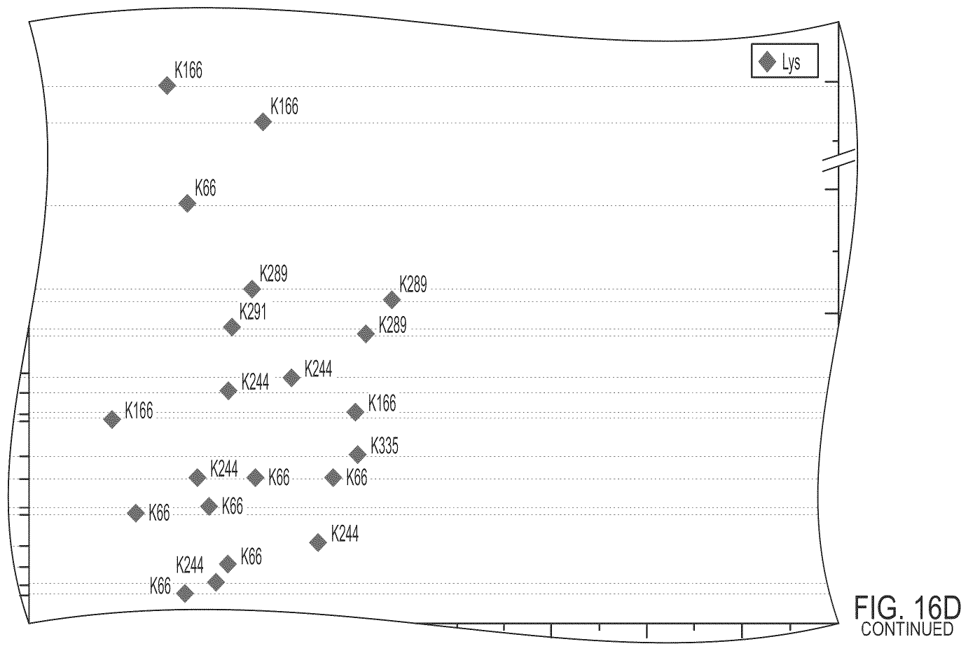

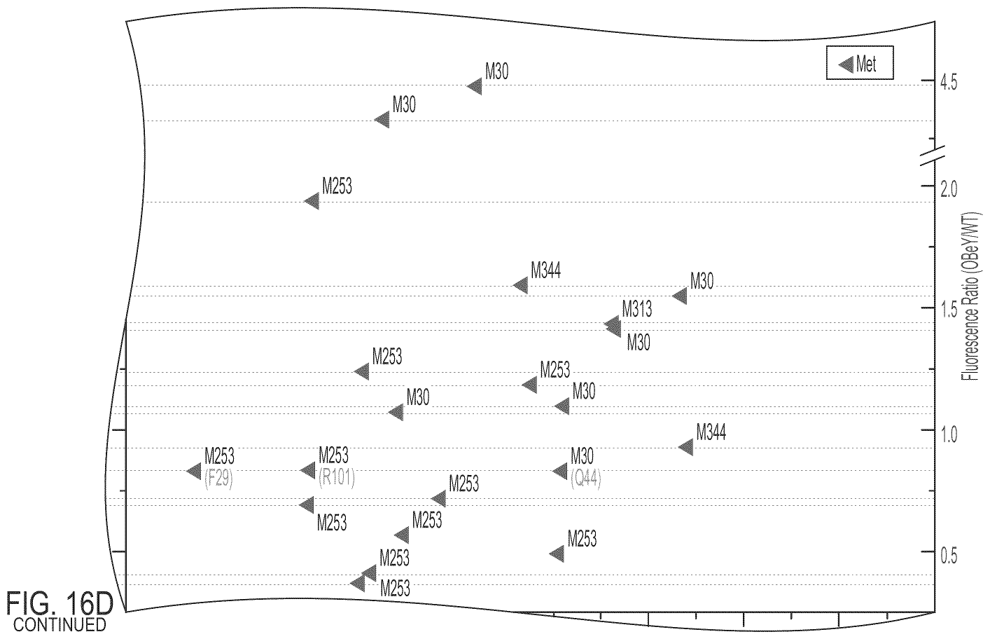

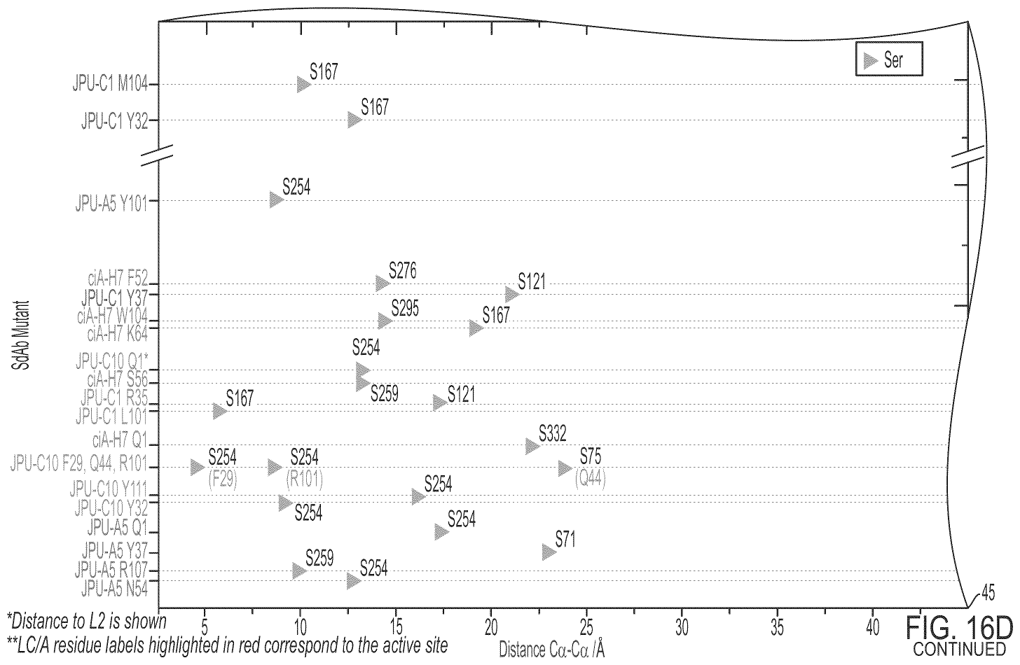

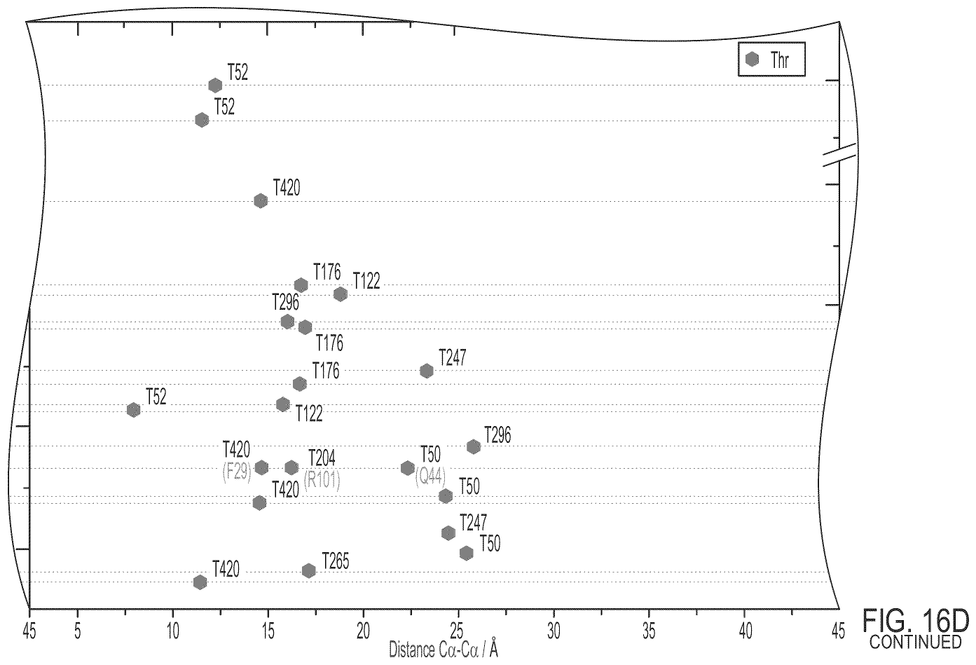

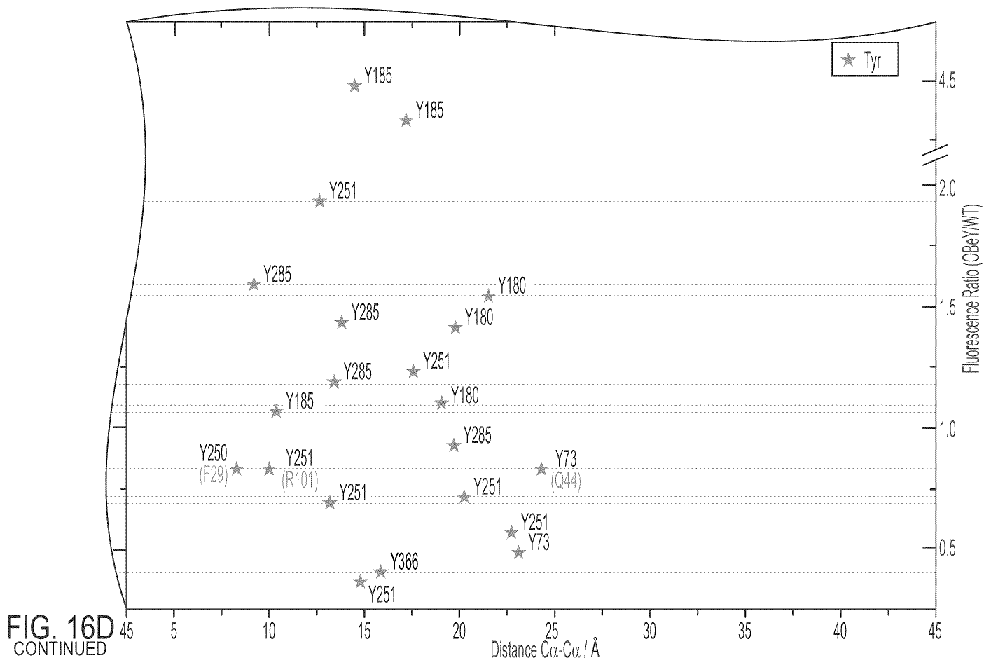

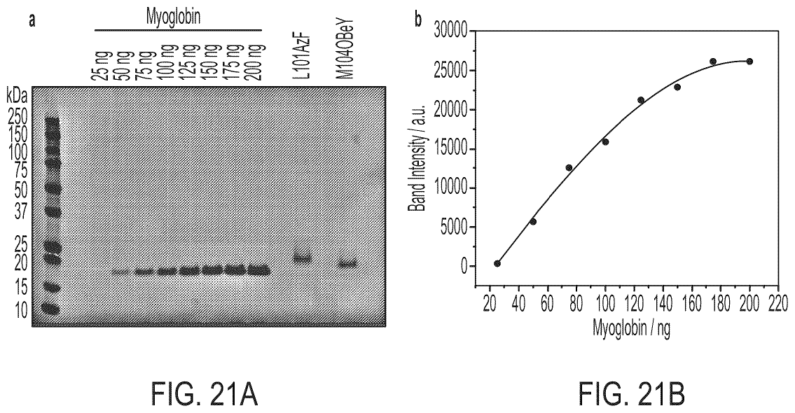

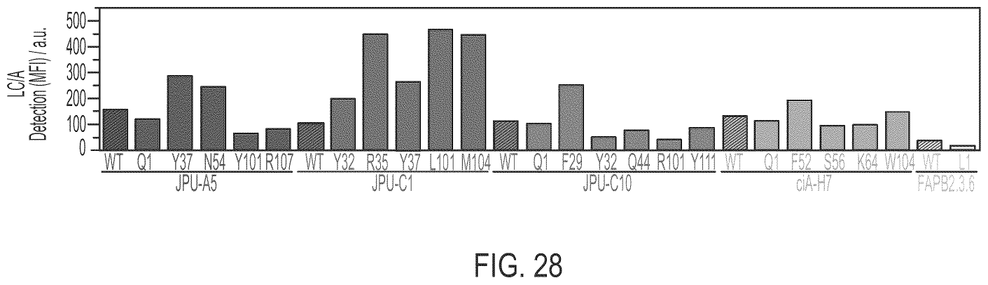

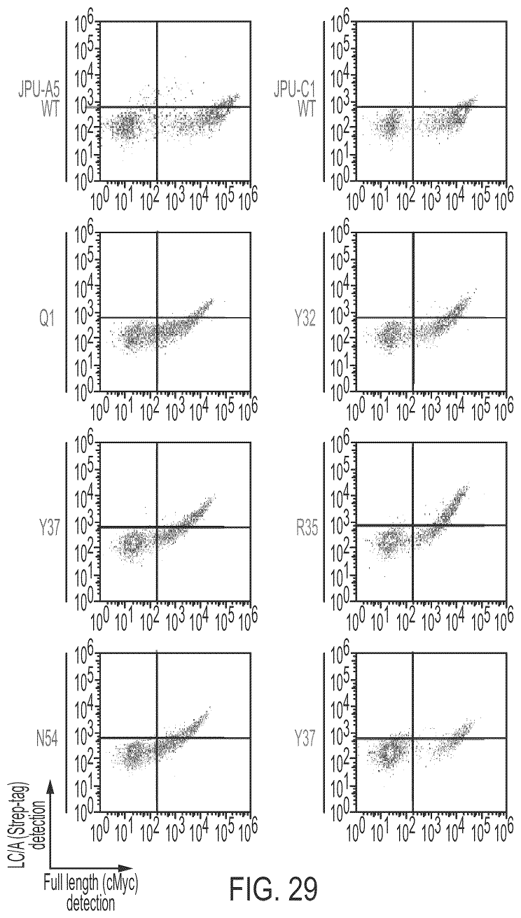

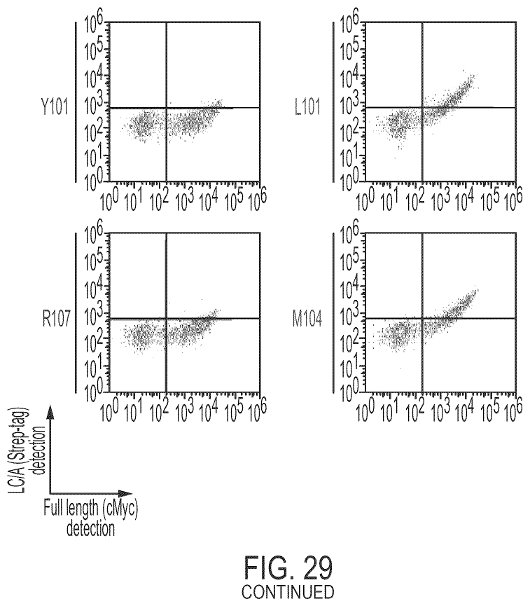

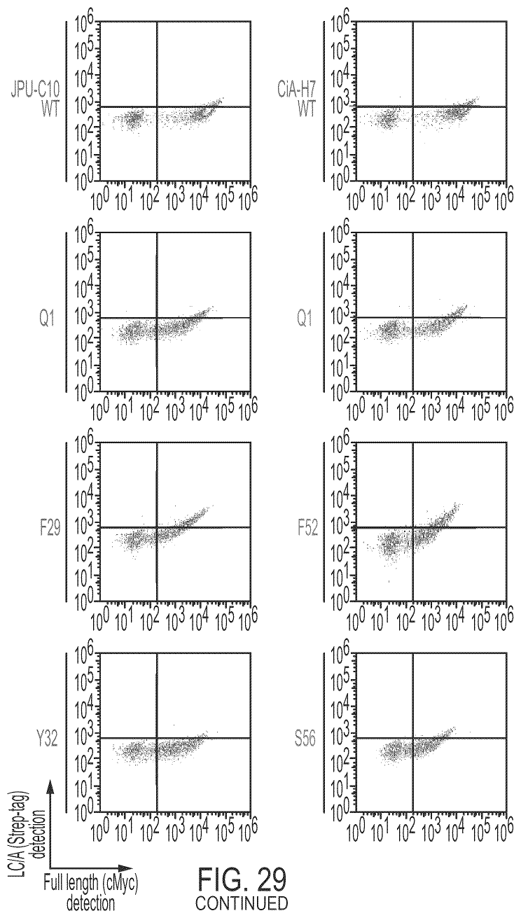

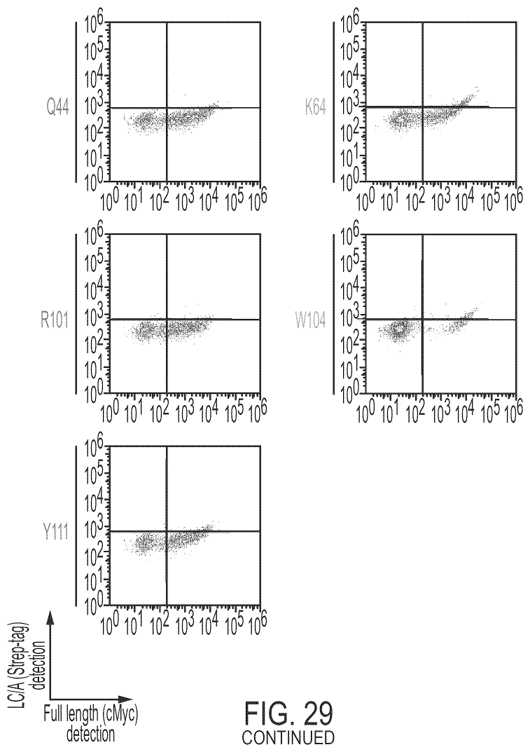

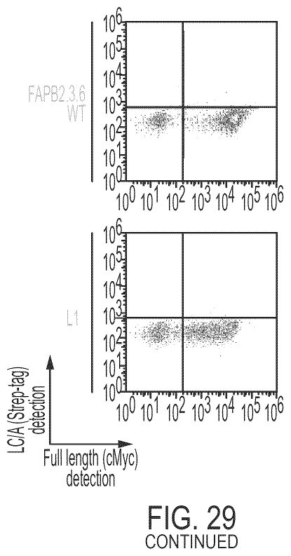

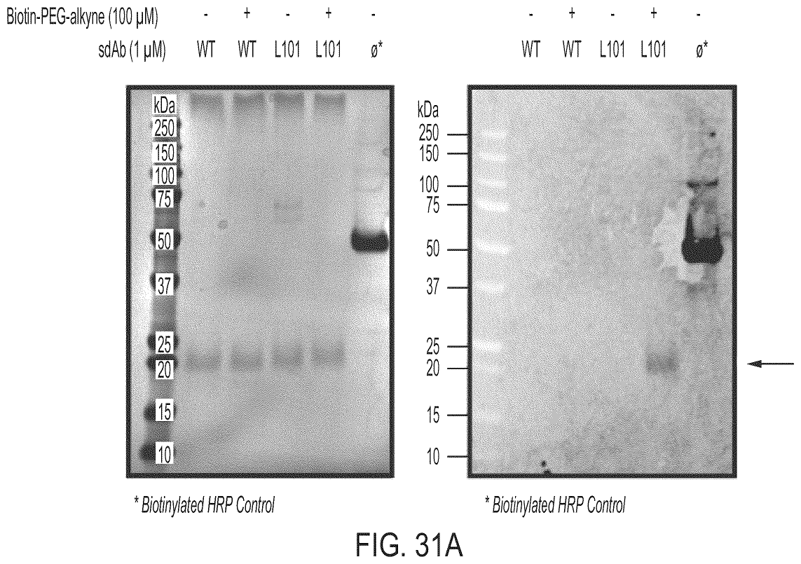

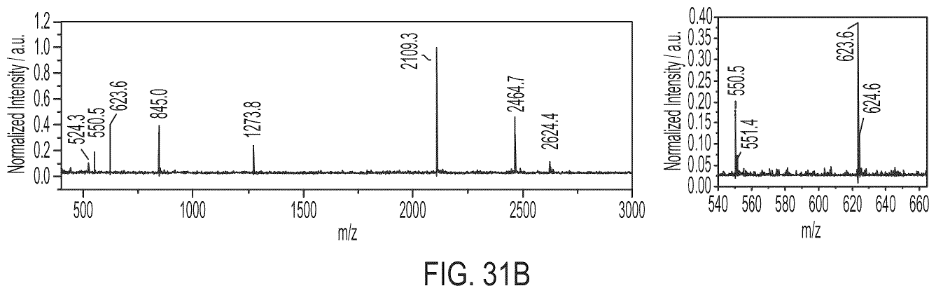

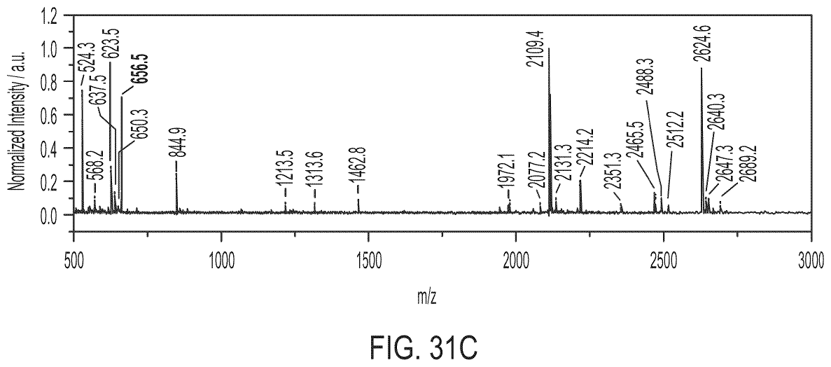

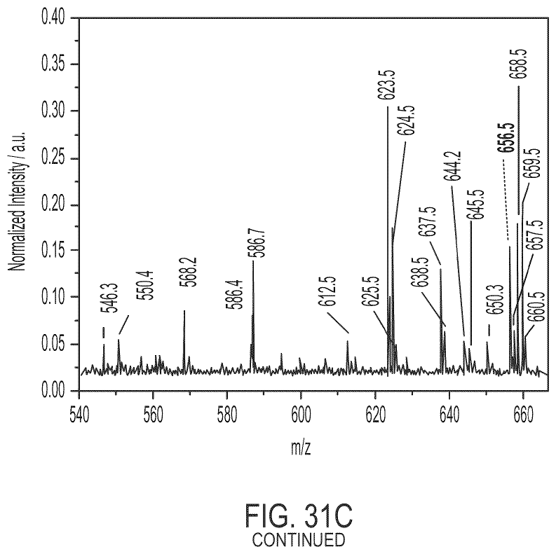

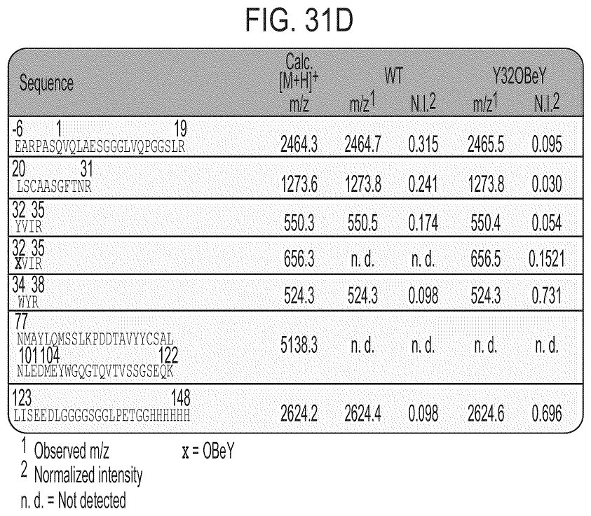

Based on reported structural studies that show a relationship between C-C distances⁴⁹, Cα-Cα distance was investigated to determine whether it was a predictor of observed crosslinking activity. Plotting putative crosslinking level versus Cα-Cα distance between the mutated amino acid and its nearest LC/A residue revealed no distinct correlation between detected LC/A levels and the distances (FIGS. 21 and 31). Given the possibility of a reactive electrophile species deriving from photoactivation of AzF, the apparent crosslinking levels were compared against the Cα-Cα distance to the nearest LC/A nucleophile, resulting in a similar plot without a clear correlation between the two variables (FIGS. 16A-16D; FIG. 16B). Of interest, even positions that appear to be distant from the sdAb-LC/A interface (JPU-A5 Q1; JPU-C10 Q1 and Q44; ciA-H7 Q1) showed some degree of apparent photocrosslinking. It is possible that dynamic protein motions could lead to successful crosslinking, or that LC/A multimerization or aggregation brings other LC/A molecules within proximity of distal portions of the sdAbs. However, it was also determined that crosslinking assays conducted using a low-power handheld UV lamp (FIG. 28 and FIG. 29) did not show detectable levels of crosslinking at these locations, thus suggesting that photocrosslinking at the most distant sites from the target occurs with only low frequencies

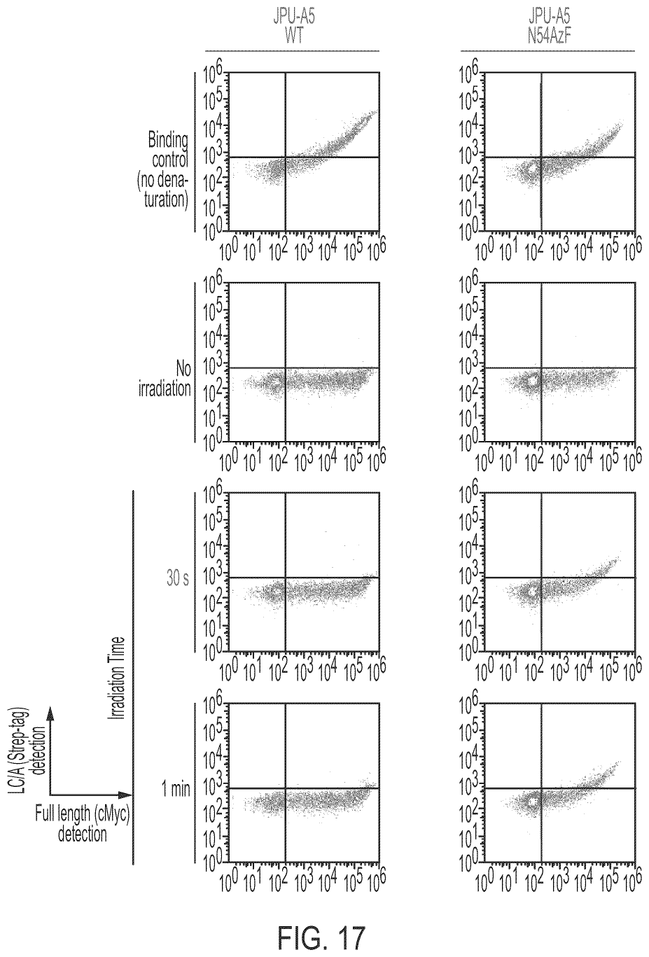

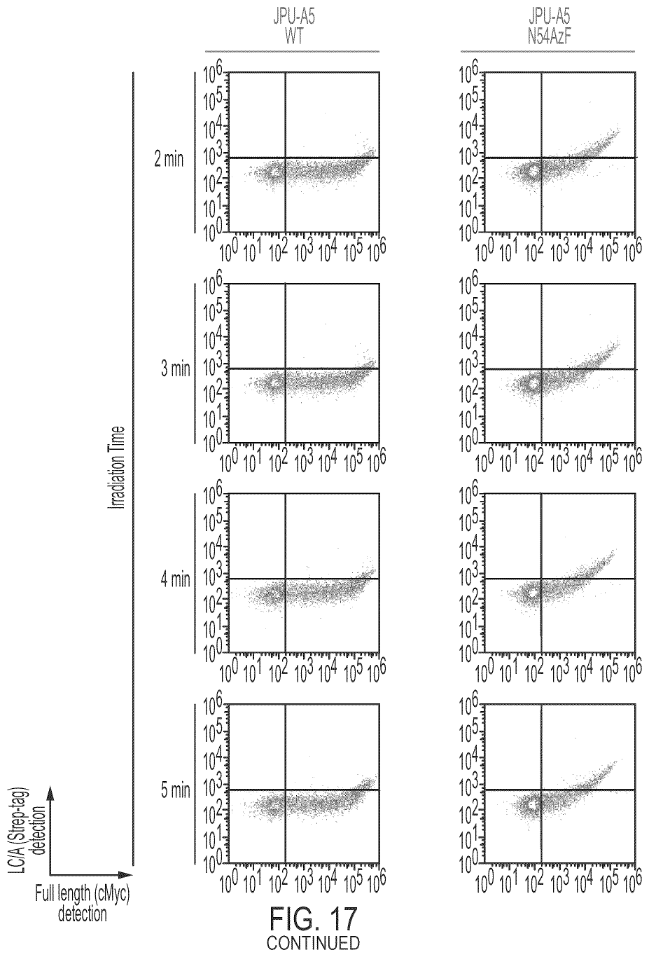

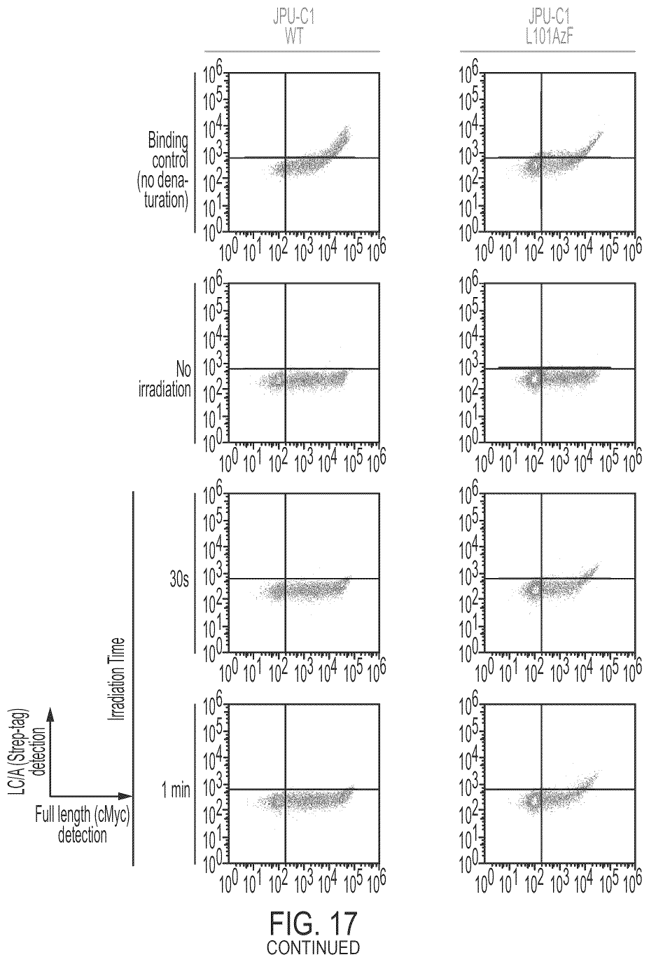

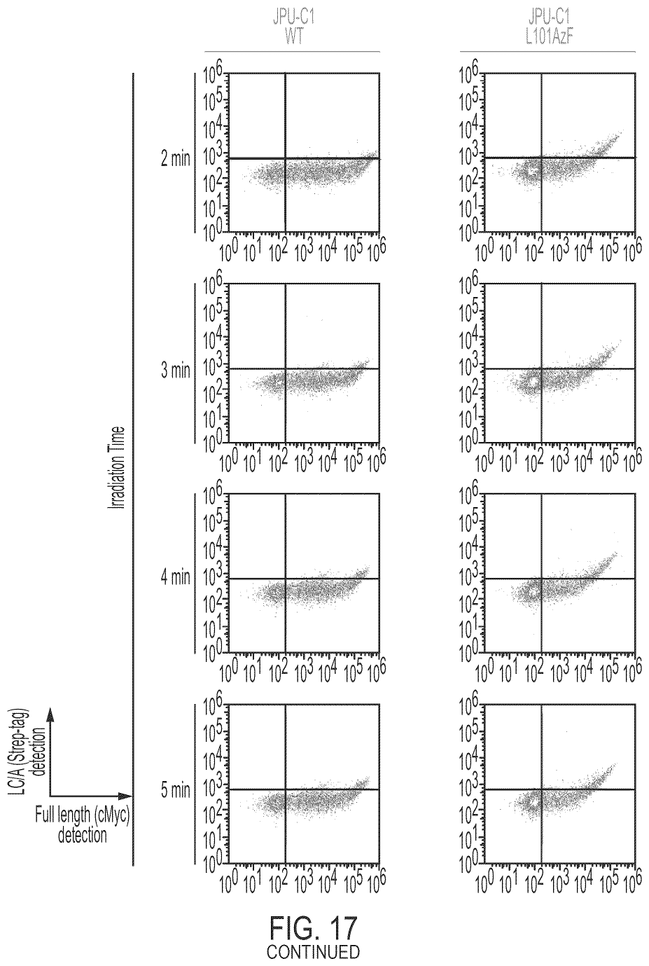

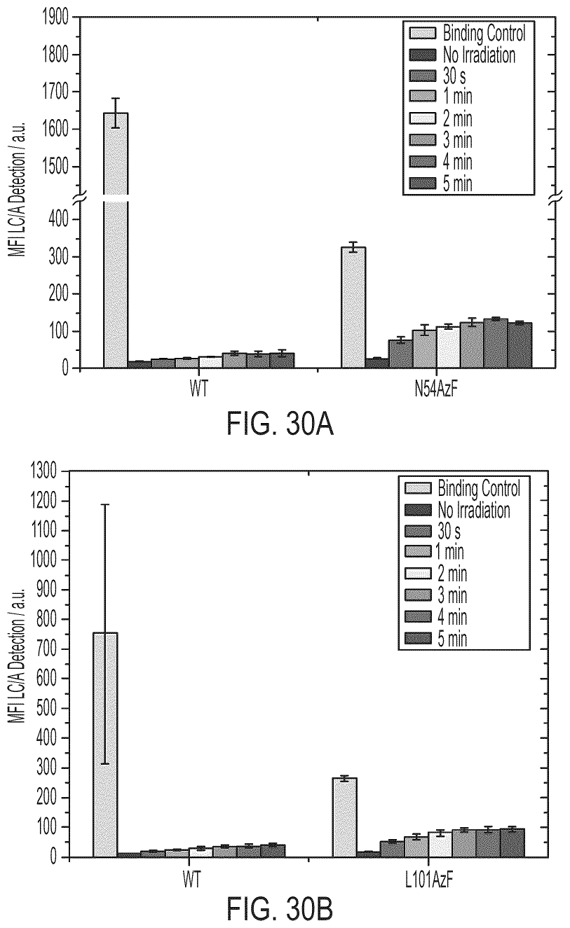

The time-dependent behavior of two promising candidates: JPU-A5 N5AzF and JPU-C1 L101AzF were next assessed. JPU-A5 N54AzF was chosen due to its apparently high target crosslinking levels (FIG. 3B). While the JPU-C1 L101AzF clone exhibited lower crosslinking levels than those of some other variants in the data shown in FIG. 3B, high crosslinking levels in a preliminary study with a handheld lamp prompted the choice of this particular mutant for further investigation (FIG. 28; FIG. 29; FIGS. 30A and 30B). The displayed camelids (sdAbs) were treated with LC/A in the dark and irradiated for up to 5 minutes, with aliquots taken at different time intervals (FIGS. 3C, 3D, and FIG. 17). The binding control experiments, i.e. without the denaturation step, confirmed the ability of the displayed clones to bind to the LC/A. Furthermore, no LC/A was detected on non-irradiated samples (0 time) after denaturation, indicating that reactivity was dependent on irradiation. The signal corresponding to LC/A increased in an irradiation time-dependent fashion for the AzF-containing clones but remained low for the WT constructs (FIG. 3D and FIG. 17). Taken together, these results are strong evidence that AzF is required to retain LC/A on the yeast surface, which is attributed to a covalent interaction formed between the camelid and the LC/A. Interestingly, the detected MFI levels of LC/A (FIG. 3D) stabilized after 3 minutes of irradiation without reaching the same levels of the binding control, suggesting that not all binding events resulted in covalent interactions. This could be attributed to the fast decay of the nitrene intermediate species into a less reactive electrophile,²⁵ resulting in a decreased yield of crosslinked adducts.

These studies further demonstrate the versatility of yeast display by expanding on the nature of photo-crosslinkable AzF-containing proteins that can be evaluated on the yeast surface and highlight its tolerance toward the experimental conditions. Additionally, the results show that this photoreactivity can be characterized in time-course experiments on the yeast surface, which can streamline the analysis of their reactivity profile without the need of expressing the proteins in soluble form. Based on such proofs of concept, the capability of the platform was extended to discover spontaneously crosslinkable sdAb variants on the yeast surface.

### Example 4: Spontaneous Crosslinking with OBeY

While photo-crosslinking has been a powerful tool for studying interactions in vitro, functional groups that require activation with short wavelengths are not generally amenable to in vivo applications. With this in mind, the variant sdAbs were investigated to determine if any of them that had been designed primarily for photo-crosslinking with AzF could support spontaneous crosslinking with OBeY. The latter ncAA contains an electrophilic bromoethyl chain that has been used for targeting nearby nucleophilic residues, particularly the thiol side chain of cysteine^{31, 61, 62}.

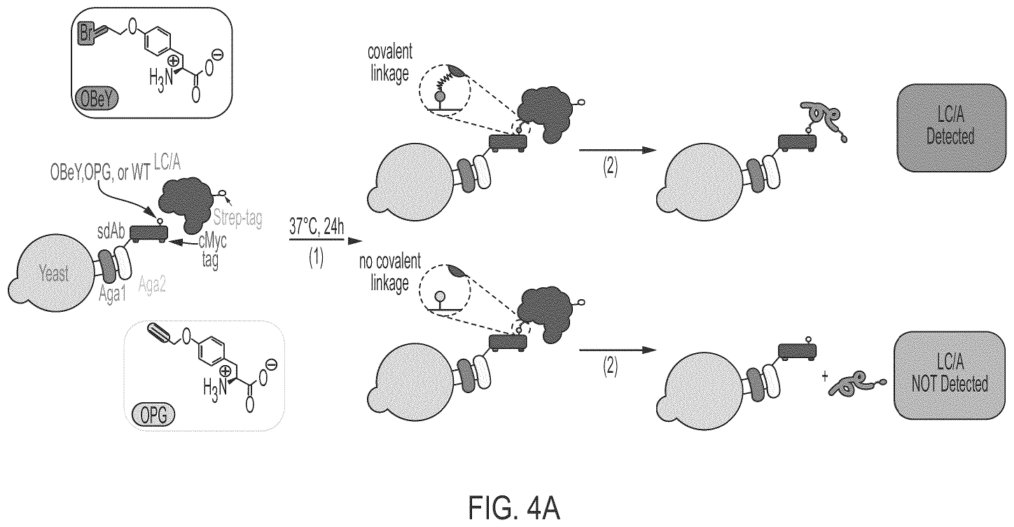

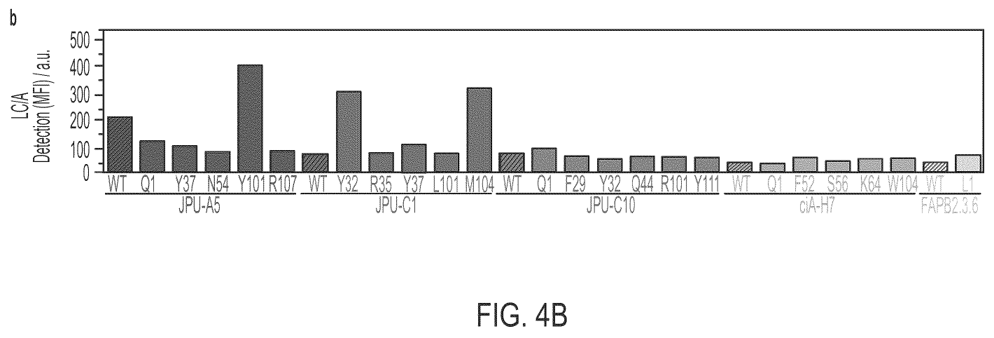

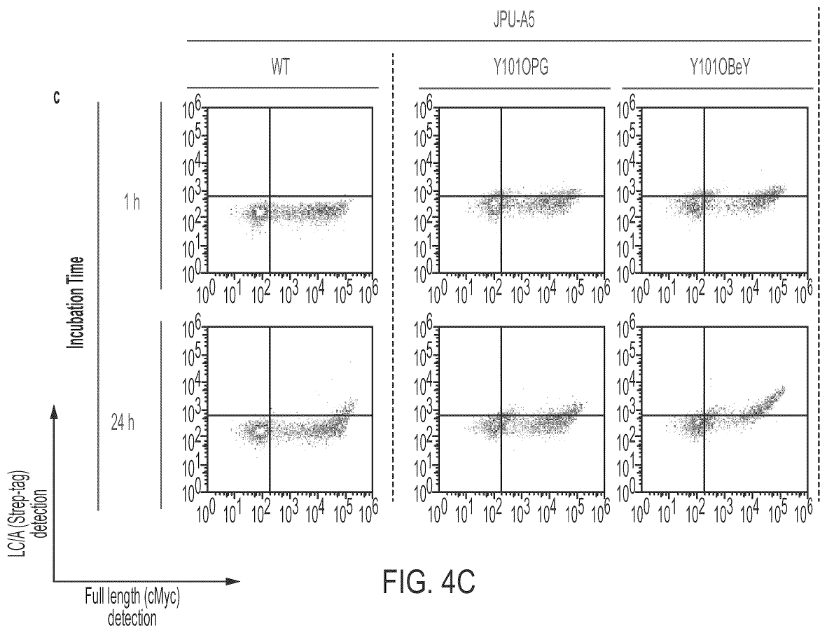

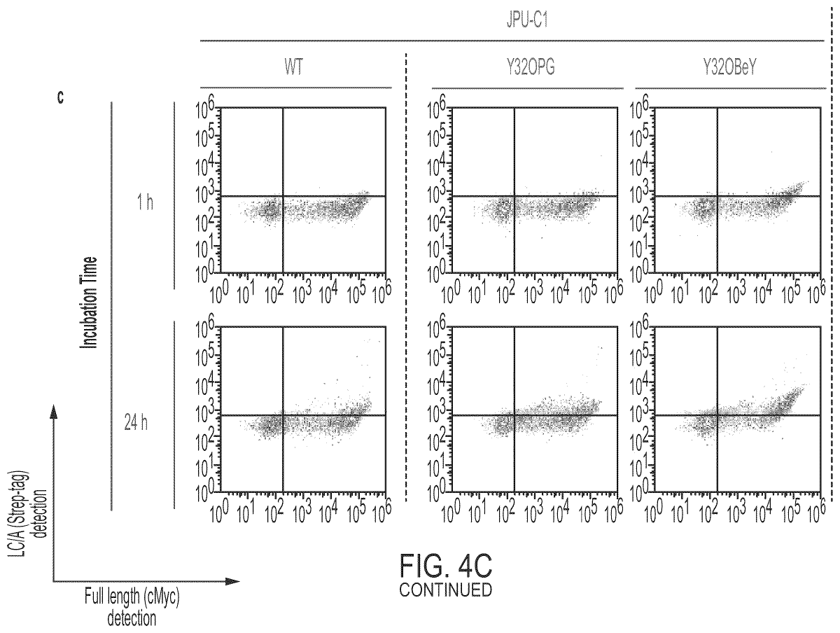

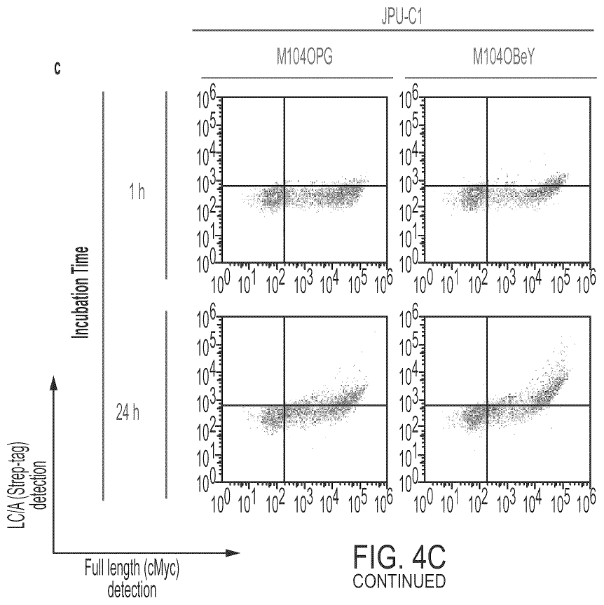

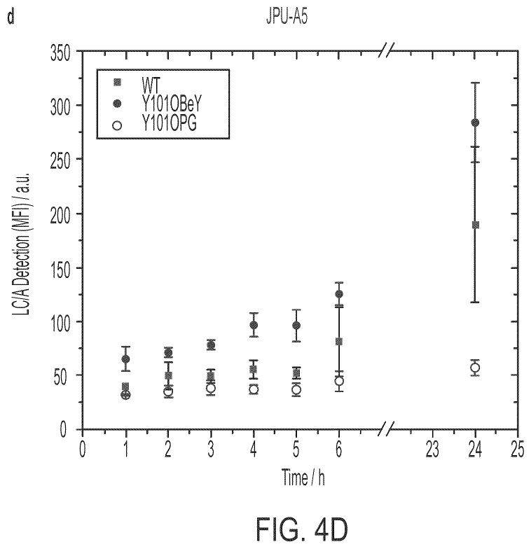

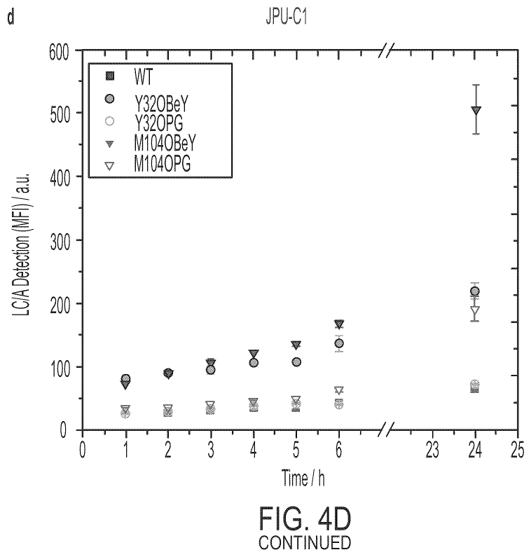

An experimental scheme similar to the one described for the photo-crosslinking studies was used to screen for OBeY mutants capable of forming covalent adducts on the yeast surface (FIG. 4A). In embodiments, both the AzP and the OBeY ncAAs were installed into an sdAb. In some cases, the ObeY reacted, thus obviating the use of photo-crosslinking via the AzP ncAA. The crosslinking was promoted by incubating displaying yeast cells with LC/A for 24 h at 37° C. Excess LC/A was then removed by washing, and cells were subjected to denaturing conditions to remove noncovalently bound LC/A. FIG. 4B shows LC/A fluorescence detection levels after flow cytometry analysis. Installing OBeY at the same positions employed for the photo-crosslinkable AzF mutants, sites resulting in successful spontaneous crosslinking with OBeY (based on LC/A levels above those of their corresponding WT versions) were very different than those found with AzF mutants (FIG. 3B). Specifically, the JPU-A5 Y101OBeY, JPU-C1 Y32OBeY, and JPU-C1 M104OBeY mutants resulted in successful spontaneous crosslinking. Additional positions that could lead to spontaneous crosslinking were identified on the JPU-C1 sdAb based on the crystallographic data and include R31, R53, D56, S57, R59, N100, E102, D103, and Y106. As in the case of photo-crosslinking, the results did not show any clear correlation between crosslinking and Cα-Cα distance between the mutated residue and the most proximal Ca in LC/A (FIG. 16C).

A similar analysis involving the nearest LC/A nucleophile revealed that that crosslinking only occurred with Cα-Cα distances between 8-10 Å, although not all mutants with a nucleophilic residue at this distance showed evidence of spontaneous crosslinking (FIG. 16B). Since alkyl halides have been shown to react with diverse nucleophiles present in proteins,^{76, 92} the Cα distances to each type of reactive nucleophile in LC/A (FIG. 16C) were also systematically examined. No discernible correlation was observed between crosslinking and distance for most nucleophiles. However, the plots corresponding to the nearest LC/A Cys or His residue show that higher levels of LC/A detection may be related to proximity to these residues, suggesting the possible identity of the reactive site in LC/A. While these trends indicate that distance to these nucleophiles is an important factor to consider in the positioning of reactive ncAAs, not all substitutions that satisfy this distance requirement resulted in detectable LC/A levels, indicating that other factors must come into play to achieve spontaneous covalent engagement.

To corroborate these findings, retention of LC/A on the yeast surface after incubation with the target for different time intervals followed by denaturation was evaluated by monitoring retention of LC/A on the yeast surface after denaturation at different time intervals. To account for the possibility that new noncovalent interactions arising from OBeY substitution led to enhanced binding (or hindered denaturation and dissociation from the yeast surface), sdAbs were included that incorporated the alkyne-containing ncAA O-propargyltyrosine (OPG) in place of OBeY as controls. OPG structurally resembles OBeY while lacking the reactive electrophilic center; it can also be incorporated into proteins in yeast using the AcFRS OTS⁴⁹.

FIG. 4C shows the flow cytometry dot plots corresponding to the analyzed yeast display samples after incubation for 1 and 24 h followed by removal of noncovalently bound LC/A. These plots qualitatively show that only OBeY-containing proteins retained binding to LC/A after denaturation. Without intending to be bound by theory, since the OPG-containing constructs showed binding to LCA but not retention after denaturation, this suggested that the reactive 2-bromoethyl chain of OBeY appeared to be necessary to retain binding following denaturation, in line with the proposed covalent bond formation via its electrophilic side chain in contrast with the inertness of OPG. FIG. 4D summarizes the findings by showing the fluorescence levels as a function of incubation time before denaturation. Quantitative evaluations of LC/A detection levels following a range of incubation times and standard denaturation (FIG. 4D) indicate a clear time dependence of the amount of LC/A retained on the yeast surface, with detectable increases in LC/A levels observed within the first several hours with OBeY-substituted clones. Only after times of 6 h or longer is LC/A detectable in some control samples. The comparatively short time scales over which LC/A levels rise in OBeY-substituted samples and relatively low background levels of LC/A detected for controls under similar conditions are consistent with the notion of spontaneous covalent interaction formation mediated by OBeY.

After long incubation times, yeast displaying JPU-A5 WT- or JPU-C1 M1040PG-containing clones exhibited low but detectable levels of LC/A. While not intending to be bound by theory, long-term incubation of LC/A in the presence of yeast may lead to aggregation or other conformationally altered states that are difficult to remove with denaturation procedures. Additionally, recent reports show that terminal alkynes, such as the one present in OPG, can engage in covalent interactions with cysteines via proximity-enhanced reactivity⁷⁷⁻⁷⁸. Given the proximity between position 104 in the JPU-C1 sdAb to LC/A Cys165, this could account for some of the LC/A retention in the case of M1040PG.

Overall, these results highlight the robustness of yeast display as a platform for the discovery of irreversible-binding antibodies, promoted by proximity-enhanced crosslinking. These experiments on the yeast surface show that this platform can be exploited to discover sdAbs capable of covalently engaging their target both via photo-crosslinking and via proximity-enhanced reactivity and allows for systematic evaluations that support the identification of multiple types of putative covalent interactions prior to the production of promising soluble candidates for solution-phase characterization.

### Example 5: In-Solution Crosslinking

Given the success in identifying covalent antibodies on the yeast surface, the findings were corroborated in soluble form. sdAb-encoding genes and TAG-containing variants were cloned into a modified version of pRS314,^{63, 64} a secretory plasmid incorporating C-terminal hexahistidine and cMyc tags into the encoded constructs.^{63, 64} Due to low expression levels of JPU-A5 WT in yeast, the analysis focused on the L101AzF, Y32OBeY, and M104OBeY derivatives of JPU-C1, which could be expressed at levels sufficient to facilitate solution-phase assays. The purities and concentrations of all soluble sdAbs were evaluated using SDS-PAGE analysis. In addition to the functional data presented in the described studies, characterizations via bioorthogonal chemistry and MALDI-TOF mass spectrometry⁷⁹⁻⁸¹ provided direct evidence for ncAA incorporation in the L101AzF and Y32OBeY clones, respectively (FIGS. 21A, 21B and FIGS. 31A-31D).

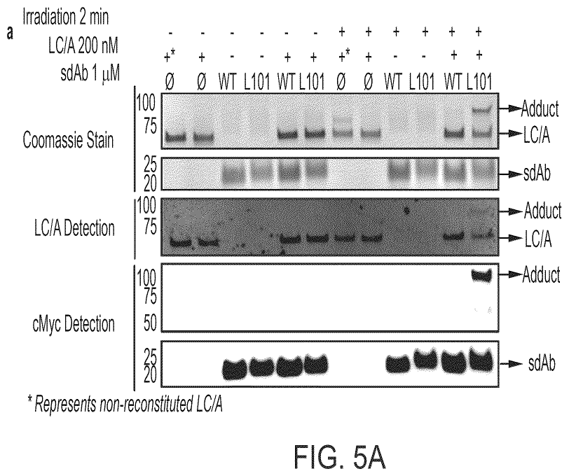

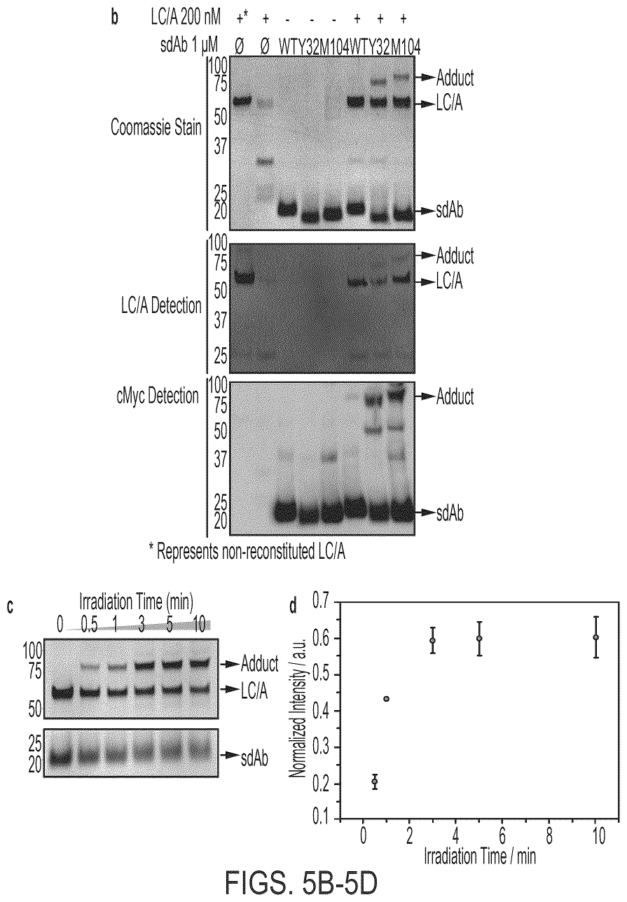

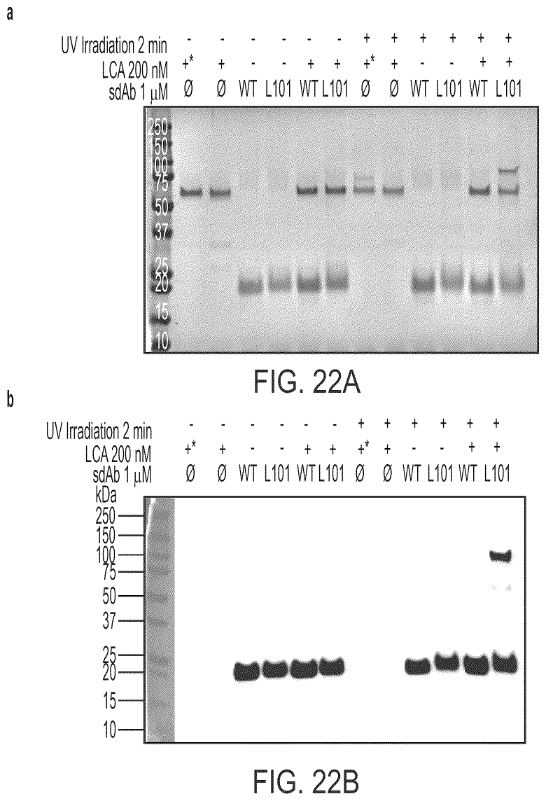

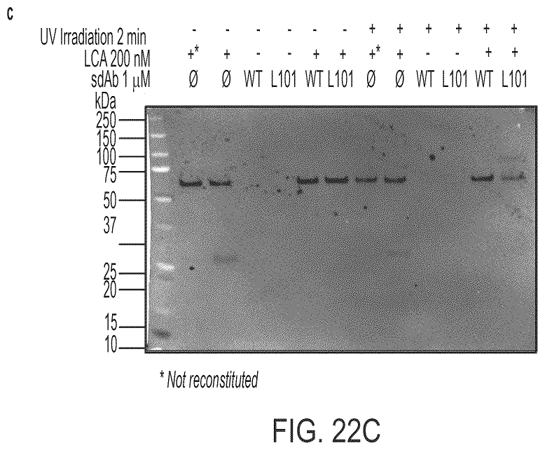

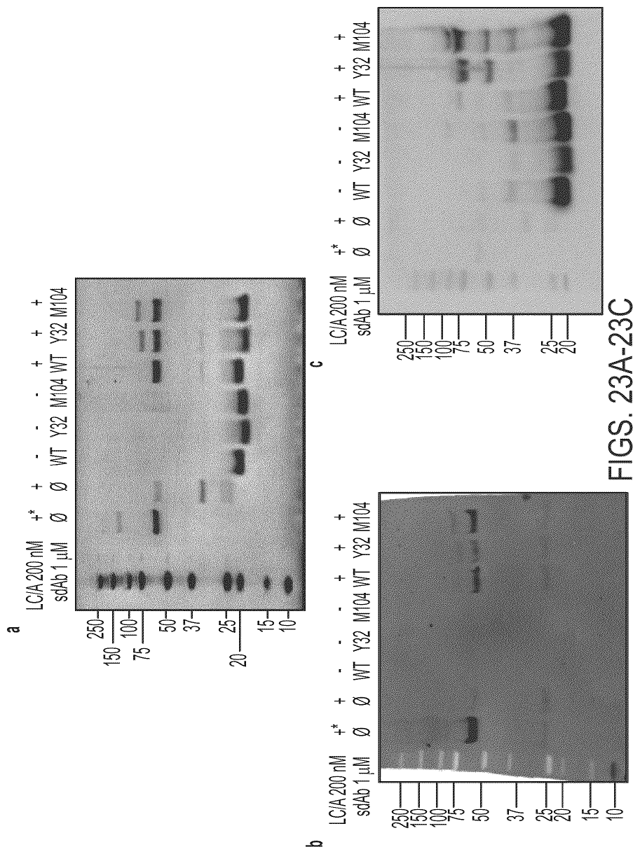

Following expression and purification of all derivatives, the crosslinking properties of the soluble sdAb variants were determined. Photo-crosslinking was performed by incubating either the WT or L101AzF sdAbs in the presence of LC/A, followed by UV irradiation of the samples in a photoreactor for 2 minutes (min). Spontaneous crosslinking was assessed by incubating WT, Y32OBeY, or M104OBeY with LC/A at 37° C. for 24 h. Analysis was carried out via SDS-PAGE, as the denaturing conditions disrupt noncovalent interactions between the sdAb and the LC/A, and covalent adducts are detected as a new band with increased molecular weight. FIGS. 5A and 5B show the gel images resulting from the photo-crosslinking and spontaneous crosslinking, respectively (FIGS. 22A-22C and FIGS. 23A-23C). In both situations, when LC/A was incubated with WT sdAb, only the bands corresponding to each separate protein were visible after Coomassie staining. However, when LC/A was incubated with ncAA-containing sdAbs in their corresponding conditions (irradiation for 2 minutes or 24 hours incubation), an additional band of a molecular weight consistent with the expected size of the LC/A-sdAb adduct was detected. Densitometric analysis revealed that the band intensities for the crosslinked adducts were 38.1±2.9% (L101AzF), 35.5±4.4% (Y32OBeY) and 38.1±5.7% (M104OBeY) when compared to the non-reconstituted LCA control. These values represent an upper-limit estimate of the crosslinking yield under the experimental conditions used here, as LC/A degradation, differences in staining of different molecular weight proteins, and signal saturation limit the accuracy of this calculation.

In order to confirm the composition of the presumptive adduct, the C-terminal tags present in LC/A (Strep-tag) and the sdAbs (cMyc) were used. Using western blots with the two different detection antibodies, it was observed that this high-molecular weight band was apparent when probing for both tags, thus validating its identity (FIGS. 5A and 5B; FIGS. 22A-22C and FIGS. 23A-23C).

Additionally, the western blot for the cMyc epitope revealed a band at approximately 50 kDa when LCA was incubated with the OBeY-containing sdAbs. These results are consistent with a crosslinked adduct between the sdAb and a previously reported ˜35 kDa N-terminal degradation product of LCA.^{64, 65} This observation further corroborates the reactivity between the LCA and sdAbs, while also providing evidence regarding the crosslinking position on the LCA. Since the ˜35 kDa product corresponds to the 250-residue N-terminal portion of the protease,^{65, 82} the crosslinking may occur between the sdAb and amino acid(s) in the range of residues 1-249 of LC/A. This in line with the crystallographic data, which shows that interactions between WT JPU C1 and LC/A occur within this domain⁴⁷.

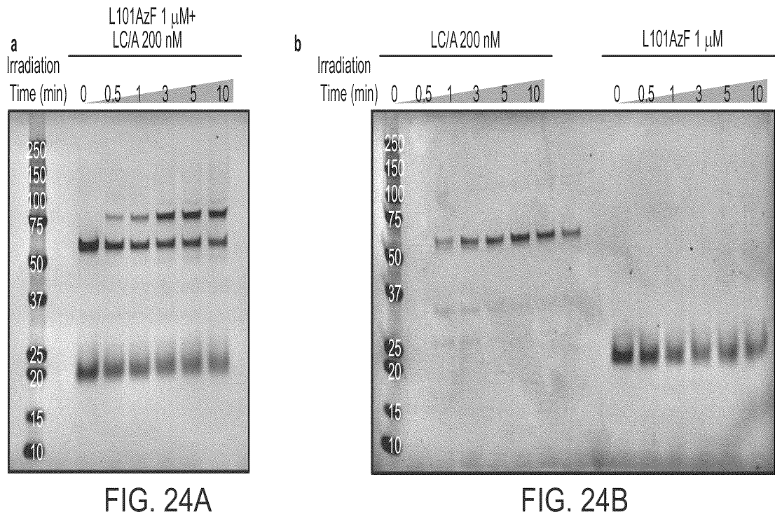

With convincing evidence for crosslinking interactions in solution, experiments were conducted to characterize the formation of the photo-crosslinked LCA-sdAb adduct as a function of irradiation time. The results in FIGS. 5C and 5D show qualitatively that the intensity of the band corresponding to the LC/A-JPU-C1 L101AzF complex increased proportionally to irradiation time until reaching a maximum after 3 minutes of irradiation, consistent with the observed increase in covalently bound LCA detected on the yeast surface (See, FIGS. 24A and 24B—full image).

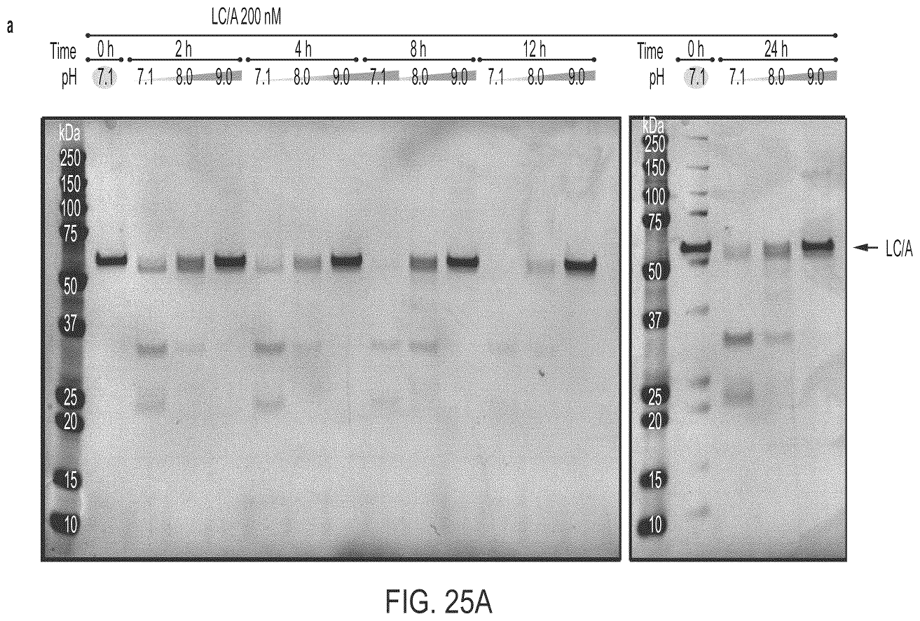

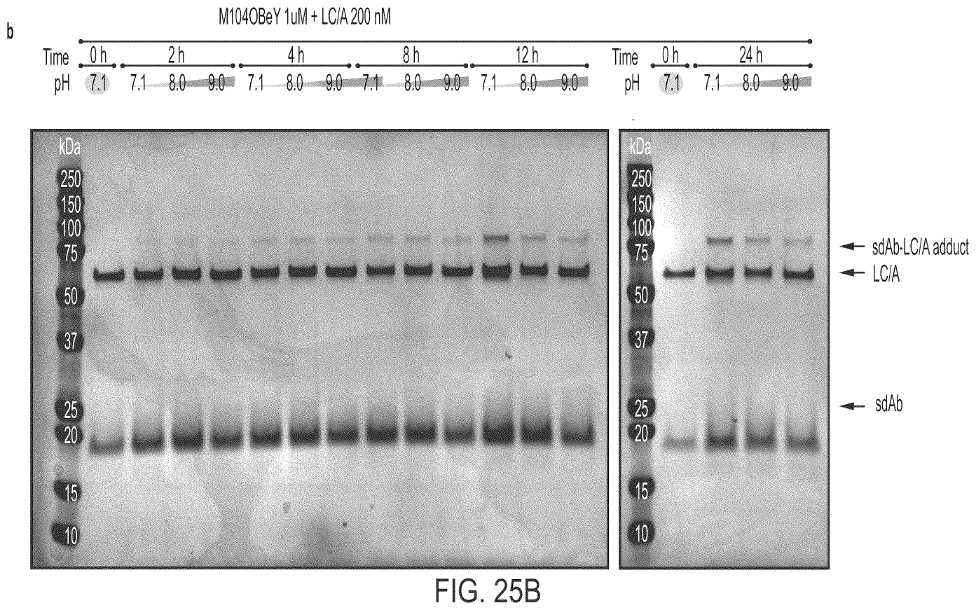

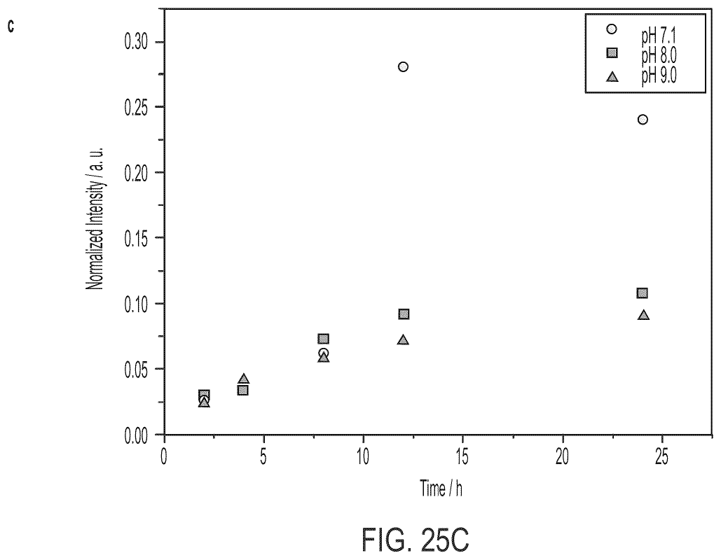

A similar experiment was carried out with JPU-C1 M104OBeY by following adduct formation over time at different pH values (FIGS. 25A-25C). Interestingly, the intensity of the crosslinked adduct increased over the first 12 h of incubation in buffer at pH 7.1, while increasing for only the first 8 h in buffers at pH values of 8.0 or 9.0 and yielding lower final quantities of adducts. This is surprising given that the bromoethyl functionality typically reacts with nucleophilic thiols or amines, and thus reactivity is expected to increase at high pH values. Alternatively, the distance analysis (FIG. 16C) revealed the possibility of crosslinking to His residues, which may not show noticeable increases in nucleophilicity at the studied pH values. Furthermore, it was also observed that LC/A autolysis was impaired at higher pH values (FIG. 25A). Hence, decreased crosslinking at higher pH values may be attributable to structural changes in LC/A that hinder sdAb binding and thus interfere with covalent engagement.

The soluble characterizations performed here confirm the crosslinking capabilities of several sdAb variants initially identified on the yeast surface and indicate that crosslinking time scales are similar on yeast and in solution. These findings further validate the use of the yeast display format to conduct initial experiments prior to moving to solution-based assays.

### Example 6: Inhibitory and Selectivity Properties of sdAbs

During the previous experiments, it was noticed that the band intensity corresponding to active LC/A decreased during the incubation conditions used for spontaneous crosslinking. Additionally, new bands appeared: one slightly below the intact LC/A, another one ˜35 kDa, and two near the 25 kDa marker (FIG. 5B). This pattern was consistent with previous reports on the autoproteolysis of LC/A.⁶⁵ Of interest, when LC/A was incubated in presence of WT or OBeY sdAbs, the band corresponding to intact LC/A was similar to non-reconstituted LC/A, while those corresponding to the degradation products were less evident. This strongly suggests that both WT and OBeY-containing sdAbs are capable of inhibiting the autoproteolytic activity of LC/A, implying that OBeY substitution does not overtly disrupt the demonstrated inhibitory properties of WT JPU-C1⁴⁷.

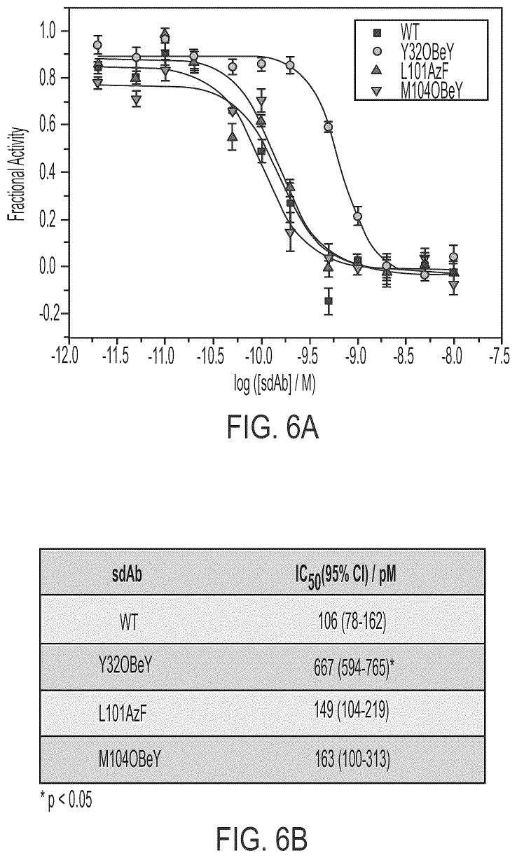

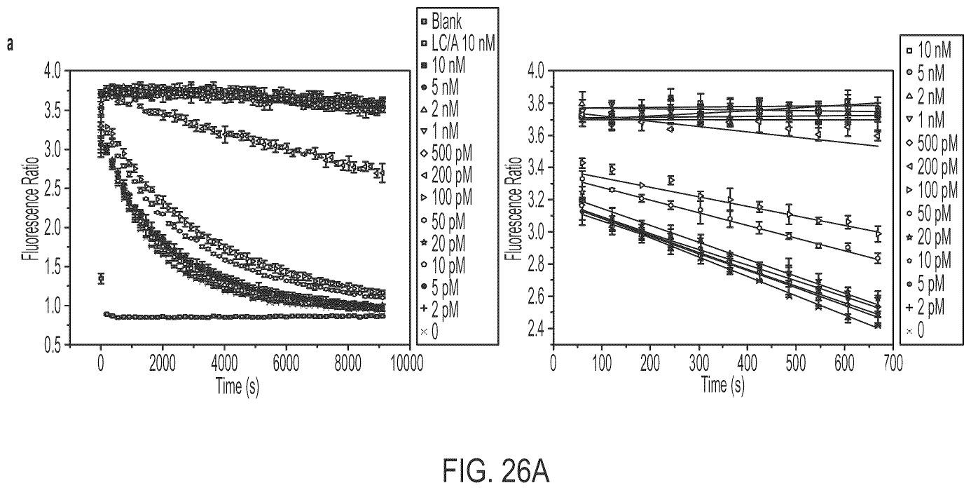

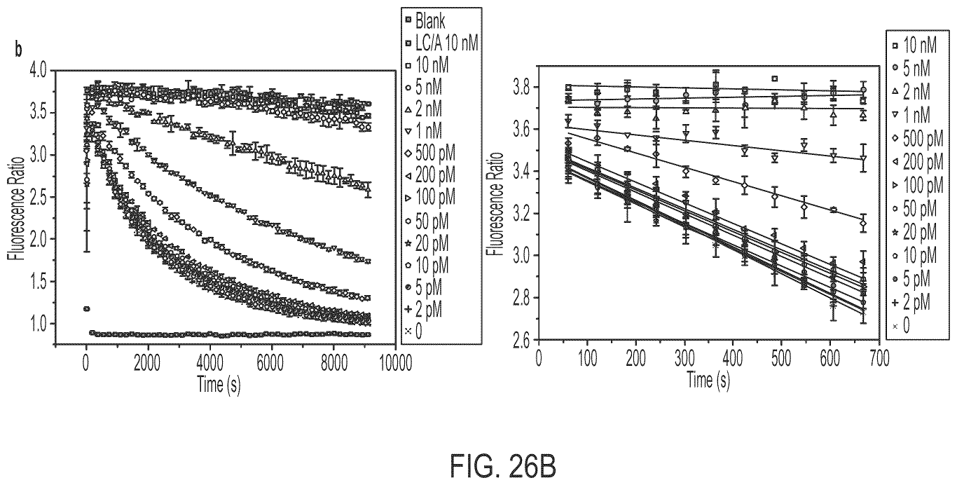

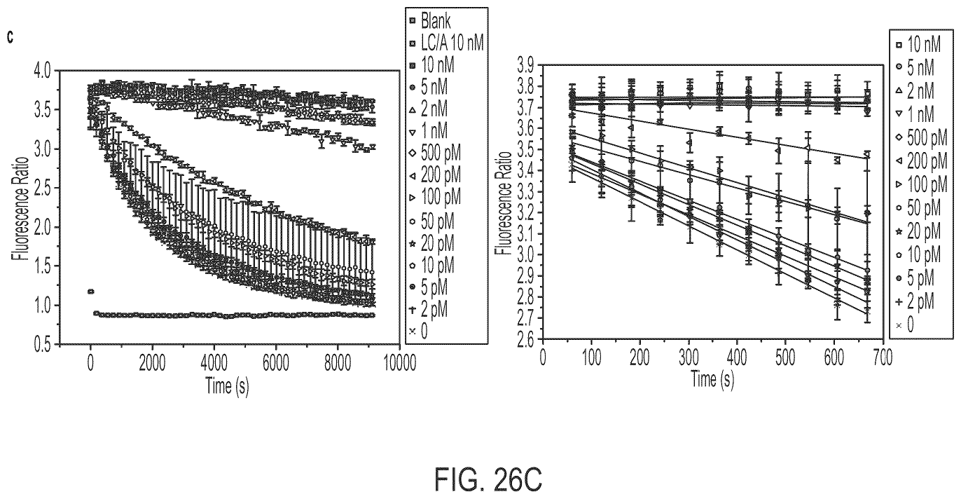

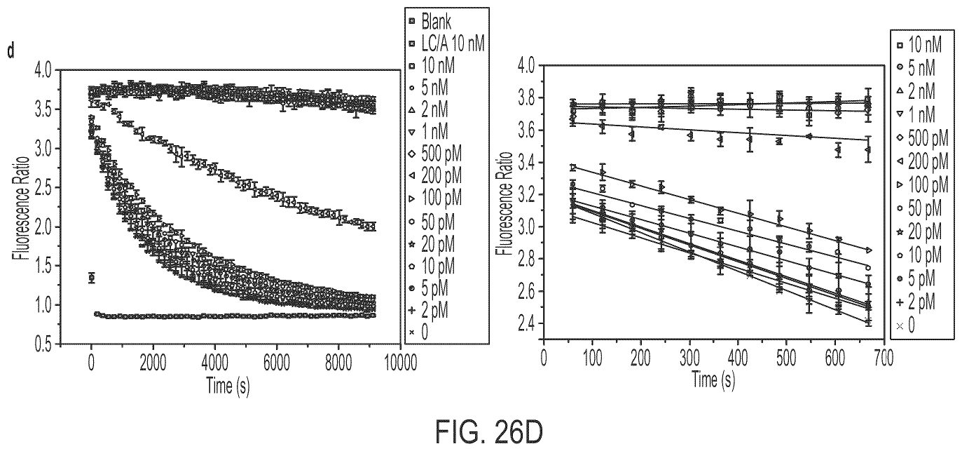

To characterize quantitatively the inhibitory capabilities of all three ncAA-sdAb variants considered here, a commercial FRET-based LC/A substrate was used to monitor enzymatic activity over time. By monitoring changes in reporter fluorescence over time in mixtures of a fixed LC/A concentration and varying sdAb amounts, the initial rates of reaction were determined. The rate in presence of each inhibitor concentrator could then be correlated to the fractional LC/A activity by normalizing by the rate of enzyme in the absence of sdAb. Fitting these data to a dose-response model allowed an estimation of the IC₅₀ values for each sdAb (FIGS. 6A and 6B; FIGS. 26A-26D). The results indicate that the sdAbs retained their inhibitory capabilities when incorporating these ncAAs, with only JPU-C1 Y32OBeY showing an approximately (˜) 5-fold decrease in inhibitory potency when compared to WT JPU-C1. The residue at position 32 is tyrosine in each of JPU-A5, JPU-C1, and JPU-C10, which may indicate that Y32 is a preferred amino acid at this sdAb position. Substitutions at this position could affect antibody stability and thus binding affinity to LC/A, resulting in the decreased inhibitory potency of JPU-C1 Y32OBeY. On the other hand, positions L101 and M104 are located within the highly variable CDR3 loop and are therefore more likely to tolerate ncAA substitutions without a loss of binding affinity or inhibitory potency. While it was attempted to characterize the effect of crosslinking in inhibition using this assay, the fast autoproteolysis of LC/A and its photodegradation when irradiated in the absence of sdAb prevented the ability to quantify the effect of crosslinking on enzymatic activity. Nonetheless, in-solution inhibition assays confirmed that ncAA-substituted variants of JPU-C1 retain inhibitory properties.

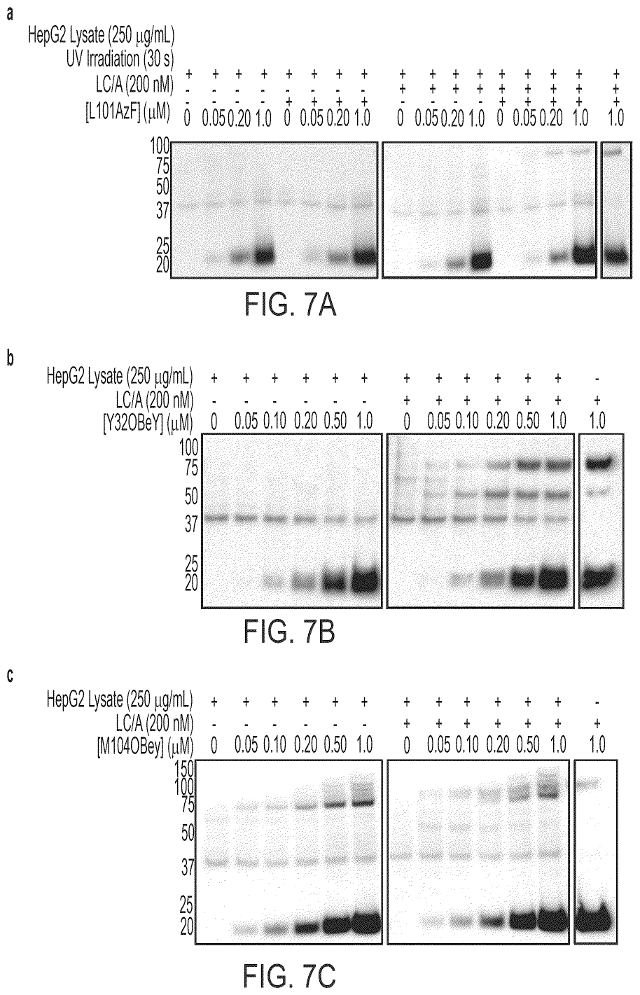

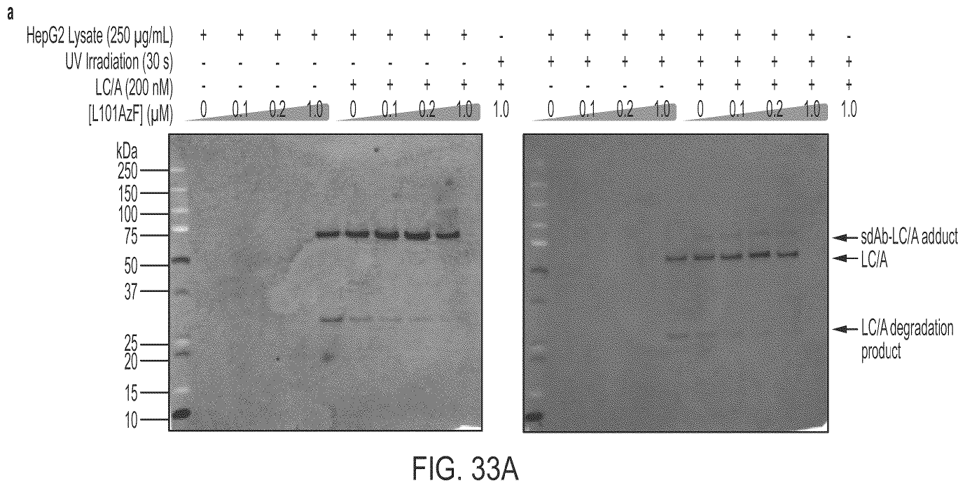

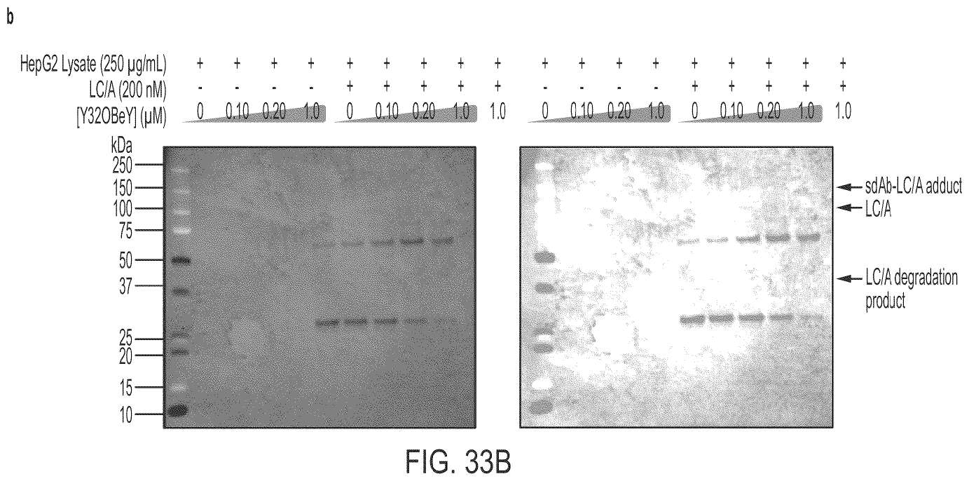

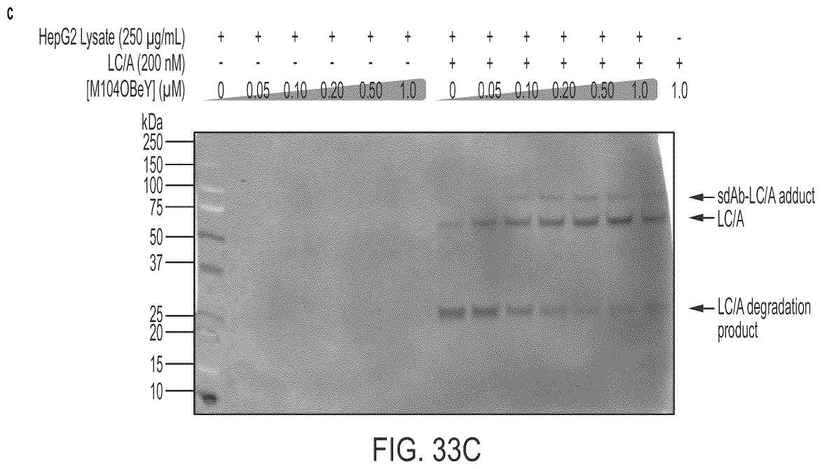

In addition to evaluating the behavior of the chemically reactive sdAb in “ideal” conditions, their selectivity when used to bind LC/A in complex mixtures to determine whether the introduction of crosslinkable groups leads to detectable levels of off-target interactions was investigated. Here, mammalian cell lysates were used to present a range of potential intracellular off targets, as any potential BoNT protease inhibitor would need to exhibit selectivity in the neuronal cytosol to prevent off-target crosslinking, thus reducing efficacy and possibly causing harm. Starting with the photo-crosslinkable JPU-C1 L101AzF sdAb, photo-crosslinking experiments were performed with varying concentrations of sdAb in the presence of HepG2 cell lysate spiked with a fixed concentration of LC/A. The solutions were then transferred to nitrocellulose membranes and probed for the c-Myc tag present in the sdAb. As observed in FIG. 7A, which shows the resulting blots before and after irradiating the samples for 30 s, a band near 37 kDa appears with similar intensity in all lysate-containing samples, which is attributed to a lysate protein that cross-reacts with the cMyc detection antibody (this also serves as an internal standard for evaluating sdAb selectivity). The appearance of the band corresponding to the LC/A-sdAb adduct (FIG. 33A) is dependent on concentration and requires sample irradiation. This provides a strong indication that the presence of lysate does not prevent crosslinking between the intended binding partners. Furthermore, the expected crosslinked adduct represents the major new band formed following irradiation. In the presence of LC/A, the appearance of additional bands remains at close to undetectable levels, while in the absence of LC/A, new bands only appear at very high concentrations of sdAb after irradiation. The intensities of these bands are noticeably lower than the intensities of the LC/A adduct; detailed densitometry (FIG. 33A) confirms all of these observations. These data indicate that the AzF-substituted sdAb exhibits high target selectivity even in a complex sample. Furthermore, new bands only appeared at very high concentrations of the sdAbs after irradiation, and their band intensity was noticeably lower than that found for the LC/A adduct.

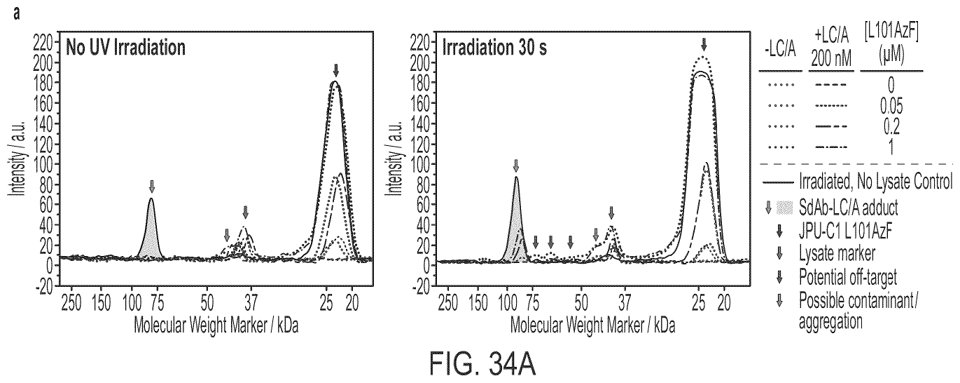

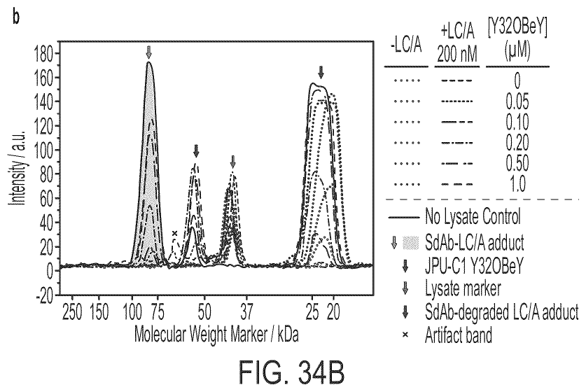

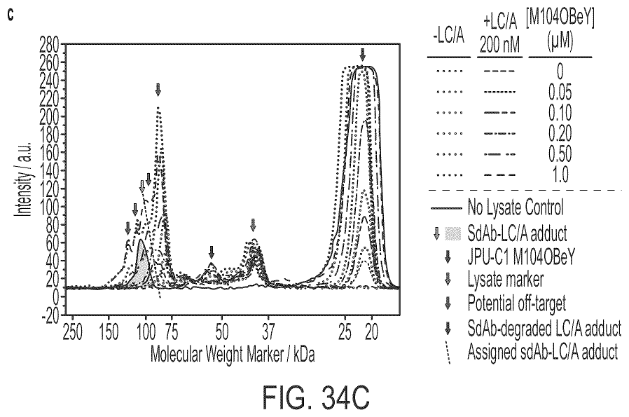

Similar experiments were performed with the OBeY containing sdAbs JPU-C1 Y32OBeY and JPU-C1 M104OBeY. The sdAbs were incubated with HepG2 lysate, and some samples were spiked with LC/A. FIG. 7B highlights the appearance of the LC/A-JPU-C1 Y32OBeY adduct with a concentration dependent band at ˜75 kDa (FIG. 33B), as well as the adduct corresponding to partially hydrolyzed LC/A-JPU-C1 Y32OBeY complex near the 50 kDa marker. Notably, no other bands other than the internal control were detected in this assay (See, FIG. 34B for detailed densitometry), indicating again that the presence of foreign proteins did not prevent association and reaction between JPU-C1 Y32OBeY and LC/A. Importantly, JPU-C1 Y32OBeY did not show detectable levels of off-target protein binding under the conditions used as described.

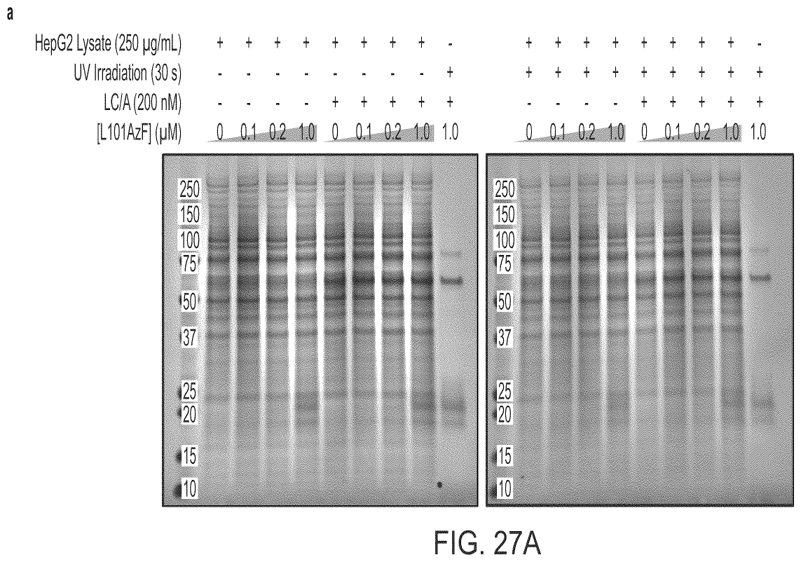

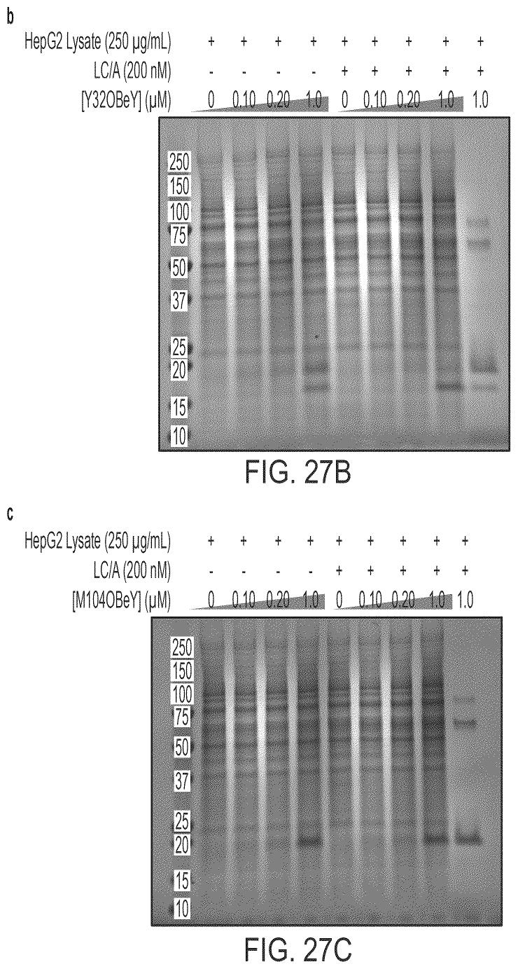

FIG. 7C shows the analysis of the western blotting experiment performed using JPU-C1 M104OBeY. The crosslinked sdAb-LC/A adduct (˜75 kDa) could be detected when blotting against the LC/A even at substoichiometric concentrations of sdAb, again showing that the presence of foreign proteins did not preclude the covalent interaction between them. The appearance of an additional band slightly below the 75 kDa marker was observed with increasing concentrations of M104OBeY in the absence of LC/A, suggesting an unintended reaction of the sdAb with a protein in the lysate. Furthermore, a few lesser intense bands in the 75-100 kDa appeared following incubations at high concentrations of sdAb. (Coomassie gels are shown in FIGS. 27A-27C). While this may indicate off-target interactions of this substituted sdAb, by comparing the intensity profiles in the absence and presence of LC/A via densitometry (FIG. 34C), the appearance of these side products in the presence of LC/A was observed to be suppressed, appearing only at sdAb concentrations similar to or greater than the concentration of the added LC/A. This suggests that the sdAb exhibits a distinct crosslinking preference for LC/A, although its selectivity is lower than the selectivity of the other soluble ncAA-sdAbs characterized here.

Overall, these assays indicate that ncAA-substituted clones retained their inhibitory properties and can further exhibit target selectivity, although the extent to which selectivity is observed appears to depend on the ncAA substitution site. Thus, chemically augmented sdAbs initially identified on the yeast surface in this work employing two different chemistries exhibited crosslinking functionalities in solution while retaining inhibition and, in some cases, high target selectivity.

### Summary of Results

In the studies described herein, a combination of yeast display and ncAAs was used to discover and characterize sdAbs capable of covalently binding to their targets in one of two ways: via irradiation with short-wavelength light or via proximity-induced reaction. Using crystallographic data (e.g., Chen, S. et al., 2021, Nature Biotechnology 2021, 39 (4), 490-498. DOI: 10.1038/s41587-020-0733-7; McCarthy, K. A. et al., 2018, Journal of the American Chemical Society 140 (19), 6137-6145. DOI: 10.1021/jacs.8b02461), two reactive ncAAs were installed, namely, AzF (for photocrosslinking) and OBeY (for spontaneous crosslinking), at selected positions in four sdAbs known to bind to the botulinum neurotoxin light chain A1. Conducting assays in yeast display format allowed for efficient flow cytometric characterization of more than 40 sdAb variants, all without protein purification, and revealed that sdAbs exhibit high functional tolerance toward single site substitution with ncAAs. It was further determined that this approach streamlines the identification of crosslinkable candidates via flow cytometry, both to identify sites that facilitate crosslinking and to evaluate time-dependent behaviors of these interactions.

These studies described herein include what is likely the first discovery of spontaneously crosslinkable binding agents in yeast display format. The discoveries of covalent OBeY-sdAb variants were unexpected, as the sites investigated as described herein were initially selected using the structural data with the goal of facilitating the introduction of photocrosslinkable ncAAs, not electrophilic ncAAs. Finally, solution-based experiments revealed that the sdAb variants JPU-CT LTOTAzF, Y32OBeY, and M104OBeY exhibited clear crosslinking activity in solution, retained the inhibitory activity of the parent, and possessed moderate to high levels of selectivity for LC/A when tested in the presence of complex protein mixtures.

Although a known benefit of irreversible inhibition is enhancement of inhibitor potency, converting inhibitors that already exhibit high potency (such as the parent sdAbs used herein) into covalent inhibitors may not readily reveal enhanced potency under typical conditions used during in vitro enzyme inhibition assays. Nonetheless, covalent target engagement can be advantageous in the prevention of inhibitor dissociation from the target and reactivation of the target. In the case of BoNT, covalent inhibitors are desirable to permanently inactivate the light chain protease, which exhibits an exceptionally long-lived half-life (˜months) in the intracellular space.

In order to gauge the success of the predicted reactive sdAb positions, structural data were used to estimate distances between sdAb and LC/A residues, and then values were compared with the apparent reactivities of sdAb variants determined on the yeast surface. Interestingly, many of these analyses revealed no clear correlations when measuring distances to the closest residue or nucleophile; even the short distances to the nearest Cys or His residues noted in sdAb clones that spontaneously crosslink to LC/A do not unambiguously reveal a single crosslinking site on the target. Observations indicate that distance measurements alone are insufficient for accurate prediction of which ncAA substitutions will lead to crosslinking events (although it is likely that expression level variability between clones accounts for some differences in apparent crosslinking levels). This suggests that factors in addition to proximity between ncAA substitution sites and nucleophiles on the target play a role in covalent target engagement. Unpredictable reactivity levels and a dearth of structural data highlight the utility of a yeast display platform, or other platforms in the high throughput technology space, to facilitate engineering and characterization of chemically expanded antibodies that exhibit high affinity and target selectivity.

The yeast-display screening tools described herein can be complemented by mechanistic investigations of successful crosslinkable antibodies using mass spectrometry to identify crosslinking site. Additionally, valuable information could be obtained from structural characterization of purified adducts, such as the identification of local environments within complexes that promote efficient crosslinking. Thus, increasing both the breadth and depth of studies with covalent antibodies will be paramount for establishing principles for covalent binding agent discovery.

Moving beyond characterizations, the high throughput capabilities of yeast display can be leveraged with libraries of ncAA-containing proteins. New and improved genetic code expansion tools in yeast make it feasible to combine established yeast display strategies with libraries encoding ncAAs bearing various reactive groups (e.g., fluorosulfates for SuFEX chemistry and various Michael acceptors) or with ncAAs that facilitate conjugations with chemical warheads. Such a combination provides a wide range of opportunities for covalent antibody discovery. These results show that the phenotype resulting from ncAA-mediated covalent bond formation can be assayed on the yeast surface, while prior work in yeast display format established high throughput screens to tune the efficiency and selectivity of covalent bond formation. A platform for discovering and characterizing covalent antibodies is advantageous for applications of covalent protein binding agents in basic biomedical research, diagnostics, and drug discovery.

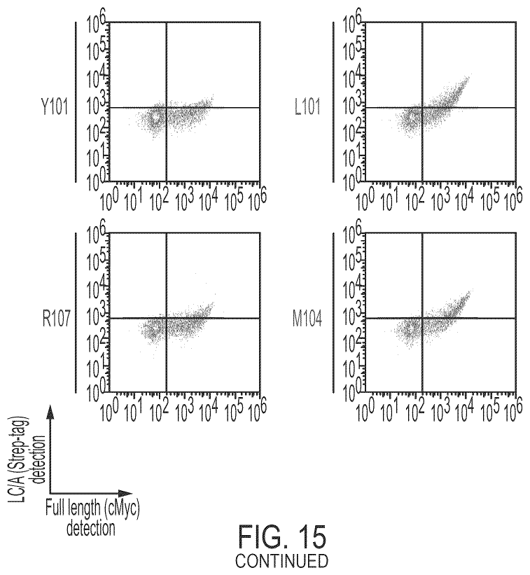

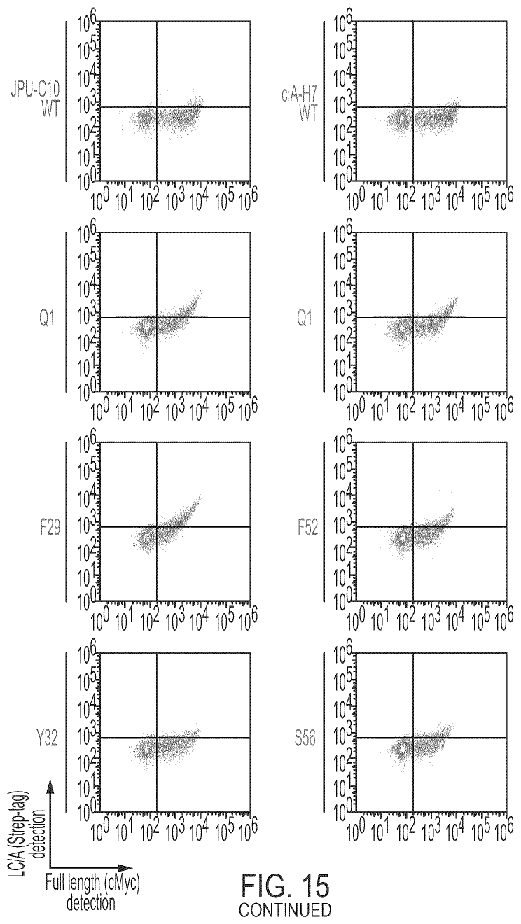

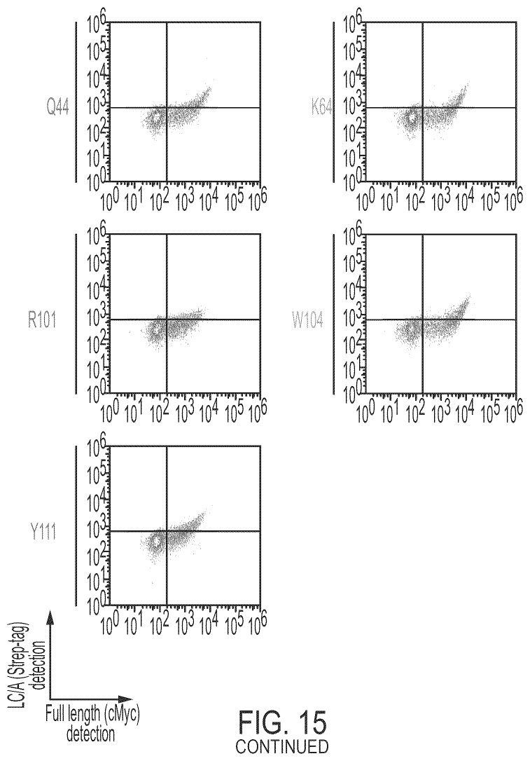

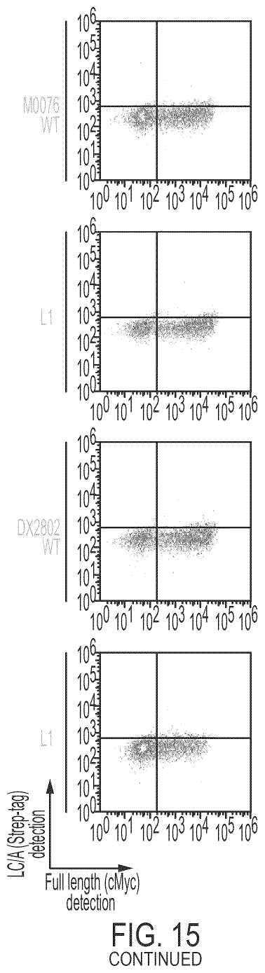

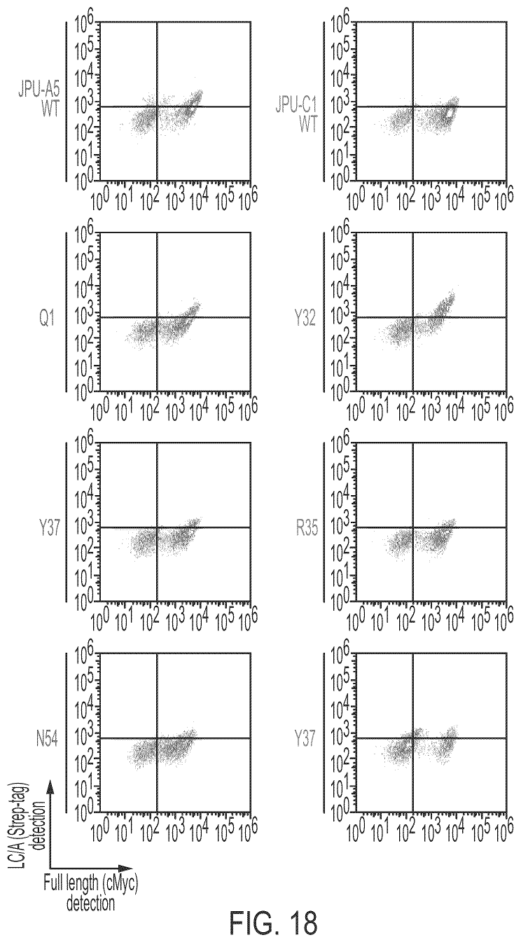

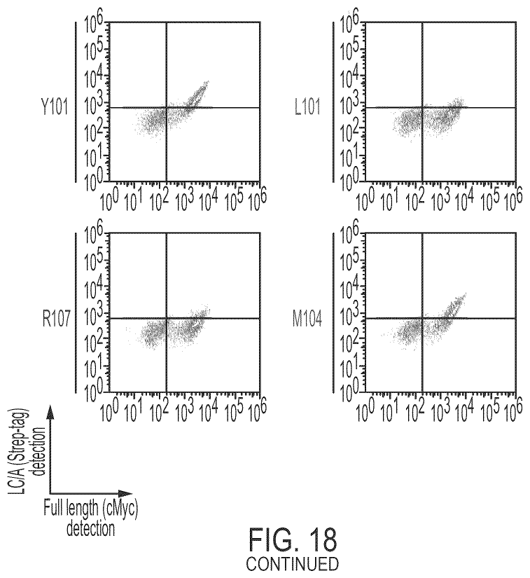

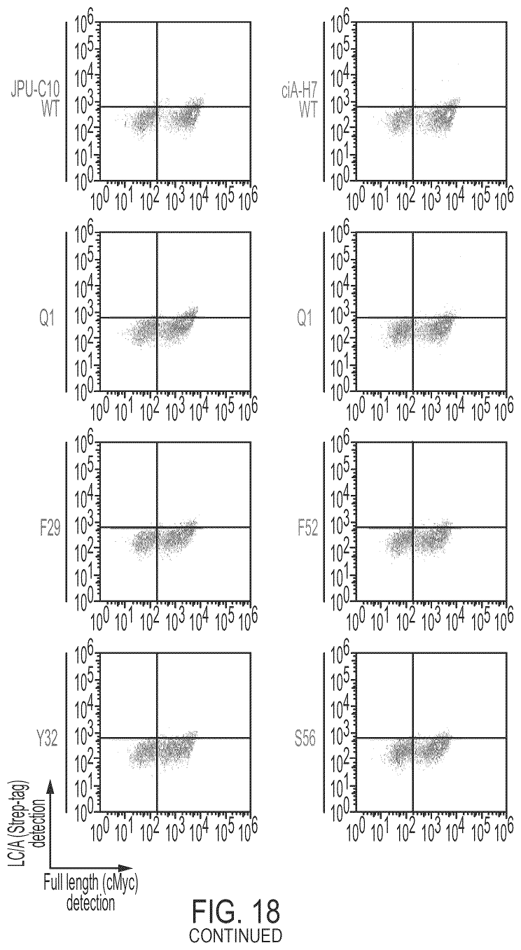

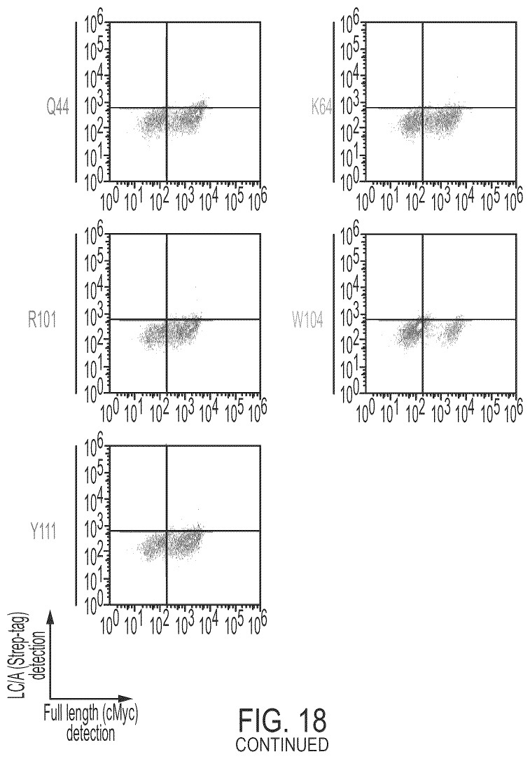

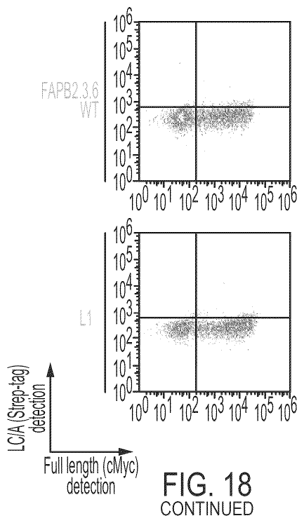

The studies described herein advance the use of yeast display as a high throughput technique for antibody engineering by employing this platform in connection with the discovery of synthetic proteins with an expanded chemical repertoire with the use of ncAAs. Similar to the current process of small molecule lead development, the robustness of yeast display and flow cytometry (e.g., FIG. 15; FIG. 18) can be employed in synergy with other promising chemical diversification strategies to enhance covalent antibodies by using established high throughput screening and directed evolution strategies. In addition, characterization of the specific reactivity sites will allow for the development of reactivity requirements and improved structure-based rational designs. The approaches described herein can enhance the identification and selection of highly potent, reactive sdAbs with faster crosslinking times, higher crosslinking yields, and decreased off-target effects for use in therapeutics and diagnostics.

### Example 7: Materials and Methods

Provided in this Example are materials and methods used in the above-described Examples and in the figures.

### Reagents

Buffers. Phosphate Buffered Saline supplemented with Bovine Serum Albumin (PBSA) was made to contain 137 mM NaCl, 2.7 mM KCl, 10 mM Na₂HPO₄, 18 mM KH₂PO₄, and 0.1% w/v bovine serum albumin, adjusted to pH 7.4 with NaOH. LC/A Reconstitution Buffer was made to contain 50 mM HEPES, 5 mM NaCl, 10 μM ZnCl₂, 15 μM MgCl₂, and 0.10% v/v Tween-20, adjusted to pH 7.1. LC/A working buffer was made to contain 50 mM HEPES, 5 mM NaCl, 10 μM ZnCl₂, and 0.1% v/v Tween-20, adjusted to pH 7.1. All buffers were filtered through 0.2 μm membranes.

NcAA preparation. NcAAs were obtained from ChemImpex (AzF; Wood Dale, IL), Advanced ChemBlocks Inc. (OBeY, Hayward, CA), and Iris Biotech (OPG; Germany). NcAAs were prepared before use as 50 mM stocks (50×) in water with addition of 6 N NaOH to aid in solubilization. Stocks were filtered through 0.2 μm membranes before use.

### Media

LB, SD-CAA, SG-CAA, SD-SCAA, SG-SCAA, and YPG media were prepared as previously described. (Hershman, R. L. et al., 2022, In Yeast Surface Display, Traxlmayr, M. W. Ed.; Springer US; pp 491-559; Stieglitz, J. T. et al., 2022, In Biomedical Engineering Technologies: Volume 2, Rasooly, A. et al. Eds.; Springer US; pp 377-432). Selective SD-SCAA and SG-SCAA media were prepared without tryptophan, uracil, and leucine (-Trp -Ura -Leu). All yeast media was supplemented with penicillin/streptomycin (100 U mL⁻¹/100 μg mL⁻¹; Corning, Corning, NY).

### Software

PyMOL was used to visualize crystallographic data. A script in Python 3.10.4 using the Biopython 1.79 library was developed to find the nearest distances to each sdAb mutant position. SnapGene Viewer and Benchling were used to design DNA sequences and to analyze DNA sequencing data. FlowJo was used to visualize and analyze flow cytometry data. Microsoft Excel 365 was used to analyze flow cytometry data. OriginLab 2022 was used to plot graphs and fit the titration curves. GraphPad Prism 9 was used to fit the dose-response curves and perform the IC50 statistical comparisons. ImageJ was used for image densitometric analysis. Adobe Illustrator was used to create figures.

### DNA and Protein Sequences

DNA primers were purchased from Genewiz (Cambridge, MA) and synthetic genes were obtained from Integrated DNA Technologies (Coralville, IA) or Genscript (for LC/A; Piscataway, NJ). Sanger DNA sequencing was carried out by Quintara Biosciences (Cambridge, MA). In the below, “FOR” indicates “forward primer”; “REV” indicates “reverse primer”.

| | | | SEQ ID |
|---|---|---|---|
| ### | Name | Sequence | NO: |
| AOF | pCTCON2_JPU-A5.FOR | CTTAGTGCTAGCCAGGTGCAGCTCG | 4 |
| | | TGGAGACAGG | |
| AOR | pCTCON2_JPU-A5.REV | GGTGCGGGATCCTGAAGAGACGGTG | 5 |
| | | ACCTGAATGCCCTGACCCCATGACT | |
| | | CAGGGTGAC | |
| BOF | pCTCON2_JPU-C1.FOR | GCCTATGCTAGCCAGGTGCAGCTGG | 6 |
| | | CGGAGTCGG | |
| BOR | pCTCON2_JPU-C1.REV | GAACACGGATCCTGAGGAGACGGTC | 7 |
| | | ACCTGGGTCC | |
| COF | pCTCON2_JPU-C10.FOR | GTGGAGGCGGTAGCGGAGGCGGAGG | 8 |
| | | GTCGGCTAGCCAACTGCAACTAGTA | |
| | | GAAAGTGGGG | |
| COR | pCTCON2_JPU-C10.REV | AAGTCCTCTTCAGAAATAAGCTTTT | 9 |
| | | GTTCGGATCCTGAGGATACTGTTAC | |
| | | TTGCGTCCC | |
| DOF | pCTCON2_ciA-H7.FOR | GCATCAGCTAGCCAGGTGCAGCTCG | 10 |
| | | TGGAGTCAGG | |
| DOR | pCTCON2_ciA-H7.REV | GGTGAGGGATCCTGAGGAGACGGTG | 11 |
| | | ACCTGGGTCC | |
| S1F | CON2SEQ.FOR | GTTCCAGACTACGCTCTGCAG | 12 |
| S1R | CON2SEQ.REV | GATTTTGTTACATCTACACTGTT | 13 |
| A1F | pCTCON2_JPU- | GGAGGGTCGGCTAGCTAGGTGCAGC | 14 |
| | A5_Q1TAG.FOR | TCGTGGAGACAG | |
| A1R | pCTCON2_JPU- | CTGTCTCCACGAGCTGCACCTAGCT | 15 |
| | A5_Q1TAG.REV | AGCCGACCCTCC | |
| A2F | pCTCON2_JPU- | CCATGGGCTGGTAGCGCCAGACTCC | 16 |
| | A5_Y37TAG.FOR | AGGAAATTCGCG | |
| A2R | pCTCON2_JPU- | CGCGAATTTCCTGGAGTCTGGCGCT | 17 |
| | A5_Y37TAG.REV | ACCAGCCCATGG | |
| A3F | pCTCON2_JPU- | GAGTTGGTCGCGGTGATGAATCTTT | 18 |
| | A5_N54TAG.FOR | AGGGCGTCATAAGCTATGGG | |
| A3R | pCTCON2_JPU- | CCCATAGCTTATGACGCCCTAAAGA | 19 |
| | A5_N54TAG.REV | TTCATCACCGCGACCAACTC | |
| A4F | pCTCON2_JPU- | GTAATGGTATGAGACTATAGACCCG | 20 |
| | A5_Y101TAG.FOR | GGGCAGTGTTCGTCACCC | |
| A4R | pCTCON2_JPU- | GGGTGACGAACACTGCCCCGGGTCT | 21 |
| | A5_Y101TAG.REV | ATAGTCTCATACCATTAC | |
| A5F | pCTCON2_JPU- | CTATATACCCGGGGCAGTGTTTAGC | 22 |
| | A5_R107TAG.FOR | ACCCTGAGTCATGGGGTCAGGGC | |
| A5R | pCTCON2_JPU- | GCCCTGACCCCATGACTCAGGGTGC | 23 |
| | A5_R107TAG.REV | TAAACACTGCCCCGGGTATATAG | |
| B1F | pCTCON2_JPU- | GGTATGTCATAAGGTGGTAGCGCCA | 24 |
| | C1_Y32TAG.FOR | GGCTCCAGGGAAG | |
| B1R | pCTCON2_JPU- | GCCTGGCGGTACCACCTTATGACCT | 25 |
| | C1_Y32TAG.REV | ACCTATTAAACGTGAATCC | |
| B2F | pCTCON2_JPU- | CACGTTTAATAGGTATGTCATATAG | 26 |
| | C1_R35TAG.FOR | TGGTACCGCCAGGCTCCAGGGAAGG | |
| B2R | pCTCON2_JPU- | CCTTCCCTGGAGCCTGGCGGTACCA | 27 |
| | C1_R35TAG.REV | CTATATGACATACCTATTAAACGTG | |
| B3F | pCTCON2_JPU- | GGTATGTCATAAGGTGGTAGCGCCA | 24 |
| | C1_Y37TAG.FOR | GGCTCCAGGGAAG | |
| B3R | pCTCON2_JPU- | CTTCCCTGGAGCCTGGCGCTACCAC | 29 |
| | C1_Y37TAG.REV | CTTATGACATACC | |
| B4F | pCTCON2_JPU- | CCGTCTATTACTGCAGCGCCCTCAA | 30 |
| | C1_L101TAG.FOR | TTAGGAAGATATGGAATATTGGG | |
| B4R | pCTCON2_JPU- | CCCAATATTCCATATCTTCCTAATT | 31 |
| | C1_L101TAG.REV | GAGGGCGCTGCAGTAATAGACGG | |
| B5F | pCTCON2_JPU- | GCAGCGCCCTCAATTTAGAAGATTA | 32 |
| | C1_M104TAG.FOR | GGAATATTGGGGCCAGGGGAC | |
| B5R | pCTCON2_JPU- | GTCCCCTGGCCCCAATATTCCTAAT | 33 |
| | C1_M104AG.REV | CTTCTAAATTGAGGGCGCTGC | |
| C1F | pCTCON2_JPU- | GGCGGAGGGTCGGCTAGCTAGCTGC | 34 |
| | C10_Q1TAG.FOR | AACTAGTAGAAAGTGGG | |
| C1R | pCTCON2_JPU- | CCCACTTTCTACTAGTTGCAGCTAG | 35 |
| | C10_Q1TAG.REV | CTAGCCGACCCTCCGCC | |
| C2F | pCTCON2_JPU- | GCCGCATCTGGGAACATCTAGTCCA | 36 |
| | C10_F29TAG.FOR | TTTATTACATGGGCTGGTATAGACA | |
| | | AGC | |
| C2R | pCTCON2_JPU- | GCTTGTCTATACCAGCCCATGTAAT | 37 |
| | C10_F29TAG.REV | AAATGGACTAGATGTTCCCAGATGC | |
| | | GGC | |
| C3F | pCTCON2_JPU- | GGGAACATCTTCTCCATTTAGTACA | 38 |
| | C10_Y32TAG.FOR | TGGGCTGGTATAGACAAGCCC | |
| C3R | pCTCON2_JPU- | GGGCTTGTCTATACCAGCCCATGTA | 39 |
| | C10_Y32TAG.REV | CTAAATGGAGAAGATGTTCCC | |
| C4F | pCTCON2_JPU- | GCTGGTATAGACAAGCCCCCGGAAA | 40 |
| | C10_Q44TAG.FOR | ATAGAGAGAAATGGTAGCTATC | |
| C4R | pCTCON2_JPU- | GATAGCTACCATTTCTCTCTATTTT | 41 |
| | C10_Q44TAG.REV | CCGGGGGCTTGTCTATACCAGC | |
| C5F | pCTCON2_JPU- | GTACTATTGTAACGCAGGAAAACTT | 42 |
| | C10_R101TAG.FOR | TAGAGAACCACCGGGTGGGGGC | |
| C5R | pCTCON2_JPU- | GCCCCCACCCGGTGGTTCTCTAAAG | 43 |
| | C10_R101TAG.REV | TTTTCCTGCGTTACAATAGTAC | |
| C6F | pCTCON2_JPU- | GGGCTAGACGATTAGTGGGGTCAGG | 44 |
| | C10_Y111TAG.FOR | GGACGC | |
| C6R | pCTCON2_JPU- | GCGTCCCCTGACCCCACTAATCGTC | 45 |
| | C10_Y111TAG.REV | TAGCCC | |
| D1F | pCTCON2_CIA- | GGAGGGTCGGCTAGCTAGGTGCAGC | 46 |
| | H7_Q1TAG.FOR | TCGTGGAGTCAG | |
| D1R | pCTCON2_CIA- | CTGACTCCACGAGCTGCACCTAGCT | 47 |
| | H7_Q1TAG.REV | AGCCGACCCTCC | |
| D2F | pCTCON2_CIA- | GAAATGGTCGCAGATATTTAGTCTG | 48 |
| | H7_F52TAG.FOR | GCGGTAGTACAGACTATGCAGGCTC | |
| | | CG | |
| D2R | pCTCON2_CIA- | CGGAGCCTGCATAGTCTGTACTACC | 49 |
| | H7_F52TAG.REV | GCCAGACTAAATATCTGCGACCATT | |
| | | TC | |
| D3F | pCTCON2_CIA- | GCAGATATTTTTTCTGGCGGTTAGA | 50 |
| | H7_S56TAG.FOR | CAGACTATGCAGGCTCCGTGAAGGG | |
| D3R | pCTCON2_CIA- | CCCTTCACGGAGCCTGCATAGTCTG | 51 |
| | H7_S56TAG.REV | TCTAACCGCCAGAAAAAATATCTGC | |
| D4F | pCTCON2_CIA- | CAGACTATGCAGGCTCCGTGTAGGG | 52 |
| | H7_K64TAG.FOR | CCGATTCACCATCTC | |
| D4R | pCTCON2_CIA- | GAGATGGTGAATCGGCCCTACACGG | 53 |
| | H7_K64TAG.REV | AGCCTGCATAGTCTG | |
| D5F | pCTCON2_CIA- | GGAGCGGTGACTACTAGGGCCAGGG | 54 |
| | H7_W104TAG.FOR | GACCCAGGTCAC | |
| D5R | pCTCON2_CIA- | GTGACCTGGGTCCCCTGGCCCTAGT | 55 |
| | H7_W104TAG.REV | AGTCACCGCTCC | |
| E0F | pRS314_cMSH6.FOR | GGAACCCTGGTCACCGTCTCCTCGG | 56 |
| | | GATCCGAACAAAAGCTTATTTCTGA | |
| | | AGAA | |
| E0R | pRS314_cMSH6.REV | GTTACATCTACACTGTTGTTATCAGATCTCG | 57 |
| | | AGCTATTAATGGTGATGGTGGTGATGG | |
| G0F | pRS314_cMSH6_JPU- | CAAGAGAGAAGCTCGGCCGGCTAGC | 58 |
| | C1.FOR | CAGGTGCAGCTGGCG | |
| G0R | pRS314_cMSH6_JPU- | TCAGAAATAAGCTTTTGTTCGGATC | 59 |
| | C1.REV | CTGAGGAGACGGTCACCTGGG | |
| S2F | pRS314_SEQ.FOR | GTTTTGATTGTCTTGTTGGC | 60 |
| S2R | pRS314_SEQ.REV | CATGGGAAAACATGTTGTTTAC | 61 |

Mutation sites are highlighted in bold font.

### Gene/Polynucleotide Sequences

| Name | Sequence | SEQ ID NO: |
|---|---|---|
| LC/A- | ATGGGTCATCATCATCATCATCATCATCACGGTGCGGGCGAGAATCTGTACT | 88 |
| Strep- | TCCAGGGCGCCGGCGGTCCGTTTGTGAACAAGCAGTTCAACTACAAAGACCC | |
| tag | GGTGAACGGCGTGGATATCGCTTACATCAAGATCCCGAACGCCGGTCAGATG | |
| | CAGCCGGTGAAGGCTTTCAAAATCCACAACAAAATCTGGGTTATCCCGGAGC | |
| | GCGACACCTTCACCAACCCGGAGGAAGGCGATCTGAACCCGCCGCCGGAGGC | |
| | CAAGCAGGTGCCGGTGAGCTACTACGATAGCACCTACCTGAGCACCGACAAC | |
| | GAGAAAGATAACTACCTGAAGGGTGTGACCAAACTGTTCGAACGTATCTACA | |
| | GCACCGACCTGGGCCGCATGCTGCTGACCAGCATCGTGCGTGGTATCCCGTT | |
| | CTGGGGCGGTAGCACCATCGACACCGAGCTGAAAGTGATCGATACCAACTGC | |
| | ATCAACGTGATCCAGCCGGACGGCAGCTACCGTAGCGAGGAGCTGAACCTGG | |
| | TTATCATCGGTCCGAGCGCCGATATCATCCAGTTCGAGTGCAAGAGCTTCGG | |
| | CCACGAAGTGCTGAACCTGACCCGCAACGGCTACGGTAGCACCCAGTACATC | |
| | CGTTTCAGCCCGGACTTCACCTTCGGTTTCGAGGAAAGCCTGGAAGTGGATA | |
| | CCAACCCGCTGCTGGGTGCTGGCAAGTTTGCTACCGACCCAGCTGTGACCCT | |
| | GGCTCACGAACTGATTCACGCTGGTCACCGTCTGTATGGTATCGCTATCAAC | |
| | CCGAACCGCGTGTTCAAGGTGAACACCAACGCCTACTACGAGATGAGCGGCC | |
| | TGGAAGTGAGCTTCGAGGAACTGCGCACCTTCGGCGGTCACGACGCGAAATT | |
| | CATCGATAGCCTGCAGGAGAACGAATTCCGTCTGTACTACTACAACAAGTTC | |
| | AAAGATATCGCCAGCACCCTGAACAAGGCGAAAAGCATCGTGGGCACCACCG | |
| | CGAGCCTGCAGTACATGAAGAACGTGTTCAAGGAGAAATACCTGCTGAGCGA | |
| | AGACACCAGCGGTAAATTCAGCGTGGACAAGCTGAAATTCGATAAGCTGTAC | |
| | AAAATGCTGACCGAGATCTACACCGAAGACAACTTCGTGAAGTTCTTCAAAG | |
| | TGCTGAACCGCAAGACCTACCTGAACTTCGATAAGGCCGTGTTCAAGATCAA | |
| | CATCGTGCCGAAAGTGAACTACACCATCTACGACGGCTTCAACCTGCGTAAC | |
| | ACCAACCTGGCCGCGAACTTCAACGGTCAGAACACCGAGATCAACAACATGA | |
| | ACTTCACCAAGCTGAAAAACTTCACCGGCCTGTTCGAATTCTACAAGCTGCT | |
| | GAGCGTGCGCGGTATCATCACCAGCAAAGGTGCGGGCGAGAACCTGTACTTC | |
| | CAGGGTGCTGGCTGGAGCCACCCGCAGTTCGAAAAGGGTGCTGGTTGGAGCC | |
| | ATCCGCAGTTTGAGAAA | |
| JPU-A5 | CAGGTGCAGCTCGTGGAGACAGGGGGAGGCTTGGTTCAGGCTGGGGGGTCTC | 89 |
| | TGAGACTCTCCTGTACAGCCTCTGGAGCCGACTTCAGTTTCTATGCCATGGG | |
| | CTGGTACCGCCAGACTCCAGGAAATTCGCGCGAGTTGGTCGCGGTGATGAAT | |
| | CTTAATGGCGTCATAAGCTATGGGGACTCCGCTCGGGGCCGATTCGACATCT | |
| | CCAGGGACGGCACCAAGAACATAGTGTTTCTGCAAATGAACAGCCTGAAACC | |
| | TGAAGACACGGGCGTTTATTACTGTAATGGTATGAGACTATATACCCGGGGC | |
| | AGTGTTCGTCACCCTGAGTCATGGGGTCAGGGGATCCAGGTCACCGTCTCTT | |
| | CA | |
| JPU-C1 | CAGGTGCAGCTGGCGGAGTCGGGAGGAGGCCTGGTGCAGCCGGGGGGGTCTC | 90 |
| | TGAGACTCTCCTGTGCAGCCTCCGGATTCACGTTTAATAGGTATGTCATAAG | |
| | GTGGTACCGCCAGGCTCCAGGGAAGGAGCGCGAATTGGTCGCAGGTATTTCG | |
| | CGGTCTGGTGATTCTGGAAGGTATGTCGACTCCGTGAAGGGCCGATTCACCA | |
| | TCTCCAGAGACAATGACAAGAACATGGCCTATCTACAAATGAGCTCCCTAAA | |
| | ACCTGACGACACGGCCGTCTATTACTGCAGCGCCCTCAATTTAGAAGATATG | |
| | GAATATTGGGGCCAGGGGACCCAGGTGACCGTCTCCTCA | |
| JPU- | CAACTGCAACTAGTAGAAAGTGGGGGCGGGCTTGTACAACCCGGTGGAAGCC | 62 |
| C10 | TACGTCTATCATGTGCCGCATCTGGGAACATCTTCTCCATTTATTACATGGG | |
| | CTGGTATAGACAAGCCCCCGGAAAACAGAGAGAAATGGTAGCTATCATCAAT | |
| | AGTAACGGTATCACTAACTATGGGGATTTCGTTAAAGGCAGATTTACCATTA | |
| | GTAGAGACAACGCTGAGAACAGTGCTTATCTACAAATGAATAACCTTACGCC | |
| | GGAGGATACCGCAGTGTACTATTGTAACGCAGGAAAACTTAGGAGAACCACC | |
| | GGGTGGGGGCTAGACGATTATTGGGGTCAGGGGACGCAAGTAACAGTATCCT | |
| | CA | |
| ciA-H7 | TGGAGGCTTGGTGCAGGTTGGGGGGTCTCTGAGACTCTCCTGTGTAGTTTCT | 91 |
| | GGAAGCGACATCAGTGGCATTGCGATGGGCTGGTACCGCCAGGCTCCAGGGA | |
| | AGCGGCGCGAAATGGTCGCAGATATTTTTTCTGGCGGTAGTACAGACTATGC | |
| | AGGCTCCGTGAAGGGCCGATTCACCATCTCCAGAGACAACGCCAAGAAGACG | |
| | AGCTATCTGCAAATGAACAACGTGAAACCTGAGGACACCGGAGTCTACTACT | |
| | GTAGGCTGTACGGGAGCGGTGACTACTGGGGCCAGG | |
| cMSH6 | GGATCCGAACAAAAGCTTATTTCTGAAGAAGACTTGGGTGGTGGAGGGTCCG | 63 |
| | GAGGACTGCCCGAAACGGGCGGCCATCACCACCATCACCATTAATAGCTCGA | |
| | GATCTGATAACAACAGTGTAG | |

### Protein Sequences

| Name | Sequence | SEQ ID NO: |
|---|---|---|
| LC/A- | MGHHHHHHHHGAGENLYFQGAGGPFVNKQFNYKDPVNGVDIA | 87 |
| Strep-tag | YIKIPNAGQMQPVKAFKIHNKIWVIPERDTFTNPEEGDLNPP | |
| “c“ | PEAKQVPVSYYDSTYLSTDNEKDNYLKGVTKLFERIYSTDLG | |
| | RMLLTSIVRGIPFWGGSTIDTELKVIDTNCINVIQPDGSYRS | |
| | EELNLVIIGPSADIIQFECKSFGHEVLNLTRNGYGSTQYIRF | |
| | SPDFTFGFEESLEVDTNPLLGAGKFATDPAVTLAHELIHAGH | |
| | RLYGIAINPNRVFKVNTNAYYEMSGLEVSFEELRTFGGHDAK | |
| | FIDSLQENEFRLYYYNKFKDIASTLNKAKSIVGTTASLQYMK | |
| | NVFKEKYLLSEDTSGKFSVDKLKFDKLYKMLTEIYTEDNFVK | |
| | FFKVLNRKTYLNFDKAVFKINIVPKVNYTIYDGFNLRNTNLA | |
| | ANFNGQNTEINNMNFTKLKNFTGLFEFYKLLSVRGIITSKGA | |
| | GENLYFQGAGWSHPQFEKGAGWSHPQFEK | |
| JPU-A5 | QVQLVETGGGLVQAGGSLRLSCTASGADFSFYAMGWYRQTPG | 64 |
| | NSRELVAVMNLNGVISYGDSARGRFDISRDGTKNIVFLQMNS | |
| | LKPEDTGVYYCNGMRLYTRGSVRHPESWGQGIQVTVSS | |
| JPU-C1 | QVQLAESGGGLVQPGGSLRLSCAASGFTFNRYVIRWYRQAPG | 65 |
| | KERELVAGISRSGDSGRYVDSVKGRFTISRDNDKNMAYLQMS | |
| | SLKPDDTAVYYCSALNLEDMEYWGQGTQVTVSS | |
| JPU-C10 | QLQLVESGGGLVQPGGSLRLSCAASGNIFSIYYMGWYRQAPG | 66 |
| | KQREMVAIINSNGITNYGDFVKGRFTISRDNAENSAYLQMNN | |
| | LTPEDTAVYYCNAGKLRRTTGWGLDDYWGQGTQVTVSS | |
| JDQ-H7 | QVQLVESGGGLVQVGGSLRLSCVVSGSDISGIAMGWYRQAPG | 67 |
| | KRREMVADIFSGGSTDYAGSVKGRFTISRDNAKKTSYLQMNN | |
| | VKPEDTGVYYCRLYGSGDYWGQGTQVTVSS | |

Mutation sites are highlighted in underlined bold font.
“c“ The full construct is shown. The LC/A protein is underlined, while the Strep-tags are shown in bold and italicized text. Residues marked in bold are active site residues.

### Vectors

The vectors used are as set forth in the chart below.

| | Antibiotic resistance | Selective marker |
|---|---|---|
| Name | (for E. coli) | (for S. cerevisiae) |
| pET28a | Kanamycin | — |
| pRS315_KanRMod_AcFRS | Kanamycin | LEU2 |
| (Islam, M. et al., 2021, ACS | | |
| Chemical Biology, Volume | | |
| 16, Number 2, pp 344-359) | | |
| pCTCON2 | Ampicillin | TRP1 |
| (Chao, G. et al., 2006, Nature | | |
| Protocols, Volume 1, Number | | |
| 2, pp 755-768) | | |
| pRS314_cMSH6 | Ampicillin | TRP1 |
| (Shusta, E. V., et al., 1998, | | |
| Nature Biotechnology, | | |
| Volume 16, Number 8, pp | | |
| 773-777. And Sikorski, R. S. | | |
| et al., 1989, In Genetics, | | |
| Volume 122, Number 1, pp | | |
| 19-27) | | |

### Cloning

Botulinum Neurotoxin Light Chain A1. The synthetic gene was cloned between the XbaI and XhoI sites of the pET28 vector under the T7 promoter. The plasmid was transformed into E. coli BL21(DE3).

Wild type yeast display plasmids. The parent pCTCON2 vector (Chao, G. et al., 2006, Nature Protocols, Vol. 1, No. 2, pp 755-768) was digested with NheI and BamHI (New England Biolabs, Ipswich, MA) using the manufacturers' recommendations and purified by agarose gel electrophoresis using a miniprep kit Epoch Life Sciences, Missouri City, TX). The genes encoding for the camelid WT constructs were amplified from pET32b plasmids containing the indicated gene sequences (JPU-A5, JPU-C1, ciA-H7) or from the corresponding synthetic gene (JPU-C10) by PCR using the corresponding primers indicated in the table above (forward and reverse primers A0, B0, C0, and D0, respectively). The amplicon was purified by agarose gel electrophoresis. The amplicons of the JPU-A5, JPU-C1, and ciA-H7 genes were digested with NheI and BamHI and purified using GenCatch GEX buffer and a miniprep kit (Epoch Life Sciences). Each digested insert was then incubated overnight with digested plasmid and T4 DNA Ligase (New England Biolabs). The mixtures were then transformed into E. coli DH5α Z1 (New England Biolabs) and plated onto LB agar plates supplemented with 50 μg mL⁻¹ ampicillin and grown at 37° C. overnight. Single colonies were used to inoculate 5 mL cultures of LB+50 μg mL⁻¹ ampicillin and grown overnight at 37° C. with 300 rpm agitation. The plasmids were extracted from the saturated cultures using an Epoch DNA miniprep kit (Epoch Life Sciences, Missouri City, TX) according to the manufacturer's instructions. Plasmid sequences were verified using primers S1F and S1R by Sanger sequencing.

The plasmid corresponding to JPU-C10 was constructed by Gibson Assembly, using the digested pCTCON2 vector and the insert amplicon. The mixture was transformed into chemically competent E. coli DH5α Z1 (New England Biolabs), grown, and the plasmid extracted in the same way as for the other three WT sdAbs. Plasmid sequences were verified using primers SIF and SIR by Sanger sequencing.

TAG yeast display plasmids. The TAG codons were introduced by amplifying two fragments from the corresponding pCTCON2 plasmid using the appropriate mutagenic primers from the table above with the appropriate S1 primer. For example, the JPU-A5 Q1TAG fragments were generated by using primers A1F and SIR in one PCR reaction, and A1R and S1F in another to obtain overlapping fragments with the desired mutation. DNA fragments were purified using agarose gel electrophoresis, and the plasmid constructed by Gibson Assembly of the two amplicons with linearized pCTCON2 as described. The mixture was transformed into E. coli DH5α Z1 cells, and the plasmids were extracted and purified as described above. The correct mutation was verified by Sanger sequencing using primers S1F and SIR.

Secretion plasmids. The parent pRS314 plasmid, was linearized with BglII and NheI (New England Biolabs) in a stepwise manner as suggested by the manufacturer. The gene cMSH6, encoding for a C-terminal cMyc/LPET Sortase A recognition site/His6 motif, was amplified by PCR using primers E0F and E0R. The plasmid was then reassembled via Gibson Assembly and recovered after transformation into E. co/i DH5α Z1 (using LB with ampicillin 50 μg mL⁻¹). While plasmid linearization might be carried out using BglII and BamHI (instead of NheI), the desired plasmid sequence was confirmed via Sanger sequencing using primers S2F and S2R. The verified plasmid was then linearized with NheI and BamHI. The gene encoding for the corresponding JPU-C1 mutant was amplified by PCR using the appropriate pCTCON2 plasmid as template and primers G0F and G0R. The gene and linearized plasmid were purified by agarose gel electrophoresis, and the plasmid was reconstructed by Gibson assembly. Plasmid amplification was carried out in E. coli DH5α Z1 as above, and the plasmid sequence verified using Sanger sequencing with primers S2F and S2R.

### LC/A Preparation

E. coli BL21 (DE3) cells transformed with the pET28 plasmid encoding LC/A were grown in 20 mL cultures shaking at 250 rpm at 37° C. overnight in a baffled flask until OD600 ˜7-12 in PG non-inducing medium (Van Deventer, J. A. et al., 2015, Protein Engineering, Design and Selection, Vol. 28, No. 10, pp 317-325.) supplemented with 400 μg/mL kanamycin. The culture was then diluted ˜10 fold into 500 mL ZYP-5052 autoinduction medium (Van Deventer, J. A. et al., 2015, Ibid.) and grown at 37° C. with agitation at 300 rpm until OD600 ˜0.8, at which point the culture was then incubated overnight at 18° C. with agitation at 275 rpm.

The cells were then harvested by centrifugation and the cell pellet was resuspended in lysis buffer (10 g pellet/100 mL buffer; 25 mM sodium phosphate, 500 mM NaCl, protease inhibitor (1 tablet/50 mL; Millipore Sigma, Burlington, MA), 5 mM aminocaproic acid, 2 mM benzamidine hydrochloride, pH 8.0). The suspension was homogenized (Avestin, Ottawa, Canada) and washed with 50 mL lysis buffer. Triton X-100 was then added to the lysed cells at 0.1% v/v. The lysate was clarified by centrifugation (30,000 g, 30 min, 4° C.), and the supernatant was loaded onto a NiNTA (Qiagen, Hilden, Germany) column preequilibrated with lysis buffer. The lysate was passed through the column 5 times. The resin was then washed with lysis buffer containing 0.1% v/v Triton X-100, followed by washes with 10 mM and 30 mM imidazole in lysis buffer. The protein was eluted with 300 mM imidazole in lysis buffer, and protease inhibitors (5 mM aminocaproic acid, 2 mM benzamidine hydrochloride, 10 μM leupeptin, 5 mM EDTA) were added to the collected fractions. The fractions containing the target protein were identified by SDS-PAGE and the appropriate eluates loaded onto a Strep-Tactin XT resin (IBA Life Sciences, Gottingen, Germany) pre-equilibrated with the same elution buffer, and passed 5 times through the resin. The resin was washed with 10-20 column volumes of 25 mM sodium phosphate, 100 mM NaCl, 10% v/v glycerol, pH 8.0, and the protein was eluted with 25 mM sodium phosphate, 100 mM NaCl, 10% v/v glycerol, 100 mM d-biotin, pH 8.0. The protease inhibitors and 2 mM EDTA were added to the elution fractions. The fractions containing the LC/A protein were identified by SDS-PAGE, combined, and concentrated in 10 kDa MWCO. The resulting solution was dialyzed against 40% v/v glycerol, 25 mM sodium phosphate, 100 mM NaCl, 2 mM EDTA, pH 8.0 using a Slide-A-Lyzer 3.5 kDa MWCO dialysis cassette (Thermo Fisher, Waltham, MA). The protein was stored in 10 mg/mL (181.6 μM) aliquots at −80° C. Before use, an aliquot of the enzyme was thawed on ice and diluted to 1 μM in reconstitution buffer. Subsequent dilutions were carried out in working buffer.

### Yeast Transformations and Stocks

S. cerevisiae RJY100 (Castro-Forero, A. et al., 2008, Journal of Applied Polymer Science, Vol. 107, No. 2, pp 881-890.) was transformed with the appropriate pCTCON2 or pRS315_cMSH6 plasmid and, for TAG mutants, pRS315_KanRMod_AcFRS encoding the orthogonal translation system. Transformations were performed using Frozen-EZ Yeast Transformation Kit II (Zymo Research, Irvine, CA) according to the manufacturer's instructions and plated onto SDCAA (for WT constructs not containing the orthogonal translation plasmid) or SDSCAA -Trp -Ura -Leu (for dual plasmid transformants). The plates were incubated for 2-3 days at 30° C. Single colonies were then used to inoculate 5 mL of SDCAA or SDSCAA -Trp -Ura -Leu media, accordingly, supplemented with penicillin/streptomycin (100 U mL⁻¹/100 μg mL⁻¹; Thermo Fisher). Cultures were grown for 3 days at 30° C. with constant agitation at 300 rpm until saturation, and these saturated cultures were stored at 4° C. for up to 4 weeks.

### Yeast Growth and Induction Involving Display Constructs

For each experiment, 200 μL of stock was pelleted, and the pellet resuspended in 5 mL of the appropriate fresh media. The cells were grown at 30° C./300 rpm overnight. The OD600 was measured using a NanoDrop OneC instrument (Thermo Fisher) and diluted to an OD of 1 in a final volume of 5 mL. The cultures were incubated for 5-6 h and the OD measured again. A volume of cells corresponding to 5 mL of culture at an OD of 1 was transferred to clean culture tubes and pelleted. The supernatant was removed, and the pellet resuspended in 5 mL of induction media supplemented with penicillin/streptomycin (100 U mL⁻¹/100 g mL⁻¹; to an OD of 1). WT constructs were induced in SGCAA media, while their TAG counterparts were induced in SGSCAA -Trp -Ura -Leu. NcAAs were freshly prepared before induction and added to the medium at a final concentration of 1 mM. Cultures were incubated at 20° C./300 rpm during induction for 16 h. OD values were measured after induction and the equivalence 1 OD=1×10⁷ cells used to aliquot the number of cells needed for each experiment. All cells were washed 3 times with PBSA (200 μL if in 96-well V-bottom plates, 1 mL if in 1.7 mL microcentrifuge tubes) before use.

### Labeling for Flow Cytometry

Cells were first labeled with 50 μL 1:500 dilution of chicken u-cMyc (Exalpha Biologicals, Shirley, MA) in PBSA for 30 min at room temperature, incubating on an orbital shaker set at 150 rpm. The suspension was then diluted with 150 μL of cold PBSA, and the cells washed 2×200 μL cold PBSA. Secondary labeling was carried out with 50 μL of 1:500 dilution of goat α-chicken-AF647 (Thermo Fisher) and 1:500 of StreptactinXT-DY488 (IBA Life Sciences, Gottingen, Germany) in cold PBSA. Cells were incubated for 15 min on ice protected from light, then diluted with 150 μL cold PBSA, and washed once with 200 μL cold PBSA. Cell pellets were kept on ice in preparation for flow cytometry.

Single-color controls were treated similarly in 1.7 mL microcentrifuge tubes, albeit using induced WT cells and performing secondary labeling with 1:500 goat a-chicken-AF488 (Thermo Fisher) or 1:500 goat a-chicken-AF647 (Thermo Fisher) as appropriate. The negative control to determine cell autofluorescence was not labeled and the cell pellet was kept on ice after washing, until analysis.

### Flow Cytometry Analysis

Flow cytometry analysis was carried out in an Attune NxT Flow Cytometer (Thermo Fisher). Cell pellets were resuspended in 200 μL (for plates) or 500 μL (for microcentrifuge tubes) cold PBSA prior to analysis. The instrument was set up to collect 10,000 total events from each sample, except for the titration experiments on the yeast surface which were set up to collect 1,000 events in the cMyc-positive quadrants.

Data analysis was performed using FlowJo and Microsoft Excel. In general, singlets were selected by using a polygonal gate on the SSC-A vs FSC-A dot plot, then gating on the FSC-W vs FSC-H plot of such population. Quadrants were drawn by gating on the negative population such that it contained at least 95% of the events in quadrant 4 (Q4), while keeping the axis as close to the population as possible.

Median fluorescence intensity (MFI) and robust coefficient of variation of BL-1H (to detect the chromophore corresponding to LC/A detection) and RL-1H (corresponding to cMyc detection via the Alexa Fluor 647 nm chromophore) were obtained for cMyc positive populations and for the Q4 to account for background fluorescence. Corrected fluorescence values were obtained by subtracting the MFI values of the Q4 population from the cMyc positive one. For experiments done in triplicate, the mean and standard deviation of the sample of the MFI values were calculated.

Determination of Relative Readthrough Efficiency (RRE) and Maximum Misincorporation Frequency (MMF) of Obey Incorporation with ACFRS

Biological triplicates of each construct transformed with pRS315_KanRMod_AcFRS and either pCTCON2 BXG or pCTCON2_BYG (Fang, K. Y. et al., 2018, In Protein Scaffolds: Design, Synthesis, and Applications, Udit, A. K. Ed.; Springer New York, pp 173-186) were grown in 5 mL SDSCAA -Trp -Ura -Leu medium as described above and induced in 2 mL SGSCAA -Trp -Ura -Leu medium in the presence or absence of 1 mM freshly prepared OBeY. Induction was carried out for 16 h at 20° C., after which time 2 million cells of each sample were removed and used for the experiment. Following washing, cells were analyzed via flow cytometry detecting for the N-terminal BFP protein (detection via the height of the VL-1 fluorescence channel on the Attune NxT) and the C-terminal GFP protein (detection via the height of the BL-1 fluorescence channel on the Attune NxT). N-terminal positive populations were gated on single cells and the MFI corresponding to each detection channel averaged over the three replicates. The relative readthrough efficiency (RRE) value either in the absence or presence of ncAA was calculated by dividing the average MFI of GFP over BFP detection for each construct (BXG or BYG), and then obtaining the ratio of these values (BXG/BYG). The maximum misincorporation frequency (MMF) value was calculated as the ratio of the RRE values in the absence of OBeY by the RRE value in presence of OBeY. The standard deviation was propagated using equations 1-4:

$$RRE = \frac{MFI GF {P}_{B X G}}{MFI BF {P}_{B X G}} / \frac{MFI GF {P}_{B Y G}}{MFI BF {P}_{B Y G}} \qquad ( 1 )$$

$$MMF = \frac{R R {E}_{- n c A A}}{R R {E}_{+ n c A A}} \qquad ( 2 )$$

$${s}_{R R E} = RRE \sqrt{{{\sum }_{i = \{ BXG , BYG \} ,}}_{j = \{ GFP , BFP \}} {( \frac{{s}_{i , j}}{{MFI}_{i , j}} )}^{2}} \qquad ( 3 )$$

$${s}_{M M F} = MMF \sqrt{{( \frac{{s}_{R R E - n c A A}}{R R {E}_{- n c A A}} )}^{2} + {( \frac{{s}_{R R {E}_{+ n c A A}}}{R R {E}_{+ n c A A}} )}^{2}} \qquad ( 4 )$$

where:
RRE Calculated Relative Readthrough Efficiency
MFI GFPᵢ MFI of GFP in N-terminal-positive populations in construct i, with i=BXG or BYG
MFI GFPᵢ MFI of BFP in N-terminal-positive populations in construct i, with i=BXG or BYG
MMF Calculated Maximum Misincorporation Frequency
RRE_{±ncAA} Calculated RRE in the absence (−) or presence (+) or ncAA
S_{RRE} Propagated standard deviation of the RRE
MFI_{i,j} Median fluorescence intensity of BFP or GFP in BXG or BYG samples
Si,j Standard deviation of the sample of the MFI of BFP or GFP in BXG or BYG samples
S_{MMF} Propagated standard deviation of the MMF
S_{RRE±ncAA} Standard deviation of the sample of the calculated RRE in the absence (−) or presence (+) of ncAA

### Binding Screening

Binding experiments were carried out with 2 million cells taken from cultures induced in the absence of ncAA, or in the presence of 1 mM AzF or 1 mM OBeY. Washed cells were resuspended in 50 μL of 200 nM reconstituted BoNT in working buffer. Cells were incubated in an orbital shaker at 150 rpm at room temperature for 1 h. The suspension was diluted with 150 μL PBSA and washed 3×200 μL PBSA. The cell pellets were then labeled for flow cytometry as described above.

### Titrations on the Yeast Surface

Titrations were carried out using cultures induced without ncAA (JPU-A5 WT and JPU-C1 WT) or in the presence of 1 mM AzF (JPU-A5 N5AzF and JPU-C1 L101AzF). A stock of 3 million washed cells was prepared in 1.7 mL microcentrifuge tubes. The cell pellet was diluted with 1 mL PBSA and serial dilutions performed to obtain a stock of 30,000 cells per mL in PBSA. 100 μL of this stock was transferred to select wells in a 96-well plate, equivalent to 3,000 cells per well. Additionally, a stock of 24 million non-induced washed cells (encoding for a non-binding protein, M0076) was similarly prepared. The cells were diluted to a concentration of 120,000 cells mL⁻¹, and 100 μL of this suspension aliquoted into the sample wells, for a total of 12,000 non-displaying cells. Cells were pelleted at 3124 rcf for 5 minutes and the supernatant was discarded. The cells were then resuspended in 50 μL of reconstituted LC/A in varying concentrations (20 μM-10 nM, including a set of samples without LC/A). The plate was incubated for 12 h at room temperature, protected from light, with agitation at 150 rpm. The samples were diluted with 150 μL PBSA and washed 3×200 μL PBSA. The washed cells were labeled as described above.

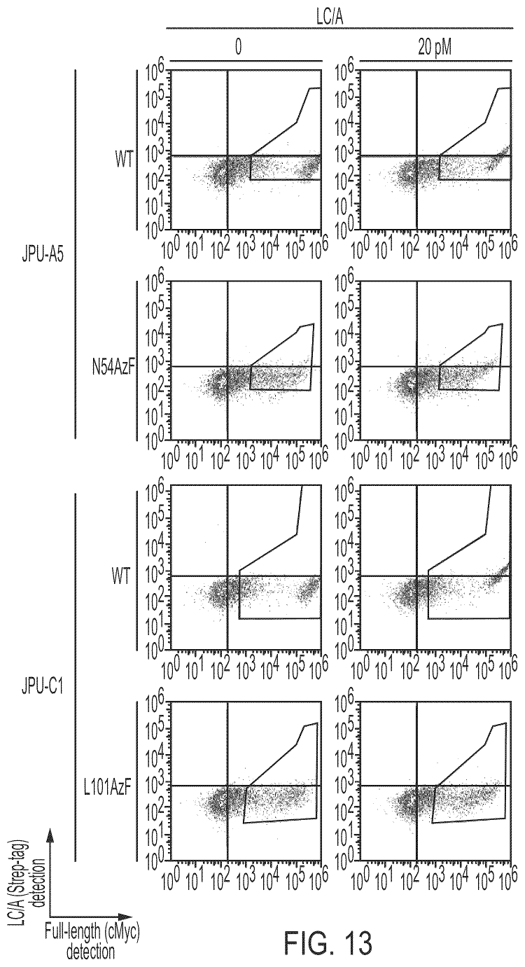

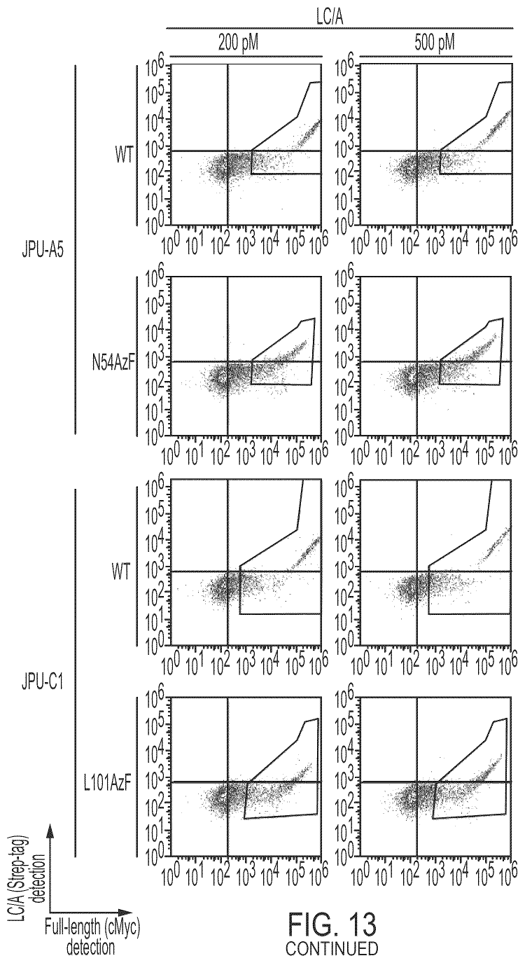

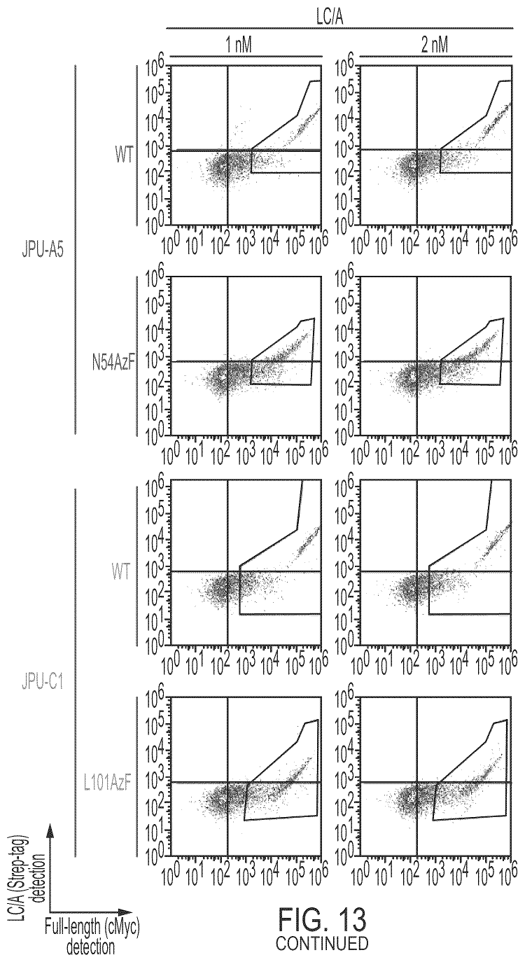

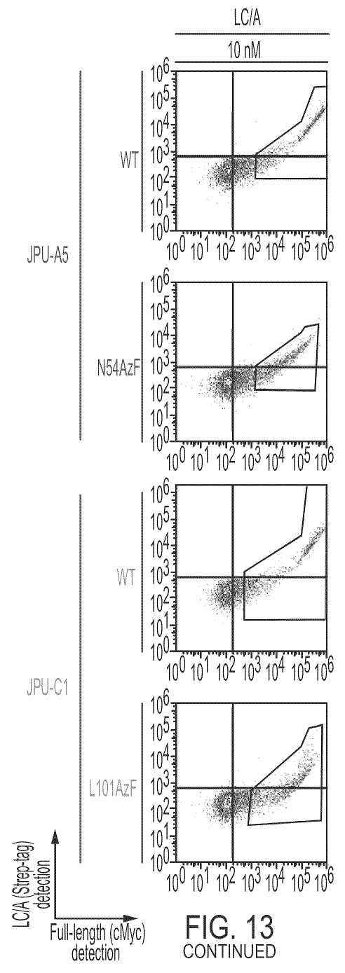

Each titration point was performed in technical triplicate. A polygonal gate was drawn to discard points that deviate from the expected binding behavior (FIG. 13). The MFI corresponding to each sample series was normalized between 0 and 1, and the mean and standard deviation of the sample for each different LC/A concentration was calculated. The data were then fitted to a single site binding model (equation 5), from which the K_{d} parameter was estimated.

$$MFI = \frac{{B}_{max} [ LC / A ]}{{K}_{d} + [ LC / A ]} \qquad ( 5 )$$

In equation 5, MFI represents the normalized median fluorescence intensity,
Bmax represents the maximum saturation value, [LC/A] is the concentration of LC/A, and K_{d} the estimated dissociation constant.
   Dissociation of Lc/a from Displayed Nanobodies

This study was carried out with the WT constructs of the four studied camelids (sdAbs or VHHs). 20 million induced cells were washed in 1.7 mL microcentrifuge tubes and resuspended in 1 mL PBSA. 250 μL of this suspension (i.e., 5 million cells) was transferred into clean tubes, pelleted, and the supernatant aspirated. The pellets were resuspended in 250 μL of 200 nM reconstituted LC/A in working buffer to a cell density of 20×10⁶ cells mL⁻¹ and incubated at 37° C. with shaking at 300 rpm for 24 h. After 24 h, 100 μL of each binding control was aliquoted to 96-well V-bottom plates, diluted to 200 μL with cold PBSA and washed 3×200 μL cold PBSA. These samples were labeled as described before and analyzed via flow cytometry. The treated samples were transferred to a clean 96-well V-bottom plate and diluted to 200 μL with PBSA. Cells were pelleted and resuspended in 200 μL 6 M urea/200 mM EDTA/25 mM Tris pH 8.5 and incubated at room temperature with agitation set at 150 rpm for 16 h. The cells were pelleted and washed 3×200 μL PBSA. Cells were labeled and analyzed via flow cytometry. The experiment was carried out in triplicate and the results are presented in FIGS. 14A and 14B.

### Screening for Photo-Crosslinking on the Yeast Surface

These experiments were carried out using cells induced in the absence of ncAA (WT) or in the presence of 1 mM AzF. 2 million washed cells were aliquoted into 96-well V-bottom plates and resuspended in 50 μL 200 nM reconstituted LC/A in working buffer. The initial photocrosslinking screening experiment was performed as described (Islam, M.; et al., 2021, Chemical Diversification of Simple Synthetic Antibodies. ACS Chemical Biology 2021, 16 (2), 344-359). Briefly, the incubated samples were transferred to a 96-well clear plate and irradiated with 365 nm light from a handheld 8 W UV lamp for 6 h. During this time, the plate was kept on ice at a distance ˜1 in from the light source, and the samples were resuspended every hour by pipetting up and down several times. The samples were then transferred back to a clean 96-well V-bottom plate and washed 3×200 μL PBSA. The samples were resuspended in 200 μL 8 M urea/200 mM EDTA/25 mM Tris pH 8.5 and incubated at 150 rpm for 16 h at room temperature. Cells were pelleted and washed 3×200 μL PBSA, and then labeled for flow cytometry analysis. The results can be seen in FIG. 28 and FIG. 29. For the results shown in FIG. 3, a 18 W 365 nm LED light source was used in a photoreactor (Hepatochem, Inc., Beverly, MA). In this experiment, after the 1 h incubation with LC/A, the samples were transferred to 300 μL round bottom glass vials containing a stir bar and loaded into the photoreactor at 4° C. Samples were irradiated at 365 nm for 2 min with magnetic stirring, and then transferred to a clean 96-well V-bottom plate. 150 μL PBSA was added, and the cells were pelleted and washed 3×200 μL PBSA. The cells were then resuspended in 200 μL 8 M urea/200 mM EDTA/25 mM Tris pH 8.5 and were incubated at 150 rpm for 16 h at room temperature. Cells were pelleted and washed 3× in 200 μL PBSA, and then were labeled for flow cytometry analysis.

### Time-Course Photo-Crosslinking on the Yeast Surface

JPU-A5 WT and N5AzF, and JPU-C1 WT and L101AzF were used for these experiments. The procedure was identical to the photoreactor irradiation procedure described in the screening section, varying the irradiation time for each sample (each time point corresponded to an independent sample). Time points were taken at 30 s and every minute for 5 minutes, for a total of 7 time points (including no irradiation). Each of these samples was treated with denaturant as described in the screening section above and washed, except that one set of each kept at 4° C. as binding controls. All cells were then labeled for flow cytometry as described above. These experiments were carried out in technical triplicate.

### Screening for Spontaneous Crosslinking on the Yeast Surface

In these experiments, cells for each mutant that were induced without ncAA (WT) or in the presence of 1 mM OBeY were used. 2 million cells were aliquoted into 96-well V-bottom plates, washed 3×200 μL PBSA, and resuspended in 100 μL 200 nM reconstituted LC/A in working buffer. The suspension was transferred to 1.7 mL microcentrifuge tubes and incubated at 37° C. with constant shaking at 300 rpm for 24 h. The suspensions were transferred to a clean 96-well V-bottom plate and 100 μL PBSA was added before pelleting. The cell pellet was washed 3×200 μL PBSA, resuspended in 200 μL 8M urea/200 mM EDTA/25 mM Tris pH 8.5 and incubated at 150 rpm for 16 h at room temperature. Cells were pelleted, washed 3×200 μL PBSA, and labeled for flow cytometry analysis.

### Time-Course of Spontaneous Crosslinking on the Yeast Surface

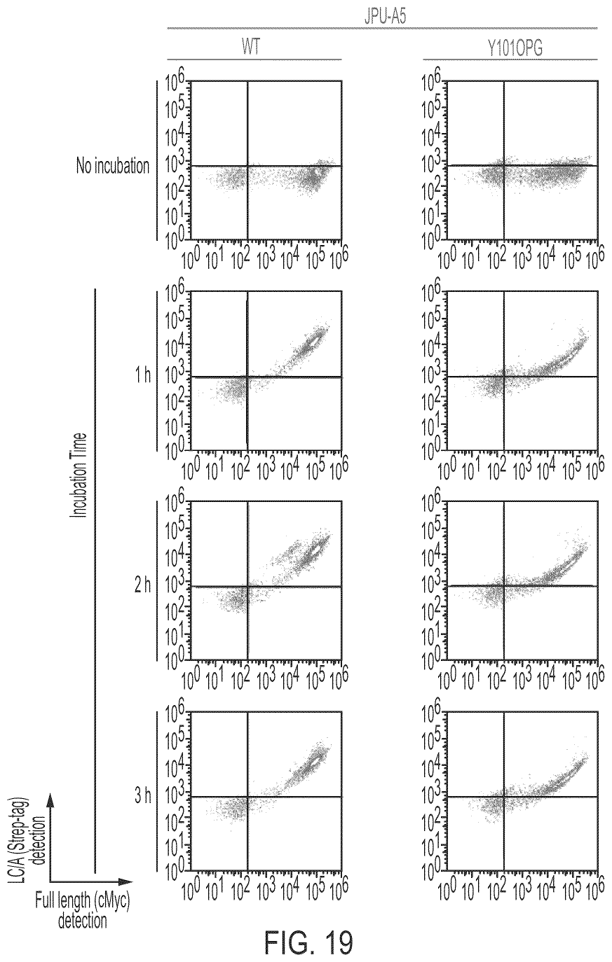

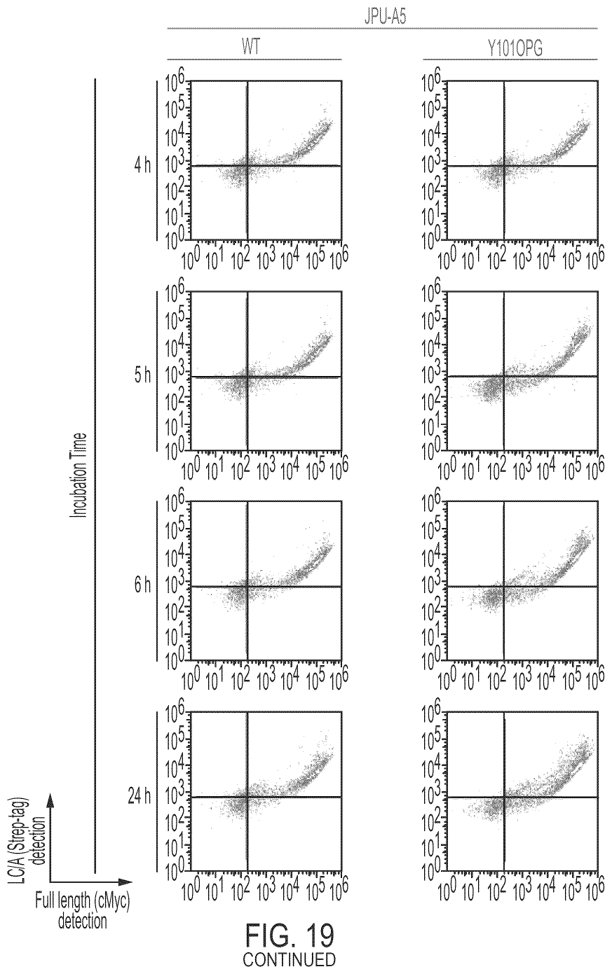

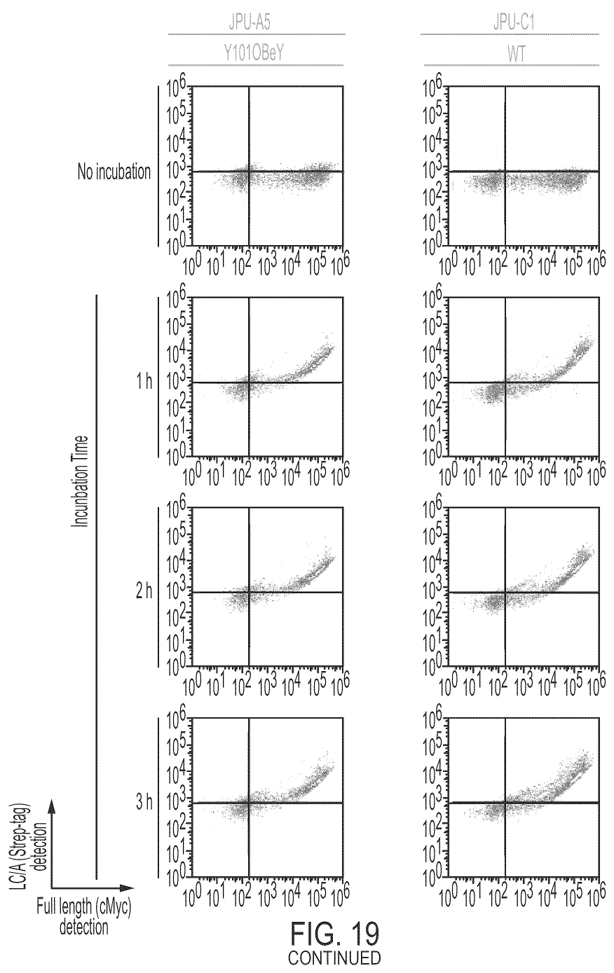

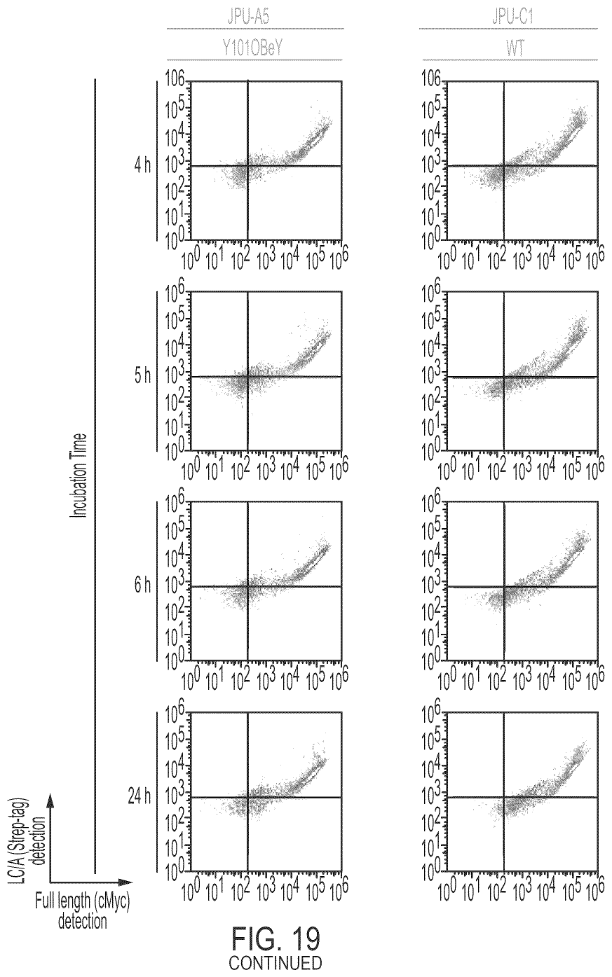

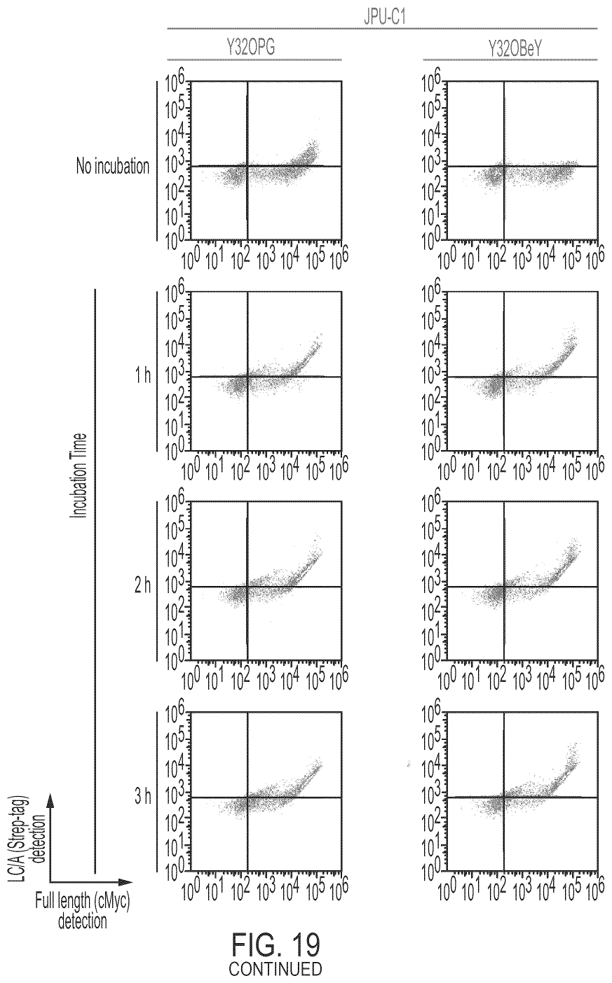

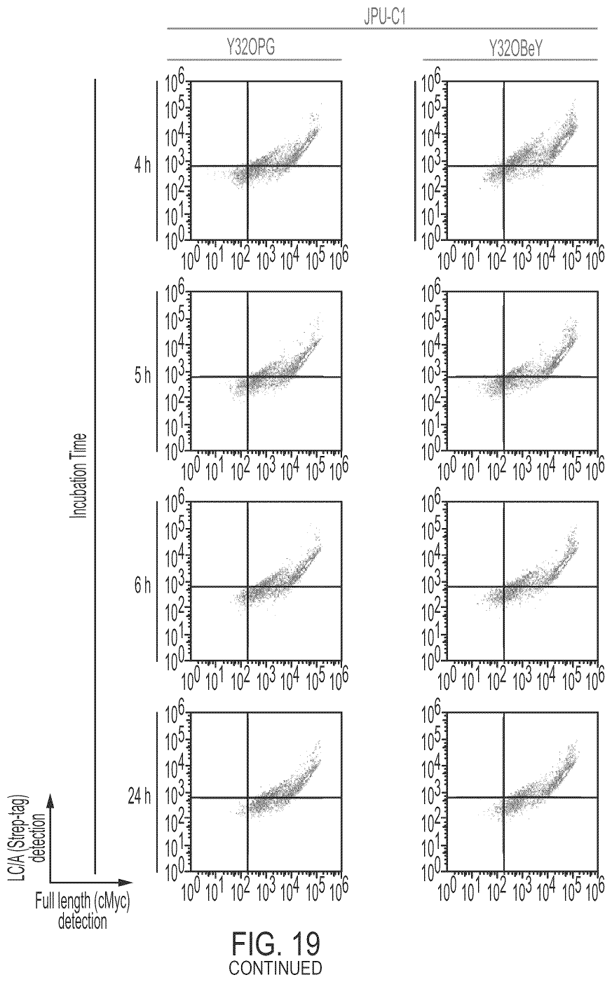

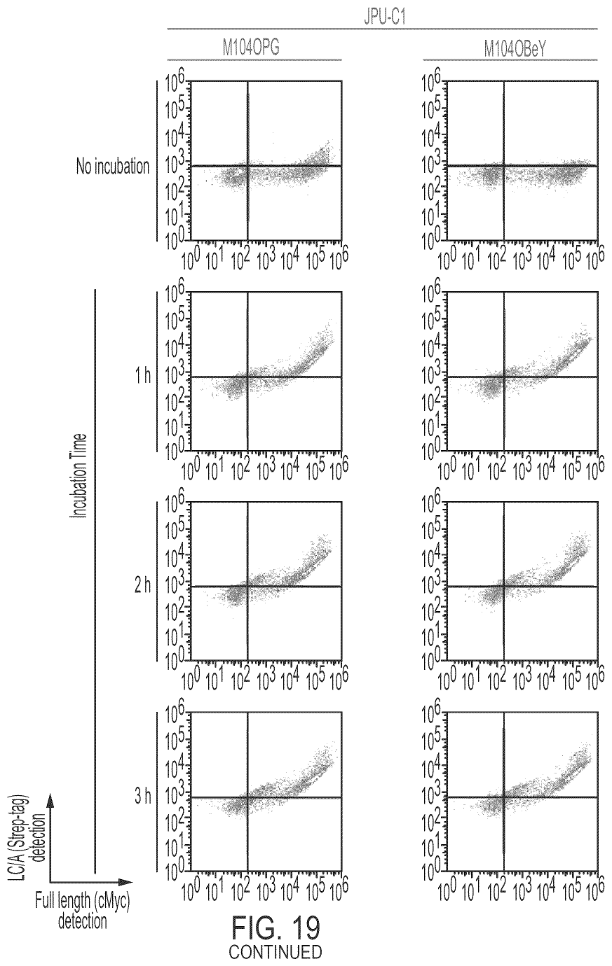

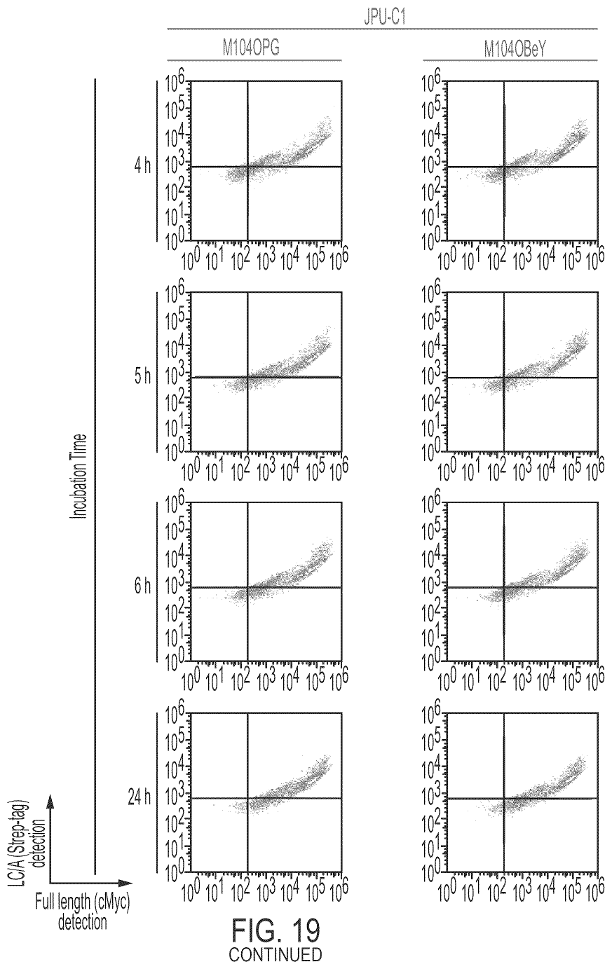

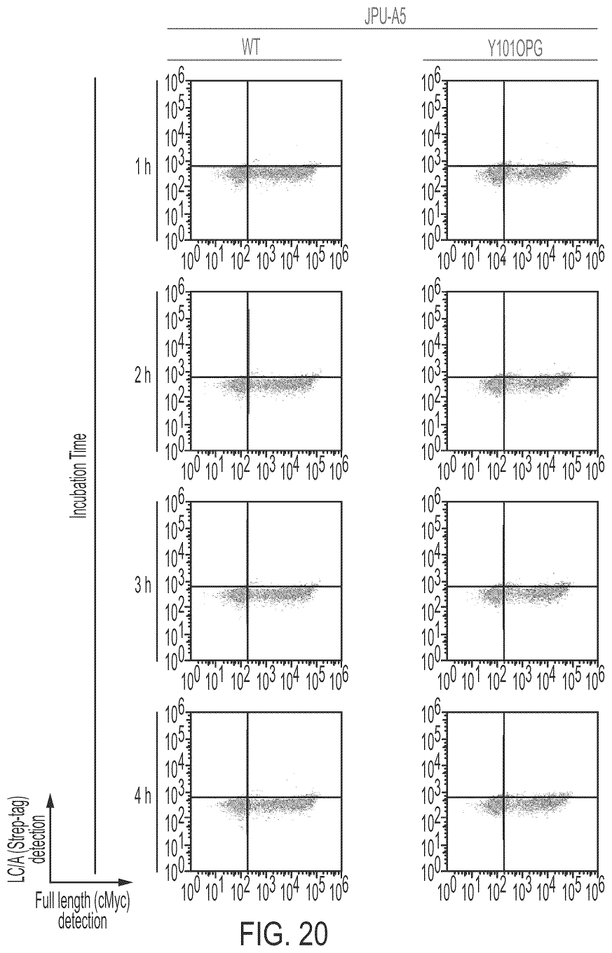

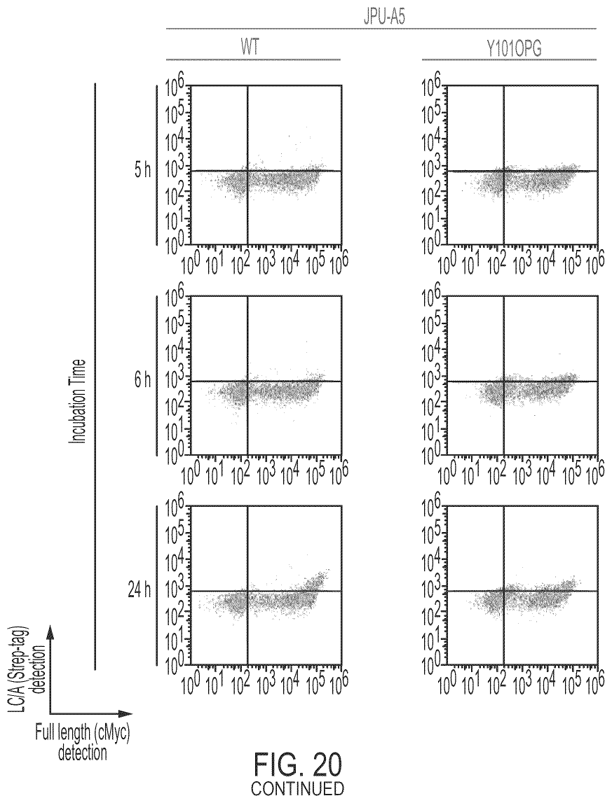

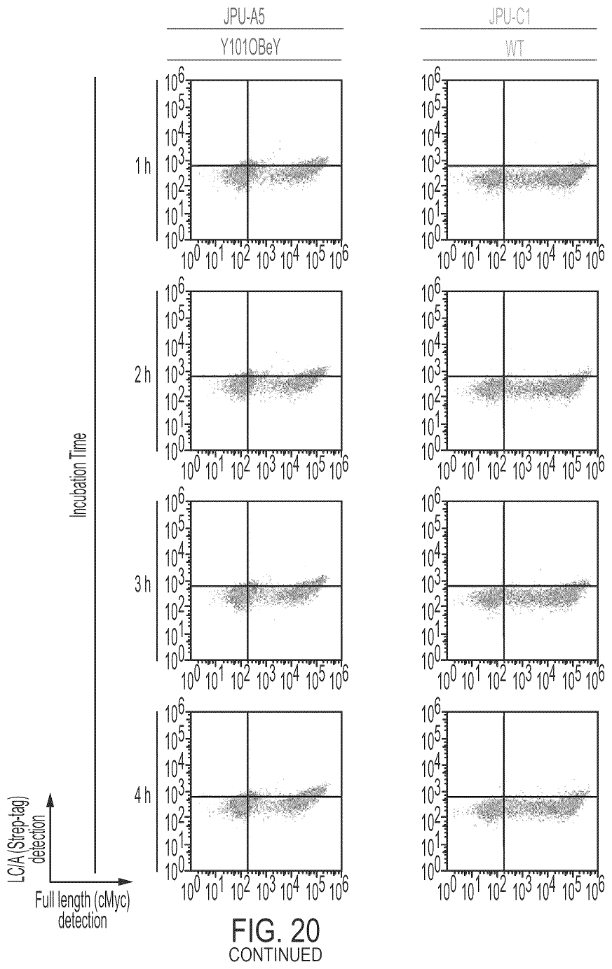

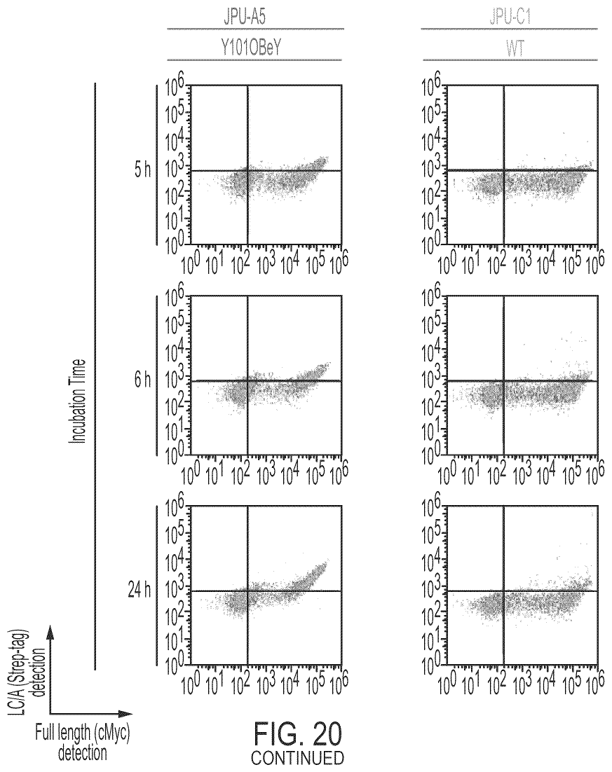

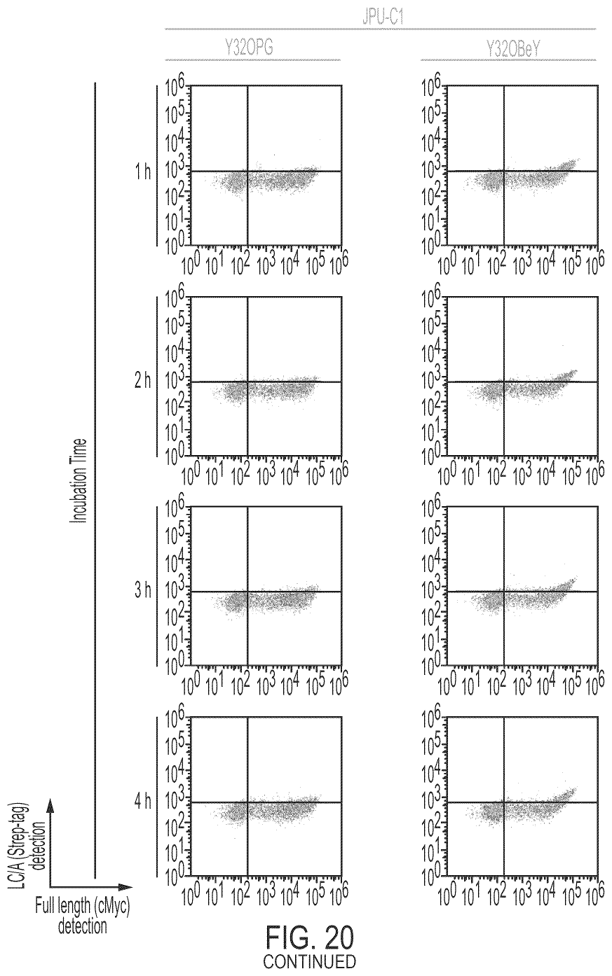

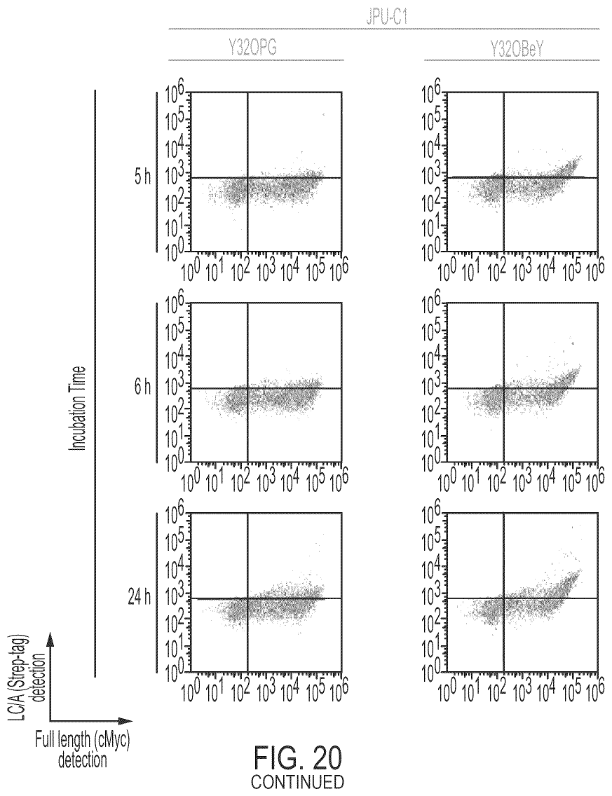

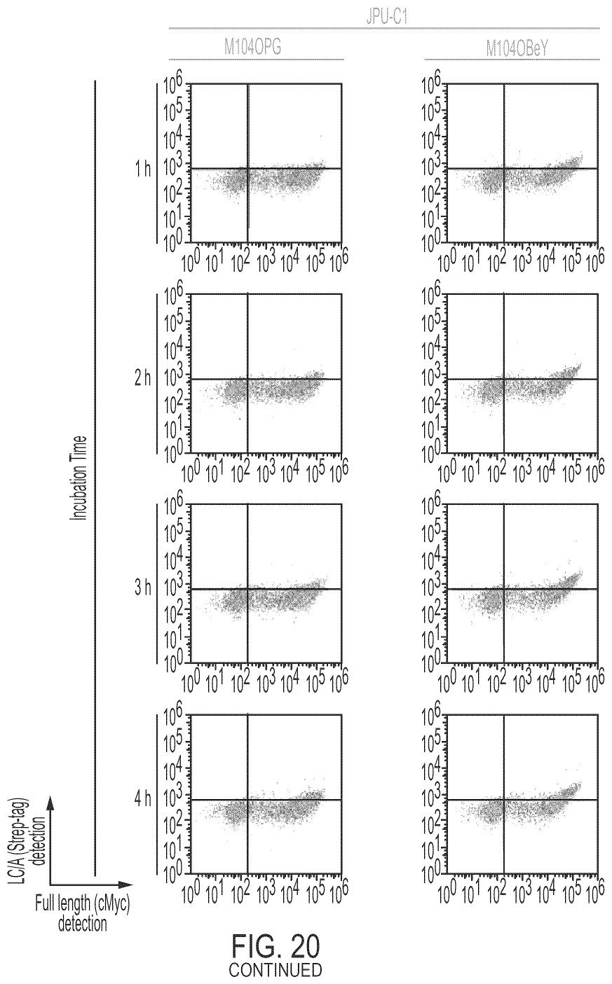

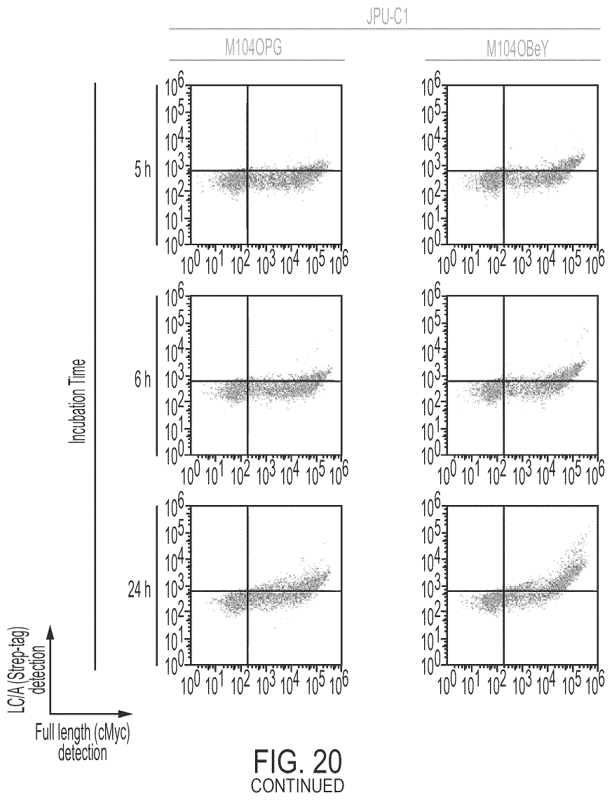

This experiment was carried out using JPU-A5 WT and Y101TAG, and JPU-C1 WT, Y32TAG, and M104TAG. WT constructs were induced in the absence of ncAA, and TAG mutants were induced in presence of 1 mM OBeY or 1 mM OPG (control), accordingly (FIG. 19). 2 million washed cells were used as a negative control (no incubation). The spontaneous crosslinking was carried out by resuspending cells in 200 nM LC/A to a concentration of 10 million cells mL⁻¹. The suspensions were incubated at 37° C. with agitation at 300 rpm. Aliquots of 2 million cells were taken every hour for 6 h and at 24 h of incubation and transferred to plates, where they were immediately washed 3×200 μL PBSA. Cell pellets were kept at 4° C. in between time points. After the 24 h time point, binding controls were labeled for flow cytometry. For denaturation, the samples were resuspended in 200 μL 8M urea/200 mM EDTA/25 mM Tris pH 8.5 and incubated at 150 rpm for 16 h at room temperature as described above. Cells were washed 3×200 μL PBSA and labeled for flow cytometry analysis. These experiments were carried out in technical triplicate.

### Protein Expression and Purification

Appropriate selection medium (5 mL SDCAA for WT, 5 mL SDSCAA -Trp -Ura -Leu for TAG mutants) was inoculated from saturated stocks of transformants as described above and grown overnight at 30° C./300 rpm to saturation. The culture was diluted 10-fold into the same medium (50 mL total) and grown again to saturation under the same conditions. The 50 mL culture was then diluted again 10-fold in the same type of medium (500 mL total) and grown overnight. The cells were then pelleted, and the supernatant was removed. Cells were resuspended in 1 L YPG supplemented with penicillin/streptomycin (100 U mL⁻¹/100 μg mL⁻¹), with the addition of freshly prepared 1 mM ncAA for TAG mutants. The medium was aliquoted in 100 mL portions and induction was carried out at 20° C./300 rpm for 4 days. The cultures were centrifuged, and the supernatant clarified by filtration through 0.2 μm membranes. The supernatant was buffered through the addition of 110 mL of 10× equilibration buffer (500 mM sodium phosphate, 3 M NaCl, pH 8.0). Purification was carried out using NiNTA resin (GenScript, Piscataway, NJ). The resin was equilibrated with 25 mL equilibration buffer (50 mM sodium phosphate, 300 mM NaCl, pH 8.0) and the supernatant passed over the resin 3 times at a rate of −5 mL min⁻¹. The resin was then washed with 50 mL equilibration buffer, and elution was carried out with an elution buffer containing a gradually increasing concentration of imidazole (5 mL fractions containing 25, 50, 75, 100, and 250 mM imidazole, respectively). Fractions were analyzed via SDS-PAGE with the protein of interest typically eluting between fractions 1 and 3. The fractions containing protein were concentrated and buffer exchanged against 50 mM HEPES/5 mM NaCl pH 7.1 using 15 mL Amicon MWCO 10 kDa spin columns (Millipore Sigma, Burlington, MA). Proteins were stored at 4° C. for immediate use or diluted 2-fold with glycerol, flash-frozen in liquid nitrogen, and kept at −80° C. for long-term storage. Frozen proteins were thawed on ice and buffer exchanged against 50 mM HEPES/5 mM NaCl pH 7.1 using 0.5 mL Amicon MWCO 10 kDa spin columns (Millipore Sigma).

### SDS-PAGE and Western Blotting

SDS-PAGE was done using BOLT™ 4-12% BisTris Mini Protein gels (Invitrogen, Thermo Fisher) following the manufacturer's recommendations using BOLT™ MES SDS Buffer (Invitrogen, Thermo Fisher) as running buffer. Samples were prepared using 4× BOLT™ LDS Sample Buffer (Invitrogen, Thermo Fisher) and 10×BOLT™ Sample Reducing Agent (Invitrogen, Thermo Fisher). Gels were stained with SIMPLY BLUE™ Safe Stain (Invitrogen, Thermo Fisher) according to the manufacturer's instructions using the maximum sensitivity protocol. Gels were imaged in an Azure 400 (Azure Biosystems, Dublin, CA) system using the visible light setting.

Western blots were done using an iBLOT®2 Dry Blotting System (Invitrogen, Thermo Fisher) with iBLOT®2 NC Regular Stacks using the preprogrammed P0 method. Membranes were blocked for 1 h at room temperature or overnight at 4° C. with 5% BSA w/v in TBS-T as blocking buffer and probed with 1:500 streptactin-DY488 (for LC/A detection) or 1:2500 Direct-BLOT™ HRP anti-c-Myc Antibody (Biolegend, San Diego, CA; for sdAb detection) in blocking buffer for 1 h at 4° C. Membranes were washed 3 times with blocking buffer and the membrane was probed for cMyc detection was treated with Pierce® ECL Western Blotting Substrate (Thermo Scientific) for 5 minutes according to the manufacturer's instructions before imaging. Membranes were visualized in an Azure 400 (Azure Biosystems, Dublin, CA) system using the blue channel (streptactin-DY488) or chemiluminescence (HRP anti-c-Myc) settings, respectively.

### Protein Quantification

Proteins were quantified via SDS-PAGE by loading a known amount of protein (typically 2 μL of a known dilution). Horse heart myoglobin (Millipore Sigma) was used as a protein standard and loaded on the same gel at different concentrations. The myoglobin stock concentration was determined by UV-Vis spectroscopy using an extinction coefficient of 188,000 M⁻¹ cm⁻¹ at 408 nm¹⁰. The gel was run and stained as described below. The gel was analyzed by densitometry using ImageJ. A calibration curve was constructed using the intensity of the myoglobin standards against the amount of protein loaded (in ng), from which the intensity of the camelid (aka sdAb or VHH) bands was used to calculate the concentration in solution.

### Sds-Page and Western Blotting

SDS-PAGE was performed using BOLT™ 4-12% BisTris Mini Protein gels (Invitrogen, Thermo Fisher) following the manufacturer's recommendations using BOLT™ MES SDS Buffer (Invitrogen, Thermo Fisher) as running buffer. Samples were prepared using 4×BOLT™ LDS Sample Buffer (Invitrogen, Thermo Fisher) and 10×BOLT™ Sample Reducing Agent (Invitrogen, Thermo Fisher). Gels were stained with SIMPLY BLUE™ Safe Stain (Invitrogen, Thermo Fisher) according to the manufacturer's instructions using the maximum sensitivity protocol. Gels were imaged in an Azure 400 (Azure Biosystems, Dublin, CA) system using the visible light setting.

Western blotting was performed using an iBLOT®2 Dry Blotting System (Invitrogen, Thermo Fisher) with iBLOT®2 NC Regular Stacks (Invitrogen, Thermo Fisher) using the preprogrammed P0 method. Membranes were blocked for 1 h at room temperature or overnight at 4° C. with 5% BSA w/v in TBS-T as blocking buffer and probed with 1:500 Strep-Tactin DY488 (for LC/A detection) or 1:2500 Direct-BLOT™ HRP anti-c-Myc Antibody (Biolegend, San Diego, CA; for sdAb detection) in blocking buffer for 1 h at 4° C. Membranes were washed 3 times with blocking buffer and the membrane probed for cMyc detection was treated with PIERCE® ECL Western Blotting Substrate (Thermo Scientific) for 5 minutes according to the manufacturer's instructions before imaging. Membranes were visualized in an Azure 400 system using the blue channel (Strep-Tactinactin DY488) or chemiluminescence (HRP anti-c-Myc) settings, respectively. (FIGS. 30A and 30B).

### Photo-Crosslinking in Solution

Photo-crosslinking experiments were set up containing 200 nM freshly reconstituted LC/A and 1 μM JPU-C1 WT or JPU-C1 L101AzF, accordingly, in working buffer. The samples were incubated protected from light at room temperature for 1 h, then transferred to 300 μL round-bottom glass vials and set up in the photoreactor at 4 C. Samples were irradiated with a 365 nm LED 18 W UV light for different amounts of time. For time course experiments, the irradiation was stopped at the indicated time point, and an aliquot was removed from the solution before continuing the irradiation. EDTA was then added to each sample to a final concentration of 5 mM to quench LC/A activity, and samples were stored at 4° C. for SDS-PAGE and/or western blot analysis. ImageJ analysis was carried out to estimate the extent of crosslinking by dividing the band intensity of the crosslinked adduct by that of the non-reconstituted LC/A control.

### Spontaneous Crosslinking in Solution

Samples were prepared containing 200 nM freshly reconstituted LC/A and 1 μM JPU-C1 WT or JPU-C1 Y32OBeY or M104OBeY, accordingly, in working buffer. The samples were incubated at 37° C. for 24 h. EDTA was then added to each sample to a final concentration of 5 mM, and the samples were stored at 4° C. for SDS-PAGE and/or western blot analysis. ImageJ analysis was used to estimate the extent of crosslinking by dividing the band intensity of the crosslinked adduct by that of the non-reconstituted LC/A control.

### Mammalian Cell Lysate Preparation

HepG2 cells (ATCC, Manassas, VA) were cultured in low glucose DMEM (Gibco, Thermo Fisher) containing 10% v/v FBS (Seradigm, VWR) and penicillin/streptomycin (100 U mL⁻¹/100 μg mL⁻¹). Cells were seeded in T25 flasks and cultured overnight at 37° C./5% CO₂ to promote adherence. Following aspiration, the cells were then collected by treating the flask with 3 mL trypsin-EDTA (0.25%, Gibco, Thermo Fisher), and the cells were washed 3 times with 10 mL cold PBS pH 7.4. Cells were resuspended in lysis buffer (25 mM Tris pH 7.4, 150 mM NaCl, 5% v/v glycerol, 1% v/v NP-40, supplemented with Protease and Phosphatase inhibitor Mini Tablets, EDTA-Free (Pierce, Thermo Fisher)), sonicated for 15 s, and incubated at 4° C. for 30 min on a rotary wheel. The lysate was clarified by centrifugation at 20,000 rcf for 20 min at 4° C. The lysate was stored at 4° C. until use. Total protein concentration was determined via the BCA method (Pierce BCA Protein Assay Kit, Thermo Fisher) following the manufacturer's recommendations.

### Specificity Assays in Solution

Samples were prepared in working buffer to contain 250 μg mL⁻¹ HepG2 cell lysate, 200 nM freshly reconstituted LC/A (where indicated), and varying concentrations of the corresponding sdAb.
Photo-crosslinking: Samples were incubated in the dark for 1 h at room temperature and an aliquot of each sample was transferred to 300 μL round-bottom glass vials equipped with a stir bar. The vials were then irradiated for 30 s at 4° C. EDTA was added to each sample to a final concentration of 5 mM to quench protease activity. The samples were then analyzed by SDS-PAGE and western blot.
Spontaneous crosslinking: Samples were incubated at 37° C. for 24 h. EDTA was added to each sample to a final 5 mM EDTA concentration to inhibit protease activity. The samples were then analyzed by SDS-PAGE and western blot.

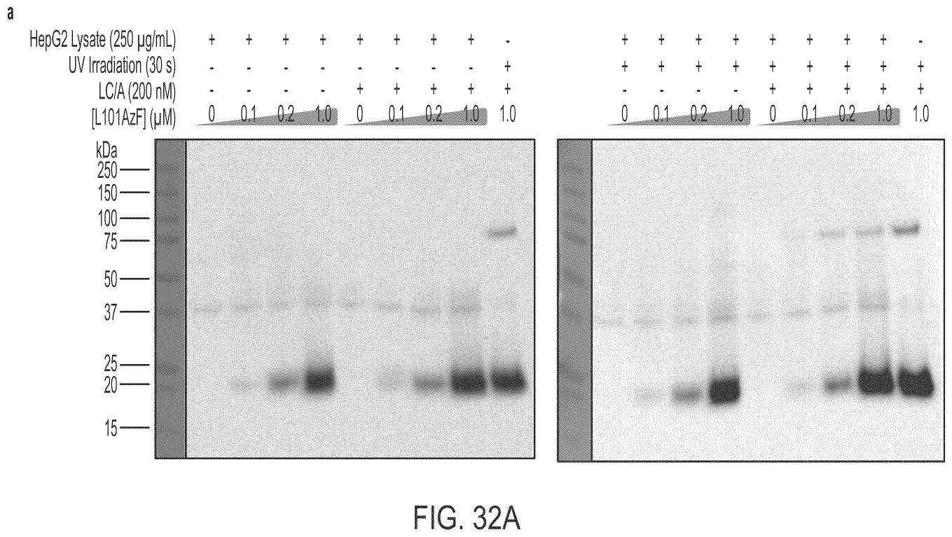

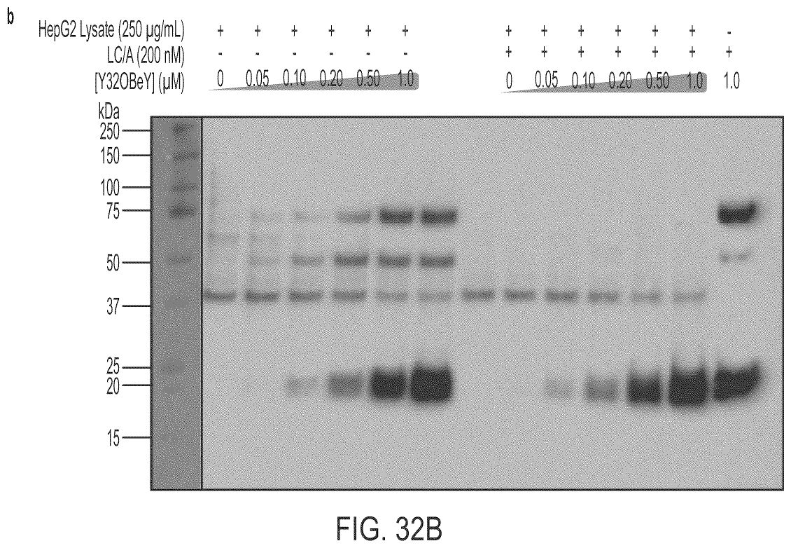

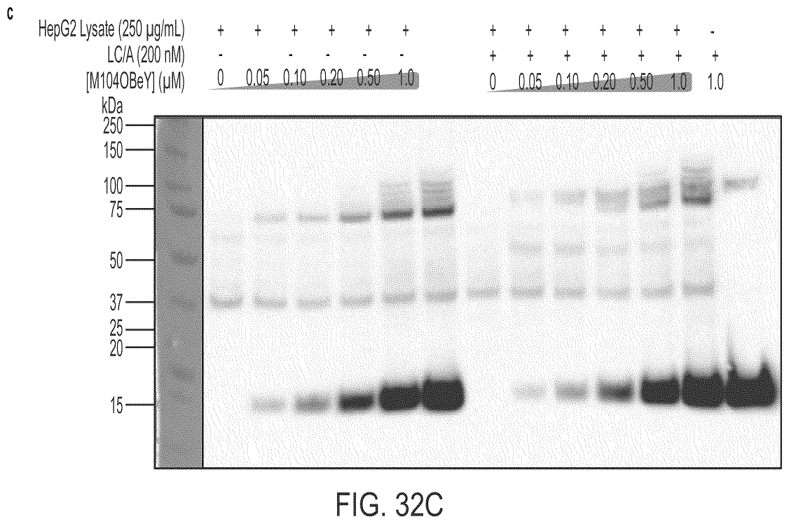

Densitometric analysis of the western blot images from FIG. 7 (FIG. 32 contains the full blot images) was performed using ImageJ. The band intensity profiles (FIG. 34) were normalized to the molecular weight of the sdAb where necessary to account for aberrant electrophoretic fronts.

### Fluorescence Substrate Inhibition Studies

Each sample was prepared in technical triplicate at 150 μL total volume in a 96-well plate. Samples contained 1 nM LC/A, freshly reconstituted at 500 nM and diluted to a 10 nM stock in working buffer. SdAbs were prepared at varying concentrations from 2 μM-10 nM in working buffer. Blank (no LC/A and no sdAb) and full-cleavage (10 nM LC/A only) controls were also prepared. Samples were incubated for 1 h at room temperature protected from light with agitation in an orbital shaker set to 150 rpm. Right before the incubation was time elapsed, the commercially available fluorescent reporter for LC/A (BioSentinel Pharma, Madison, WI) was diluted to a 2 μM stock concentration in working buffer. After the incubation time was over, 95 μL of each sample was transferred to a 96-well black-wall plate containing 5 μL of reporter stock in each well. The fluorescence of the reporter was then monitored in a SpectraMax i3× plate reader (Molecular Devices, San Jose, CA) with excitation at 434 nm and emission at 470 nm and 526 nm for 2.5 h at room temperature. The fluorescence ratio at each time point was then calculated by dividing the emission intensity at 526 nm by that at 470 nm. These ratios were averaged for each time point and sample. The initial velocity for each sample was then calculated as the negative slope of the linear fit of the first 10 min of measurements (FIG. 26). Fractional activity was then calculated for each sdAb concentration by dividing the initial velocity of the sample by that of the LC/A 1 nM control incubated in the absence of sdAb. The fractional activities were then fitted to a dose-response model (equation 6) using GraphPad Prism from where the IC₅₀ values were estimated. The fitted data sets were compared to that of WT using a sum-of-squares F-test to determine statistical differences.

$$F A = B + \frac{( T - B )}{1 + 1 {0}^{( log {IC}_{50} - log [ sdAb ] ) h}} \qquad ( 6 )$$

In the above equation 6, FA represents the fractional activity, T and B are the ordinates of the top and bottom plateaus, [sdAb] is the sdAb concentration, h is the Hill parameter, and IC₅₀ is the estimated parameter.

### pH and Time Dependence Studies of Spontaneous Crosslinking in Solution

JPU-C1 M104OBeY was analyzed in this experiment, with LC/A in the absence of sdAb as a control. Samples were prepared at pH 7.1, 8.0, or 9.0. For pH 9.0, the HEPES component of the reconstituted and working buffers was replaced by Tris-HCl at the same concentration (50 mM). The working concentration of M104OBeY was 1 μM in each sample, and LC/A, reconstituted at each different pH value at 1 μM, was added to a final 200 nM concentration. Samples were incubated at 37° C. for 24 h, and aliquots were removed at 2, 4, 8, and 12 h for analysis. At each time point, EDTA was added to a final concentration of 5 mM, and the sample was then immediately flash-frozen with liquid nitrogen and stored at −80° C. until analysis. Samples were analyzed by SDS-PAGE as described before.

### Click Chemistry in Solution

Copper-catalyzed click chemistry was used to confirm the presence of the azido group in purified JPU-C1 L101AzF. Reactions were carried out with 1 μM WT or L101AzF in water. Reactions were carried out in a 50 μL total volume. Biotin-(PEG)4-Alkyne (Click Chemistry Tools, Scottsdale, AZ) in DMSO was added to a final concentration of 100 μM, with a set of samples containing DMSO only as control. A mixture of CuSO4 (Millipore Sigma)/THPTA (Click Chemistry Tools) (for final 1 mM CuSO4 and 50 mM THPTA concentrations) were added to the samples, followed by the addition of aminoguanidine hydrochloride (final concentration 5 mM) and ascorbic acid (final concentration 5 mM). The samples were protected from light and incubated at room temperature for 1 h. The samples were analyzed by western blotting using streptavidin-AF488 (Thermo Fisher) 1:2500 as detection antibody, and the membranes were visualized using the blue channel in an Azure 400 system. Biotinylated horseradish peroxidase (Pierce, Thermo Fisher) was used as control. The results are shown in FIG. 31A.

### Mass Spectrometry of Tryptic Digests

Given the low protein expression yields of ncAA-containing sdAbs, MALDI-TOF-MS was used to corroborate the correct incorporation of the ncAA, as it has proven to be an effective tool to characterize ncAA incorporation and benefits from low analyte requirements (Duffy et al., 2010, Organic Letters, Vol. 12, No. 17, pp 3776-3779; Lee et al., 2019, Nature Communications, Vol. 10, No. 1, pp 5097; Yang, A. et al., 2016, Science, Vol. 354, No. 6312, pp 623-626; Stieglitz, J. T., et al., 2022, ACS Synthetic Biology, Vol. 11, No. 7, pp 2264-2299; and Lam, K.-h. et al., 2022, PLOS Pathogens, Vol. 18, No. 1, pp e1010169). Approximately 5 μg of purified proteins were brought to 100 μL volume with water and denatured by heating at 100° C. for 5 min. After allowing the samples to cool down to room temperature, 1 μg Trypsin Gold Mass Spectrometry Grade (Promega, Madison, WI) was added and the samples were incubated overnight at 37° C. The digested peptides were desalted with ZipTip C18 tips (Millipore Sigma) by following the manufacturer's protocol, eluting the peptides in 5 μL water/acetonitrile 1:1 containing 0.1% v/v trifluoroacetic acid. The samples were flash-frozen in liquid nitrogen and stored at −80° C. until analysis. MALDI-TOF-MS analysis was performed by the Biopolymers and Proteomics Core at the Koch Institute (Massachusetts Institute of Technology, Cambridge, MA). The samples were analyzed using u-cyano-4-hydroxycinnamic acid as matrix and measurements performed in positive mode. Results are shown in FIGS. 31B-31D.

c-MYC Detection

FIGS. 32A-32C provide full images showing the detection of c-Myc in selectivity experiments. Samples in FIG. 32A were analyzed after a 1 h incubation at room temperature and with or without 365 nm light irradiation for 30 seconds. Samples in FIG. 32B and FIG. 32C were incubated for 24 h at 37° C. Blot membranes were probed with mouse a-c-Myc-HRP conjugate 1:2500. The opacity of a rectangular portion of the left side of each blot was reduced to show the superimposed molecular weight ladder.

### Strep-Tag Detection

Samples in FIG. 33A were analyzed after 1 h incubation at room temperature and with or without 365 nm light irradiation for 30 seconds. Samples in FIG. 33B and FIG. 33C) were incubated for 24 h at 37° C. The right image I FIG. 33B shows an exposed version of the left image to better show the adduct band. Blot membranes were probed with Strep-Tactin DY488 1:500.

### Band Intensity Profile Normalization

The band intensity profiles shown in FIGS. 34A, 34B and 34C) were normalized to the molecular weight of the sdAb where necessary to account for aberrant electrophoretic fronts. FIG. 34A corresponds to photocrosslinking with L101AzF (1 h incubation at room temperature, irradiation for 30 s), while FIG. 34B and FIG. 34C correspond to incubation for 24 h at 37° C. with Y32OBeY or M104OBeY, respectively. The “No lysate control” sample was carried out in presence of 200 nM LC/A and 1 μM sdAb and irradiated 30 s (FIG. 34A) or incubated 24 h at 37° C. Both intensity plots in FIG. 34A include UV-irradiated “no lysate control” samples for reference. As can be observed in FIG. 34C, the position of the crosslinked “No lysate control” adduct in the intensity plot shown in FIG. 34C appears at a higher molecular weight than in the other band intensity profiles. This apparent shift can be attributed to a nonuniform (“smiling”) electrophoretic profile. This is also apparent in the position of the adduct in the +LC/A sample series. A line has been added to indicate the presumptive position of the adduct in this series of samples.

TABLE 1
VHH amino acid sequences

ALc-B8:
SGGGLVQPGGSLRLSCAASGSIFSIYAMGWYRQAPGKQRELVAAISSYGSTNYADSV
KGRFTISRDNAKNTVYLQMNSLKPEDTAVYYCNADIATMTAVGGFDYWGQGTQVT
VSS (SEQ ID NO: 68)
ALc-H7:
SGGGSVQPGGSLRLSCAAIGSVFTMYTTAWYRQTPGNLRELVASITDEHRTNYAASA
EGRFTISRDNAKHTVDLQMTNLKPEDTAVYYCKLEHDLGYYDYWGQGTQVTVSS
(SEQ ID NO: 69)
ciA-D1:
SGGGLVQPGGSLRLSCATSGFTLEYYAIGWFRQAPGKGREGVACMNSSGGGTNYAD
SVKGRFTISRDNAKKMVYLQMNSLKSEDTAVYYCVVDDFRCGSRWAAYLRSSWGQ
GTQVTVSS (SEQ ID NO: 70)
ciA-D12:
SGGGLVQPGGSLRLSCVVSGSDFNTYIMGWYRQVPGKPRELVADITTEGKTNYGGS
VKGRFTISRDNAKNTVYLQMFGLKPEDAGNYVCNADWKMGAWTAGDYGIDYWG
KGTLVTVSS (SEQ ID NO: 71)
ciA-F12:
SGGGLVQPGGSLRLSCAASGFTLGSRYMSWVRQAPGEGFEWVSSIEPSGTAWDGDS
AKGRFTTSRDDAKNTLYLQMSNLQPEDTGVYYCATGYRTDTRIPGGSWGQGTQVT
VSS (SEQ ID NO: 72)
ciA-H7:
SGGGLVQVGGSLRLSCVVSGSDISGIAMGWYRQAPGKRREMVADIFSGGSTDYAGS
VKGRFTISRDNAKKTSYLQMNNVKPEDTGVYYCRLYGSGDYWGQGTQVTVSS
(SEQ ID NO: 73)
JPU-A1:
SGGGLVQPGGSLRLSCAASGFTLDDYAIGWFRQVPGKEDEGVSCMSRSGDTYYPHS
VKGRFTISVDNAKNTMYLQMNNLKPEDTAVYYCAIDFPPVRPMCIQAAPKKRSRGT
QVTVSA (SEQ ID NO: 74)
JPU-A5:
TGGGLVQAGGSLRLSCTASGADFSFYAMGWYRQTPGNSRELVAVMNLNGVISYGD
SARGRFDISRDGTKNIVFLQMNSLKPEDTGVYYCNGMRLYTRGSVRHPESWGQGIQ
VTVSS (SEQ ID NO: 75)
JPU-A11:
TGGGLVQAGDSLTLSCAATGRTLDYYALGWFRQVPGNKREFVAAINWLGGSTYYA
DSVRGRFTLSRDNSKSTLYLNMNNLIPDDTAVYYCAADFSIAYSGTYPPAYAEYDYD
YWGQGTQVTVSS (SEQ ID NO: 76)
JPU-B5:
SGGLVQPGGSLKLSCAHSGSPLSIWVMGWYRQAPGKQRELVALINLNGITSYGDSV
KGRFTISRDYAENTAYLQMNSLKFEDTAVYYCNAEPLGPRGKKSGKEYWGTGTQV
TVSL (SEQ ID NO: 77)
JPU-B9:
TGGALVQPGQSLTLSCTTSENVFGIYGMAWLRQAPGRQRELVASITSRGTAHYHDSV
KGRFTISRESGKTTAYLQTTSVNPEDTAIYYCNSGPYWGQGTQVTVSS (SEQ ID NO:
78)
JPU-C1:
SGGGLVQPGGSLRLSCAASGFTFNRYVIRWYRQAPGKERELVAGISRSGDSGRYVDS
VKGRFTISRDNDKNMAYLQMSSLKPDDTAVYYCSALNLEDMEYWGQGTQVTVSS
(SEQ ID NO: 79)
JPU-C10:
SGGGLVQPGGSLRLSCAASGNIFSIYYMGWYRQAPGKQREMVAIINSNGITNYGDFV
KGRFTISRDNAENSAYLQMNNLTPEDTAVYYCNAGKLRRTTGWGLDDYWGQGTQ
VTVSS (SEQ ID NO: 80)
JPU-D12:
SGGGTVQPGGTLRLSCAASGFTLDEYAIGWFRQAPGKEREGVSCISSSASISYADSVK
GRFTISRDNAKNTVYLTMNSLKPEDTGVYYCARAFLACGPVAGWGTEYDYWGQGT
QVTVSS (SEQ ID NO: 81)
JPU-G3:
TGGGLVQPGGSLRLSCTASTTISDFYSMGWFRQTPGNQRELVAIVRRGGDTKSGDSV
KGRFTISRDNTRSTVYLQMDNLKPEDTAVYYCYANLQKSSDELGPYYWGQGTQVT
VSS (SEQ ID NO: 82)
JPU-G7:
SGGGLVQSGGSLRLSCAASLLTLEYYAIGWFRQAPGKEREGVSCTGSSGGSTVYIDS
VKGRFTVVRDNAKNMVYLQMDNLQPEDTAVYYCAADDLRCGRGWSSYFRGSWG
QETQVTVSS (SEQ ID NO: 83)
JPU-G11:
TGGGLVQPGGSLRLACVASESVFEMYTVAWYRQAPGKQRELVAGITDEGRTNYAD
FVKGRFTISRDNSKKTVHLQMDNLNPEDTAVYYCKLEHDLGYYDYWGQGTQVTVS
S (SEQ ID NO: 84)
JPU-G12:
SGGGLVQPGGSLRLSCAASGLTLDYYAIGWFRQAPGKEREGVSCISSGSSMSIHADS
VKGRFTISRDNAKNTVYLQMNSLKPEDTAVYYCAADDFTCGSRWSDWAHTFGFWG
QGTQVTVSS (SEQ ID NO: 85)
JPU-H7:
SGGLVQPGGSLTLSCVVSGGIFSTYIMGWYRQVPGRQREMVATISNHTTDYADFVQ
GRFTISRDIAKKAVYLQMHSLKPDDTGRYVCNADWMVGAWTAGDYGVDYWGKGI
LVTVSS (SEQ ID NO: 86)

### Other Embodiments

From the foregoing description, it will be apparent that variations and modifications may be made to the aspects and embodiments as described herein to adopt them to various usages and conditions. Such embodiments are also within the scope of the claims that follow.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### GENERAL REFERENCES

(1) Wellner, A.; McMahon, C.; Gilman, M. S. A.; Clements, J. R.; Clark, S.; Nguyen, K. M.; Ho, M. H.; Hu, V. J.; Shin, J.-E.; Feldman, J.; et al. Rapid generation of potent antibodies by autonomous hypermutation in yeast. Nature Chemical Biology 2021, 17 (10), 1057-1064. DOI: 10.1038/s41589-021-00832-4.
(2) Pan, Y.; Du, J.; Liu, J.; Wu, H.; Gui, F.; Zhang, N.; Deng, X.; Song, G.; Li, Y.; Lu, J.; et al. Screening of potent neutralizing antibodies against SARS-CoV-2 using convalescent patients-derived phage-display libraries. Cell Discovery 2021, 7 (1), 57. DOI: 10.1038/s41421-021-00295-w.
(3) Thie, H.; Voedisch, B.; Dibel, S.; Hust, M.; Schirrmann, T. Affinity Maturation by Phage Display. In Therapeutic Antibodies: Methods and Protocols, Dimitrov, A. S. Ed.; Humana Press, 2009; pp 309-322.
(4) Yang, Z.; Wan, Y.; Tao, P.; Qiang, M.; Dong, X.; Lin, C.-W.; Yang, G.; Zheng, T.; Lerner, R. A. A cell-cell interaction format for selection of high-affinity antibodies to membrane proteins. Proceedings of the National Academy of Sciences 2019, 116 (30), 14971-14978. DOI: 10.1073/pnas.1908571116.
(5) Van Deventer, J. A.; Wittrup, K. D. Yeast Surface Display for Antibody Isolation: Library Construction, Library Screening, and Affinity Maturation. In Monoclonal Antibodies: Methods and Protocols, Ossipow, V., Fischer, N. Eds.; Humana Press, 2014; pp 151-181.
(6) Könning, D.; Kolmar, H. Beyond antibody engineering: directed evolution of alternative binding scaffolds and enzymes using yeast surface display. Microbial Cell Factories 2018, 17 (1), 32. DOI: 10.1186/s12934-018-0881-3.
(7) Boder, E. T.; Wittrup, K. D. Yeast surface display for screening combinatorial polypeptide libraries. Nature Biotechnology 1997, 15 (6), 553-557. DOI: 10.1038/nbt0697-553.
(8) Rappazzo, C. G.; Tse, L. V.; Kaku, C. I.; Wrapp, D.; Sakharkar, M.; Huang, D.; Deveau, L. M.; Yockachonis, T. J.; Herbert, A. S.; Battles, M. B.; et al. Broad and potent activity against SARS-like viruses by an engineered human monoclonal antibody. Science 2021, 371 (6531), 823-829. DOI: doi:10.1126/science.abf4830.
(9) McConnell, A. D.; Zhang, X.; Macomber, J. L.; Chau, B.; Sheffer, J. C.; Rahmanian, S.; Hare, E.; Spasojevic, V.; Horlick, R. A.; King, D. J.; et al. A general approach to antibody thermostabilization. MAbs 2014, 6 (5), 1274-1282. DOI: 10.4161/mabs.29680 PubMed.
(10) Warszawski, S.; Borenstein Katz, A.; Lipsh, R.; Khmelnitsky, L.; Ben Nissan, G.; Javitt, G.; Dym, O.; Unger, T.; Knop, O.; Albeck, S.; et al. Optimizing antibody affinity and stability by the automated design of the variable light-heavy chain interfaces. PLOS Computational Biology 2019, 15 (8), e1007207. DOI: 10.1371/journal.pcbi.1007207.
(11) Brockmann, E.-C. Selection of Stable scFv Antibodies by Phage Display. In Antibody Engineering: Methods and Protocols, Second Edition, Chames, P. Ed.; Humana Press, 2012; pp 123-144.
(12) Wang, T.; Badran, A. H.; Huang, T. P.; Liu, D. R. Continuous directed evolution of proteins with improved soluble expression. Nature Chemical Biology 2018, 14 (10), 972-980. DOI: 10.1038/s41589-018-0121-5.
(13) Bogen, J. P.; Hinz, S. C.; Grzeschik, J.; Ebenig, A.; Krah, S.; Zielonka, S.; Kolmar, H. Dual Function pH Responsive Bispecific Antibodies for Tumor Targeting and Antigen Depletion in Plasma. Frontiers in Immunology 2019, 10, Original Research. DOI: 10.3389/fimmu.2019.01892.
(14) Hinz, S. C.; Elter, A.; Rammo, O.; Schwammle, A.; Ali, A.; Zielonka, S.; Herget, T.; Kolmar, H. A Generic Procedure for the Isolation of pH- and Magnesium-Responsive Chicken scFvs for Downstream Purification of Human Antibodies. Front Bioeng Biotechnol 2020, 8, Original Research. DOI: 10.3389/fbioe.2020.00688.
(15) Könning, D.; Zielonka, S.; Sellmann, C.; Schröter, C.; Grzeschik, J.; Becker, S.; Kolmar, H. Isolation of a pH-Sensitive IgNAR Variable Domain from a Yeast-Displayed, Histidine-Doped Master Library. Marine Biotechnology 2016, 18 (2), 161-167. DOI: 10.1007/s10126-016-9690-z.
(16) Garuti, L.; Roberti, M.; Bottegoni, G. Irreversible protein kinase inhibitors. Curr Med Chem 2011, 18 (20), 2981-2994. DOI: 10.2174/092986711796391705 From NLM.
(17) González-Bello, C. Designing Irreversible Inhibitors-Worth the Effort?ChemMedChem 2016, 11 (1), 22-30. DOI: https://doi.org/10.1002/cmdc.201500469.
(18) Sutanto, F.; Konstantinidou, M.; Dömling, A. Covalent inhibitors: a rational approach to drug discovery. RSC Medicinal Chemistry 2020, 11 (8), 876-884, 10.1039/DOMD00154F. DOI: 10.1039/DOMD00154F.
(19) Walter, A. O.; Sjin, R. T. T.; Haringsma, H. J.; Ohashi, K.; Sun, J.; Lee, K.; Dubrovskiy, A.; Labenski, M.; Zhu, Z.; Wang, Z.; et al. Discovery of a Mutant-Selective Covalent Inhibitor of EGFR that Overcomes T790M-Mediated Resistance in NSCLC. Cancer Discovery 2013, 3 (12), 1404-1415. DOI: 10.1158/2159-8290.Cd-13-0314 (acccessed 4/18/2022).
(20) Engel, J.; Lategahn, J.; Rauh, D. Hope and Disappointment: Covalent Inhibitors to Overcome Drug Resistance in Non-Small Cell Lung Cancer. ACS Medicinal Chemistry Letters 2016, 7 (1), 2-5. DOI: 10.1021/acsmedchemlett.5b00475.
(21) Singh, J.; Petter, R. C.; Baillie, T. A.; Whitty, A. The resurgence of covalent drugs. Nature Reviews Drug Discovery 2011, 10 (4), 307-317. DOI: 10.1038/nrd3410.
(22) Shiota, T.; Nishikawa, S.-i.; Endo, T. Analyses of Protein-Protein Interactions by In Vivo Photocrosslinking in Budding Yeast. In Membrane Biogenesis: Methods and Protocols, Rapaport, D., Herrmann, J. M. Eds.; Humana Press, 2013; pp 207-217.
(23) Chin, J. W.; Schultz, P. G. In Vivo Photocrosslinking with Unnatural Amino Acid Mutagenesis. ChemBioChem 2002, 3 (11), 1135-1137. DOI: https://doi.org/10.1002/1439-7633(20021104)3:11<1135::AID-CBIC1135>3.0.CO; 2-M.
(24) Islam, M.; Kehoe, H. P.; Lissoos, J. B.; Huang, M.; Ghadban, C. E.; Berumen Sánchez, G.; Lane, H. Z.; Van Deventer, J. A. Chemical Diversification of Simple Synthetic Antibodies. ACS Chemical Biology 2021, 16 (2), 344-359. DOI: 10.1021/acschembio.0c00865.
(25) Preston, G. W.; Wilson, A. J. Photo-induced covalent cross-linking for the analysis of biomolecular interactions. Chemical Society Reviews 2013, 42 (8), 3289-3301, 10.1039/C3CS35459H. DOI: 10.1039/C3CS35459H.
(26) Coin, I. Application of non-canonical crosslinking amino acids to study protein-protein interactions in live cells. Current Opinion in Chemical Biology 2018, 46, 156-163. DOI: https://doi.org/10.1016/j.cbpa.2018.07.019.
(27) Lancia, J. K.; Nwokoye, A.; Dugan, A.; Joiner, C.; Pricer, R.; Mapp, A. K. Sequence context and crosslinking mechanism affect the efficiency of in vivo capture of a protein-protein interaction. Biopolymers 2014, 101 (4), 391-397. DOI: 10.1002/bip.22395 PubMed.
(28) Chen, H.; Wilson, J.; Ottinger, S.; Gan, Q.; Fan, C. Introducing noncanonical amino acids for studying and engineering bacterial microcompartments. Current Opinion in Microbiology 2021, 61, 67-72. DOI: https://doi.org/10.1016/j.mib.2021.03.004.
(29) Neumann, H.; Neumann-Staubitz, P.; Witte, A.; Summerer, D. Epigenetic chromatin modification by amber suppression technology. Current Opinion in Chemical Biology 2018, 45, 1-9. DOI: https://doi.org/10.1016/j.cbpa.2018.01.017.
(30) Chin, J. W.; Santoro, S. W.; Martin, A. B.; King, D. S.; Wang, L.; Schultz, P. G. Addition of p-Azido-l-phenylalanine to the Genetic Code of Escherichia coli. Journal of the American Chemical Society 2002, 124 (31), 9026-9027. DOI: 10.1021/ja027007w.
(31) Xiang, Z.; Lacey, V. K.; Ren, H.; Xu, J.; Burban, D. J.; Jennings, P. A.; Wang, L. Proximity-Enabled Protein Crosslinking through Genetically Encoding Haloalkane Unnatural Amino Acids. Angewandte Chemie International Edition 2014, 53 (8), 2190-2193. DOI: https://doi.org/10.1002/anie.201308794.
(32) Chen, X.-H.; Xiang, Z.; Hu, Y. S.; Lacey, V. K.; Cang, H.; Wang, L. Genetically Encoding an Electrophilic Amino Acid for Protein Stapling and Covalent Binding to Native Receptors. ACS Chemical Biology 2014, 9 (9), 1956-1961. DOI: 10.1021/cb500453a.
(33) Cheng, Y.; Wu, J.; Han, Y.; Xu, J.; Da, Y.; Zhao, Q.; Guo, G.; Zhou, Y.; Chen, Y.; Liu, J.; et al. A CDR-based approach to generate covalent inhibitory antibody for human rhinovirus protease. Bioorganic & Medicinal Chemistry 2021, 42, 116219. DOI: https://doi.org/10.1016/j.bmc.2021.116219.
(34) Xiang, Z.; Ren, H.; Hu, Y. S.; Coin, I.; Wei, J.; Cang, H.; Wang, L. Adding an unnatural covalent bond to proteins through proximity-enhanced bioreactivity. Nature Methods 2013, 10 (9), 885-888. DOI: 10.1038/nmeth.2595.
(35) Li, Q.; Chen, Q.; Klauser, P. C.; Li, M.; Zheng, F.; Wang, N.; Li, X.; Zhang, Q.; Fu, X.; Wang, Q.; et al. Developing Covalent Protein Drugs via Proximity-Enabled Reactive Therapeutics. Cell 2020, 182 (1), 85-97.e16. DOI: https://doi.org/10.1016/j.cell.2020.05.028.
(36) Yang, B.; Wu, H.; Schnier, P. D.; Liu, Y.; Liu, J.; Wang, N.; DeGrado, W. F.; Wang, L. Proximity-enhanced SuFEx chemical cross-linker for specific and multitargeting cross-linking mass spectrometry. Proceedings of the National Academy of Sciences 2018, 115 (44), 11162-11167. DOI: doi:10.1073/pnas.1813574115.
(37) Zhang, H.; Han, Y.; Yang, Y.; Lin, F.; Li, K.; Kong, L.; Liu, H.; Dang, Y.; Lin, J.; Chen, P. R. Covalently Engineered Nanobody Chimeras for Targeted Membrane Protein Degradation. Journal of the American Chemical Society 2021, 143 (40), 16377-16382. DOI: 10.1021/jacs.1c08521.
(38) Chen, S.; Lovell, S.; Lee, S.; Fellner, M.; Mace, P. D.; Bogyo, M. Identification of highly selective covalent inhibitors by phage display. Nature Biotechnology 2021, 39 (4), 490-498. DOI: 10.1038/s41587-020-0733-7.
(39) McCarthy, K. A.; Kelly, M. A.; Li, K.; Cambray, S.; Hosseini, A. S.; van Opijnen, T.; Gao, J. Phage Display of Dynamic Covalent Binding Motifs Enables Facile Development of Targeted Antibiotics. Journal of the American Chemical Society 2018, 140 (19), 6137-6145. DOI: 10.1021/jacs.8b02461.
(40) Liu, C. C.; Mack, A. V.; Brustad, E. M.; Mills, J. H.; Groff, D.; Smider, V. V.; Schultz, P. G. Evolution of Proteins with Genetically Encoded “Chemical Warheads”. Journal of the American Chemical Society 2009, 131 (28), 9616-9617. DOI: 10.1021/ja902985e.
(41) Garland, M.; Babin, B. M.; Miyashita, S.-I.; Loscher, S.; Shen, Y.; Dong, M.; Bogyo, M. Covalent Modifiers of Botulinum Neurotoxin Counteract Toxin Persistence. ACS Chemical Biology 2019, 14 (1), 76-87. DOI: 10.1021/acschembio.8b00937.
(42) Turner, L. D.; Nielsen, A. L.; Lin, L.; Pellett, S.; Sugane, T.; Olson, M. E.; Johnson, E. A.; Janda, K. D. Irreversible inhibition of BoNT/A protease: proximity-driven reactivity contingent upon a bifunctional approach. RSC Medicinal Chemistry 2021, 12 (6), 960-969, 10.1039/D1MD00089F. DOI: 10.1039/D1MD00089F.
(43) Lin, L.; Olson, M. E.; Sugane, T.; Turner, L. D.; Tararina, M. A.; Nielsen, A. L.; Kurbanov, E. K.; Pellett, S.; Johnson, E. A.; Cohen, S. M.; et al. Catch and Anchor Approach To Combat Both Toxicity and Longevity of Botulinum Toxin A. Journal of Medicinal Chemistry 2020, 63 (19), 11100-11120. DOI: 10.1021/acs.jmedchem.Oc01006.
(44) Lin, L.; Olson, M. E.; Eubanks, L. M.; Janda, K. D. Strategies to Counteract Botulinum Neurotoxin A: Nature's Deadliest Biomolecule. Acc Chem Res 2019, 52 (8), 2322-2331. DOI: 10.1021/acs.accounts.9b00261 PubMed.
(45) Miyashita, S.-I.; Zhang, J.; Zhang, S.; Shoemaker, C. B.; Dong, M. Delivery of single-domain antibodies into neurons using a chimeric toxin& #x2013; based platform is therapeutic in mouse models of botulism. Science Translational Medicine 2021, 13 (575), eaaz4197. DOI: doi:10.1126/scitranslmed.aaz4197.
(46) McNutt, P. M.; Vazquez-Cintron, E. J.; Tenezaca, L.; Ondeck, C. A.; Kelly, K. E.; Mangkhalakhili, M.; Machamer, J. B.; Angeles, C. A.; Glotfelty, E. J.; Cika, J.; et al. Neuronal delivery of antibodies has therapeutic effects in animal models of botulism. Science Translational Medicine 2021, 13 (575), eabd7789. DOI: doi:10.1 126/scitranslmed.abd7789.
(47) Lam, K.-h.; Tremblay, J. M.; Perry, K.; Ichtchenko, K.; Shoemaker, C. B.; Jin, R. Probing the structure and function of the protease domain of Botulinum neurotoxins using single-domain antibodies. PLOS Pathogens 2022, 18 (1), e1010169. DOI: 10.1371/journal.ppat.1010169.
(48) Lam, K.-h.; Tremblay, J. M.; Vazquez-Cintron, E.; Perry, K.; Ondeck, C.; Webb, R. P.; McNutt, P. M.; Shoemaker, C. B.; Jin, R. Structural Insights into Rational Design of Single-Domain Antibody-Based Antitoxins against Botulinum Neurotoxins. Cell Reports 2020, 30 (8), 2526-2539.e2526. DOI: https://doi.org/10.1016/j.celrep.2020.01.107.
(49) Stieglitz, J. T.; Kehoe, H. P.; Lei, M.; Van Deventer, J. A. A Robust and Quantitative Reporter System To Evaluate Noncanonical Amino Acid Incorporation in Yeast. ACS Synthetic Biology 2018, 7 (9), 2256-2269. DOI: 10.1021/acssynbio.8b00260.
(50) Van Deventer, J. A.; Le, D. N.; Zhao, J.; Kehoe, H. P.; Kelly, R. L. A platform for constructing, evaluating, and screening bioconjugates on the yeast surface. Protein Engineering, Design and Selection 2016, 29 (11), 485-494. DOI: 10.1093/protein/gzw029 (acccessed 2/1/2022).
(51) Potts, K. A.; Stieglitz, J. T.; Lei, M.; Van Deventer, J. A. Reporter system architecture affects measurements of noncanonical amino acid incorporation efficiency and fidelity. Molecular Systems Design & Engineering 2020, 5 (2), 573-588, 10.1039/C9ME00107G. DOI: 10.1039/C9ME00107G.
(52) Chao, G.; Lau, W. L.; Hackel, B. J.; Sazinsky, S. L.; Lippow, S. M.; Wittrup, K. D. Isolating and engineering human antibodies using yeast surface display. Nature Protocols 2006, 1 (2), 755-768. DOI: 10.1038/nprot.2006.94.
(53) Van Deventer, J. A.; Kelly, R. L.; Rajan, S.; Wittrup, K. D.; Sidhu, S. S. A switchable yeast display/secretion system. Protein Engineering, Design and Selection 2015, 28 (10), 317-325. DOI: 10.1093/protein/gzv043 (acccessed 2/2/2022).
(54) Rimbault, C.; Maruthi, K.; Breillat, C.; Genuer, C.; Crespillo, S.; Puente-Muñoz, V.; Chamma, I.; Gauthereau, I.; Antoine, S.; Thibaut, C.; et al. Engineering selective competitors for the discrimination of highly conserved protein-protein interaction modules. Nature Communications 2019, 10 (1), 4521. DOI: 10.1038/s41467-019-12528-4.
(55) Nicholson, S.; Wood, C.; Devy, L. Use of mmp-9 and mmp-12 binding proteins for the treatment and prevention of systemic sclerosis. WO 2010/045388 Å2, 2010.
(56) Wood, C. R. Combination treatments comprising protease binding proteins for inflammatory disorders. Canada CA 2741492 A1, 2009.
(57) Yang, B.; Tang, S.; Ma, C.; Li, S.-T.; Shao, G.-C.; Dang, B.; DeGrado, W. F.; Dong, M.-Q.; Wang, P. G.; Ding, S.; et al. Spontaneous and specific chemical cross-linking in live cells to capture and identify protein interactions. Nature Communications 2017, 8 (1), 2240. DOI: 10.1038/s41467-017-02409-z.
(58) Futran, A. S.; Kyin, S.; Shvartsman, S. Y.; Link, A. J. Mapping the binding interface of ERK and transcriptional repressor Capicua using photocrosslinking. Proceedings of the National Academy of Sciences 2015, 112 (28), 8590-8595. DOI: doi:10.1073/pnas.1501373112.
(59) Mishra, P. K.; Yoo, C.-M.; Hong, E.; Rhee, H. W. Photo-crosslinking: An Emerging Chemical Tool for Investigating Molecular Networks in Live Cells. ChemBioChem 2020, 21 (7), 924-932. DOI: https://doi.org/10.1002/cbic.201900600.
(60) Coin, I.; Katritch, V.; Sun, T.; Xiang, Z.; Siu, Fai Y.; Beyermann, M.; Stevens, Raymond C.; Wang, L. Genetically Encoded Chemical Probes in Cells Reveal the Binding Path of Urocortin-I to CRF Class B GPCR. Cell 2013, 155 (6), 1258-1269. DOI: https://doi.org/10.1016/j.cell.2013.11.008.
(61) Tang, H.; Dai, Z.; Qin, X.; Cai, W.; Hu, L.; Huang, Y.; Cao, W.; Yang, F.; Wang, C.; Liu, T.

Proteomic Identification of Protein Tyrosine Phosphatase and Substrate Interactions in Living Mammalian Cells by Genetic Encoding of Irreversible Enzyme Inhibitors. Journal of the American Chemical Society 2018, 140 (41), 13253-13259. DOI: 10.1021/jacs.8b06922.
(62) Wang, N.; Wang, L. Genetically encoding latent bioreactive amino acids and the development of covalent protein drugs. Current Opinion in Chemical Biology 2022, 66, 102106. DOI: https://doi.org/10.1016/j.cbpa.2021.102106.
(63) Shusta, E. V.; Raines, R. T.; Phückthun, A.; Wittrup, K. D. Increasing the secretory capacity of Saccharomyces cerevisiae for production of single-chain antibody fragments. Nature Biotechnology 1998, 16 (8), 773-777. DOI: 10.1038/nbt0898-773.
(64) Sikorski, R. S.; Hieter, P. A system of shuttle vectors and yeast host strains designed for efficient manipulation of DNA in Saccharomyces cerevisiae. Genetics 1989, 122 (1), 19-27. DOI: 10.1093/genetics/122.1.19 (acccessed 2/4/2022).
(65) Ahmed, S. A.; Byrne, M. P.; Jensen, M.; Hines, H. B.; Brueggemann, E.; Smith, L. A. Enzymatic Autocatalysis of Botulinum A Neurotoxin Light Chain. Journal of Protein Chemistry 2001, 20 (3), 221-231. DOI: 10.1023/A:1010952025677.
(66) Lindstedt, P. R.; Aprile, F. A.; Sormanni, P.; Rakoto, R.; Dobson, C. M.; Bernardes, G. J. L.; Vendruscolo, M. Systematic Activity Maturation of a Single-Domain Antibody with Non-canonical Amino Acids through Chemical Mutagenesis. Cell Chemical Biology 2021, 28 (1), 70-77.e75. DOI: https://doi.org/10.1016/j.chembiol.2020.11.002.
(67) Ekanayake, A. I.; Sobze, L.; Kelich, P.; Youk, J.; Bennett, N. J.; Mukherjee, R.; Bhardwaj, A.; Wuest, F.; Vukovic, L.; Derda, R. Genetically Encoded Fragment-Based Discovery from Phage-Displayed Macrocyclic Libraries with Genetically Encoded Unnatural Pharmacophores. Journal of the American Chemical Society 2021, 143 (14), 5497-5507. DOI: 10.1021/jacs.1c01186.
(68) Lewis, A. K.; Harthorn, A.; Johnson, S. M.; Lobb, R. R.; Hackel, B. J. Engineered protein-small molecule conjugates empower selective enzyme inhibition. Cell Chemical Biology 2021. DOI: https://doi.org/10.1016/j.chembiol.2021.07.013.
(69) Dotter, H.; Boll, M.; Eder, M.; Eder, A.-C. Library and post-translational modifications of peptide-based display systems. Biotechnology Advances 2021, 47, 107699. DOI: https://doi.org/10.1016/j.biotechadv.2021.107699.
(70) Rezhdo, A.; Islam, M.; Huang, M.; Van Deventer, J. A. Future prospects for noncanonical amino acids in biological therapeutics. Current Opinion in Biotechnology 2019, 60, 168-178. DOI: https://doi.org/10.1016/j.copbio.2019.02.020.
(71) Cao, L.; Wang, L. New covalent bonding ability for proteins. Protein Science 2022, 31 (2), 312-322. DOI: https://doi.org/10.1002/pro.4228.
(72) Liu, J.; Cao, L.; Klauser, P. C.; Cheng, R.; Berdan, V. Y.; Sun, W.; Wang, N.; Ghelichkhani, F.; Yu, B.; Rozovsky, S.; et al. A Genetically Encoded Fluorosulfonyloxybenzoyl-l-lysine for Expansive Covalent Bonding of Proteins via SuFEx Chemistry. Journal of the American Chemical Society 2021, 143 (27), 10341-10351. DOI: 10.1021/jacs.1c04259.
(73) McConnell, A. D.; Zhang, X.; Macomber, J. L.; Chau, B.; Sheffer, J. C.; Rahmanian, S.; Hare, E.; Spasojevic, V.; Horlick, R. A.; King, D. J.; Bowers, P. M. A general approach to antibody thermostabilization. mAbs 2014, 6 (5), 1274-1282. DOI: 10.4161/mabs.29680.(74) de Picciotto, S.; Dickson, P. M.; Traxlmayr, M. W.; Marques, B. S.; Socher, E.; Zhao, S.; Cheung, S.; Kiefer, J. D.; Wand, A. J.; Griffith, L. G.; Imperiali, B.; Wittrup, K. D. Design Principles for SuCESsFul Biosensors: Specific Fluorophore/Analyte Binding and Minimization of Fluorophore/Scaffold Interactions. Journal of Molecular Biology 2016, 428 (20), 4228-4241. DOI: 10.1016/j.jmb.2016.07.004.
(75) Sato, S.; Mimasu, S.; Sato, A.; Hino, N.; Sakamoto, K.; Umehara, T.; Yokoyama, S. Crystallographic Study of a Site-Specifically Cross-Linked Protein Complex with a Genetically Incorporated Photoreactive Amino Acid. Biochemistry 2011, 50 (2), 250-257. DOI: 10.1021/bi1016183.
(76) Kramer, J. R.; Deming, T. J. Reversible chemoselective tagging and functionalization of methionine containing peptides. Chemical Communications 2013, 49 (45), 5144-5146. DOI: 10.1039/C3CC42214C.
(77) Mons, E.; Jansen, I. D. C.; Loboda, J.; van Doodewaerd, B. R.; Hermans, J.; Verdoes, M.; van Boeckel, C. A. A.; van Veelen, P. A.; Turk, B.; Turk, D.; Ovaa, H. The Alkyne Moiety as a Latent Electrophile in Irreversible Covalent Small Molecule Inhibitors of Cathepsin K. Journal of the American Chemical Society 2019, 141 (8), 3507-3514. DOI: 10.1021/jacs.8b11027.
(78) Mons, E.; Kim, R. Q.; van Doodewaerd, B. R.; van Veelen, P. A.; Mulder, M. P. C.; Ovaa, H. Exploring the Versatility of the Covalent Thiol-Alkyne Reaction with Substituted Propargyl Warheads: A Deciding Role for the Cysteine Protease. Journal of the American Chemical Society 2021, 143 (17), 6423-6433. DOI: 10.1021/jacs.0c10513.
(79) Fang, K. Y.; Lieblich, S. A.; Tirrell, D. A. Incorporation of Non-Canonical Amino Acids into Proteins by Global Reassignment of Sense Codons. In Protein Scaffolds: Design, Synthesis, and Applications, Udit, A. K. Ed.; Springer New York, 2018; pp 173-186.
(80) Yang, A.; Ha, S.; Ahn, J.; Kim, R.; Kim, S.; Lee, Y.; Kim, J.; Söll, D.; Lee, H.-Y.; Park, H.-S. A chemical biology route to site-specific authentic protein modifications. Science 2016, 354 (6312), 623-626. DOI: doi:10.1126/science.aah4428.
(81) Stieglitz, J. T.; Van Deventer, J. A. High-Throughput Aminoacyl-tRNA Synthetase Engineering for Genetic Code Expansion in Yeast. ACS Synthetic Biology 2022, 11 (7), 2284-2299. DOI: 10.1021/acssynbio.1c00626.
(82) Segelke, B.; Knapp, M.; Kadkhodayan, S.; Balhorn, R.; Rupp, B. Crystal structure of Clostridium botulinum neurotoxin protease in a product-bound state: Evidence for noncanonical zinc protease activity. Proceedings of the National Academy of Sciences 2004, 101 (18), 6888-6893. DOI: 10.1073/pnas.0400584101.
(83) Cao, L.; Coventry, B.; Goreshnik, I.; Huang, B.; Sheffler, W.; Park, J. S.; Jude, K. M.; Markovic, I.; Kadam, R. U.; Verschueren, K. H. G.; Verstraete, K.; Walsh, S. T. R.; Bennett, N.; Phal, A.; Yang, A.; Kozodoy, L.; DeWitt, M.; Picton, L.; Miller, L.; Strauch, E.-M.; DeBouver, N. D.; Pires, A.; Bera, A. K.; Halabiya, S.; Hammerson, B.; Yang, W.; Bernard, S.; Stewart, L.; Wilson, I. A.; Ruohola-Baker, H.; Schlessinger, J.; Lee, S.; Savvides, S. N.; Garcia, K. C.; Baker, D. Design of protein-binding proteins from the target structure alone. Nature 2022, 605 (7910), 551-560. DOI: 10.1038/s41586-022-04654-9.
(84) Baek, M.; Baker, D. Deep learning and protein structure modeling. Nature Methods 2022, 19 (1), 13-14. DOI: 10.1038/s41592-021-01360-8.
(85) Xu, J.; Tack, D.; Hughes, R. A.; Ellington, A. D.; Gray, J. J. Structure-based non-canonical amino acid design to covalently crosslink an antibody-antigen complex. Journal of Structural Biology 2014, 185 (2), 215-222. DOI: 10.1016/j.jsb.2013.05.003.
(86) Zhang, M.; McEwen, J. M.; Sjoblom, N. M.; Kotewicz, K. M.; Isberg, R. R.; Scheck, R. A. Members of the Legionella pneumophila Sde family target tyrosine residues for phosphoribosyl-linked ubiquitination. RSC Chemical Biology 2021, 2 (5), 1509-1519. DOI: 10.1039/D1CB00088H.
(87) Liu, J.; Cao, L.; Klauser, P. C.; Cheng, R.; Berdan, V. Y.; Sun, W.; Wang, N.; Ghelichkhani, F.; Yu, B.; Rozovsky, S.; Wang, L. A Genetically Encoded Fluorosulfonyloxybenzoyl-L-lysine for Expansive Covalent Bonding of Proteins via SuFEx Chemistry. Journal of the American Chemical Society 2021, 143 (27), 10341-10351. DOI: 10.1021/jacs.1c04259.
(88) Hershman, R. L.; Rezhdo, A.; Stieglitz, J. T.; Van Deventer, J. A. Engineering Proteins Containing Noncanonical Amino Acids on the Yeast Surface. In Yeast Surface Display, Traxlmayr, M. W. Ed.; Springer US, 2022; pp 491-559.
(89) Stieglitz, J. T.; Van Deventer, J. A. Incorporating, Quantifying, and Leveraging Noncanonical Amino Acids in Yeast. In Biomedical Engineering Technologies: Volume 2, Rasooly, A., Baker, H., Ossandon, M. R. Eds.; Springer US, 2022; pp 377-432.
(90) Stieglitz, J. T.; Lahiri, P.; Stout, M. I.; Van Deventer, J. A. Exploration of Methanomethylophilus alvus Pyrrolysyl-tRNA Synthetase Activity in Yeast. ACS Synthetic Biology 2022, 11 (5), 1824-1834. DOI: 10.1021/acssynbio.2c00001.
(91) Podracky, C. J.; An, C.; DeSousa, A.; Dorr, B. M.; Walsh, D. M.; Liu, D. R. Laboratory evolution of a sortase enzyme that modifies amyloid-β protein. Nature Chemical Biology 2021, 17 (3), 317-325. DOI: 10.1038/s41589-020-00706-1.
(92) Shu, X.; Asghar, S.; Yang, F.; Li, S.-T.; Wu, H.; Yang, B. Uncover New Reactivity of Genetically Encoded Alkyl Bromide Non-Canonical Amino Acids. Frontiers in Chemistry 2022, 10. DOI: 10.3389/fchem.2022.815991.

What is claimed is:

1. A single domain antibody (sdAb) comprising at least one reactive, non-canonical amino acid (ncAA) that is capable of forming a covalent interaction between the ncAA of the sdAb and a target antigen bound by the sdAb.

2. The sdAb of claim 1, wherein the sdAb specifically binds to Botulinum neurotoxin (BoNT).

3. The single domain antibody (sdAb) of claim 2, wherein Botulinum neurotoxin is the target antigen bound by the sdAb.

4. The sdAb of claim 1, wherein the ncAA is photoreactive or spontaneously reactive.

5. The sdAb of claim 4, wherein the ncAA comprises 4-azidophenylalanine (AzF) or O-(2-bromoethyl)tyrosine (ObeY), respectively.

6. The sdAb of claim 2, wherein the BoNT is Botulinum neurotoxin light chain A (LC/A).

7. The sdAb of claim 5, wherein the at least one ncAA is AzF which is incorporated in the sdAb amino acid sequence at one or more amino acid positions photo-crosslinkable to the target antigen LC/A selected from Q1, Y32, Y37, R35, F29, F52, N54, S56, Y101, L101, Q44, K64, R107, M104, R101, W104, or Y111.

8. The sdAb of claim 7, wherein ncAAs are incorporated at the following amino acid positions:
(i) Q1, Y37, N54, Y101, R107 in sdAb JPU-A5 comprising the amino acid sequence
(SEQ ID NO: 64)
QVQLVETGGGLVQAGGSLRLSCTASGADFSFYAMGWYRQTPGNSRELVA
VMNLNGVISYGDSARGRFDISRDGTKNIVFLQMNSLKPEDTGVYYCNGM
RLYTRGSVRHPESWGQGIQVTVSS;
(ii) Y32, R35, Y37, L101, M104 in sdAb JPU-C1 comprising the amino acid sequence
(SEQ ID NO: 65)
QVQLAESGGGLVQPGGSLRLSCAASGFTFNRYVIRWYRQAPGKERELVA
GISRSGDSGRYVDSVKGRFTISRDNDKNMAYLQMSSLKPDDTAVYYCSA
LNLEDMEYWGQGTQVTVSS;
(iii) Q1, F29, Y32, Q44, R101, Y111 in sdAb JPU-C10 comprising the amino acid sequence
(SEQ ID NO: 66)
QLQLVESGGGLVQPGGSLRLSCAASGNIFSIYYMGWYRQAPGKQREMVA
IINSNGITNYGDFVKGRFTISRDNAENSAYLQMNNLTPEDTAVYYCNAG
KLRRTTGWGLDDYWGQGTQVTVSS;
and
(iv) Q1, F52, 556, K64, W104 sdAb JDQ-H7 comprising the amino acid sequence
(SEQ ID NO: 67)
QVQLVESGGGLVQVGGSLRLSCVVSGSDISGIAMGWYRQAPGKRREMVA
DIFSGGSTDYAGSVKGRFTISRDNAKKTSYLQMNNVKPEDTGVYYCRLY
GSGDYWGQGTQVTVSS.

9. The sdAb of claim 8, wherein the at least one ncAA is ObeY which is incorporated in the sdAb amino acid sequence at one or more amino acid positions spontaneously crosslinkable to the target antigen LC/A selected from R31, R53, D56, 557, R59, N100, E102, D103, and Y106.

10. An isolated polynucleotide encoding the sdAb of claim 1.

11. A vector comprising the isolated polynucleotide of claim 10.

12. A cell comprising the vector of claim 11.

13. A composition comprising the sdAb of claim 1.

14. A method of treating or preventing intoxication by a Botulinum toxin, the method comprising administering to a subject in need thereof an effective amount of the sdAb of claim 1 or a pharmaceutically acceptable composition thereof.

15. A method of neutralizing toxicity of a Botulinum toxin, the method comprising administering to a subject in need thereof an effective amount of the sdAb of claim 1 or a pharmaceutically acceptable composition thereof.

16. A method of reducing, ameliorating, abating, abrogating, or eradicating intoxication of a cell by a Botulinum neurotoxin, the method comprising contacting the cell with an effective amount of the sdAb of claim 1; or a pharmaceutically acceptable composition thereof, thereby reducing, ameliorating, abating, abrogating, or eradicating intoxication of the cell by the Botulinum neurotoxin.

17. A kit comprising the sdAb of claim 1 or a pharmaceutically acceptable composition thereof, for treating or protecting against disease or intoxication and/or the symptoms thereof caused by Botulinum neurotoxin protease; and optionally comprising instructions for use.

18. A method of selecting single domain antibody (sdAb) which covalently crosslinks with a target antigen, comprising:
expressing on yeast cells a detectable sdAb comprising at least one reactive non-canonical amino acid (ncAA) capable of forming a covalent interaction with a target antigen bound by the sdAb;
contacting the sdAb with the target antigen;
activating the ncAA in the sdAb to crosslink the sdAb and the target antigen, thereby forming a covalent complex comprising the sdAb and the target antigen;
denaturing the complex to remove unbound target antigen; and
detecting and selecting the sdAb.

19. A method of detecting a target antigen, comprising:
expressing on yeast cells a detectable sdAb comprising at least one reactive non-canonical amino acid (ncAA) capable of crosslinking to a target antigen;
contacting a sample with the yeast cells;
activating the ncAA in the sdAb to crosslink the sdAb with the target antigen if present in the sample, thereby forming a covalent complex comprising the sdAb and the target antigen;
denaturing the complex to remove unbound target antigen; and
detecting the target antigen crosslinked to the sdAb, if present in the sample.

20. Use of yeast display expression system for expressing and selecting reactive, non-canonical amino acid (ncAA)-containing single-domain antibodies (sdAbs) that bind and crosslink to a target antigen.
